(19) United States
(12) Patent Application Publication
(10) Pub. No.: US 2025/0108218 A1
(43) Pub. Date: Apr. 3, 2025

(54) IMPLANTABLE STIMULATION ELEMENTS AND METHODS FOR SLEEP DISORDERED BREATHING (SDB) CARE

(71) Applicant: INSPIRE MEDICAL SYSTEMS, INC., Golden Valley, MN (US)

(72) Inventors: Kevin Verzal, Lino Lakes, MN (US); John Rondoni, Plymouth, MN (US); Wondimeneh Tesfayesus, New Brighton, MN (US)

(73) Assignee: INSPIRE MEDICAL SYSTEMS, INC., Golden Valley, MN (US)

(21) Appl. No.: 18/834,549

(22) PCT Filed: Feb. 1, 2023
(86) PCT No.: PCT/US2023/012105

§ 371 Date: Jul. 30, 2024

Related U.S. Application Data
(60) Provisional application No. 63/306,033, filed on Feb. 2, 2022.

(51) Int. Cl. A61N 1/36; A61N 1/05

(52) U.S. Cl. CPC A61N 1/3611; A61N 1/0553; A61N 1/0558; A61N 1/0556

(57) ABSTRACT

An example devices comprises at least one implantable stimulation element to be in stimulating relation to at least one hypoglossal nerve portion, and optionally comprising a control portion configured to stimulate, via the at least one implantable stimulation element, the at least one hypoglossal nerve portion.

### BACKGROUND

Sleep disordered breathing, such as obstructive sleep apnea, may cause significant health problems and is common among the adult population. Some forms of treatment of sleep disordered breathing may include electrical stimulation of nerves and/or muscles relating to upper airway patency.

### BRIEF DESCRIPTION OF THE DRAWINGS

FIG. 1A is a block diagram schematically representing an example method and/or example device for treating sleep disordered breathing, including stimulating airway patency-related tissue (e.g. upper airway patency-related tissue).

FIG. 1B is a block diagram schematically representing an example method and/or example device for treating sleep disordered breathing (e.g. obstructive sleep apnea), including a sensing element, a pulse generator, and/or a stimulation element.

FIG. 1C is a block diagram schematically representing an example method and/or example device for treating sleep disordered breathing (e.g. obstructive sleep apnea), including an anchor portion, a stimulation portion, and a strain relief portion.

FIG. 2A is a diagram schematically representing an example method and/or example device for implantation within a head-neck region of a patient's body via an implant-access incision.

FIG. 2B is a diagram schematically representing further details of example patient anatomy.

FIGS. 2C-2D are diagrams schematically representing further example implementations of, and/or consistent with, the implant-access incision 609A of FIG. 2A.

FIG. 3 is a diagram schematically representing an example method and/or example device for treating sleep disordered breathing as implanted within a patient's body.

FIG. 4 is a diagram including a side view schematically representing an example stimulation lead with connection features.

FIGS. 5A-5B are diagrams including a side view schematically representing example anchor elements.

FIGS. 6A, 6B, and 7 are diagrams including a side view schematically representing example stimulation leads with bifurcation features and/or related delivery tools.

FIG. 8A is a diagram including a bottom view of a patient's submandibular region and schematically representing an example method and/or example device for treating sleep disordered breathing including a pair of implanted stimulation elements.

FIG. 8AA is a top plan view of a stimulation element indicating dimensional parameters.

FIG. 8B is a sectional view as taken along lines 8B-8B of FIG. 8A schematically representing a stimulation element, including individually addressable contact electrodes, in stimulating relation to target tissues.

FIG. 8C is a diagram including a side view schematically representing an example stimulation element including arms configurable into different angular orientations relative to each other.

FIG. 8D is a diagram a side sectional view schematically representing an example method and/or example device including electrodes of an example stimulation element in stimulating relation relative to a nerve portion(s), muscle portion(s), combinations of nerve portion(s) and muscle portion(s), neuromuscular junctions of nerve portion(s) and muscle portion(s), and/or combinations thereof.

FIGS. 8E-8H are diagrams including a side view schematically representing example devices and/or example methods of implantation, including access and delivery tools for stimulation elements, some of which include anchor elements.

FIG. 8I is a diagram including a side view schematically representing an example method and/or example device including a stimulation element implanted in stimulating relation to nerve portion(s) at least partially via a superior orientation and anterior orientation.

FIG. 8J is a diagram including a front view schematically representing an example method and/or example device including a pair of stimulation elements implanted in stimulating relation to nerve portion(s) at least partially oriented in a superior orientation relative to a mandibular plane.

FIG. 8K is a diagram including a bottom view of a patient's submandibular region and schematically representing an example method and/or example device for treating sleep disordered breathing including a pair of implanted stimulation elements.

FIG. 8L is a diagram including a side view schematically representing an example method and/or example device including a stimulation element implanted in stimulating relation to target tissues including at least nerve portion(s) at least partially via a superior orientation and a posterior orientation.

FIG. 9A is a diagram schematically representing an example method and/or example device for treating sleep disordered breathing via a stimulation portion including a connected array of stimulation elements, each of which are independently positionable into stimulating relation to at least different portions of upper airway patency-related tissues, including nerve portion(s).

FIG. 9B is a sectional view schematically representing an example lead segment including multiple signal electrical conductors extending within and through at least a portion of the lead segment.

FIG. 9C is a top plan view schematically representing an example portion of a lead body for strain relief and/or other purposes.

Each of FIGS. 10A-10B are a diagram schematically representing an example method and/or example device including a connected array of stimulation elements each of which are independently positionable into close proximity with, and stimulating relation to, at least different target upper airway patency-related nerve portion(s), muscle portion(s), combinations of such nerve and muscle portions, neuromuscular junctions, and/or combinations thereof.

FIG. 10AA is a diagram including a perspective view schematically representing an example stimulation element in association with the example method and/or example device of FIGS. 10A-10B.

FIG. 11A is a diagram including a side view schematically representing an example device including a connected array of stimulation elements with an anchor structure interposed between at least some of the adjacent stimulation elements.

FIG. 11B is a diagram including a side view schematically representing an example device including a connected array of stimulation elements with anchor portions located adjacent opposite ends of at least some stimulation elements.

FIGS. 11C-11D are diagrams each including a top view schematically representing example stimulation elements.

FIG. 12A is a top plan view, and FIG. 12B is a diagram including a patient's body, which together schematically represent an example method and/or example device for treating sleep disordered breathing via a stimulation portion including a connected array of stimulation elements, each of which are independently positionable into stimulating relation to at least different portions of upper airway patency-related tissues, including nerve portion(s).

FIG. 13A is a diagram including a side view schematically representing a stimulation lead comprising an example stimulation portion including an anchor structure.

FIGS. 13B-13C are each a sectional view schematically representing an example implementation of the example stimulation portion of FIG. 13A.

FIGS. 14A-14C are each a diagram including a side view schematically representing an example portion of a stimulation lead including an anchor structure.

FIG. 14D is a sectional view schematically representing an example stimulation element including an anchor structure.

FIGS. 15A-15B are diagrams including a top view and a side view, respectively, schematically representing example anchor structures.

FIG. 15C is a sectional view schematically representing a portion of an example stimulation lead including tines as an anchor structure.

FIGS. 15D-15H are each a diagram including a side plan view schematically representing an example stimulation element including example tines as part of an anchor structure.

FIG. 16A is a diagram schematically representing an example method and/or example device for treating sleep disordered breathing and including a pair of independently positionable, elongate stimulation elements each including an axial array of electrodes.

FIG. 16B is a diagram schematically representing a relative degree of angular orientation of the pair of stimulation elements of FIG. 16A relative to each other.

FIG. 16C is a diagram including a bottom view of at least a patient's submandibular region and schematically representing an example method and/or example device for treating sleep disordered breathing including a pair of implanted stimulation elements in stimulating relation to an upper airway patency-related target tissues.

FIG. 16D is a diagram including a side view schematically representing an example method and/or example device including a stimulation element implanted in stimulating relation to upper airway patency-related target tissue(s) with at least a portion of the stimulation element extending in a superior orientation relative to a mandibular plane.

FIG. 16E is a diagram including a sectional view as taken along lines 16E-16E in FIG. 16D and schematically representing an example stimulation element.

FIG. 16F is a diagram including a side view like FIG. 16D, except schematically representing an access pathway for an example stimulation element.

FIG. 16G is a diagram including a front view schematically representing an example method and/or example device including a pair of stimulation elements implanted in stimulating relation to target tissues, including nerve portion(s), in at least a superior orientation relative to a mandibular plane.

FIG. 16H is a diagram including a bottom view of at least a patient's submandibular region and schematically representing an example method and/or example device for treating sleep disordered breathing including a pair of implanted stimulation elements in stimulating relation to target tissues including nerve portion(s).

FIG. 16I is a diagram including a side view schematically representing an example method and/or example device including a stimulation element implanted in stimulating relation to upper airway patency-related tissue(s) with at least a portion of the stimulation element extending in a superior orientation relative to a mandibular plane.

FIGS. 16J-16K are each a diagram including a side plan view schematically representing an example stimulation element including an anchor structure.

FIG. 17A is a diagram schematically representing an example method and/or example device for treating sleep disordered breathing and including a pair of independently positionable, elongate stimulation elements each including an axial array of electrodes and extending from a common carrier portion.

FIG. 17B is a diagram schematically representing a relative degree of angular orientation of the pair of stimulation elements of FIG. 17A relative to each other.

FIG. 18A is a diagram schematically representing an example method and/or example device for treating sleep disordered breathing and including a pair of example independently positionable, paddle-style stimulation elements each including an array of electrodes, with the stimulation elements juxtaposed relative to patient anatomy including target nerves.

FIG. 18B is a top plan view, and FIG. 18C is a side sectional view, schematically representing an example paddle-style element.

FIGS. 19A, 19C, and 19D are each a diagram including a plan view (side, top, side, respectively) schematically representing an example device (and/or example method) including the paddle-style stimulation elements of FIG. 18A with a first stimulation element and second stimulation element rotated according to a first axis, a second axis, and a third axis relative to each other, respectively.

FIG. 19B is diagram including an end view schematically representing relative rotation of the first and second stimulation elements.

FIG. 19E is a diagram like FIG. 19D and further schematically representing the example stimulation elements in stimulating relation to upper airway patency-related tissue(s).

FIGS. 20A, 21A, and 21B are each a diagram including a plan view (top, side, top, respectively) schematically representing an example device including the paddle-style stimulation elements of FIG. 18A with a first stimulation element and second stimulation element translated along or parallel to a first, a second, and a third axis relative to each other, respectively.

FIG. 20B is a diagram schematically representing a length of a fully extended connector portion, respectively, of example device (and/or example method).

FIGS. 22A-22AA are diagrams schematically representing an example device and/or an example method including a pair of independently positionable paddle-style stimulation elements like that of FIG. 18A including a more proximal bifurcation portion from which independent flexible connection segments extend.

FIG. 22B is a diagram schematically representing an example device and/or an example method including a pair of independently positionable, paddle-style stimulation elements like that of FIG. 18A including asymmetrically arranged independent flexible connection segments.

FIGS. 22C and 22D are each a diagram schematically representing example stimulation elements including example split contact electrodes and an example grid of contact electrodes, respectively.

FIGS. 23A-23C are each a diagram including a sectional view of an example paddle-style stimulation element including example anchor portions.

FIGS. 24A, 24D and FIGS. 24B-24C are diagrams including a top plan view and bottom plan view (respectively) of an example paddle-style stimulation element including example anchor portions.

FIG. 24E is a diagram including a side plan view of an example connector portion for extension between respective first and second paddle-style stimulation elements with the connector portion including a helical-shaped anchor structure.

FIG. 25A is a diagram schematically representing an example method and/or example device for treating sleep disordered breathing and including an example paddle-style stimulation portion including an array of electrodes.

FIG. 25B is a top plan view, and FIG. 25C is a side sectional view, schematically representing an electrode portion of the example paddle-style stimulation portion.

FIG. 25D is a sectional view as taken along lines 25D-25D of FIG. 25C.

FIG. 25DD is a sectional view schematically representing an example stimulation portion having a circular cross-sectional shape.

FIG. 25E is a side view schematically representing an example device and/or example method including an example paddle-style stimulation portion in a bent configuration with two arms at angle relative to each other, and FIG. 25F is a similar schematic representation with the stimulation portion deployed relative to upper airway patency-related tissues.

FIG. 25G is a side view schematically representing an example device and/or example method including an example paddle-style stimulation portion in a bent configuration with two arms at angle relative to each other.

FIG. 25H is a side view schematically representing an example device and/or example method including an example paddle-style stimulation portion and anchor structures integrated with the stimulation portion.

FIGS. 251, 25J, and 25K are each a top plan view schematically representing an example device including an example paddle-style stimulation portion with a particular pattern of spaced apart electrodes.

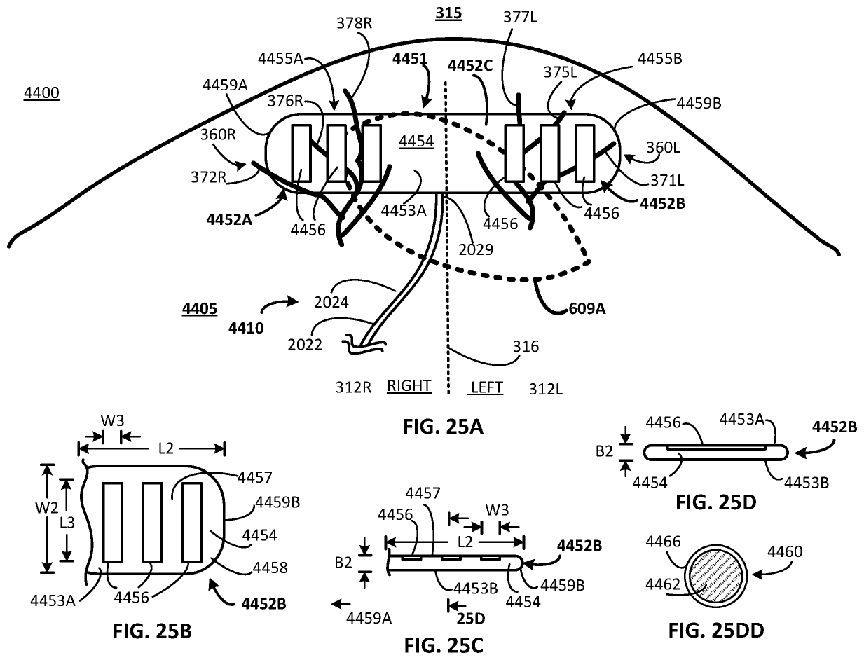

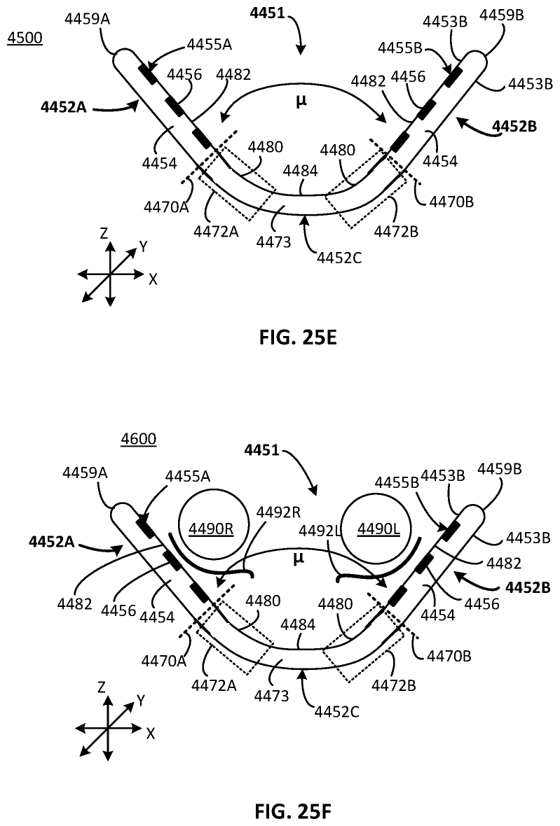

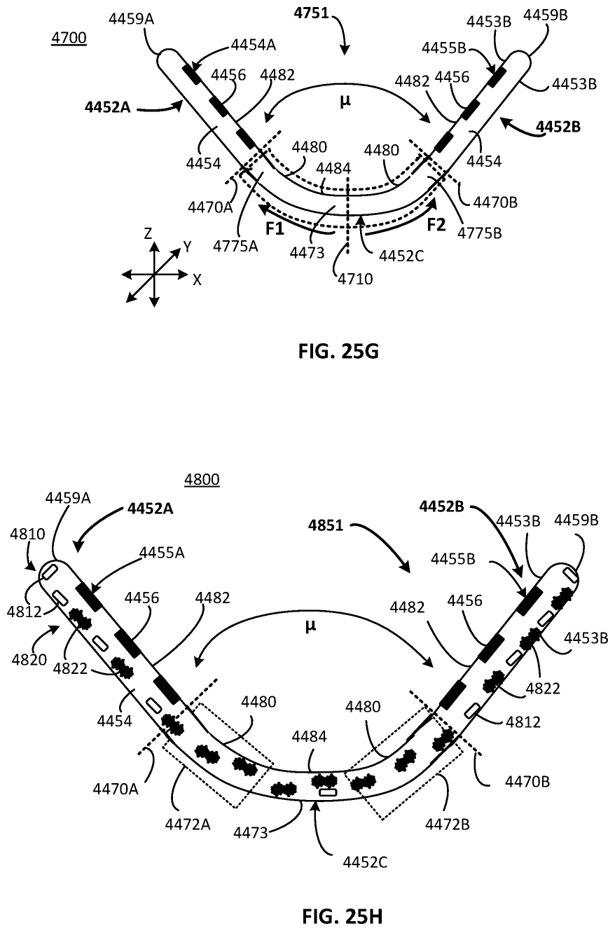

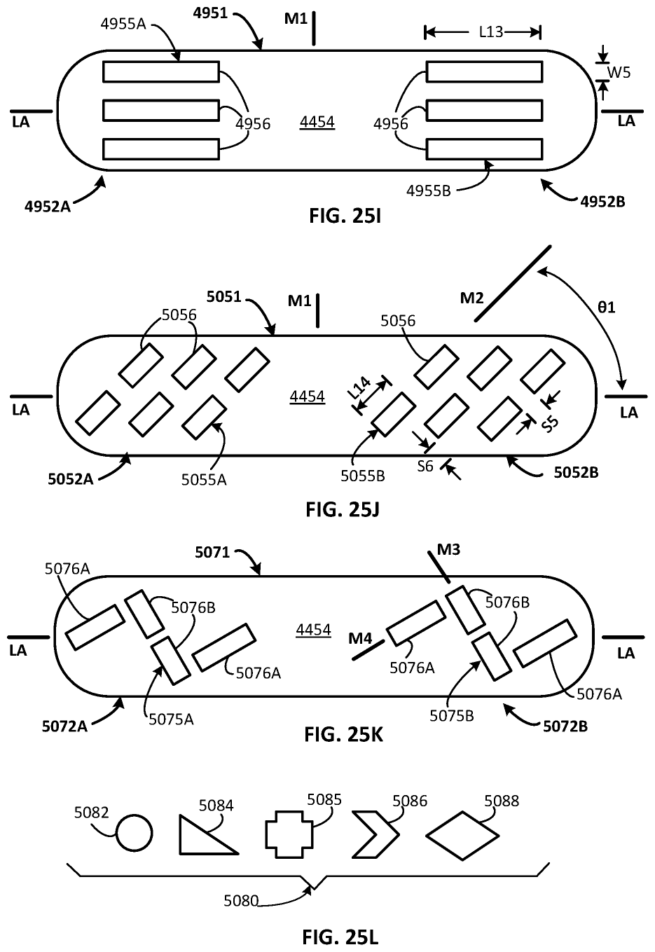

FIG. 25L is a diagram schematically representing an array of differently shaped electrodes for deployment on a stimulation element.

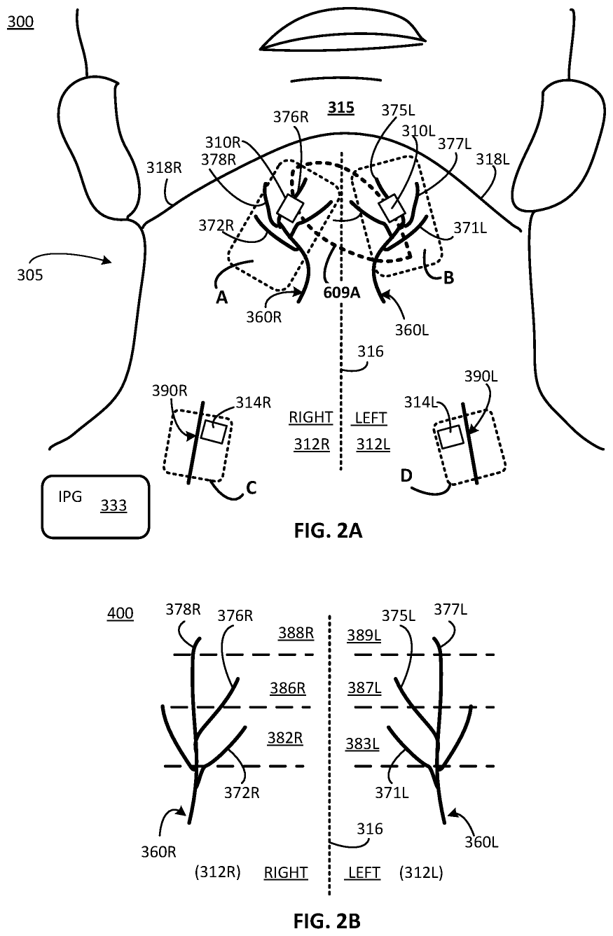

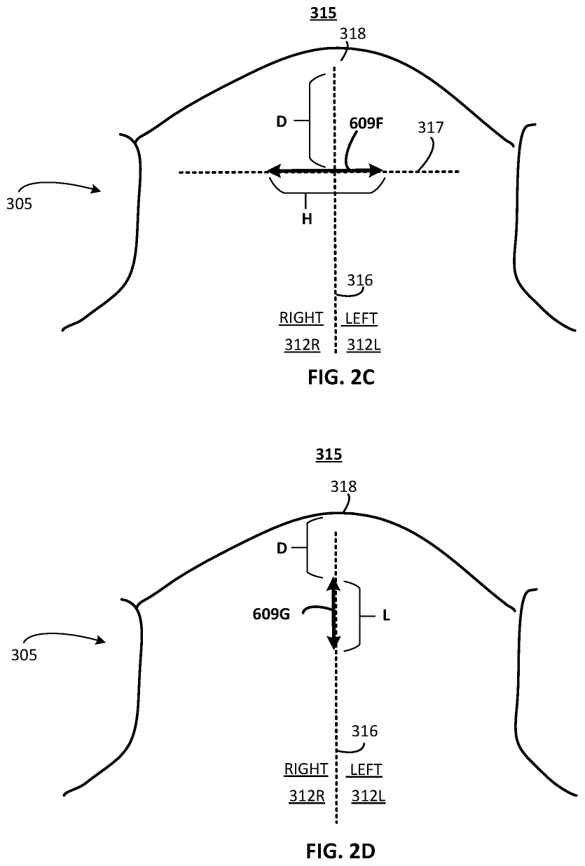

FIG. 26 is a diagram schematically representing patient anatomy and an example device and/or example method like that of FIG. 2A, while explicitly illustrating sensing elements.

FIG. 27 is a diagram schematically representing an example device including an implantable medical device and a patient remote control which can communicate with each other.

FIG. 28 is a diagram schematically representing patient anatomy and an example device and/or example method for stimulating an infrahyoid muscle (IHM)-innervating nerve and/or hypoglossal nerve.

FIG. 29A is a block diagram schematically representing an example care engine.

FIGS. 29B-29E are block diagrams schematically representing example control portions, a user interface, and associated devices.

FIG. 30 is a block diagram schematically representing a care engine of a control portion.

FIG. 31 is a diagram schematically representing a patient's body, implantable components, and/or external elements of an example device and/or for use in an example method.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure.

The following detailed description, therefore, is not to be taken in a limiting sense.

It is to be understood that features of the various examples described herein may be combined, in part or whole, with each other, unless specifically noted otherwise.

At least some examples of the present disclosure are directed to example devices for, and/or example methods of, therapy for sleep disordered breathing (SDB).

In some example methods comprise implanting a stimulation element(s) via an implant-access incision in a submental region (e.g. under the chin) of a patient's head-and-neck region. In some examples, the implant-access incision is located along or in close proximity to a sagittal midline of a patient's body, and as such may sometimes be referred to as a midline implant-access incision. Via such access, in some examples the stimulation element may be implanted at more distal portions of a hypoglossal nerve, which may (among other aspects) facilitate positioning the stimulation element in stimulating relation to target tissues most directly related to causing tongue protrusion and/or stiffening upper airway musculature to increase or maintain upper airway patency. The target tissue may comprise nerve portions, muscle portions, combinations of nerve portions and muscle portions, neuromuscular junctions of nerve portions and muscle portions, and/or combinations thereof. In some examples, the target tissues may comprise a first nerve portion innervating a genioglossus oblique (GGo) muscle portion, the genioglossus oblique (GGo) muscle portion, and/or a neuromuscular junction of the first nerve portion and the genioglossus oblique (GGo) muscle portion. In some examples, the target tissues may comprise a second nerve portion innervating a genioglossus horizontal (GGh) muscle portion, the genioglossus horizontal (GGh) muscle portion, and/or a neuromuscular junction of the second nerve portion and the genioglossus horizontal oblique (GGh) muscle portion. In some examples, the target tissues may comprise a third nerve portion innervating a geniohyoid (GH) muscle portion, the geniohyoid (GH) muscle portion, and/or a neuromuscular junction of the third nerve portion and the geniohyoid (GH) muscle portion. In some examples, the target tissues may comprise a nerve portion innervating the intrinsic muscles of the tongue, the intrinsic muscle portions of the tongue, and/or neuromuscular junctions of the nerve portion and intrinsic tongue muscles portion.

Moreover, the midline implant-access incision may enable direct visualization (or near direct visualization) of target tissues at which a stimulation element may be implanted in stimulating relation to such target tissues. This arrangement may ease delivery of the stimulation elements to the target locations, increase accuracy and precision in implementing placement at such locations, etc.

In some examples, the sleep disordered breathing may comprise obstructive sleep apnea, while in some examples, the sleep disordered breathing may comprise multiple-type sleep apneas including obstructive sleep apnea and/or central sleep apnea. In yet other examples, the sleep disordered breathing may comprise complex sleep apnea.

Moreover, at least some of the general principles associated with the example arrangements of the present disclosure relating to sleep disordered breathing may be applied in other areas of a patient's body to treat conditions other than sleep disordered breathing. For instance, at least some aspects of the example arrangements of the present disclosure may be deployed within a pelvic region to treat urinary and/or fecal incontinence or other disorders, such as via stimulating the pudendal nerve and/or other pelvic nerves, which may cause contraction of the external urinary sphincter, external anal sphincter, and/or pelvic muscles.

Other body regions and/or disorders also may be suitable candidates for an example arrangements in which multiple target tissues are available to be stimulated to treat one type or class of physiologic conditions. It will be further understood that example sensing arrangements of the present disclosure (for sensing physiologic data relative to the condition of interest) may be deployed in association with the various example stimulation arrangements.

In some examples, at least one stimulation element may be implanted on just one side of the patient's body for unilateral stimulation.

In some examples, a first stimulation element may be implanted on a first side of the patient's body and a second stimulation element may be implanted on an opposite second side of the patient's body. In some such examples, both of the first and second stimulation elements may be used for applying bilateral stimulation, which may be alternating, simultaneous, etc. However, in some such examples, just one of the first and second stimulation elements may be used such that unilateral stimulation may be applied in some instances.

In some examples, whether implanted on just one side or on both sides of the patient's body, each implanted stimulation element may comprise multiple spaced apart electrodes and/or multiple implanted stimulation elements (each including at least one electrode) may be spaced apart within the patient's body.

Stimulation vectors may be applied among the multiple electrodes (or multiple stimulation elements) on just one side of the patient's body, on both sides of the patient's body (e.g. bilateral stimulation), and/or from one side to another side of the patient's body (e.g. cross-lateral stimulation).

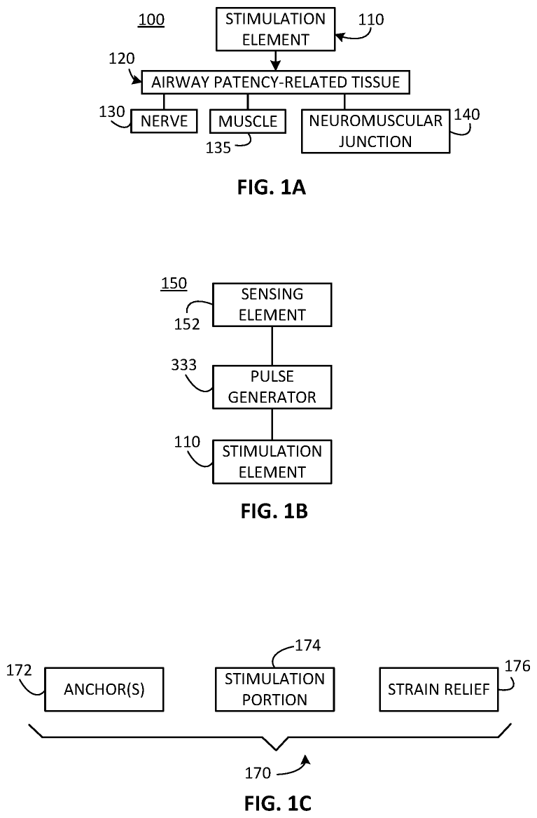

These examples, and additional examples, are described in association with at least FIGS. 1A-31.

As shown in FIG. 1A, some example methods 100 (and/or example devices) may comprise stimulating, via at least one stimulation element 110, at least one airway patency-related tissue 120, which may comprise nerve(s) 130, muscle(s) 135, and/or neuromuscular junction(s) 140. In some examples, the tissue 120 may sometimes be referred to as target tissue, which may encompass one of, or a combination of, the target nerve(s) 130, target muscle(s) 135, and/or target neuromuscular junction(s) 140.

In some examples, the target tissue 120 may comprise upper airway patency-related tissue 120. Accordingly, in some examples, nerve 130 may comprise a hypoglossal nerve, which is further represented as 360R, 360L in at least FIG. 2A. In some such examples, the target nerve(s) 130 may comprise at least protrusor-related branches of the hypoglossal nerve. As further described later throughout various examples including FIG. 2A, in some examples nerve(s) 130 may comprise nerves in addition to, or instead of, the hypoglossal nerve, such as but not limited to infrahyoid muscle (IHM)-innervating nerves 390R, 390L.

In some examples, an IHM-innervating nerve may comprise a nerve or nerve branch which innervates (directly or indirectly) at least one infrahyoid muscle, which may sometimes be referred to as an infrahyoid strap muscle. In some examples, IHM-innervating nerves/nerve branches extend from (e.g. originate) from a nerve loop called the ansa cervicalis (AC) or the “AC loop nerve”, which stems from the cervical plexus, e.g. extending from cranial nerves C1-C3. Accordingly, in some examples, at least some IHM-innervating nerves may correspond to an AC-related nerve in the sense that such nerves/nerve branches (e.g. IHM-innervating nerves) do not form the AC loop nerve but extend from the AC loop nerve. At least because the AC loop nerve is the origin for some nerves which innervate muscles other than the infrahyoid muscles, some AC-related nerves do not comprise IHM-innervating nerves. Moreover, it will be understood that in some examples, stimulation applied to a portion of the AC loop nerve (and/or to nerves from which the AC loop nerve originates) may activate IHM-innervating nerves/nerve branches, which extend from the AC loop nerve. However, implementing stimulation (e.g. to influence upper airway patency) occurring at more proximal locations, such as along the superior root of the AC loop nerve, may be more complex because of the number/type of different nerves and number/type of different muscles innervated via a superior root of the AC loop nerve such that selective activation of a particular infrahyoid muscle (via stimulation along the superior root) may be quite challenging in some circumstances.

Further details regarding these anatomical structures and relationships are described later in association with at least FIG. 28.

Meanwhile, in some examples the target muscle(s) 135 may comprise a genioglossus muscle, which is innervated by the hypoglossal nerve. In particular, the target muscle(s) 135 may comprise at least the protrusor muscles of the genioglossus muscles, which causes tongue protrusion, which in turn may result in maintaining or increasing upper airway patency. Moreover, as further described later throughout various examples, the target muscle(s) 135 may comprise upper airway musculature in addition to, or instead of, the genioglossus muscle, such as but not limited to such as the omohyoid, sternothyroid, sternohyoid muscle groups innervated by the IHM-innervating nerve. Among other effects, stimulation of such IHM-innervating nerves and/or muscles act to bring the larynx inferiorly, which may increase upper airway patency.

In some examples, the neuromuscular junction(s) 140 may comprise a neuromuscular junction between portions of the hypoglossal nerve and portions of muscles (e.g. genioglossus) innervated by those nerve portions. In addition to or instead of involving the hypoglossal nerve and genioglossus muscle (and associated neuromuscular junctions), in some examples the neuromuscular junction(s) 140 may comprise a neuromuscular junction (or junctions) between portions of an IHM-innervating nerve/nerve branch and portions of muscles innervated by those nerve portions.

It will be understood that the stimulation element 110 may comprise a single stimulation element or multiple stimulation elements, and that each stimulation element 110 may comprise a single or multiple contact electrodes for delivering a stimulation signal. Any given stimulation element 110 may be implanted in a position to be in stimulating relation to just a nerve portion 130, just a muscle portion 135, or both a nerve portion and a muscle portion. Moreover, any given stimulation element 110 may be in an implanted position to be in stimulating relation to a neuromuscular junction 140 without necessarily being in primary stimulating relation to the nerve portion 130 and/or the muscle portion 135 associated with the particular neuromuscular junction 140.

As further described later, in some examples at least some of multiple stimulation elements 110 may be spaced apart and positioned to cause a target tissue 120 (e.g. nerve portion(s), muscle portion(s), and/or neuromuscular junction(s), etc.) to be in stimulation relation to the stimulation elements 110 (e.g. contact electrodes of a stimulation element) by stimulation vector(s) applied through the target tissue 120 from the spaced apart stimulation elements 110 (e.g. contact electrodes of the stimulation elements). It will be further understood that the term contact electrodes refers to electrodes through (or from which) stimulation may be applied and that in some examples, such electrodes may physically contact the tissue to be stimulated while in some examples, such electrodes may be in close proximity to but not actually physically touch the tissue to be stimulated.

However, in some examples, a stimulation element 110 (or multiple stimulation elements) may be in stimulating relation to all three of a first nerve portion 130, a first muscle portion 135, and a neuromuscular junction 140 of the first nerve portion 130 and the first muscle portion 135. On the other hand, in some examples, a first stimulation element 110 may be in stimulating relation to a first nerve portion 130, a second stimulation element 110 may be in stimulating relation to a second nerve portion 130, and a third stimulation element 110 may be in stimulating relation to a neuromuscular junction 140 of a third nerve portion 130 and a third muscle portion 135. These examples, and further examples, will be apparent throughout the various examples described in association with at least FIGS. 1A-31.

In one aspect, stimulation of one or more of such example target tissues may serve to increase or maintain patency of the upper airway of the patient, and hence may sometimes be referred to as upper airway patency-related tissue(s). In some examples, such stimulation may be referred to as direct electrical stimulation of such nerves and/or such muscles, whether or not the stimulation element is in direct contact with such nerve and/or muscle. Such direct electrical stimulation may directly cause a muscle contraction of upper airway muscles resulting in tongue protrusion and/or stiffening of upper airway muscles and/or increased airway patency. Such direct electrical stimulation stands in contrast to direct electrical stimulation of other nerves or muscles, such as the phrenic nerve which does not cause contraction of upper airway muscles (e.g. the genioglossus muscle). Instead, electrical stimulation of the phrenic nerve (or diaphragm muscle) may trigger complex respiratory mechanisms including neural pathways which have a remote or rather indirect effect on upper airway patency such that the phrenic nerve may not be considered an upper airway patency-related nerve.

In some examples, as shown in FIG. 1B, an example method and/or example device 150 may comprise the stimulation element 110, a pulse generator 333, and/or a sensing element 152. As more fully described later, the pulse generator 333 may comprise, among other things, stimulation circuitry to deliver stimulation via the stimulation element 110 to target tissues. In some examples, the sensing element 152 may sense various physiologic parameters, which may enable determining a degree of upper airway patency, sleep apnea burden, among other parameters related to care of sleep disordered breathing with at least some further details regarding sensing described in association with at least FIG. 26 and/or FIGS. 30-31. The sensed physiologic information may be received by the pulse generator 333 (and/or other elements), and may be used to influence the delivery of stimulation and/or for other purposes. In some examples, operation of the stimulation element 110, pulse generator 333, and/or sensing element 152 (in relation to providing care for sleep disordered breathing) may be implemented via a control portion (and associated elements) such as but not limited to, care engine 10000, control portion 10500, 10528, and related arrangements as described in association with at least FIGS. 29A-29E and/or FIGS. 30-31.

FIG. 1C is a block diagram schematically representing an example method and/or example device 170 including an anchor portion 172, a stimulation portion 174, and a strain relief portion 176.

In some examples, the example method and/or example device 170 may comprise an example implementation of, and/or at least some of substantially the same features and attributes as, the example devices and/or example methods as described in association with at least FIGS. 1A-1B and FIGS. 2A-31.

In some examples, the example device 170 comprises varying combinations of anchor portions 172, stimulation portions 174, and/or strain relief portions 176. As described in association with at least FIGS. 2A-31, the stimulation portion 174 may comprise a single stimulation element or multiple stimulation elements. Depending on the particular example implementation, the stimulation portion 174 may provide for unilateral stimulation (on just one side of a patient's body), bilateral stimulation (on both sides of a patient's body), and/or cross-lateral stimulation. In some examples, cross-lateral stimulation may comprise providing stimulation to tissues on both sides of the patient's body and tissues in between the opposite sides of the patient's body, such as tissues at or near the sagittal midline 316. In some examples, these varying stimulation arrangements may be implemented via at least bilateral parameter 2512 of care engine 2500 in later described FIG. 30 and/or other examples throughout the present disclosure.

In some examples, anchor portion(s) 172 may be located on or near such stimulation elements (e.g. 110) and/or portions of a lead on which the stimulation portion 174 is supported and mounted. In some examples, the anchor portions 172 may provide for fixation relative to surrounding non-nerve structures and/or fixation relative to a nerve portion(s) which may form part of the target tissues. In some examples, a stimulation portion (e.g. stimulation element(s) 110) may be configurable or have a structure which secures the stimulation portion relative to target tissue, such as a nerve portion. For example, a stimulation portion 174 comprising a cuff electrode (e.g. which includes flaps, flanges, and the like to wrap about a circumference of a nerve to be stimulated) also may be considered to self-anchoring at least to the extent that the stimulation portion 174 includes its own components to secure the stimulation portion to the target tissue. A self-anchoring stimulation portion 174 may be implanted without an additional anchor portion 172 or may be chronically implanted with an additional anchor portion 172, which is secured relative to non-nerve tissues.

In some examples, strain relief portions 176 may be incorporated as a portion of a lead, with the strain relief being implemented as a dedicated shape, size, and/or length of a lead portion. In some examples, the strain relief portion 176 may be implemented as a natural effect from the particular configuration of the anchor portions 172 and/or the stimulation portion 174 (and lead portions supporting the anchor portions 172 and/or stimulation portion 174) relative to patient anatomy.

In some examples, the anchor portion 172 may be implemented in a manner in which the anchor portion 172 includes anchor elements which are sized, shaped, located, etc. relative to the stimulation portion 174 (and supporting lead portions) and/or the strain relief portion 176 such that the anchor portion 172 may be considered as a non-discrete element, i.e. as integrated with the stimulation portion 174 and/or strain relief portion 176. Among other aspects, these example anchor arrangements may enable new and different example methods of implanting the stimulation portion 174 (and associated lead portions) and/or strain relief portion(s) 176.

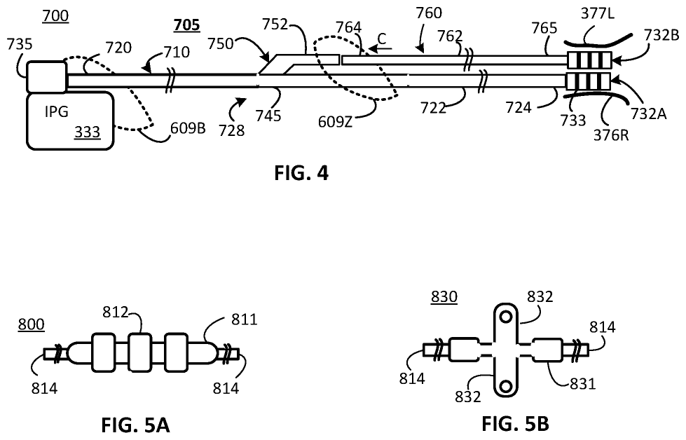

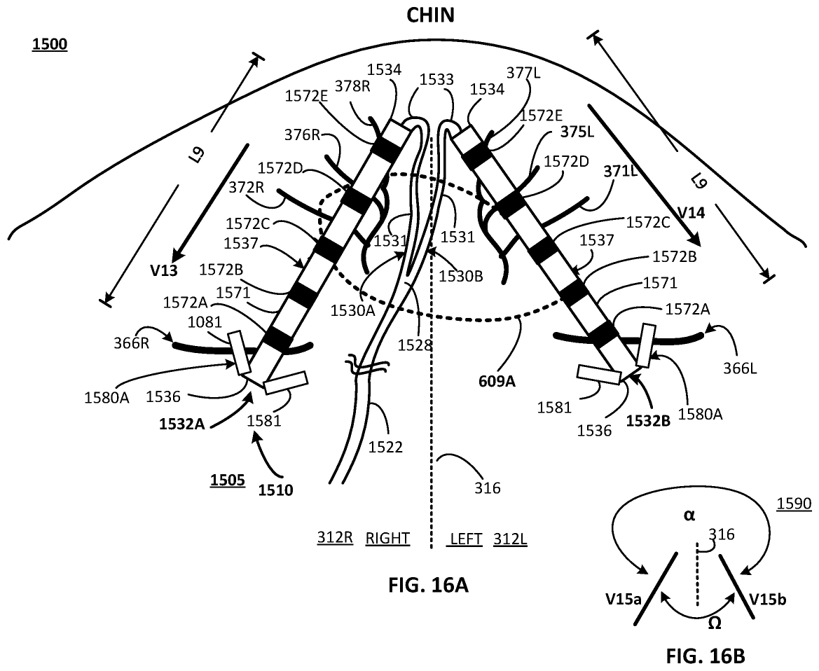

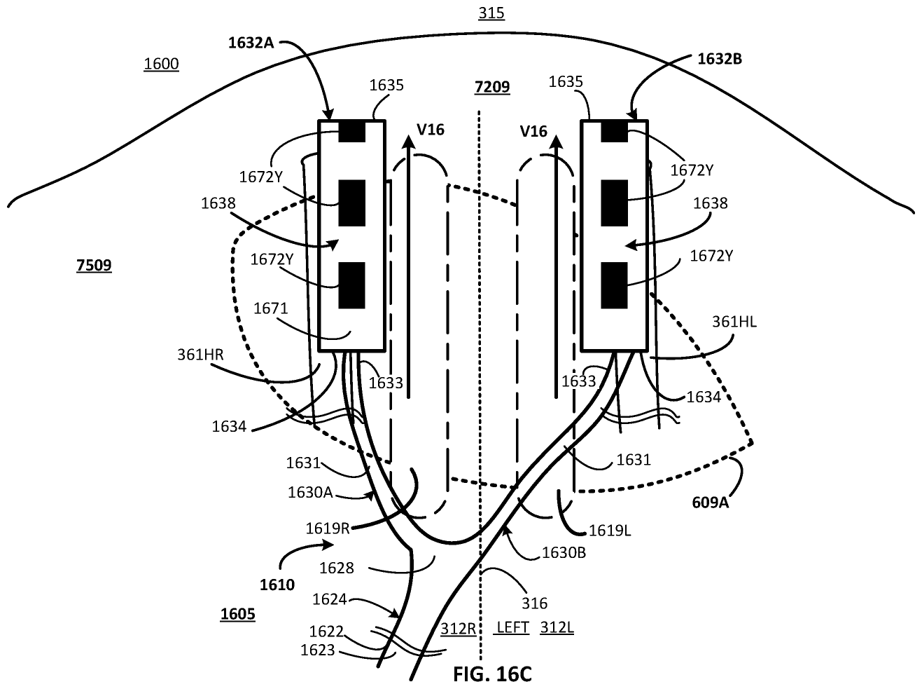

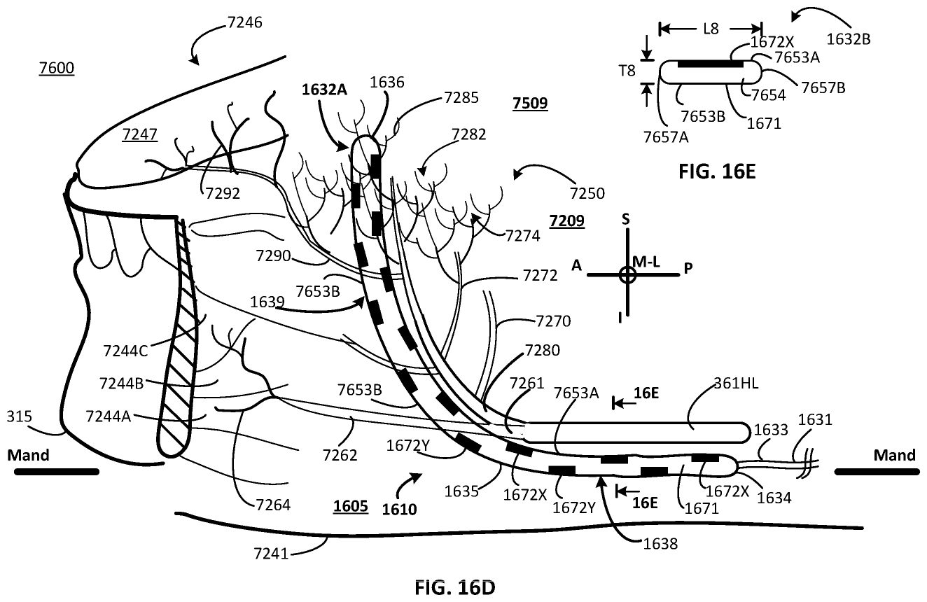

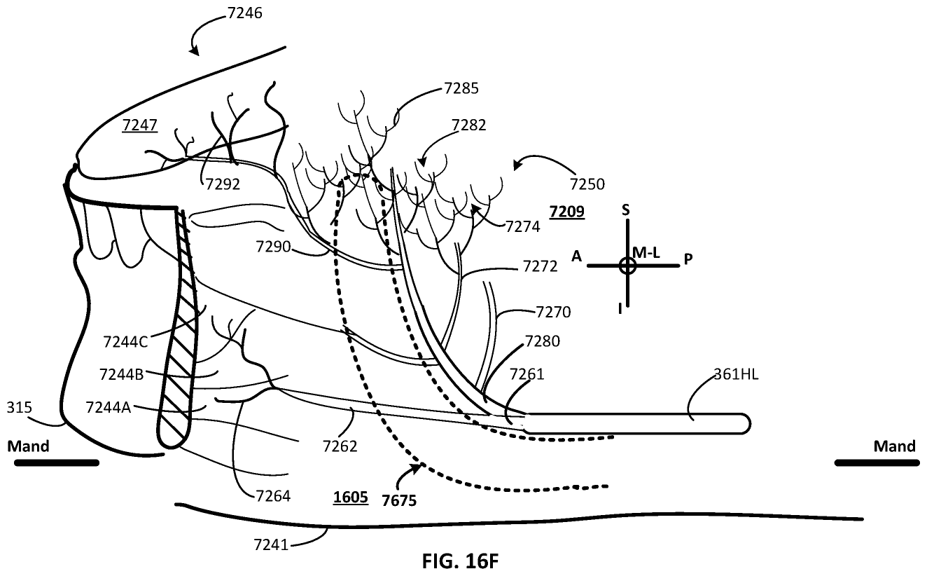

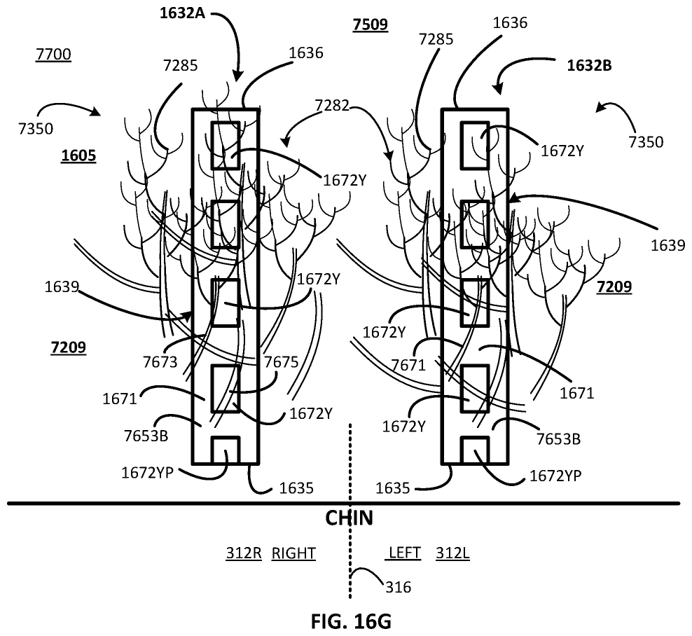

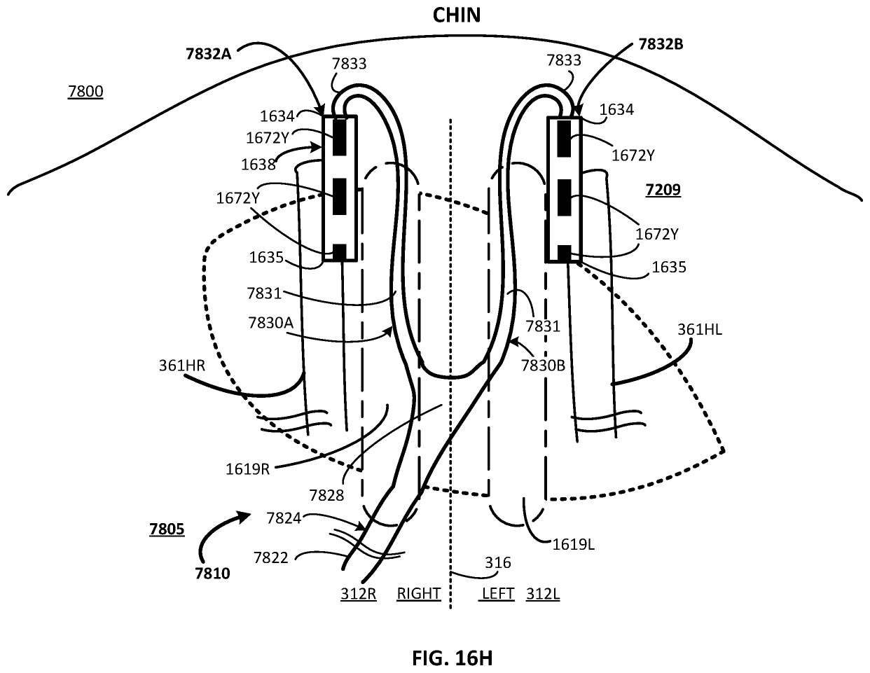

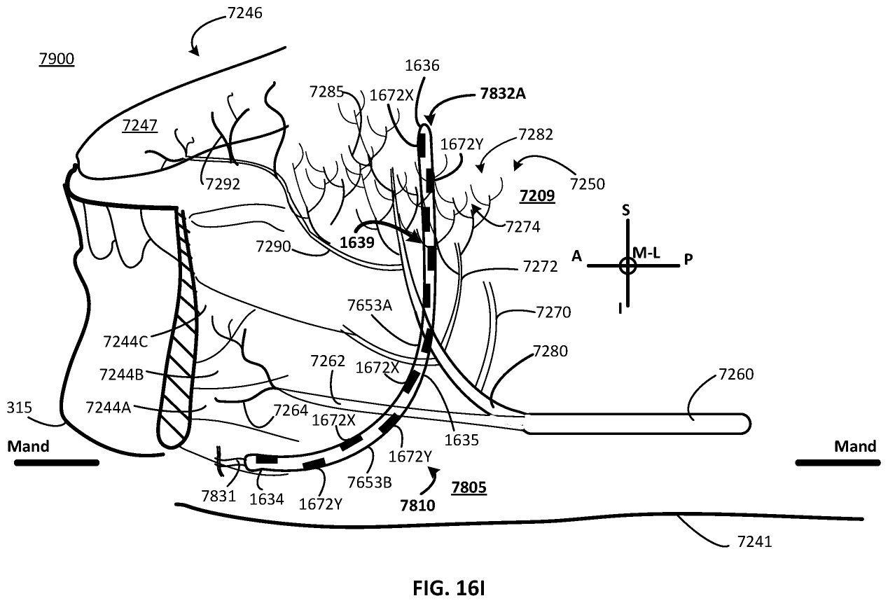

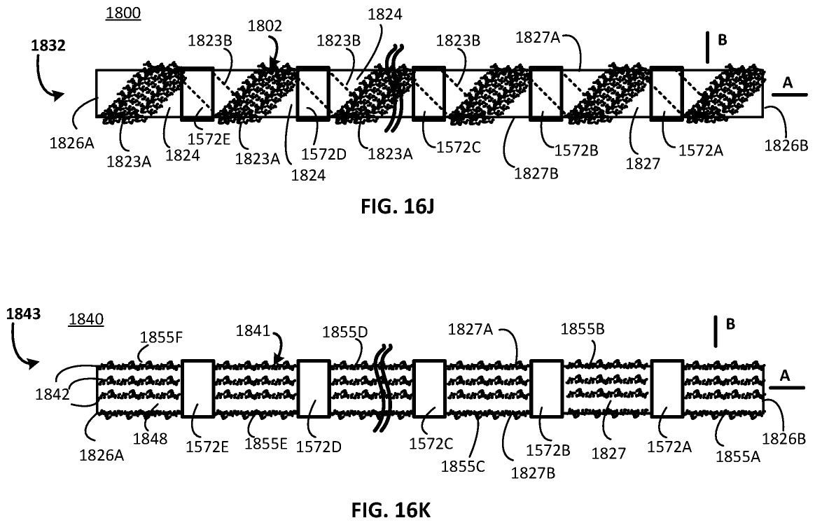

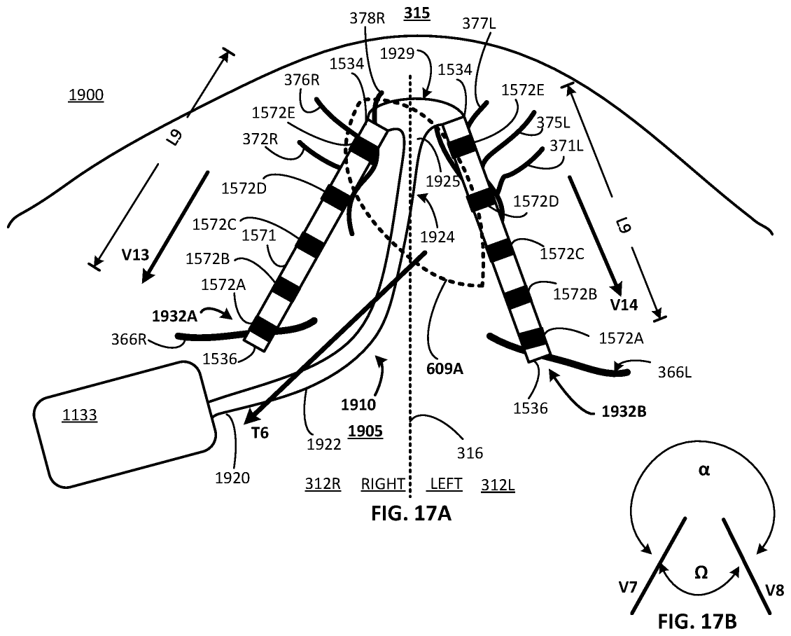

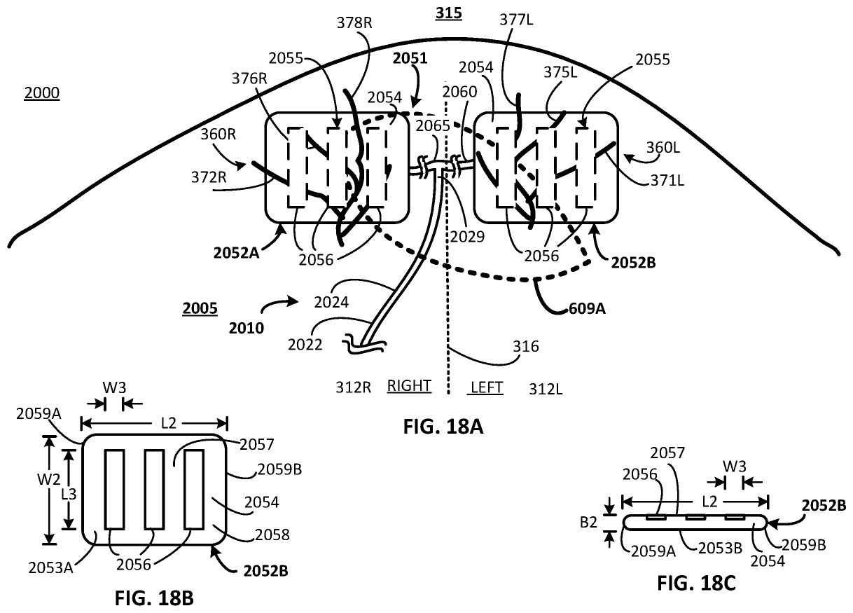

With further reference to at least FIG. 1C, in some examples one type of a stimulation element 110 (of stimulation portion 174), such as a cuff electrode (e.g. FIG. 4), may be implanted on a first side of the patient's body while a different, second type of a stimulation element 110 (of stimulation portion 174), e.g. an axial electrode (FIGS. 16A, 17A), paddle-style electrode (e.g. FIGS. 18A-22D) may be implanted on an opposite second side of the patient's body. In some such examples, the anchor portion 172 and/or strain relief portion 176 deployed on the first side of the patient's body to support implantation of the first stimulation element may be the same as or may be different from the anchor portion 172 and/or strain relief portion 176 deployed on the second side of the patient's body. As further described later, these types of combinations (and/or other types of combinations embodied throughout various examples of the present disclosure) may provide for an overall more robust implantation, therapy, etc.

With further reference to FIGS. 1A and 2A, various example implementations of stimulating different portions of the hypoglossal nerve (e.g. 360R, 360L) to activate different associated muscle groups associated with the tongue and/or other upper airway musculature are described later in association throughout various examples of the present disclosure.

While stimulation of just the hypoglossal nerve (e.g. 360R, 360L in FIG. 2A) (or some branches thereof) may be effective in increasing upper airway patency to a sufficient degree to ameliorate obstructive sleep apnea in a large majority of appropriate patients when using certain types of implantable neurostimulation devices, some patients may benefit from stimulation of an IHM-innervating nerve (e.g. 390L and/or 390R in FIG. 2A) in addition to, or instead of, stimulation of the hypoglossal nerve (e.g. 360L and/or 360R). Moreover, for a single patient, obstructive sleep apnea arising from certain positions of the head-and-neck and/or of their body (e.g. supine, lateral decubitis, etc.) and/or of their body-mass index (BMI) may be treated more effectively by stimulating an IHM-innervating nerve (e.g. 390L, 390R), with or without stimulation of the hypoglossal nerve (e.g. 360R and/or 360L). In some such examples, upon detecting that a patient is in a certain body position (e.g. supine), stimulation of the IHM-innervating nerve (e.g. 390R, 390L) may be implemented.

In addition, because each of the upper airway patency-related nerves (e.g. the hypoglossal nerve 360R, 360L, IHM-innervating nerve 390L, 390R) innervates several different muscle groups which may influence upper airway patency, stimulation may be applied at several different locations (e.g. different nerve portions) of the branches of the particular upper airway patency-related nerve. Such stimulation at the respective different locations may occur simultaneously, sequentially, alternately, etc., depending on which nerves (or muscles) are being stimulated, depending on when the stimulation occurs relative to the respective respiratory phases (or portions of each phase) of a respiratory period of the patient's breathing, and/or based on other factors. Moreover, stimulation may be alternated, sequenced, etc. between portions of a single nerve (e.g. hypoglossal) and/or may be alternated, sequenced, etc. among multiple different nerves including the hypoglossal nerve 360R, 360L and the IHM-innervating nerve 390R, 390L. At least some of these examples are further described later in association with at least FIGS. 30-31 and/or various examples of the present disclosure.

Many different examples of various stimulation locations of hypoglossal nerve portions are described below throughout the present disclosure. Of these various potential stimulation locations, FIG. 2A generally illustrates example stimulation regions A and B (shown in dashed lines) in which at least some of the stimulation locations may be located. In some examples, at least one stimulation element 310R, 310L may be implanted within each of the stimulation regions A and B, which are located on the respective right and left sides (312R, 312L) of the patient's head-and-neck region 305. However, in some examples, such stimulation elements may be implanted in just one of the example stimulation regions (e.g. A or B) on just one side of the patient's body. Within each stimulation region (e.g. A or B), a wide variety of types of stimulation elements (e.g. cuff electrode, axial array, paddle electrode) may be implanted depending on the particular delivery path, method, etc.

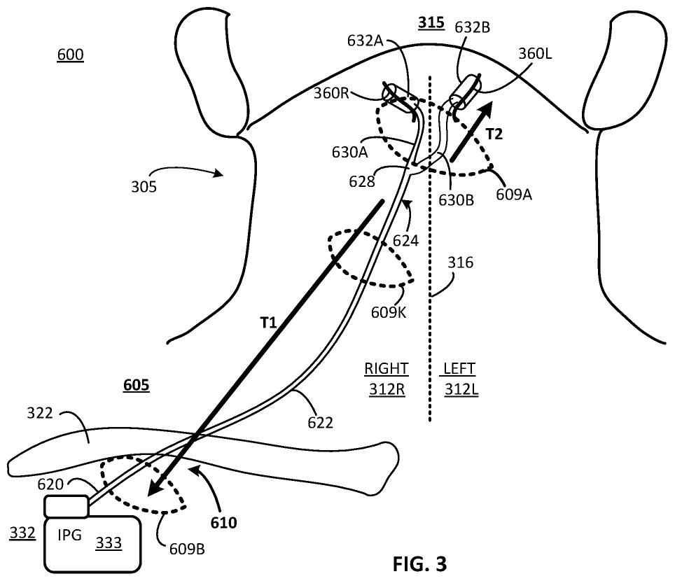

It will be understood that the respective stimulation elements 310R, 310L as shown in FIG. 2A are generally representative of a wide variety of different arrangements of stimulation elements, contact electrodes, carriers, anchor structures, etc. which may be deployed within each of the respective stimulation regions A (right side) and/or B (left side), with FIGS. 3-31 further illustrating at least some more specific example arrangements of stimulation elements relative to the different nerve portions of the respective right and left hypoglossal nerves 360R, 360L.

While FIG. 2A primarily depicts target tissues as nerve portions, it will be understood that the target tissues of each stimulation region A, B may comprise muscle portions, combinations of nerve portions and muscle portions, neuromuscular junctions of nerve portions and muscle portions, and/or combinations thereof) in addition to the nerve portions. As with other implantation methods described herein, the implantation may comprise monitoring nerves during the stimulation delivery and doing so via a nerve integrity monitor (NIM) in some examples. In some examples, a NIM may be used to verify that stimulation of the nerve(s) (e.g. hypoglossal nerve) avoids capturing target tissues promoting retraction of the tongue, such as retractor muscles. For example, the NIM may be used to verify that stimulation of the nerves(s) captures target tissues promoting protrusion of the tongue and avoids capture of target tissues promoting retraction of the tongue, rather than or in addition to monitoring for nerve impairment caused by the stimulation. In some examples, in addition to or instead of use of a NIM, direct visualization of at least some of the target tissues, such as via an implant-access incision and/or other imaging techniques, may be used to implant a stimulation element.

In some examples, the implantation methods described herein may be performed without use of a NIM. Because the implant-access incision 609A is along and/or in close proximity to the sagittal midline 316 of the patient, in some examples stimulation provided proximate to the implant-access incision 609A may avoid capture of tissues promoting retraction of the tongue such that use of a NIM may be unnecessary in some examples. For instance, in some example methods the implant-access incision 609A is in a selected location which is not in proximity to tissue (e.g. retractor nerve branches) whose stimulation would promote retraction of the tongue. Stated differently, in some example methods, the selected location of the implant-access incision 609A (e.g., along or in close proximity to the sagittal midline 316) enables placing a stimulation element (e.g. 310L, 310R) in stimulation relation to nerves (e.g. terminal end fibers, distal portions, etc.), muscles, and/or nerve-muscle junctions which may exclusively (or nearly exclusively) activate protrusor muscles to cause protrusion of the tongue, such as without activating retractor muscles, in some examples.

Moreover, in some of these examples, performing nerve monitoring (e.g. via a NIM) on nerve(s) proximate to the implant-access incision 609A may be difficult due to the shape and/or orientation of the target nerves. For example, the implant-access incision 609A may be associated with distal terminal nerve portion(s) of the target nerve(s) which are narrower in diameter than other portion(s) of the target nerves that are more proximal or superior to the implant-access incision 609A. The narrower diameter may cause difficulty for use of the NIM, such as challenges in mechanically and/or electrically coupling a monitoring electrode of the NIM relative to the nerve.

In some examples, use of a NIM may be avoided at least because the selected location (e.g. along or in close proximity to the sagittal midline 316) of the implant-access incision 609A may allow for direct visualization of protrusor muscles (and their contraction), which may aid, which may aid in an implantable stimulation element 310L, 310R minimizing and/or avoiding electrical capture of the retractor muscles.

It will be understood that these example stimulation regions A and B are not limiting and that other portions of the hypoglossal nerve 360R, 360L may comprise suitable stimulation locations, depending on the particular objectives of the stimulation therapy, the available access/delivery issues, etc.

With further reference to FIG. 2A, diagram 300 includes a front view schematically representing an example arrangement including one or more stimulation elements forming part of an example device and/or example method for increasing and/or maintaining upper airway patency or for other purposes. According, as previously mentioned and as shown in FIG. 2A, the patient's body comprises a right hypoglossal nerve 360R on the right side 312R of the patient's body and a left hypoglossal nerve 360L on a left side 312L of the patient's body. In some examples, each respective hypoglossal nerve 360R, 360L comprises a medial branch (respectively), each of which in turn comprises multiple distal branches (e.g. distal nerve portions) 372R, 376R, 378R and 371L, 375L, 377L. In some examples, the branches 372R, 376R, and/or 378R on one side (e.g. right side) of the patient's body may correspond directly to an analogous branch 371L, 375L, and/or 377L on the other side (e.g. left side) of the patient's body. However, in some examples, for at least some of the branches 372R, 376R, and/or 378R on one side (e.g. right side) of the patient's body, the branches 371L, 375L, and/or 377L on the other side (e.g. left side) of the patient's body may not comprise a directly analogous branch.

Moreover, in at least some examples, the various branches, sub-branches, terminal portions of the hypoglossal nerve 360R, 360L may sometimes be referred to as nerve portions, whether the particular branch is more distal or more proximal, etc. Stated differently, the term “nerve portion” may sometimes refer to any portion of one of the respective hypoglossal nerves 360R, 360L.

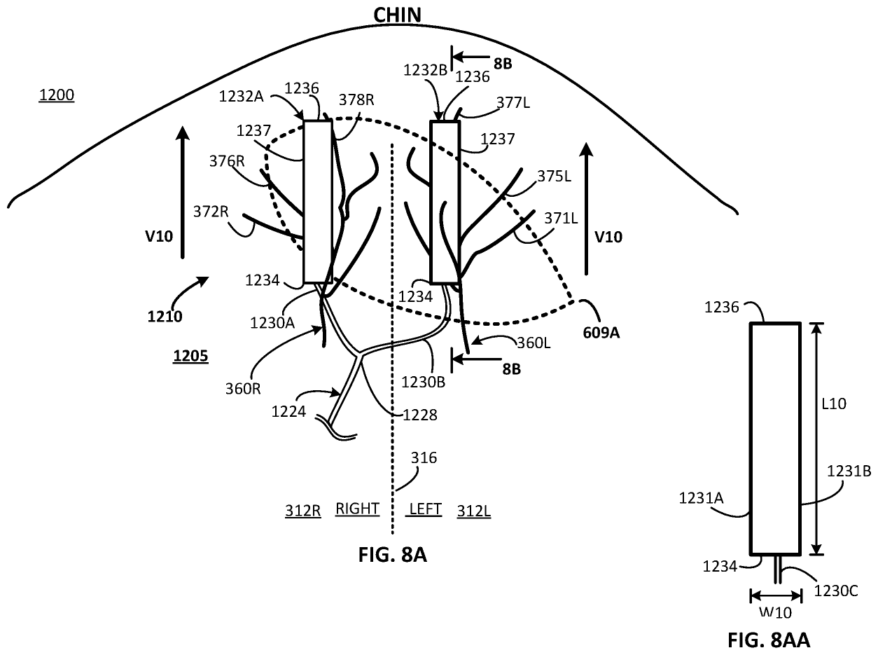

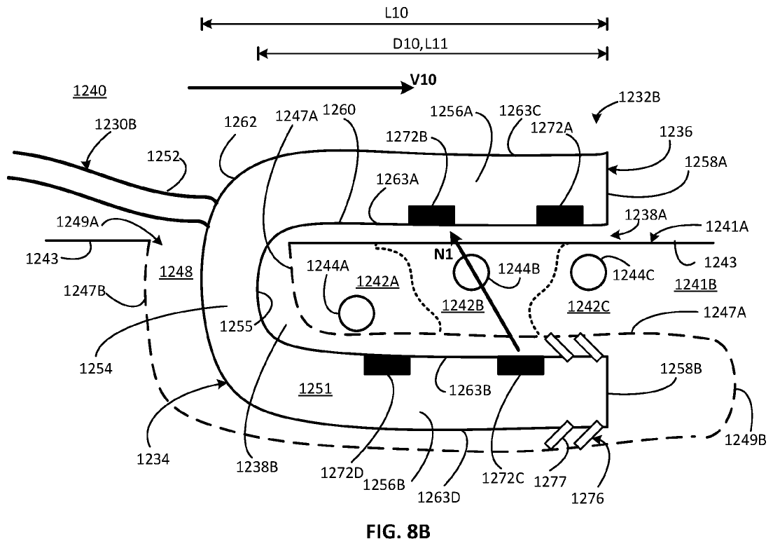

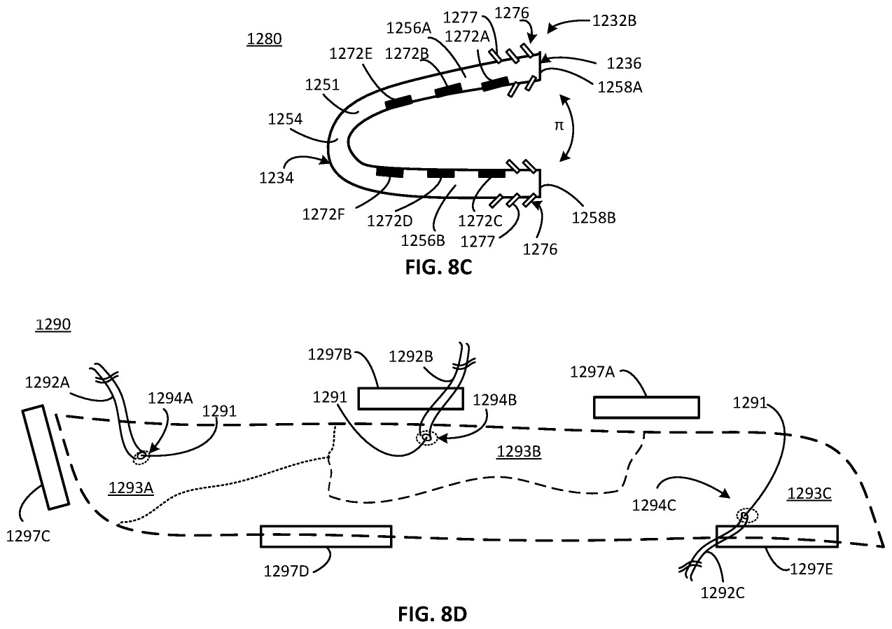

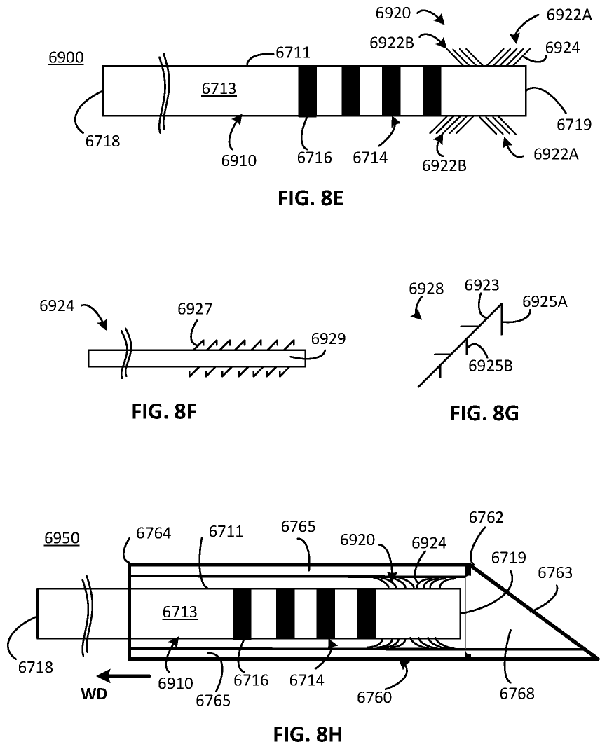

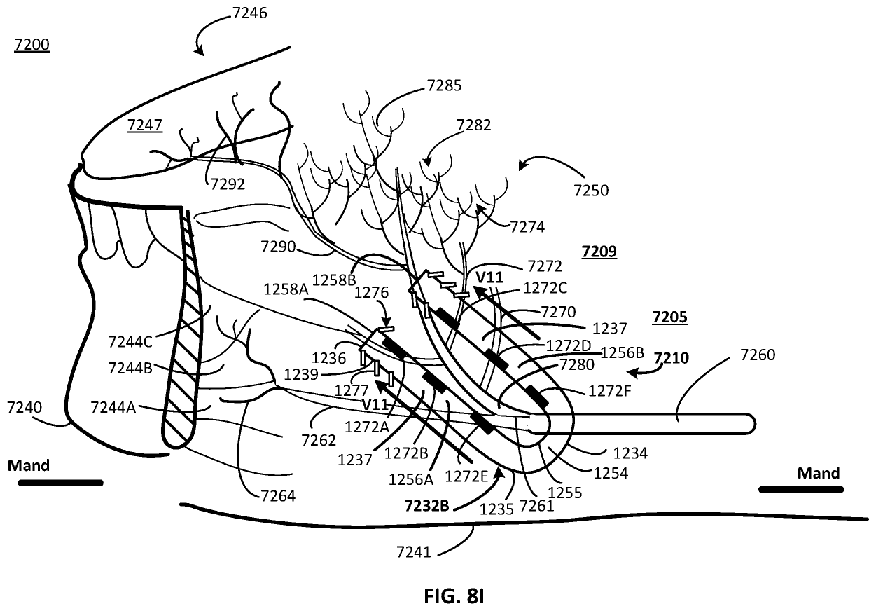

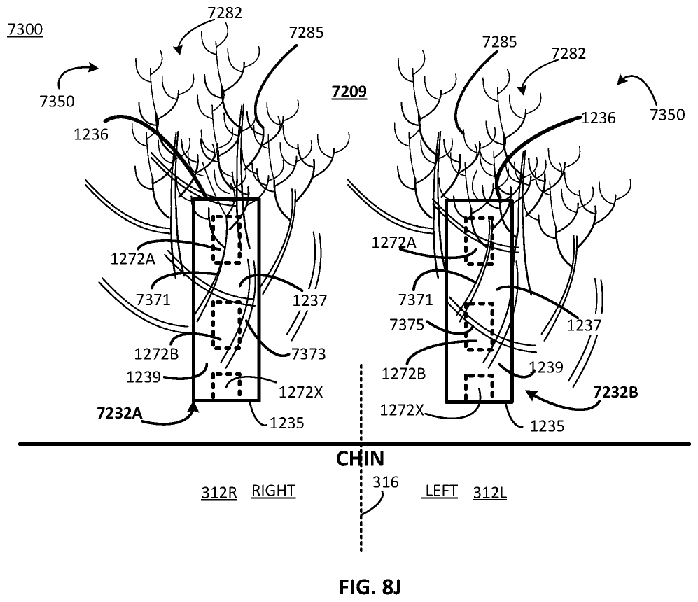

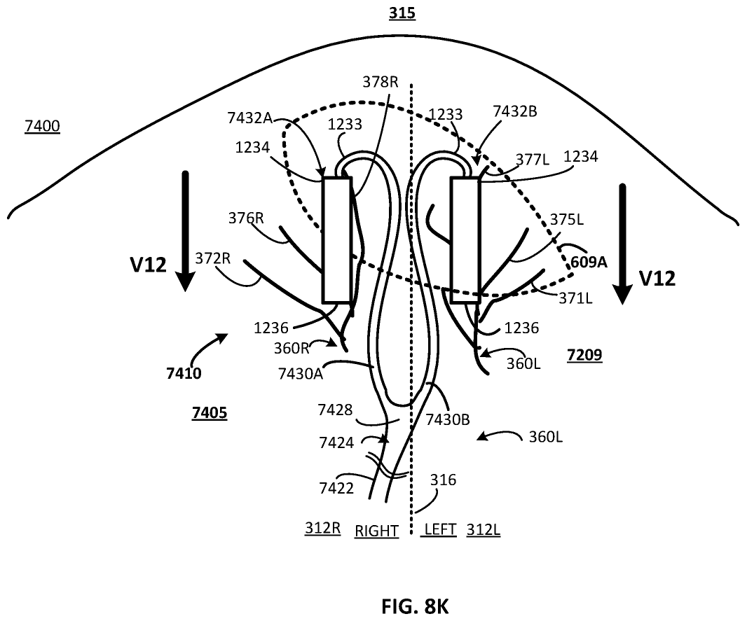

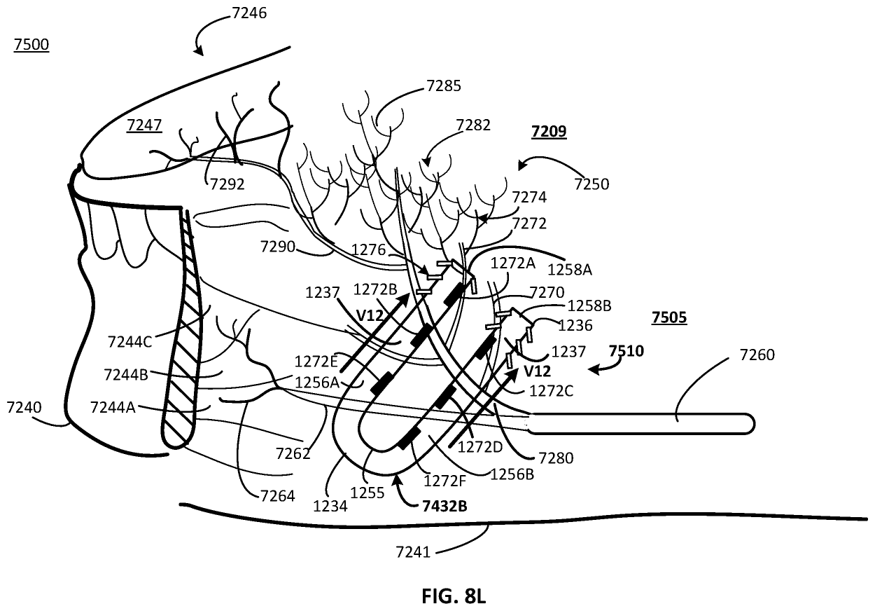

In some examples, the portion of the upper airway patency-related nerve (e.g. hypoglossal, IHM-innervating) which terminates at a muscle which the nerve portion innervates may sometimes be referred to as a terminal branch or terminal nerve portion of the nerve. In one aspect, the interface between the terminal nerve portion and the innervated muscle may sometimes be referred to as the neuromuscular junction (e.g. 140 in FIG. 1A). An example arrangement described later in association with FIG. 8D provides one example illustration of such neuromuscular junctions 140.

In further reference to at least FIG. 2A, the stimulation regions A and B (shown in dashed lines) are not strictly confined to the example arrangement nerve portions schematically depicted in FIG. 2A. Rather, the stimulation regions A and B are generally representative of a plurality of nerve portions which may be in stimulating relation to a stimulation element (or various portions of a stimulation element), which may depend on patient anatomy, the number and spatial distribution of stimulation elements (and/or supporting structures, such as anchors) among the nerve portions. Moreover, the particular nerve portions which are “in stimulating relation” on one side of the body (e.g. right side) may not necessarily correspond to the particular nerve portions which are “in stimulating relation” on the other side of the patient's body (e.g. left side).

As further shown in FIG. 2A, in some examples a stimulation region C (e.g. on right side 312R) and/or stimulation region D (e.g. on left side 312L) comprises a region for which at least a portion of a stimulation element is implanted to be in stimulating relation to other nerves such as (but not limited to) an IHM-innervating nerve 390R, 390L. Moreover, as further shown in FIG. 2A, in some examples, a third stimulation element 314R is positioned at an IHM-innervating nerve 390R on a first side 312R (e.g. right side) of the head-and-neck region 305 and a fourth stimulation element 314L is positioned at an IHM-innervating nerve 390L on an opposite second side (e.g. left side 312L) of the head-and-neck region, and therefore spaced apart from the third stimulation element 314R. As shown in FIG. 2A, the stimulation elements 314R, 314L are depicted as being in stimulating relation to the first and second IHM-innervating nerve 390L, 390R at a position (e.g. just superior to the clavicles 322 of FIG. 3A) spaced apart from the location of the hypoglossal nerves 360R, 360L. However, for illustrative simplicity, it will be understood that this depiction is also representative of the stimulation elements 314R, 314L being positioned at any portion of an IHM-innervating nerve, etc. with at least FIG. 28 providing more detailed illustrations of an IHM-innervating nerve, such as but not limited to nerve/branch 9931 (e.g. branches 9932, 9942, 9952) and/or the relationship of such IHM-innervating nerves/branches relative to an AC loop nerve. As apparent from FIG. 2A, the stimulation element 310R is also spaced apart from stimulation element 314R, while stimulation element 310L is spaced apart from stimulation element 314L.

It will be understood that in some examples a portion(s) of the IHM-innervating nerve 390R, 390L may be in proximity to a portion(s) of the hypoglossal nerve 360R, 360L such that a stimulation region (e.g. A) for one nerve (e.g. hypoglossal nerve 360R) may be in close proximity to and/or overlap with a stimulation region (e.g. C) for another nerve (e.g. IHM-innervating nerve 390R) despite FIG. 2A depicting generous spacing between such stimulation regions A and C. However, in some examples in which a particular portion of the hypoglossal nerve (e.g. 360R) being stimulated is not in close proximity to a particular portion of the IHM-innervating nerve (e.g. 390R), then the stimulation region A may be spaced apart from and not in close proximity to the other stimulation region C.

As shown in FIG. 2A, in some examples a stimulation element 310R, 310L, 314R, 314L may comprise or may be connected to a pulse generator 333 (e.g. implantable pulse generator (IPG)), which in some examples may comprise a microstimulator. One example microstimulator is later described in association with at least FIG. 17A, but is applicable to the other examples throughout the examples of FIGS. 1A-31 in some examples. In some examples, the pulse generator 333 comprises a power source, such as a non-rechargeable power source or a rechargeable power source. If the power source is rechargeable, it may be configured for wireless power transfer and communication relative to an external power source. In some examples, the pulse generator 333 comprises a control portion, which may comprise memory and circuitry to executed stored instructions to perform stimulation. The circuitry may comprise a controller or processor to cause execution of the stored instructions (which may include stimulation protocols) and/or other circuitry to generate stimulation signals, communicate with external devices (e.g. remote control, programmer, etc.) or internal devices (e.g. sensor), and/or to sense physiologic phenomenon. In some examples, the pulse generator 333 may comprise at least some of substantially the same features and attributes as control portion 10500 (FIG. 29B) and/or comprise an example implementation of at least some aspects of the control portion 10500 (FIG. 29B).

In some examples, at least some of these components, portions, etc. (e.g. power, control, circuitry, stimulation circuitry, etc.) of the pulse generator 333 (whether or not a microstimulator) may be located external to the patient's body, as further described in association with at least FIGS 17A and 31, with complementary components, portions being implantable within the patient's body.

In some examples, the implant-access incision 609A may sometimes be referred to as a midline implant-access incision in that the implant-access incision is located along or in close proximity to a sagittal midline of the patient's body, such as within a submental region (e.g. under the patient's chin 315 and inferior to the right and left mandible 318R, 318L). In some such examples, a sagittal midline location may correspond to a center of the area defined by the implant-access incision being located along the sagittal midline or located within close proximity to the sagittal midline. In this context, in some examples the term “close proximity” may comprise a distance of less than about 5 millimeters to about 5 centimeters (or less than about 5 centimeters) from the sagittal midline 316 of the patient's body. In some examples, this distance may comprise less than about 5 millimeters, less than about 10 millimeters, less than about 50 millimeters, less than about 100 millimeters, less than about 250 millimeters, less than about 500 millimeters, less than about 750 millimeters, less than about 1 centimeter, less than about 1.5 centimeters, less than about 2 centimeters, less than about 2.5 centimeters, less than about 3 centimeters, less than about 3.5 centimeters, less than about 4 centimeters, less than about 4.5 centimeters, and less than about 5 centimeters. In this context and elsewhere throughout the application, when “about” is utilized to describe a value, this includes, refers to, and/or encompasses variations (up to +/−2%) from the stated value. In some examples, the various ranges provided herein include the stated range and any value or sub-range within the stated range.

In some examples, the implant-access incision 609A comprises an area having a greatest cross-sectional dimension of about 3 to about 5 centimeters with the area comprising a suitable shape (e.g. generally triangular, generally elliptical, etc.) in some examples. The area may be bounded on its sides by inferior border of the mandible.

In some examples, the hypoglossal nerves 360R, 360L are the sole type of upper airway patency-related nerves which are accessed from a midline implant-access incision 609A and/or which are to be stimulated to treat sleep disordered breathing. However, as further described later, other nerves related at least to upper airway patency may be accessed via the midline implant-access incision 609A.

It will be further understood that for illustrative purposes to clearly delineate separate nerve portions and the manner in which distinct portions of a stimulation element may engage such different nerve portions, FIG. 2A may depict the nerve portions 372R, 376R, 378R in a pattern which appears to have a more anterior-posterior distribution than might actually occur. For example, in some examples in which each nerve portion 372R, 376R, 378R may innervate a different muscle group or subgroup related to the tongue, it will be understood that the particular pattern of nerve portions 372R, 376R, 378R may have a pattern which exhibits a more superior-inferior orientation than shown in FIG. 2A. For example, as further described later, FIG. 2B depicts a view schematically representing the different muscle groups in a superior-inferior orientation/pattern more closely corresponding to actual anatomical arrangement in which the respective muscle subgroups are stratified vertically.

While FIG. 2A depicts stimulation elements for both the hypoglossal nerve (e.g. elements 310R, 310R) and for the IHM-innervating nerve (e.g. elements 314R, 314L), it will be understood that in some examples, stimulation of an upper airway patency-related tissue may comprise stimulation of solely of the hypoglossal nerves 360R and/or 360L. In such arrangements, stimulation of the IHM-innervating nerve 390R, 390L does not occur at all or at least does not occur during specified time periods, situations, etc. Moreover, in some such examples, stimulation elements 314R, 314L may be omitted entirely, i.e. such stimulation elements are not even implanted in the patient's body and do not form part of an implanted device.

Stimulation of just one hypoglossal nerve (e.g. 360R or 360L) may sometimes be referred to as unilateral stimulation, while stimulation of both such hypoglossal nerves (e.g. 360R and 360L) may sometimes be referred to as bilateral stimulation. It will be further understood that in some instances of unilateral stimulation, just one of the respective stimulation elements 310R, 310L has been implanted. However, in other examples of unilateral stimulation, both stimulation elements 310R, 310L may be implanted, but just one of them is stimulated to provide unilateral stimulation.

In some examples of bilateral stimulation of the hypoglossal nerves (e.g. 360R, 360L), the stimulation may be implemented simultaneously, alternately, and/or in other patterns.

Furthermore, in some examples in which one or both of stimulation elements 310R, 310L are implanted (to stimulate hypoglossal nerve(s)), neither of the stimulation elements 314R, 314L (for stimulating an IHM-innervating nerve) are implanted.

However, in some examples in which one or both of stimulation elements 310R, 310L are implanted (to stimulate hypoglossal nerve(s)), one or both of the stimulation elements 314R, 314L (for stimulating an IHM-innervating nerve) may be implanted. In some such examples, even though such stimulation elements 314R, 314L may be implanted, such stimulation elements 314R, 314L may be not necessarily be activated in some examples in which just stimulation of one or both of the hypoglossal nerve(s) 360R, 360L is to be provided.

While FIG. 2A depicts stimulation elements for both the hypoglossal nerve (e.g. elements 310R, 310L) and for the IHM-innervating nerve (e.g. elements 314R, 314L), it will be understood that in some examples, stimulation of an upper airway patency-related tissue may comprise stimulation of solely one or both of the IHM-innervating nerves 390R, 390L. In such arrangements, stimulation of the hypoglossal nerve 360R, 360L does not occur at all or at least does not occur during specified time periods, situations, etc.

Stimulation of just one IHM-innervating nerve (e.g. 390R or 390L) may sometimes be referred to as unilateral stimulation, while stimulation of both such nerves (e.g. 390R and 390L) may sometimes be referred to as bilateral stimulation. It will be further understood that in some instances of unilateral stimulation, just one of the respective stimulation elements 314R, 314L has been implanted. However, in other examples of unilateral stimulation, both stimulation elements 314R, 314L may be implanted, but just one of them is stimulated to provide unilateral stimulation.

In some examples of bilateral stimulation of IHM-innervating nerves (e.g. 390R, 390L), the stimulation may be implemented simultaneously, alternately, and/or in other patterns.

Furthermore, in some examples in which one or both of stimulation elements 314R, 314L are implanted (to stimulate the IHM-innervating nerve(s)), neither of the stimulation elements 310R, 310L (for stimulating a hypoglossal nerve) are implanted.

However, in some examples in which one or both of stimulation elements 314R, 314L are implanted (to stimulate IHM-innervating nerve(s)), one or both of the stimulation elements 310R, 310L (for stimulating a hypoglossal nerve) may be implanted. In some such examples, even though such stimulation elements 310R, 310L may be implanted, such stimulation elements 310R, 310L may be not activated in some examples in which just stimulation of one or both of the IHM-innervating nerve(s) 390R, 390L is to be provided.

In some examples, stimulation of just the IHM-innervating nerve(s) 390R and/or 390L may be implemented for particular collapse patterns of the upper airway or less than complete collapse behaviors.

With further reference to the example arrangement in FIG. 2A, in some examples just one stimulation element is implanted at a left side 312L of the head-and-neck region 305 to stimulate a first type of nerve (e.g. hypoglossal, IHM-innervating, or other) and just one stimulation element is implanted at right side 312R of the head-and-neck region 305 to stimulate a different second type of nerve (e.g. hypoglossal, IHM-innervating, other). For instance, in some examples just stimulation element 310R is implanted to stimulate a right hypoglossal nerve 360R and just stimulation element 314L is implanted to stimulate a left IHM-innervating nerve 390L, or vice versa.

Alternatively, in some examples all stimulation elements 310R, 310L, 314R, 314L of example arrangement may be implanted, but stimulation is implemented solely via stimulation element 310R for right hypoglossal nerve 360R and solely via stimulation element 314L for the left IHM-innervating nerve 390L, or vice versa, in some examples. In some such examples, the stimulation elements (e.g. a combination of 310R and 314L, or a combination of 310L and 314R) may be activated to deliver stimulation simultaneously to the respective hypoglossal and IHM-innervating nerves. However, in some examples, the stimulation elements (e.g. a combination of 310R and 314L, or a combination of 310L and 314R) may be activated to deliver stimulation alternately to the respective hypoglossal and IHM-innervating nerves. In yet other examples, various stimulation patterns may be implemented in which one stimulation element (e.g. 310L) is activated multiple times within a selectable period of time and then the other stimulation element (e.g. 314R) is activated one or more times.

In further examples, stimulation applied via the respective stimulation elements 310R, 310L, 314R, 314L may be implemented in an interleaving manner.

It will be further understood that the various stimulation elements 310R, 310L, 314R, 314L illustrated in FIG. 2A may be embodied as part of a lead, a microstimulator, etc., and may be anchored to a non-nerve tissue or structure within the patient's body via various anchor elements, as described more fully below in association with at least FIGS. 8B-8L, 11A-11B, 13A-16A, 16J-16K, 23A-24E, 25H, and 30-31.

Similarly, the respective stimulation elements 310R, 310L, 314R, 314L may be embodied as one of the various electrode arrays, cuff electrodes, paddle electrodes, etc. as described more fully below in various example arrangements of the present disclosure. The respective stimulation elements may be embodied in a unipolar configuration, a bipolar configuration or multi-polar configuration.

In some examples, the various stimulation arrangements described in association with at least FIG. 2A may be implemented and stimulation performed without any sensing at all or with limited sensing, such as (but not limited to) just sensing to evaluate effectiveness of the stimulation but not using the sensing to time or trigger the stimulation. In some such examples, arrangements which do not use the sensing to time or trigger the stimulation may sometimes be referred to as open loop stimulation. Some examples of open loop stimulation may completely omit the presence of (and/or use of) a sensing element used for timing stimulation. Whether sensing is provided or not, in some examples stimulation may be applied simultaneously to both an IHM-innervating nerve and a hypoglossal nerve. Further details are described later in association with at least FIGS. 28-31.

In some examples, the target nerve 130, target muscle 135, and/or target neuromuscular junction 140 may comprise target tissues which are respiratory-related tissues (such as a phrenic nerve, diaphragm muscle, and/or neuromuscular junctions of the phrenic nerve and diaphragm) but which are not necessarily upper airway patency-related nerves.

FIG. 2B is a diagram 400 schematically representing example patient anatomy including right and left hypoglossal nerves 360R, 360L in a manner similar to diagram 300 in FIG. 2A except further depicting various muscle groups associated with various distal nerve portions of the hypoglossal nerves 360R, 360L. Accordingly, in some examples, nerve portion 372R may innervate muscle portion 382R, nerve portion 376R may innervate muscle portion 386R, and nerve portion 378R may innervate muscle portion 388R. In some examples, nerve portion 371L may innervate muscle portion 383L, nerve portion 375L may innervate muscle portion 387L, and nerve portion 377L may innervate muscle portion 389L. In some examples, on the right side 312R of the patient's body, the muscle portion 382R may comprise at least a portion of the genioglossus oblique (GGO) muscle group, the muscle portion 386R may comprise at least a portion of the genioglossus horizontal muscle group (GGH), the muscle portion 388R may comprise at least a portion of the geniohyoid (GGH) muscle group. In some examples, on the left side 312L of the patient's body, the muscle portion 383L may comprise at least a portion of the genioglossus oblique (GGO) muscle group, the muscle portion 387L may comprise at least a portion of the genioglossus horizontal muscle group (GGH), and the muscle portion 389L may comprise at least a portion of the geniohyoid (GGH) muscle group.

It will be further understood that deployment of the example devices and/or example methods of the present disclosure are not limited to a number and/or position of the example nerve portions, muscle portions, and/or neuromuscular junctions shown in FIG. 2B as the particular anatomical features represented are non-limiting examples.

FIGS. 2C-2D are diagrams schematically representing further example implementations of, and/or consistent with, the implant-access incision 609A of FIG. 2A.

In some examples, as shown by FIG. 2C, a primary portion 609F of the implant-access incision 609A (FIG. 2A) extends along a transverse plane 317. In some examples, the primary portion 609F may correspond to a primary orientation in which a cut to form the implant-access incision (e.g. 609A in FIG. 2A) is made and/or may correspond to a longitudinal axis of the formed implant-access incision 609A. The transverse plane 317 may be perpendicular (or substantially perpendicular) to the sagittal midline 316. In some such examples, the primary portion 609F of implant-access incision 609A may extend along the transverse plane 317 and may be intersect the sagittal midline 316. In some examples, the primary portion 609F of implant-access incision 609A may be inferior to the mandible 318 by a distance D and extend horizontally along the transverse plane 317 for a distance H. In some examples, the distances (e.g., distance D, distance H, etc.) may refer to a measurement/distance before an incision is made (e.g., a line where a cut is made). In some examples, the distance D may be between about 0.5 centimeters (cm) and about 2.0 cm, about 0.75 cm and about 2.0 cm, about 0.75 cm and about 1.5 cm, about 0.75 and about 1.25, about 0.5 and about 1.5, or about 1 cm inferior to the mandible. In some examples, distance D is about 1 cm inferior to the mandible. In some examples, the distance H may be between about 2 cm and about 5 cm, about 2 cm and about 4.5 cm, about 2 cm and about 4 cm, about 2 cm and about 3.5 cm, about 2 cm and about 3 cm, about 2.5 cm and about 5 cm, about 2.5 cm and about 4.5 cm, about 2.5 cm and about 4 cm, about 2.5 cm and about 3.5 cm, or about 3 cm.

In some such examples, the distance H may sometimes be referred to as a length of the primary portion 609F. While FIG. 2C shows a midpoint of the primary portion 609F of the implant-access incision 609A extending along the transverse plane 317 as being centered on the sagittal midline 316, examples are not so limited and the midpoint of the primary portion 609F of the implant-access incision 609A may be offset from the sagittal midline 316 by a threshold, such as up to 1 cm.

In some examples, as shown by FIG. 2D, a primary portion 609G (e.g. primary aspect) of the implant-access incision 609A extends along (or in close proximity to and parallel to) the sagittal midline 316. In some examples, the primary portion 609F may correspond to a primary orientation in which a cut to form the implant-access incision (e.g. 609A in FIG. 2A) is made and/or may correspond to a longitudinal axis of the formed implant-access incision 609A. In some examples, the primary portion 609G of the implant-access incision 609A may extend vertically (e.g. in a superior-inferior and/or anterior-posterior orientation) along (or in close proximity to and parallel to) the sagittal midline 316 for a distance L. In some such examples, a first end of the primary portion 609G of the implant-access incision 609A may be inferior to the mandible 318 by a distance D and the entire primary portion 609G ay extend vertically along (or in close proximity to and parallel to) the sagittal midline 316 for the distance L. In some examples, the distance D may be between about 0.5 centimeters (cm) and about 2.0 cm, about 0.75 cm and about 2.0 cm, about 0.75 cm and about 1.5 cm, about 0.75 and about 1.25, about 0.5 and about 1.5, or about 1 cm inferior to the mandible. In some examples, distance D is about 1 cm inferior to the mandible.

In some examples, the distance L may be between about 2 cm and about 5 cm, about 2 cm and about 4.5 cm, about 2 cm and about 4 cm, about 2 cm and about 3.5 cm, about 2 cm and about 3 cm, about 2.5 cm and about 5 cm, about 2.5 cm and about 4.5 cm, about 2.5 cm and about 4 cm, about 2.5 cm and about 3.5 cm, or about 3 cm. While FIG. 2D shows the primary portion 609G of the implant-access incision 609A extending along and/or being centered on the sagittal midline 316, examples are not so limited and the primary portion 609G of the implant-access incision 609A may be offset from (e.g. in close proximity to, and parallel to) the sagittal midline 316 by a threshold, such as up to 1 cm.

In some examples, an implant-access incision (e.g. 609A in FIG. 2A) may include both portions 609F, 609G (e.g., orientation of cuts forming the implant-access incision) illustrated by FIGS. 2C and 2D (e.g. along a transverse plane 317 and, e.g., along the sagittal midline 316). In some such examples, after the intended cuts are made, this arrangement may form a triangular-shaped incision, an elliptical-shaped incision, and the like. In some examples, a primary portion of the implant-access incision 609A may be aligned on a plane that is between and intersects both the transverse plane 317 and the sagittal midline 316.

FIG. 3 is a diagram 600 including a front view schematically representing an example method and/or example device 605 for treating sleep disordered breathing via stimulation elements 632A, 632B. In some examples, the example method (and/or example device) comprises at least some of substantially the same features and attributes as the examples previously described in association with at least FIGS. 1A-2D. In some examples, the stimulation elements 632A, 632B may comprise cuff-type stimulation elements in which a carrier body may be wrapped about a nerve or multiple nerve branches to secure contact electrodes into contact with, and stimulating relation to, the nerve.

As shown in FIG. 3, in some examples this example method may involve tunneling (T1) between a first incision 609A and a second incision 609B, wherein an implantable pulse generator (IPG) 333 is implanted via first incision 609B and a stimulation element (e.g. 632A, 632B) is implanted via second incision 609A. In some examples, as later described, tunneling T1 may be omitted or simplified such as when pulse generator (e.g. IPG 333) embodied as a microstimulator (e.g. 1133 in FIG. 17A) which is implanted via the same implant-access incision 609A that stimulation elements are implanted, which may in some examples be the sole/single implant-access incision for implanting the entire implantable stimulation device, as previously mentioned. In some such examples, the microstimulator 1133 may be implanted without tunneling or via tunneling (e.g. T6) as shown in FIG. 17A.

With further reference to FIG. 3, in some examples general midline access-implant incision 609A defines an area A1 which is sized and shaped to allow direct visualization of a subcutaneous location at which a stimulation element 632A, 632B may be implanted in stimulating relation to a nerve portion, such as but not limited to terminal branches of hypoglossal nerve, muscles (e.g. protrusor muscles) innervated by those terminal branches, and/or neuromuscular junctions of such terminal branches and the associated innervated muscles.

In some examples, the midline access-implant incision 609A may enable direct visualization of implantation of some elements, such as stimulation elements into stimulating relation to nerves, muscles, etc., while tools/methods may be used to implant other portions, elements (e.g. pulse generator) of the device within the patients' body where such implant locations cannot be directly visualized from the implant-access incision 609A. However, in some examples, the midline access-implant incision 609A may enable direct visualization (or near direct visualization) during implantation of all elements of an example device, such as stimulation elements into stimulating relation to nerves, muscles, etc., and implantation of other portions, elements (e.g. pulse generator) of the device within the patients' body.

It will be further understood that some example methods deploying a midline implant-access incision may comprise at least some stimulation elements being implanted via tools (e.g. needle probes, stylets, guidewires, introducers, etc.) deployed within the patient's body. For example, one stimulation element may be implanted (in stimulating relation to a target tissue) in close proximity to the midline implant-access incision 609A, while another stimulation element may be implanted further from the midline implant-access incision 609A such that some tools may be employed to implement such implantation.

In some examples, such tools also may be used to help confirm desired anatomical positioning of a stimulation element relative to target tissue(s). For instance, at least some components of such tools may be used to perform test stimulations of a stimulation element at various anatomical locations, orientations, etc. and then, as desired, a position, orientation, etc. of the stimulation element may be adjusted before a final location of chronic implantation is selected and implemented. In some such examples, imaging may be used to observe the respective anatomical locations, positions/orientations of the stimulation element and/or related tools, wherein the imaging may comprise live visualization tools such as, but not limited to, ultrasound imaging used during such positioning, implanting, etc. of the stimulation element(s), access tools, etc.

In some of the foregoing examples regarding test stimulation, the stimulation testing also may be used to determine a desired location, orientation, etc. while implanting multiple stimulation elements, which may be used to apply stimulation signals individually or in vectors among multiple stimulation elements.

As shown in FIG. 3, in some examples the device 605 comprises a stimulation lead 610 and an IPG 333. The stimulation lead 610 comprises a lead body 622 including a distal portion 624 and a proximal portion 620, which is electrically and mechanically connectable to the IPG 333. The distal portion 624 of lead body 622 comprises a bifurcation portion 628 at which the distal portion 624 comprises a first distal segment 630A and a second distal segment 630B which are separate from each other and independently positionable within the patient's anatomy. In some examples, as shown in FIG. 3, one distal segment 630B may be implantably positioned within a left side 312L of the patient's anatomy and the other distal segment 630A may be implantably positioned within a right side 312R of the patient's anatomy.

However, in some examples both distal segments 630A, 630B may be implantably positioned on a same side (e.g. left side or right ride) of the patient's body with each distal segment 630A, 630B being implantably positioned to place stimulation electrode(s) on the respective segment 630A, 630B in stimulating relation to different nerve portions of a nerve (e.g. 360R on right side or 360L on left side) on a single side of the patient's body.

In some examples, each lead segment 630A, 630B comprises a stimulation element 632A, 632B, respectively. Each stimulation element 632A, 632B comprises an array of stimulation contact electrodes for delivering a stimulation therapy signal to the target tissue, such as a nerve, muscle, or combination thereof. In some examples, at least one of the stimulation elements 632A, 632B may comprise a cuff electrode implantably mounted relative to a nerve portion of the respective nerves 360R, 360L. However, it will be understood that the stimulation elements 632A, 632B may comprise other types and forms of stimulation elements other than cuff electrodes, such as but not limited to the examples later described in association with at least FIGS. 4-31.

In some examples, each stimulation element 632A, 632B may comprise a large surface area stimulation element to be implanted in the vicinity of target tissue. The stimulation element 632A, 632B may comprise a plurality of individually addressable contact electrodes to deliver the stimulation. In some examples, a desired implant location may be confirmed via intraoperative testing including test stimulation delivery to the target tissues in stimulating relation with the stimulation element 632A, 632B at the intended implant location.

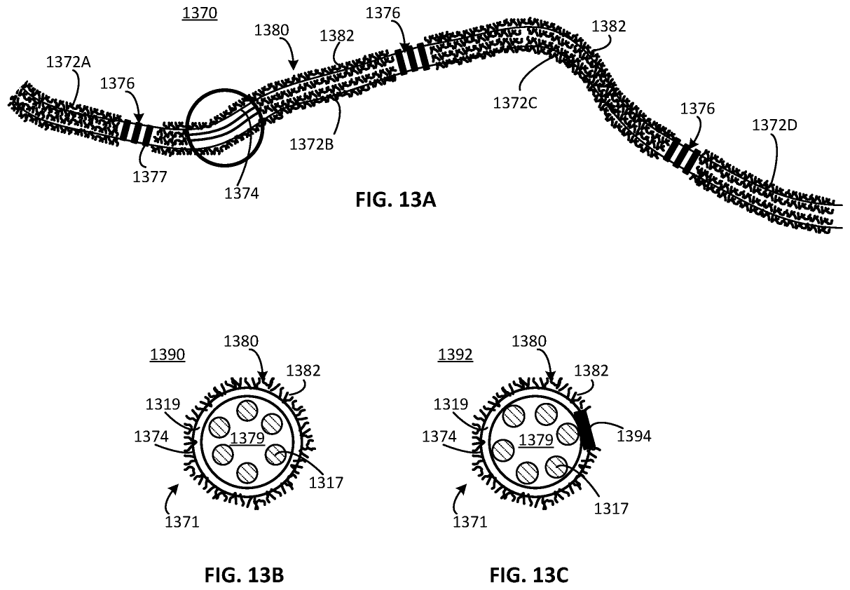

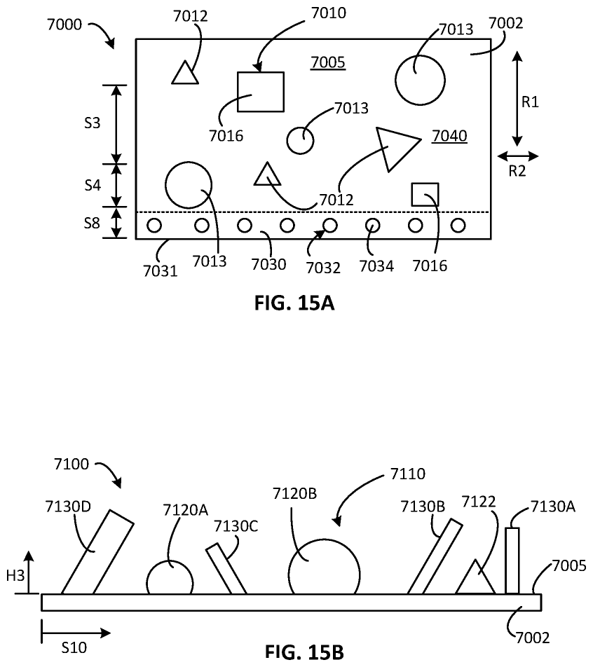

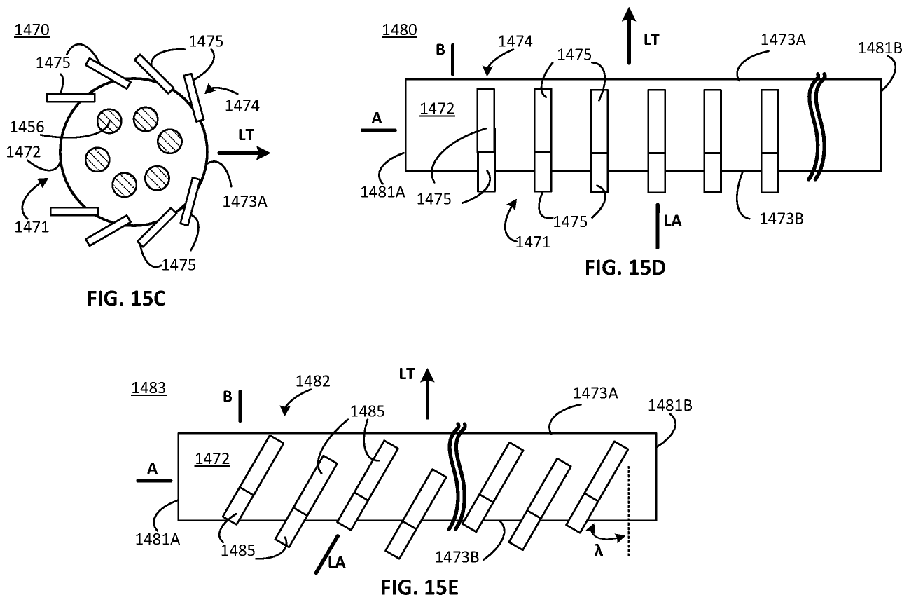

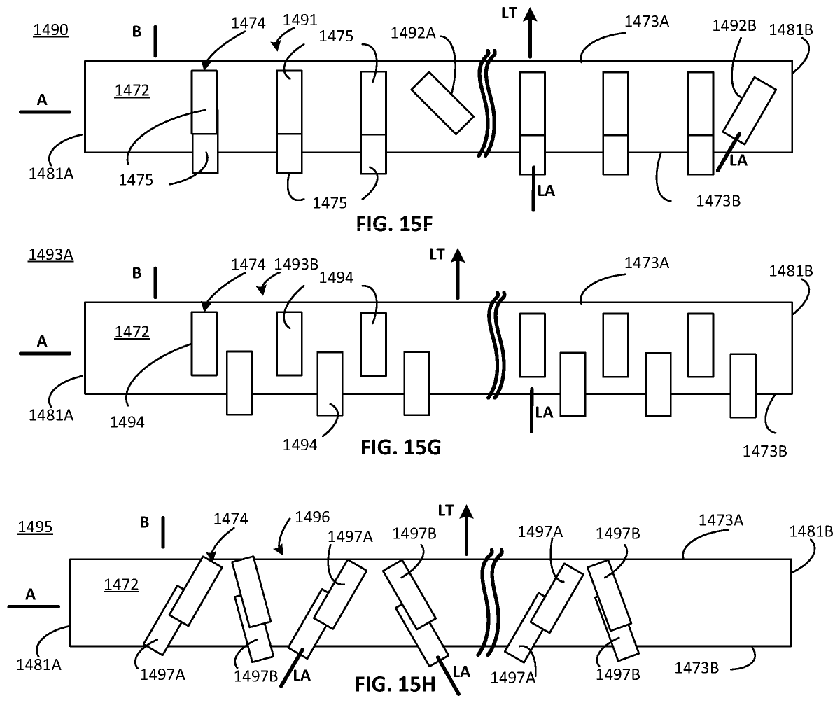

The lead body 622 comprises a plurality of electrical conductors (e.g. wires) which extend the length of the lead body 622 and which are housed in an insulative jacket, with each electric conductor being insulated to be independent of each other. In some examples, at least some of the aspects of this construction of lead body 622 may correspond to at least some of substantially the same features and attributes of a lead body construction depicted later in association with at least FIGS. 13B, 13C, and 15C. Accordingly, the lead body 622 establishes an electrical communication between the IPG 333 (or other pulse generator such as 1133 in FIG. 17A) and the electrode contacts of the respective stimulation elements 632A, 632B such that stimulation signals generated and applied by IPG 333 will be delivered to the target tissue.

In some examples, the bifurcation portion 628 may be located substantially closer to the stimulation elements 632A, 632B than to the IPG 333, such that during implant the bifurcation portion 628 can be maneuvered into and through (or is accessible within) the midline implant-access incision 609A. In some such examples, the bifurcation portion 628 may be located within a distance of the stimulation elements 632A, 632B at least about 2 times a length of a stimulation element (e.g. 632A). In some examples, the distance may be at least about 3 times a length, at least about 4 times a length, at least about 5 times and so on up to at least about 10 times a length of a stimulation element (e.g. 632A).

In some examples, the bifurcation portion 628 may be located substantially closer to an IPG 333 than to the stimulation elements 632A, 632B such that during implant a bifurcation portion (e.g. like 628) can be maneuvered into and through (or is accessible within) an implant-access incision 609B or a differently located implant-access incision. In some such examples, the bifurcation portion 628 may be located within a distance of the IPG 333 of at least about 2 times a length (or width) of an IPG 333. In some examples, the distance may be at least about 3 times a length, at least about 4 times a length, at least about 5 times and so on up to at least about 10 times a length of an IPG 333.

In general terms, these relationships regarding a location of a bifurcation portion described for the example arrangement of FIG. 3 may be applicable, in some examples, to a location of a bifurcation portion in other examples throughout the present disclosure, unless specifically noted otherwise or apparent from the particular context.

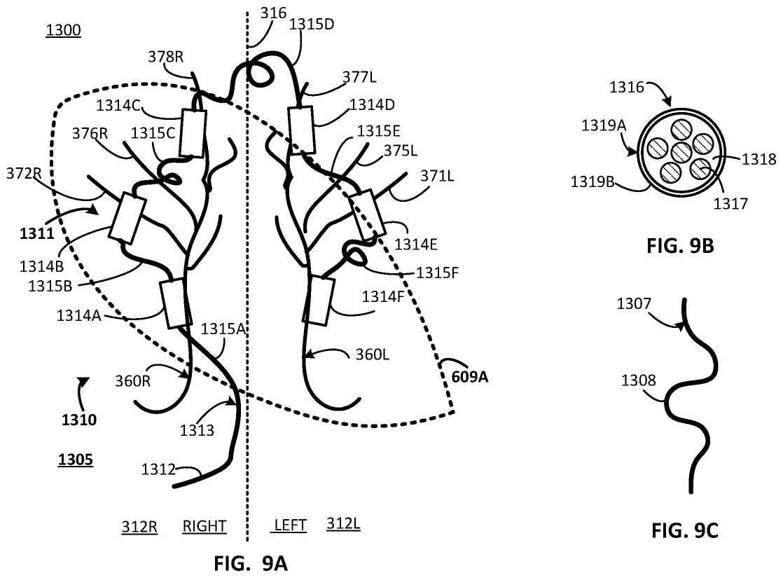

In some examples, one of the stimulation elements 632A, such as cuff electrodes (on first side 312R) or 632B (on opposite second side 312L), may be replaced with a different type of stimulation element which is not a cuff electrode, i.e. replaced by a non-cuff electrode, stimulation element. In some examples, the substitute stimulation element may comprise any one of the types of stimulation elements of the later-described examples of the present disclosure such as, but not limited to: (1) a clamp-style stimulation element 1232A or 1232B in FIGS. 8A-8L; (2) a connected array of stimulation elements similar to the arrangement in FIG. 9A-12B (with FIGS. 13A-15H) which is sized for implantation on just one side of the patient's body; (3) an axial array of electrodes on an elongate carrier such as stimulation element 1532A or 1532B in FIGS. 16A-17B; (4) a paddle-style carrier such as stimulation elements 2052A or 2052B in FIGS. 18A-24E.

In some of these examples, by providing a stimulation element on opposite sides of the body, one can apply unilateral stimulation, bilateral stimulation, or cross-lateral stimulation. In some of these examples, by providing different types of stimulation elements on different/opposite sides of the patient's body, one may be able to provide overall more robust therapy at least because each of the different types of stimulation elements may provide a different way of being in stimulating relation to target tissues, with some types of stimulation elements (e.g. a cuff electrode as in FIG. 3) potentially being more effective for direct stimulation of a nerve portion, while other types of stimulation elements (e.g. a paddle-style carrier 2052A or 2052B as in FIG. 18A-24E) may be more effective at being in stimulating relation to a combination of nerve portions, muscle portions, combinations of nerve portions and muscle portions, a neuromuscular junction of such nerve portions and muscle portions, and/or combinations thereof. In such arrangements, one particular type of stimulation element may provide more efficacious therapy for certain patients, may provide more comfortable electrical stimulation for certain patients, etc. Accordingly, after implantation, the patient and care team can customize their electrical stimulation therapy with a greater range of options available to achieve a desired combination of comfort, efficacy, etc.

FIG. 4 is a diagram 700 including a side view schematically representing an example device 705 which comprises a stimulation lead 710 comprising a body 722 extending between a proximal portion 720 and an opposite distal portion 724, which supports a first stimulation element 732A. In some examples, the first stimulation element 732A may comprise one example implementation of a respective one (e.g. 632A) of the stimulation elements (e.g. 632A, 632B) in FIG. 3. With further reference to FIG. 4, the first stimulation element 732A may comprise a linear array of electrodes 733 adapted to stimulate a hypoglossal nerve portion (e.g. 360R on one side 312R of the patient's body as shown by FIG. 3). However, it will be understood that the first stimulation element 732A may comprise other types of electrode configurations (e.g. cuff, paddle, etc.). Meanwhile, the proximal portion 720 of lead 710 is connectable to a port in header 735 of implantable pulse generator (IPG) 333. As further represented by the dashed lines in FIG. 4, the IPG 333 may be implanted via implant access-incision 609B, which may be in the pectoral region 332 as shown in FIG. 3. The same implant-access incision 609B also may be used to implantably position at least the proximal portion 720 of lead 710 and its connection to IPG 333.

With this in mind, the first stimulation lead 710 may be introduced and advanced subcutaneously via implant access-incision 609A for positioning stimulation element 732A in stimulating relation to a hypoglossal nerve portion (e.g. 376R in some examples) on a first side (e.g. 312R) of the patient's body, with lead body 722 extending between the hypoglossal nerve portion 376R and the IPG 333 in the pectoral region 332.

Moreover, via the same midline implant-access incision 609A, a second stimulation lead 760 may be introduced and advanced subcutaneously via implant access-incision 609A for positioning a second stimulation element 732B (at distal end 765 of lead 760) in stimulating relation to a hypoglossal nerve portion (e.g. 377L in some examples) on a second side (e.g. 312L) of the patient's body, with lead body 762 extending between the stimulation element 732B at hypoglossal nerve portion 377L and intermediate portion 745 of the first stimulation lead 710, as further described below.

With this in mind and as further shown in FIG. 4, the second stimulation lead 760 comprises a proximal portion 764 for connection to an intermediate portion 745 of the first stimulation lead 710. In particular, in some examples, the intermediate portion 745 of the first stimulation lead 710 comprises a port interface 750 including an extension arm 752 including a connection port to receive the proximal portion 764 of second stimulation lead 760 in order to establish electrical connection (and mechanical connection) of the lead 760 (and stimulation element 732B) to the IPG 333. In some examples, the arrangement of the port interface 750 extending from and being juxtaposed with the intermediate portion 745 of the first stimulation lead 710 may sometimes be referred to as a bifurcation portion 728 at least to the extent that the stimulation lead 760 and distally extending portion of the first stimulation lead 710 bifurcate from each other at or near the region of the port interface 750.

As shown in FIG. 4, the stimulation lead 710 may have a length such that its intermediate portion 745 (and port interface 750) is positioned in close proximity to the midline implant-access incision 609A to permit its accessibility via the midline implant-access incision 609A and generally contemporaneous with the implantation of the first stimulation lead 710. In some examples, like the first stimulation lead 710, the second stimulation lead 760 may be implanted using direct visualization techniques to introduce and advance the stimulation element 732B of stimulation lead 760 to the target nerve portion (e.g. 377L) on the second side (e.g. 312L) of the patient's body. In some examples, this implantation may be performed with no tunneling or minimal tunneling from the implant-access incision 609A to the implant location of stimulation element 732B.

As represented via FIG. 4, the second stimulation lead 760 comprises a length such that upon its implementation, the second stimulation element 732B will extend from the IPG 333 by a distance similar to the distance by which the first stimulation element 732A extends from the IPG 333. It will be understood that the second stimulation lead 760 may have a greater relative length or shorter relative length than shown in FIG. 4, with its predetermined length depending, at least in part, on the location of port interface 750 along the first stimulation lead 710.

As noted previously, the first stimulation lead 710 and second stimulation lead 760 may be operated to treat sleep disordered breathing via bilateral stimulation (or selective unilateral stimulation and other variations) of the nerve portions, such as portions of the hypoglossal nerve 360R (e.g. at 376R), 360L (e.g. at 377L) as illustrated by at least FIGS. 2A-2B and 3.

It will be understood that in some examples, just one (e.g. 732A) of the stimulation elements (e.g. 732A, 732B) may be implanted in an initial implantation procedure via midline implant-access incision 609A with the expectation that unilateral stimulation via the stimulation element 732A on one side (e.g. 312R) of the body will be sufficient to treat the particular patient's sleep disordered breathing. Upon a later determination that the patient exhibits an unsatisfactory level of sleep disordered breathing despite stimulation via the first stimulation lead 710 via stimulation element 732A at the hypoglossal nerve portion (e.g. 376R, in some examples), an example method schematically represented via FIG. 4 may comprise implanting the second stimulation lead 760 including second stimulation element 732B to be positioned in stimulating relation to a hypoglossal nerve portion (e.g. 377L, in some examples) on the other (e.g. second, opposite) side 312L of the patient's body. In some examples, the second stimulation element 732B may be similar to the first stimulation element 732A (including a linear array of electrodes 733) or may have a different configuration (e.g. cuff, paddle, etc.).

Via this arrangement, once it is determined that bilateral stimulation of the hypoglossal nerves (360R, 360L) is desirable in view of insufficient treatment of sleep disordered breathing from unilateral stimulation for a particular patient, then at a time period after implantation of the first stimulation lead 710, the second stimulation lead 760 may be implanted for connection to the IPG 333 via releasable connection of second stimulation lead 760 to the port interface 750 of stimulation lead 710.

As further represented in FIGS. 3-4, in order to add the second stimulation lead 760, one can use one of the implant-access-incisions 609A, 609B, (or in some examples, a new implant-access incision 609K intermediate between the implant-access incisions 609B, 609A) to access the port interface 750 of the already implanted lead 710. Such access may depend on a length of lead 710, a position of the port interface 750 along such length of the lead 710, etc. The variability in using one of the different implant-access incisions (e.g. 609B, 609K, 609A) is schematically represented by reference numeral 609Z in FIG. 4, which indicates that any one of the implant-access incisions 609A, 609B, 609K may be used to access the port interface 750 depending on the circumstances for the particular patient, particular lead, etc. In some such examples, as further represented in FIG. 3, tunneling T2 may be performed from the location of the port interface 750 of lead 710 to the intended implant location of the stimulation element 732B on second stimulation lead 760, such as at implant access-incision 609A, in some examples.

Via the example arrangement provided via example device 705 in FIG. 4, neurostimulation therapy can be conveniently expanded to include additional nerves when desired, such as to address a change in a patient's underlying condition, to enhance therapy, etc.

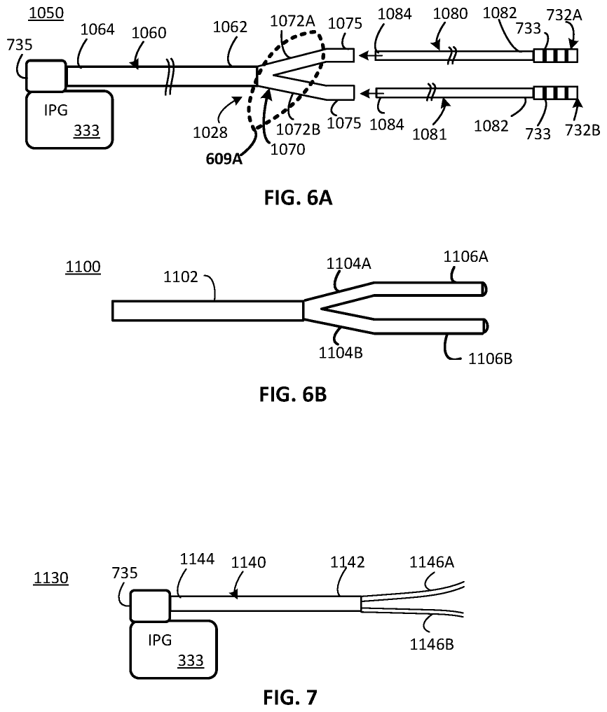

It will be understood that the various leads, stimulation elements, port interfaces, etc. described in association with at least FIGS. 1A-4 may be secured with sutures and/or a wide variety of anchors. FIG. 5A-5B provide example implementations of just some such anchors, while other types of anchors are described in association with at least FIGS. 8B-8L, 11A-11B, 13A-16A, 16J-16K, 23A-24E, and/or 25H. It will be further understood that the example anchors in FIGS. 5A-5B (e.g. wings, holes for tissue growth, etc.) may employed on any of the various stimulation elements, leads, etc. as appropriate. Moreover, in some examples, some of the anchor features (e.g. suture-friendly surfaces, wings, holes for sutures, holes for tissue growth, etc.) may be incorporated into implantable structures, such as a port interface (e.g. 750 in FIG. 4, 1070 in FIG. 6A, and the like) to facilitate their anchoring relative to non-nerve tissues and structures to stabilize the respective element within the patient's body.

FIG. 5A is side view schematically representing an elongate suture anchor element 800, which comprises a body 811 and a linear array of spaced-apart protrusions 812 to facilitate securing the anchor element, via sutures, relative to a non-nerve tissue. As further shown in FIG. 5A, the anchor element 800 may be fixed on a portion of a lead body 814 or slidable movable along the portion of the lead body 814 to be secured.

FIG. 5B is a side view schematically representing an anchor element 830, which comprises a body 831 and pair of wings 832 extending perpendicular outward from body 831 to facilitate securing the anchor element 830, via sutures, relative to a non-nerve tissue. As further shown in FIG. 5B, the anchor element 830 may be fixed on a portion of a lead body 814 or slidable movable along the portion of the lead body 814 to be secured.

Upon securing the anchor element 800 or 830, the lead body 814 becomes secured relative to non-nerve tissue within the patient's body. It will be understood that similar types of anchor features may be incorporated into portions of a lead, such as the various example port interfaces (e.g. FIG. 4, 6A, etc.) described in several examples of the present disclosure.

FIG. 6A is a diagram including a top view schematically representing an example arrangement 1000 including an IPG 333, bifurcated port interface 1070, and removably insertable stimulation leads 1080, 1081. In some examples, the example arrangement 1000 comprises at least some of substantially the same features and attributes as the example arrangements described in association with at least FIGS. 1A-5B, such as (but not limited to) the example arrangement 1000 including a bifurcation portion 1028 from which separate and independent stimulation lead (segments) 1080, 1081 extend to support stimulation elements 732A, 732B. In addition, in some examples, the example arrangement 1000 provides for flexibility in a sequence or timing of implanting the respective stimulation leads 1080, 1081 according to patient conditions, anatomy encountered during implantation, changing health overtime, etc.

As shown in the diagram 1050 of FIG. 6A, a lead support portion 1060 includes a proximal portion 1064 and a distal portion 1062. The proximal portion 1064 is electrically connectable to IPG 333 via header 735 and the distal portion 1062 supports a bifurcated port interface 1070. The port interface 1070 comprises two spaced apart prongs 1072A, 1072B which diverge from each other, with each prong 1072A, 1072B comprising a connection port 1075 to removably receive electrical (and mechanical) connection from a proximal portion 1084 of each stimulation lead 1080, 1081. In some examples, just one of the two prongs can be removably received from the proximal portion. Each stimulation lead 1080, 1081 comprises a distal portion 1082 supporting a respective stimulation element 732A, 732B, each of which comprise a linear array of electrodes 733. As in other examples, the stimulation elements 732A, 732B can take a wide variety of electrode configurations (e.g. cuff, paddle, etc.) other than the axial array of electrodes depicted in FIG. 6A.

In some examples, the IPG 333 and lead support portion 1060 (including port interface 1070) may be implanted to support the generally contemporaneous implantation of both stimulation leads 1080, 1081, such as via midline implant-access incision 609A. For instance, in a manner similar to those previously described, the stimulation lead (e.g., lead support portion 1060) can be introduced and positioned via midline implant-access incision 609A with port interface 1070 remaining in close proximity to the midline implant-access incision 609A to facilitate introduction and advancement of stimulation leads 1080, 1081 to place stimulation elements 732A, 732B into stimulating relation with target tissues (e.g. 376R, 377L) and connection of the proximal portion 1084 of leads 1080, 1081 to ports 1075 of bifurcated port interface 1070.

However, in some examples, in a manner similar to that previously described in association with at least FIG. 4, in some examples, just one of the stimulation leads 1080, 1081 may be implanted in an initial implantation procedure to be in stimulating relation to a first nerve portion (e.g. 376R). At a later point in time, such as a later determination upon significant use by the patient revealing insufficient treatment, the other respective one of the stimulation leads 1080, 1081 may be implanted to be in stimulating relation to a second nerve portion (e.g. 377L). In such arrangements, upon obtain access via the midline implant-access incision 609A, the port interface 1070 conveniently permits selective addition of the second stimulation lead (e.g. 1080 or 1081) during the second implant procedure by insertion of the proximal portion 1084 of the respective stimulation lead.

In some examples, implantation of port interface 1070 and/or stimulation leads 1080, 1081 may be facilitated via use of tunneling tool 1100 schematically represented in FIG. 6B. As shown in FIG. 6B, the tunneling tool 1100 comprises a proximal main portion 1102, which supports diverging portions 1104A, 1104B, from which extend spaced apart prongs 1106A, 1106B. The prongs 1106A are insertable into, and may be advanced through, subcutaneous tissue to form tunnels for implantation of stimulation leads and related structures.

In general terms, in some example methods, the tunneling tool 1100 may be deployed via a midline implant-access incision 609A, such as (but not limited to) some situations in which it may be difficult to reach a desired implant location for target tissue(s).

In some examples, the tunneling tool 1100 can be employed with example lead arrangements other than shown in FIG. 6A, and in which two different tunnels are to be formed subcutaneously to provide path for implantation of leads, stimulation elements, etc. It will be further understood that prongs 1106A, 1106B may have lengths with differ from each other, and may have tips which are steerable in some examples.

It will be understood that, in some examples, an alternative or additional implant-access incision (e.g. 609B, 609K in FIG. 3 or 609Z in FIG. 4) may be used to access the port interface 1070, depending on a length of the lead support portion 1060, a length of the stimulation leads 1080, 1081, and/or other factors.

FIG. 7 is a diagram including a top view schematically representing an example arrangement 1130 including a stimulation lead 1140. In some examples, the stimulation lead 1140 may comprise at least some of substantially the same features and attributes as, comprise an example implementation of, and/or be usable with the example arrangements described in association with at least FIGS. 1A-6B.

As shown in FIG. 7, in some examples, the stimulation lead 1140 comprises a proximal support portion 1144 electrically (and mechanically) connectable to an IPG 333 via header 735. Meanwhile, a distal portion 1142 of lead 1140 comprises a bifurcated pair of distal stimulation elements 1146A, 1146B. While just a portion of the distal stimulation elements 1146A, 1146B are shown for illustrative simplicity, it will be understood that each distal stimulation element 1146A, 1146B may support a stimulation element, such as stimulation elements 732A, 732B as described throughout the previously described examples or such as some of the later described stimulation elements. In some examples, just one of the two leads may be removably connected.

Moreover, in some example methods, the stimulation lead 1140 may be introduced and advanced subcutaneously via the midline implant-access incision 609A.

In some examples, at least some of substantially the same features and attributes (e.g. bifurcation, ports, connectability, etc.) of the examples of FIGS. 4-7 may be incorporated into, implemented as part of, applied to, etc. other examples throughout the present disclosure in association with at least FIGS. 1A-31.

FIG. 8A is a diagram 1200 schematically representing an example method and/or example device 1205 for treating sleep disordered breathing as implanted within a patient's body. As shown in FIG. 8A, one example device and/or example device 1205 comprises a stimulation lead 1210 comprising at least some of substantially the same features and attributes as in FIGS. 1A-7 and being implanted via a midline implant-access incision 609A via at least some of substantially the same features and attributes as in FIGS. 1A-7, except with each of a pair of stimulation elements 1232A, 1232B chronically implanted to be in stimulating relation to multiple nerve portions of a respective upper airway patency-related nerve 360R, 360L (e.g. hypoglossal nerve). In a manner similar to other examples of the present disclosure, it will be understand that some example devices and/or example methods comprise just one of the stimulation elements 1232A, 1232B instead of both stimulation elements 1232A, 1232B.

In a manner similar that shown in FIG. 3, the stimulation lead 1210 comprises a proximal portion (e.g. 620 in FIG. 3) and a distal portion 1224 including a bifurcation portion 1228 from which extends a pair of distal lead segments 1230A, 1230B each of which support a respective stimulation element 1232A, 1232B. At least the distal portion 1224 (including the stimulation elements 1232A, 1232B) may be implanted via the implant-access incision 609A, which may be located at or near the sagittal midline 316 in a manner as previously described for FIG. 3. It will be further understood that in examples including just one stimulation element (e.g. one of 1232A, 1232B), the bifurcation portion 1228 would be omitted and just one distal lead segment (1230A or 1230B) would extend from the distal portion 1224 of a leady body.

With further reference to FIG. 8A, among other aspects, by providing the bifurcation portion 1228 in a location (along the distal portion 1224 of lead 1210) proximally spaced apart from a proximal end 1234 of the respective stimulation elements 1232A, 1232B, the distal lead segments 1230A, 1230B (of lead 1210) have a length and flexibility suited for independent positioning of the stimulation element 1232A and stimulation element 1232B relative to each other. Among other aspects, the independent positionability of the stimulation elements 1232A, 1232B may enhance positioning and orienting of each stimulation element 1232A, 1232B as desired on each of the left and right sides (e.g. 312L, 312R) of the patient's body. For example, the distal lead segments 1230A, 1230B in this example of the present disclosure may enable more degrees of freedom in rotational orientations and/or translational orientations than some types of devices which include a central connector which is relatively rigid and/or which has a relatively bulky shape (e.g. square), significant size, etc.

In contrast, the independent positioning of the stimulation elements 1232A, 1232B as enhanced by the flexible distal lead segments 1230A, 1230B (and bifurcation portion 1228), in turn, may increase desired engagement with target tissues to increase the effectiveness of placing contact electrodes of the stimulation element 1232A, 1232B into stimulating relation to the target tissues. In some such examples, the distal lead segments 1230A, 1230B (and/or bifurcation portion 1228) omit circuitry other than conductors extending to the contact electrodes of the stimulation elements 1232A, 1232B such that the distal lead segments 1230A, 1230B (and/or bifurcation portion 1228) omit wireless communication circuitry (e.g. coils), wireless power transmission circuitry (e.g. coils), stimulation circuitry (e.g. stimulation pulse generation circuitry), and the like. Among other things, by providing the bifurcation portion 1228 with flexibility and a low profile (e.g. relatively smaller size, relatively narrow shape), the distal portion 1224 of lead 1210 (including stimulation elements 1232A, 1232B) can be more easily introduced and advanced via the implant-access incision 609A during implantation.

As shown in FIG. 8A, in some examples one or both of the stimulation elements 1232A, 1232B may comprise an elongate shape extending between a proximal end 1234 and a distal end 1236. As further shown in FIG. 8AA, each stimulation element 1232A, 1232B has a length L10 and a width W10, with width W10 corresponding to a width of one of the arms (e.g. 1256A, 1256B in FIG. 8B) forming each U-shaped stimulation element 1232A, 1232B. The length L10 extends between distal and proximal ends 1236, 1234, while the width W10 extends between opposite side edges 1231A, 1231B. Further details regarding these relationships are further described in association with at least FIG. 8B. As shown in FIG. 8AA, a distal lead portion 1230C (e.g. a distal end of distal lead segment 1230A or 1230B) may be electrically and mechanically connected to the proximal end 1234 (e.g. closed base portion) of the stimulation element (e.g. 1232A, 1232B). In some such examples, the distal lead portion 1230C extends generally perpendicular to an external surface of the closed proximal end 1234, which in some examples may facilitate introduction and advancement of the stimulation element 1232A, 1232B in generally alignment with a longitudinal axis of at least the distal lead portion of a stimulation lead. This example arrangement of the present disclosure may stand in contrast to some other designs (e.g. beyond the scope of present disclosure) in which a lead portion is connected to a distal open end of an open cuff electrode, and/or a side edge of an open cuff electrode, among other differences.

In some such examples, the elongate dimension of the stimulation elements 1232A, 1232B may extend generally parallel to the sagittal midline 316 of the patient's body. With this in mind, such an implant orientation of the stimulation elements 1232A, 1232B relative to the midline 316 may cause the stimulation element 1232A to be implantably positioned to extend generally transverse to (or at an non-parallel angle or generally parallel to) to an orientation (long axis) of multiple nerve portions such as (but not limited to) nerve portions 372R, 376R, and/or 378R (FIG. 8A) in some examples and to cause stimulation element 1232B to be implantably positioned to extend generally transverse to (or at an non-parallel angle or parallel to) to an orientation (long axis) of multiple nerve portions such as (but not limited to) nerve portions 371L, 375L, and/or 377L, as shown in FIG. 8A, in some examples. However, it will be understood that in some examples, upon implantation the elongate shape of at least one of the stimulation elements 1232A, 1232B may not be parallel to the patient's sagittal midline 316 but still may extend generally transverse to (or at a non-parallel angle to or parallel to) at least some target nerve portions (e.g. 372R, 376R, 378R, 371L, 375L, 377L, other).

In some examples, at least some of the respective target nerve portions 372R, 376R, and/or 378R may comprise terminal branches of nerve 360R. In some examples, the stimulating element 1232A is in stimulating relation to the target nerve portions 372R, 376R, and 378R, muscle portions innervated by those target nerve portions, both such target nerve portions and the innervated muscle portions, and/or a target neuromuscular junction of the target nerve portion and target muscle portion. In some such examples, the location of stimulating relation may include but is not necessarily exclusively defined at (or by) a neuromuscular junction of such target nerve portions (e.g. 372R, 376R, 378R) and the corresponding innervated muscle portions.

With this in mind, it will be understood that in some examples, the stimulation elements 1232A, 1232B of example stimulation lead 1210 may extend primarily in a mandibular plane (e.g. Mand in FIG. 8I) such that the contact electrodes (e.g. 1272A, 1272B, etc. in FIGS. 8B-8C) of stimulation elements 1232A, 1232B may be positioned to be in stimulating relation to few, if any, neuromuscular junctions (e.g. terminal distal ends) as target tissues and instead may primarily target nerve portion(s), muscle portion(s), and/or combinations of nerve portion(s) and muscle portion(s) affecting upper airway patency. Moreover, it will be understood that at least some of the nerve portions (e.g. 372R, 376R, 378R, 371L, 375L, 377L) considered to be target tissues may actually have a more superior orientation than shown in FIG. 8A and may be more likely to be in stimulating relation to electrode contacts of stimulation elements (e.g. 1232B in FIG. 8I or 8L) which are oriented in a more superior orientation and anterior orientation, such as shown in the examples of FIGS. 81-8J and/or in a more superior orientation and posterior orientation, such as shown in the examples of FIGS. 8K-8L. Nevertheless, the example nerve portions (e.g. 372R, 376R, 378R, 371L, 375L, 377L) shown in FIG. 8A represent at least a reference point along an anterior-posterior orientation of the patient's body at which the stimulation elements 1232A, 1232B may be implanted whether the stimulation elements 1232A, 1232B are to primarily extend along (e.g. in) a mandibular plane or to primarily extend superiorly and at an upward angle relative to the mandibular plane (e.g. Mand in FIGS. 81-8J or FIGS. 8K-8L).

FIG. 8B is a sectional view as taken along lines 8B-8B of FIG. 8A schematically representing a stimulation element 1232B in stimulating relation to target tissues (e.g. target nerve portions, target muscle portions, and/or target neuromuscular junctions). In some instances, the stimulation element 1232B may comprise individually addressable contact electrodes (e.g. 1272A-1272D) at least to the extent that in some examples, the contact electrodes may be in stimulating relation via a target tissue, such as being in direct contact with a target nerve portion(s), target muscle portion(s), combination of a target nerve portion(s) and target muscle portion(s), neuromuscular junction of the target nerve portion and target muscle portion, and/or combinations thereof. It will be understood that the example stimulation element 1232B shown in FIG. 8B also may be representative of the stimulation element 1232A in FIG. 8A.

In being described as being individually addressable, the contact electrodes 1272A-1272D can be activated separately (e.g. independently) of each other as controllable by an IPG (e.g. 333 in FIG. 2A, 1133 in FIG. 17A). In general terms the contact electrodes described in the various examples throughout the present disclosure comprise individually addressable contact electrodes, unless specifically noted otherwise.

However, in some examples, one or more of the contact electrodes (e.g. 1272A-1272D) may be in stimulating relation to a target tissue even when the contact electrode is in close proximity to a target tissue but does not directly contact the target tissue. With this in mind, such electrodes may sometimes be referred to more specifically as proximity electrodes, near contact electrodes, stimulation electrodes, and/or the like.

In some examples, the one or both of the stimulation elements 1232A, 1232B (FIGS. 8A-8B) may protrude relatively deeply into the available tissues (e.g. such as into the muscle base) to access desired target tissues, such as target nerve portions, target muscle portions, target combinations of nerve and muscle portions, target neuromuscular junctions (of nerve portions and muscle portions), and/or combinations thereof.

With further reference to the sectional view of FIG. 8B, in some examples stimulation element 1232B may comprise a distal portion 1252 of distal lead segment 1230B with the stimulation element 1232B acting to mechanically and electrically support contact electrodes to engage target tissues. In some examples, the tissue-engaging stimulation element 1232B comprises a U-shaped body 1251 including a base 1254 (at proximal end 1234 of stimulation element 1232B) and a pair of arms 1256A, 1256B which extend from base 1254 in a spaced apart relationship in some examples. In some examples, the spaced apart, arms 1256A, 1256B may be in a generally parallel relationship as shown in FIG. 2B before, during, and/or upon chronic implantation, while in some examples, the spaced apart arms 1256A, 1256B may form a non-parallel angle before, during, and/or upon chronic implantation such as later shown in FIG. 8C.

The U-shaped body 1251 comprises an outer surface 1262 and an inner surface 1260, which acts as a contact surface against or with target tissue portions. An end 1258A, 1258B of the respective arms 1256A, 1256B of the tissue-engaging stimulation element 1232B corresponds to the distal end 1236 of the stimulation element 1232B. Moreover, a distal opening 1238A (i.e. distal open end) of the U-shaped body 1251 is defined by the open space between the ends 1258A, 1258B of the arms 1256A, 1256B at the distal end 1236. In some examples, a channel 1238B is defined by the space between the arms 1256A, 1256B and having a depth D10 extending from the distal end 1236 to the inner surface 1255 of the closed base 1254 at the proximal end 1234 of the U-shaped body 1251. In general terms, the depth D10 corresponds to a length L11 of each arm 1256A, 1256B, which may be slightly less than an entire length of the stimulation element (e.g. 1232A). In some examples, the depth D10 of the open channel 1238B is substantially greater than (e.g. at least 2×, at least 3×, at least 4×, and so on) the width W10 (FIG. 8AA) of an arm (e.g. 1256A) of the U-shaped body 1251. In some examples, the channel 1238B is also open to the external environment at the side edges 1231A, 1231B (FIG. 8AA), such that the channel 1238B also may be considered as defining side openings at the opposite side edges 1231A, 1231B.

In some examples, the stimulation element 1232B comprises a plurality of contact electrodes 1272A, 1272B, 1272C, 1272D arranged on inner surface 1260, such as along the surfaces (e.g. 1263A, 1263B) of arm 1256A, base 1254, and/or arm 1256B which faces the target tissues 1241A (among other tissue 1241B). In the particular example shown in FIG. 8B, the contact electrodes 1272A, 1272B are located on the surface 1263A of arm 1256A while contact electrodes 1272C, 1272D are located on the surface 1263B of arm 1256B, with the surfaces 1263A, 1263B of the respective arms 1256A, 1256B facing each other. In some examples, similar contact electrodes may be located on the outer surface 1263C, 1263D of one or both arms 1256A, 1256B of a respective stimulation element 1232A, 1232B.

As further shown in FIG. 8B, the stimulation element 1232B may comprise an anchor structure 1276 on the body 1251 to secure the stimulation element 1232B relative to tissues 1241A, 1241B, which may comprise non-nerve tissue in some examples. As shown in FIG. 8B, in some examples the anchor structure 1276 may be located on just one arm 1256B as shown in FIG. 8B, or may be located on arm 1256A, or located on both arms 1256A, 1256B. In some examples, the anchor structure 1276 may comprise tines 1277 or other elements which engage surrounding non-nerve tissues to anchor the respective arm (e.g. 1256B) relative to the non-nerve tissue, and therefore relative to target tissue(s) 1241A, which comprises muscle portions 1242A, 1242B, 1242C and nerve portions 1244A, 1244B, 1244C (shown in cross-section).

Moreover, while FIG. 8B shows generous spacing between the inner surface 1260 of the tissue-engaging, stimulation element 1232B and the target tissue 1241A, it will be understood that each contact electrode 1272A, 1272B, 1272C, and/or 1272D is in sufficiently close proximity to the target tissue to be in stimulating relation to: (A) the target nerve portions 1244A, 1244B, and/or 1244C; (B) the target muscle portion(s) 1242A, 1242B, and/or 1242C; and/or (C) a combination of at least one of the target nerve portions and at least one of the target muscle portion(s) 1242A, 1242B, 1242C. As further illustrated in the later described FIG. 8D, in some examples each contact electrode 1272A, 1272B, 1272C, and/or 1272D (FIG. 8B) also may be in sufficiently close proximity to the target tissue to be in stimulating relation to a neuromuscular junction of at least some of the respective target portions and target muscle portions, which may be in addition or instead of, being in stimulating relation to (A) the target nerve portions 1244A, 1244B, and/or 1244C; (B) the target muscle portion(s) 1242A, 1242B, and/or 1242C; and/or (C) a combination of at least one of the target nerve portions 1244A, 1244B, 1244C and at least one of the target muscle portion(s) 1242A, 1242B, 1242C.

In some examples, the example target nerve portions 1244A, 1244B, 1244C shown in FIG. 8B may correspond to at least some of the example target nerve portions shown in FIG. 2B (e.g. 372R, 376R, 378R, 371L, 375L, 377L) or may correspond to other target nerve portions. Similar, in some examples, the example muscle portions 1242A, 1242B, 1242C shown in FIG. 8B may correspond to at least some of the example muscle portions shown in FIG. 2B (e.g. 382R, 386R, 388R, 383L, 387L, 389L).

As further shown in FIG. 8B, in some examples at least some of the contact electrodes such as, but not limited to, contact electrodes 1272B and 1272D may be operated to apply a stimulation vector N1 across and through tissue (e.g. nerve portion 1244B, muscle portion 1242B, etc.) in between the respective contact electrodes 1272B, 1272D. In some such examples, the stimulation vector N1 also may capture and deliver stimulation to a neuromuscular junction between the nerve portion 1244B and muscle portion 1242B, with such type of stimulation being further described later in association with at least FIG. 8D.

With further reference to at least FIG. 8B, it will be understood that the stimulation vector N1 is just one example, and that a wide variety of different stimulation vectors may be applied between or among the various available contact electrodes (e.g. 1272A, 1272B, 1272C, 1272D) with each different stimulation vector capturing and delivering stimulation to a different arrangement of target nerve portion(s), target muscle portion(s), combinations of target nerve portion(s) and target muscle portion(s), neuromuscular junctions of the target nerve portions and target muscle portions, and/or combinations thereof. By applying test stimulation signals according to the various stimulation vectors, it can be determined which stimulation vector(s) produce the desired effect on maintaining or increase upper airway patency according to a particular arrangement of stimulating the captured nerve portion(s), muscle portion(s), combinations of nerve portion(s) and muscle portion(s), neuromuscular junctions, and/or combinations thereof. Moreover, application of such test stimulation signals also may be used during implantation of the stimulation element (e.g. 1232B) in order to determine which positions, orientations, configurations (e.g. angle between arms 1256A, 1256B), depth of implant, etc. within and among the target tissues may produce the desired influence on maintaining or increasing upper airway patency upon applying a stimulation therapy signal via the contact electrodes.

As apparent from the foregoing description of examples associated with at least FIGS. 8A-8B and/or the following FIGS. 8C-8L, a relatively large volume of target tissue becomes encompassed (e.g. sandwiched) between the arms 1256A, 1256B of the stimulation element 1232A (and/or 1232B) with the target tissue comprising muscle portion(s), nerve portion(s), other tissues, neuromuscular junction(s), etc. This example stands in sharp contrast to non-example arrangements in which a cuff body of a cuff electrode may encompass just a nerve portion upon implantation after dissection (and maneuvering away of) other tissues (e.g. connective, muscle, etc.) near the nerve.

With this in mind, in some examples of the present disclosure implanting the stimulation element (e.g. 1232A and/or 1232B) may comprise positioning and advancing the arms (e.g. as represented by directional arrows V10 in FIGS. 8A, 8B), while maintaining their spaced apart relationship, to encompass a first cross-sectional area of the target tissue portion within and between the respective arms of a respective one of the stimulation element. In some such examples, the first cross-sectional area of the target tissue portion is substantially greater than a second cross-sectional area of at least one hypoglossal nerve portion (which resides within the target tissue portion) extending within and between the respective arms of a respective one of the stimulation element.

With this in mind, in some examples of the present disclosure implanting the stimulation element (e.g. 1232A and/or 1232B) comprises positioning and advancing the arms, while maintaining their spaced apart relationship, to encompass a first volume of the target tissue portion within and between the respective arms of a respective one of the stimulation element. In some such examples, the first volume of the target tissue portion is substantially greater than a second volume of at least one hypoglossal nerve portion (which resides within the target tissue portion) extending within and between the respective arms of the respective one of the stimulation element.

FIG. 8C is a diagram 1280 including a side view schematically representing an example implementation of a stimulation element 1232B of FIGS. 8A-8B, except with the arms 1256A, 1256B (e.g. arms) shown spaced apart in a non-parallel configuration such that the respective arms 1256A, 1256B extend at an angle (π) relative to each other. As previously noted, in some examples the spaced apart arms 1256A, 1256B of stimulation element 1232B may form a non-parallel angle (π) before, during, and/or upon chronic implantation within and relative to the target tissue.

In some examples, the angle (π) between the respective arms 1256A, 1256B may be selected (e.g. vary) depending on parameters such as a type, shape, location, orientation, etc. of the target tissue into which (and relative to which) the stimulation element 1232B is being advanced and implanted. In one such example, the particular angle (π) may depend on whether the stimulation element 1232B is being introduced from a more posterior location and advanced anteriorly and superiorly, as shown in FIG. 8I, or being introduced from a more anterior location (e.g. closer to the chin) and advanced posteriorly and superiorly, as shown in FIG. 8L.

In some examples, whether before, during, or upon chronic implantation, the angle (π) may be an acute angle (i.e. between 1 and 89 degrees) and/or an obtuse angle (i.e. between 91 and 179 degrees). To facilitate such bending of the arms 1256A, 1256B relative to each other, the body 1251 and/or at least the base 1254 of the stimulation element 1232B may comprise a flexible material, which may in some examples be resilient and biased such that the arms 1256A, 1256B tend to return to their original, default configuration, which may be a generally parallel relationship in some examples or other non-parallel configuration in some examples.

In some examples, the flexible material may be formed of shape-retaining materials which permit flexing or bending the arms 1256A, 1256B relative to each other during implantation but then enable retaining a particular shape or configuration (e.g. angle (π)) into which the respective arms 1256A, 1256B have been manipulated to implement a particular orientation etc. for chronic implantation relative to target tissues.

It will be further understood that the arms 1256A, 1256B themselves also may comprise a resilient flexible material to facilitate their flexing, bending, etc. in order to help advance, position, maneuver, etc. the stimulation element 1232B into its desired location, orientation, etc. to be in stimulating relation to target tissues. In some examples, the ends 1258A, 1258B of each respective arm 1256A, 1256B also may rounded or otherwise shaped to facilitate entry and passage into and/or through tissue.

With further reference to FIGS. 8A-8B, instead of, or addition to the previously described potential target nerve portions and/or target muscle portions (and/or combination of such targets), in some examples at least one of the contact electrodes (e.g. 1272A, 1272B, 1272C, and/or 1272D may be in sufficiently close proximity to the target tissue (e.g. 1240) to be in stimulating relation to a neuromuscular junction of a respective one of the target nerve portions and a respective one of the target muscle portions.

With this in mind, FIG. 8D is a diagram similar to the example arrangement in FIG. 8B, except schematically representing an example arrangement in which at least some of contact electrodes 1297A, 1297B, 1297C, 1297D, 1297E of a stimulation element 1290 (like 1232B or 1232A) are an implanted positon to be in stimulating relation to: (A) a neuromuscular junction 1294A of the respective target nerve portion 1292A and target muscle portion 1293A; (B) a neuromuscular junction 1294B of the respective target nerve portion 1292B and target muscle portion 1293B; and/or (C) a neuromuscular junction 1294C of the respective target nerve portion 1292C and target muscle portion 1293C. In some examples, a terminal portion of a respective target nerve portion (e.g. 1292A) partially defines the neuromuscular junction (e.g. 1294A) and may comprise a terminal end 1291. In some such examples, the portion of a target nerve portion which partially defines a neuromuscular junction may sometimes be referred to as a target distal terminal nerve portion.

It will be understood that in the example positions shown in FIG. 8D, at least some of the contact electrodes 1297A, 1297B, 1297C, 1297D, 1297E also may in stimulating relation to a corresponding nerve portion (e.g. e.g. 1292A, 1292B, 1292C, respectively) and/or muscle portion (e.g. 1293A, 1293B, 1293C, respectively) associated with the particular neuromuscular junction 1294A, 1294B, 1294C.

With further reference to FIG. 8B, in order for at least arm 1256B of the stimulation element 1232B to be placed into stimulating relation to target tissue 1240, in some examples an implant path 1248 is formed and comprises opposite side walls 1247A, 1247B with implant path 1248 comprising an opening 1249A formed in a tissue surface 1243 and comprising a closed end 1249B. In some instances, the implant path 1248 may sometimes comprise a tunnel or partial tunnel. However, to the extent that in some examples, the implant path may be relatively shallow and/or relatively short (as compared to a longer tunnel), the implant path 1248 may sometimes comprise or be implemented via an incision having a size and/or shape approximately the desired implant path. It will be fuller understood that the implant path 1248 may comprise part of a larger implant path which enables implantable delivery and positioning of arm 1256A, 1256B, etc.

FIG. 8E is a diagram 6900 including a side view schematically representing an example stimulation element 6910 of an example stimulation lead. In some examples, the example stimulation element 6910 comprises at least some of substantially the same features and attributes as various stimulation elements described in association with the FIGURES of the present disclosure, while also comprising an anchor structure 6920 instead of other anchoring arrangement such as tines 1277 in FIG. 8B or other types of anchor elements. In some examples, the stimulation element 6910 may comprise a distal portion of a stimulation lead body which extends proximally from the proximal end 6718 of the stimulation element 6910. It will be understood that anchor structure 6920 may be substituted for the tines 1277 in the example arrangement of FIG. 8B and/or for other anchor structures in various examples throughout the present disclosure.

As shown in FIG. 8E, in some examples the anchor structure 6920 comprises a plurality of anchor elements 6924 which protrude from the sides 6711 of the body 6713 of the stimulation element 6910. In some examples, the anchor elements 6924 may be grouped into different arrays 6922A, 6922B while in some examples, the anchor structure 6920 may comprise a single cluster of anchor elements 6924.

It will be understood that in some examples, the elements 6924 may extend about an entire periphery (e.g. circumference of body 6713).

As shown in FIG. 8E, the anchor structure 6920 is positioned distal to the electrode array 6714, being between the electrode array 6714 and the distal end 6719 of the body 6713 of the stimulation element 6910.

In this configuration, the position of the anchor structure 6920 on just one end (e.g. the distal end) of the electrode array 6714 may prevent or minimize “lead elongation”, i.e. elongation of the lead body 6713 which may potentially be caused by muscle movement when anchor elements (e.g. tines) are present on opposite ends of the electrode array 6714.

In some examples, the elements 6924 may comprise a filament (e.g. fine thread) which is flexible and resilient, and biased to extend outward from the side 6711 of body 6713. The filament may be formed of a polymer material, such as but not limited to, nylon, propylene, silk, polyester, trimethylene carbonate, and the like. In some examples, such filaments may be resorbable or may be non-resorbable.

In some examples, each element 6924 may comprise a diameter (or greatest cross-sectional dimension) of about 0.05 to about 0.60 millimeters. In some examples, each element 6924 may comprise a length of about 0.2 to about 2 millimeters. In some examples, each element 6924 may comprise a length about 0.5 percent to about 50 percent of a diameter of the lead body 6713 in the region of the electrode array 6714 and/or at distal end 6719. In some examples, at least some or all of the anchor elements 6924 may have generally the same shape, size, orientation, material, configuration, etc. such that the anchor elements 6924 may sometimes be referred to as being generally homogeneous anchor elements, i.e. being generally the same as each other.

However, in some examples, the anchor structure 6920 may be embodied as a matrix (e.g. grouped arrangement) of heterogeneous elements via filaments having pseudo-random sizes, shapes, orientations and/or positions exhibiting more variation than a plurality of identical or substantially similar discrete elements (e.g. 6927 in FIG. 8F), which may be visually recognizable.

Meanwhile, in some examples, all of the various features of the matrix of heterogeneous elements may not be readily visually recognizable. Among other features, this heterogeneous matrix may enable fixation in both (e.g. opposite) orientations (along length of stimulation element/lead) and ease deliverability of the lead, lead portions. At least some example implementations of anchor structures 7000, 7100 comprising a matrix of heterogeneous elements are described later in association with at least FIGS. 15A-15B. In some examples, the heterogeneous elements may sometimes be referred to as heterogeneous fixation elements.

In some examples, the term matrix connotes a grouped arrangement of the fixation elements (e.g. anchor elements) in which the fixation elements are (structurally) independent from each other even though some of the fixation elements may at least partially contact each other in (at least) some instances.

Stated differently, in some examples the grouped fixation elements do not interconnect with each other in a latticework or mesh format. In some examples, the fixation elements may be homogeneous relative to each other or in some examples, the fixation elements may be heterogeneous relative to each other. In some examples, the fixation elements may be oriented in near parallel planes, and in other examples, the fixation elements could be in orientations with intersecting planes. In some examples, the relative orientation of the fixation elements can be random.

In some examples, the anchor structure 6920 may comprise a plurality of well-defined, discrete elements but with at least some of the discrete elements comprising a size, shape, orientation, and/or position different from a size, shape, orientation, and/or position of other respective discrete elements of the anchor structure 6920.

In some examples, the anchor structure 6920 may enhance some example methods of implantation of a stimulation device at least because the respective elements 6924 exhibit a low profile relative to an outer diameter of the body 6713 of the stimulation element 6910 such that the stimulation element 6910 (FIG. 8E-8H) can be delivered via hollow insertion needle 6760 without a sleeve or similar elements while still robustly securing the stimulation element 6910.

As further shown in the greatly enlarged side view of just one element 6924 in FIG. 8F, in some examples, at least some (or all) of the elements 6924 may comprise protrusions 6927 on their surfaces, which in some examples may comprise barbs, hooks, or other sharp tipped structures. In some examples, the protrusions 6927 may be present on just a portion of the element 6924, such as but not limited to a distal portion 6929 of the element 6924. However, in some examples, the protrusions 6927 may be present on the entire or substantially entire surface of the element 6924. In yet other examples, groups of protrusions 6927 may be positioned in spaced apart clusters, which are spaced apart from each other along and around the surface of the element 6924.

It will be further understood that the protrusions 6927 are not strictly limited to structures having a sharp-tip or hook but may comprise structures comprising a rounded edge while including a sticky surface coating or formed as a non-sharp tipped member which can securely engage a surrounding non-nerve tissue in close proximity to a target stimulation site.

FIG. 8G is a diagram including a side view schematically representing an example protrusion 6928. In some examples, the protrusion 6928 may comprise at least some of substantially the same features and attributes as protrusion 6927 described in association with at least FIG. 8F and/or may comprise one example implementation of protrusion 6927. As shown in FIG. 8G, in some examples protrusion 6928 may comprise a main element 6923 for protruding outward (e.g. biased to extend outwardly at an angle) from an outer surface of a lead to function as part of an anchor structure, with protrusion 6928 including a first secondary element 6925A extending at an angle relative to the main element 6923. The combination of the first secondary element 6925A and the main element 6923 may sometimes be referred to as a barb at least to the extent that the respective main and secondary elements 6923, 6925A form a sharp point with the secondary element 6925A having an orientation which is at least partly opposite of the general orientation of the main element 6923. In some examples, the protrusion 6928 may further comprise additional secondary elements 6925B spaced apart from each other along a length of the main element 6923 and also extending outward at angle relative to the main element 6923. In some examples, each secondary element 6925B also may comprise a barb, e.g. a further protrusion extending at an angle relative to the secondary element.

With regard to the example stimulation element 6910 in FIGS. 8E-8H, it will be understood that in some examples the anchor structure 6920 may be located solely proximally of the electrode array 6714 such that no similar anchor structure 6920 is located distal to the electrode array 6714.

However, in some examples, a first anchor structure 6920 may be present distal to the electrode array 6714 as shown in FIGS. 8E, 8H and a second anchor structure, similar to anchor structure 6920, may be present proximal to the electrode array 6714 so that at least some anchor structure or elements are present on opposite ends (e.g. sides when seen in the view of FIG. 8E) of the electrode array 6714. In some examples, elements 6924 of an anchor structure 6920 may be located between adjacent electrodes 6716 of an array 6714 of electrodes.

FIG. 8H is a diagram including a side view schematically representing an example arrangement 6950 of an example device and/or example method of implantation including the stimulation element 6910 slidably, removably inserted within the hollow insertion needle 6760.

As shown in FIG. 8H, upon insertion of stimulation element 6910 into and within lumen 6768 of hollow insertion needle 6760 (such as insertion via/through a proximal end 6764 of needle 6760), the elements 6924 of anchor structure 6920 become at least partially collapsed against side 6711 of stimulation element 6910 as forced by side wall 6765 of lumen 6768. The lumen extends between proximal end 6764 and opposite distal end 6762. The needle 6760 also may include a beveled portion 6763 at distal end 6762 to facilitate penetration of the needle 6760 into pertinent tissues at, and through, which the stimulation device 6710 is to be implanted.

In a manner similar to previously-described examples, with the stimulation element 6910 carried within the hollow insertion needle 6760, the combination of these elements are finally positioned within the vicinity of a target stimulation location. Needle 6760 is then withdrawn (represented by directional arrow WD) to leave the stimulation element 6910 in stimulation relation to the target stimulation location and to enable the elements 6924 of anchor structure 6920 to engage surrounding non-nerve tissues to robustly secure the stimulation element (e.g. electrode array 6714) in the stimulating relation position.

FIG. 8I is diagram 7200 including a side view schematically representing an example method and/or example device 7205 including a stimulation lead 7210, which comprises a stimulation element 7232B chronically implantable in stimulating relation to target tissues 7250 of a head-and-neck region, such as tissues which may affect upper airway patency and hence which sometimes be referred to as upper airway patency-related tissue. In some examples, stimulation element 7232B may comprise at least some of substantially the same features and attributes as the stimulation element 1232B (or 1232A) of FIGS. 8A-8D and/or one example implementation of the stimulation element 1232B (or 1232A).

As shown in FIG. 8I, the stimulation element 7232B may be introduced and advanced into and through implant-access incision 609A (FIG. 8A) in an orientation along the directional arrows V11 such that the stimulation element 7232B becomes chronically implanted with the distal end 1236 of the arms 1256A, 1256B of the stimulation element 7232B extending superiorly away from the general mandibular plane (see plane designators Mand) and extending anteriorly toward a front of the mouth, while a proximal end 1234 (e.g. base) of the stimulation element 7232B remains at or near the mandibular plane (see indicator Mand). In this regard, FIG. 8I generally schematically represents a bottom of a mandible bony portion via indicator 7241.

While FIG. 8I depicts the arms 1256A, 1256B of the stimulation element 7232B as being in a generally parallel relationship as one example, it will be understood that in their chronically implanted configuration, the arms 1256A, 1256B may extend at other non-parallel angles relative to each other, as previously described in association with at least FIG. 8C.

As further shown in FIG. 8I, in its chronically implanted position the arms 1256A, 1256B of the stimulation element 7232B may straddle a nerve portion 7280, which may comprise a more proximal portion of a medial branch of the hypoglossal nerve 7260. With the nerve portion 7280 extending for a length between the arms 1256A, 1256B, several different stimulation vectors may be applied among the various contact electrodes 1272A, 1272B, 1272C, 1272D, 1272E, and/or 1272F. As can be further seen in FIG. 8B, other branches (e.g. nerve portions 7270, 7272, 7262, etc.) extend outwardly from the nerve portion 7280 and also can be captured and stimulated via different stimulation vectors between arms 1256A, 1256B which may be applied via the plurality of contact electrodes 1272A-1272F.

Among other aspects, this example arrangement may enable stimulating various nerve portion(s), muscle portion(s), etc. without necessarily driving the distal end 1236 (of the arms 1256A, 1256B) of the stimulation element 7232B to the most distal segments of the nerve portion(s) and associated muscle portion(s), etc. For example, as shown in FIG. 8I, one example distal terminal nerve portion 7285 of a group or region 7282 of such distal terminal nerve portions is located superior to the stimulation element 7232B such that the contact electrodes (e.g. 1272A-1272F) may not be in direct stimulating relation to such distal terminal nerve portions 7285, 7292, 7264. However, by capturing the more proximal nerve portions (e.g. 7280, 7261) of the hypoglossal nerve via various potential stimulation vectors between arms 1256A, 1256B, the stimulation element 7232B may be provide therapeutic stimulation therapy signals indirectly to the distal terminal nerve portions 7264, 7285, 7292 and/or less proximal nerve portions (e.g. 7290 which supports distal terminal nerve portion 7292).

As further shown in FIG. 8I, the more distal terminal nerve portions (e.g. 7264) extending from nerve portion 7262 may innervate muscle portions 7244A, 7244B, 7244C which originate from an interior portion of chin 7240. Moreover, the more distal terminal nerve portions (e.g. 7292) may innervate muscle portions 7247 closer to a top surface portion of the tongue 7246. Other groups 7282, 7274 of distal terminal nerve portions 7285 may innervate more proximal muscle portions of the tongue (genioglossus muscle), at least some of which are involved in causing protrusion of the tongue and hence which may sometimes be referred to as protrusor muscles.

With this general arrangement in mind for the example method and/or example device 7205 including stimulation lead 7210, FIG. 8J provides a diagram 7300 including a front view schematically representing the example stimulation elements 7232A, 7232B in a chronically implanted position generally corresponding to the example of FIG. 8I with some aspects of the stimulation elements 7232A, 7232B shown in a simplified manner for illustrative clarity.

Consistent with FIGS. 8A-8D, the stimulation elements 7232A, 7232B are located on opposite sides of a sagittal midline 316 to be positioned for bilateral stimulation, or unilateral stimulation as desired. As shown in FIG. 8J, a distal portion 1237 of each stimulation element 7232A, 7232B extends upward in a general superior orientation with FIG. 8J depicting an outer surface 1239 of one arm 1256A facing forward in an anterior orientation. In this configuration, the plurality of contact electrodes 1272A, 1272B, etc. extend, in a vertically spaced apart relationship, in a column generally vertically upward in the superior orientation to be positioned to be in stimulating relation with target tissues (e.g. nerve portion(s), muscle portion(s), combinations of nerve portion(s) and muscle portion(s), neuromuscular junctions of nerve portion(s) and muscle portion(s), and combinations thereof).

As further shown in FIG. 8J, example contact electrode 1272X represents a visible distal portion of a contact electrode (e.g. 1272E) with the proximal portion of the contact electrode being unseen in FIG. 8J at least because the more proximal portions of the respective arms 1256A of the respective stimulation elements 7232A, 7232B extend posteriorly out of view. Moreover, portion 1235 represents a transition portion at which each arm 1256A extends posteriorly to a degree such that the remaining proximal portion of each arm 1256A is no longer visible in FIG. 8J.

As further shown in FIG. 8J, at least some of the nerve portions (e.g. 7371, 7373, 7375, 7285, etc.) may extend along a path intersecting with, or being in sufficiently close proximity, to be in stimulating relation to at least some of the contact electrodes (e.g. 1272A, 1272B, etc.) and/or a stimulation vector between various combinations of contact electrodes, as previously described in association with at least FIGS. 8A-8D.

FIG. 8K is a diagram 7400 including a bottom view like that of FIG. 8A which schematically represents an example method and/or example device 7405 including a stimulation lead 7410, which may comprise (in some examples) at least some of substantially the same features and attributes as the example method/device 7405 (and stimulation lead 7410) of FIGS. 8A-8J, except with the stimulation elements 7432A, 7432B in FIG. 8K being implanted with an opposite orientation from stimulation elements 7232A, 7232B of FIG. 8I, 8J, among other differences. In particular, whereas the stimulation elements 7232A, 7232B in FIGS. 81, 8J were implanted with a distal end 1236 oriented generally anteriorly (and superiorly), a distal portion 1237 of the stimulation elements 7432A, 7432B in FIGS. 8K and 8K are oriented generally posteriorly (and superiorly) with their proximal end 1234 facing the chin 315, as further described below.

As shown in FIG. 8K, in some examples the stimulation lead 7410 comprises a proximal portion 7422 and distal portion 7424, which includes distal lead segments 7430A, 7430B. A distal end 1233 of the distal lead segments 7430A, 7430B support, and are connected to, a proximal end 1234 of the respective stimulation elements 7432A, 7432B. In some examples, the distal lead segments 7430A, 7430B have a length substantially greater than a length of each stimulation element 7432A, 7432B, which may enhance independent positioning of the stimulation elements 7432A, 7432B during introduction into and through the implant-access incision and during their positioning, orienting, etc. into stimulating relation to target tissues. For example, the length of the distal lead segments 7430A, 7430B (extending from bifurcation portion 7428 to distal end 1233) is sized so that with the bifurcation portion 7428 in a more inferior position along or near the sagittal midline 316, the stimulation elements 7432A, 7432B can be oriented with their proximal end 1234 pointed toward chin 315 (e.g. anteriorly and/or superiorly) and with their distal end 1236 pointed inferiorly and/or posteriorly (as also represented by directional arrows V12), which is in an opposite direction from the primarily superior orientation of the distal lead segments 7430A, 7430B as they extend distally from the bifurcation portion 7428.

In some such examples, the implantation may sometimes be referred to as being chin-centric in the sense that the midline implant-access incision 609A is located relatively closer to chin 315 with the stimulation elements 7432A, 7432B being introduced and advanced through the implant-access incision 609A near chin 315 to be advanced in an orientation away from the chin 315 (as represented by directional arrows V12).

FIG. 8L is a diagram 7500 including a side view like that of FIG. 8I except schematically representing a stimulation element 7432B of stimulation lead 7510 instead of the stimulation element 7232B in FIG. 8I. According, the diagram 7500 of FIG. 8L comprises at least some of substantially the same features and attributes as the diagram 7200 of FIG. 8I, except with the stimulation element 7432B (of device 7505) having a generally opposite orientation, such as with its distal end 1236 being oriented primarily in a posterior, superior orientation.

It will be understood that when considered from a front view like FIG. 8J, the stimulation elements 7432A, 7432B of FIGS. 8K-8L would have an appearance, function, and/or operation similar to the stimulation elements 7232A, 7232B as shown in FIG. 8J except for the stimulation elements 7432A, 7432B having the primarily posterior, superior orientation of FIG. 8L. In this arrangement, the stimulation element 7432B may be implanted via a more chin-centric implant-access incision (e.g. 609A) as described in association with FIG. 8K. Otherwise, as represented via directional arrows V12, upon advancing the stimulation elements 7432A, 7432B with this orientation, at least some of the contact electrodes 1272A-1272F of the stimulation element 7432B (and similarly 7432A) become positioned in stimulating relation to various nerve portion(s) (e.g. 7260, 7270, 7280, 7272, etc.) such stimulation may be applied to the target tissues (e.g. at least nerve portions) via various stimulation therapy vectors across, between, and among the contact electrodes 1272A-1272B.

FIG. 9A is a diagram 1300 schematically representing an example method and/or example device 1305 for treating sleep disordered breathing via a stimulation portion 1311 comprising a connected array of stimulation elements 1314A-1314F. As shown in FIG. 9A, one example method and/or example device 1305 comprises a stimulation lead 1310 comprising at least some of substantially the same features and attributes as in FIGS. 1A-7 and being implanted via a midline implant-access incision 609A via at least some of substantially the same features and attributes as in FIGS. 1A-7, except with lead 1310 including a single strand of stimulation elements 1314A-1314F which omits a bifurcation portion like portion 628 in FIG. 3, 728 in FIG. 4A, 1228 in FIG. 8A, etc. among other differences.

Among other aspects, the example device 1305 may provide for a highly customizable implantation of multiple stimulation elements among several different target tissues (e.g. target nerve portions, target muscle portions, combinations of nerve portions and muscle portions, neuromuscular junctions, and/or combinations thereof) in order to apply stimulation selectively and independently to different target tissues and/or in order to apply stimulation via various combinations of the implanted stimulation elements to achieve desired stimulation vectors. The stimulation may applied among the different stimulation elements in different manners, such as (but not limited to) sequentially, simultaneously, alternating, bilaterally, unilaterally, and/or via other patterns.

In some examples, the example method and/or example device 1305 comprises the stimulation portion 1311 of a connected array of spaced apart stimulation elements 1314A-1314F with each stimulation element 1314A-1314F being independently positionable into stimulating relation to different portions of upper airway patency-related tissue. As shown in FIG. 9A, the stimulation portion 1311 comprising the connected array may be supported by or on a distal portion 1313 of stimulation lead 1310. In some examples, the distal portion 1313 may sometimes be referred to as distal lead portion 1313.

While it will be understood that the distal lead portion 1313 provides a continuous length including electrical conductors (covered by an insulative jacket), when viewed from a proximal to distal orientation, the distal lead portion 1313 may referred to as including various lead segments (e.g. 1315A, 1315B, etc.) which are exposed (e.g. present) prior to, between, or after a stimulation element (e.g. 1314A, 1314B, etc.). For instance, in some examples the distal lead portion 1313 may comprise one exposed lead segment 1315A being located just proximal to a stimulation element 1314A, an exposed lead segment 1315B being located between stimulation elements 1314A, 1314B, and exposed lead segment 1315C located between stimulation elements 1314B, 1314C, and so on. It will be further understood that the distal lead portion 1313 comprises an array of electrically independent conductors extending therethrough with each independent conductor in electrical communication and connection with a corresponding stimulation element (e.g. 1314A, 1314B, etc.) such that the respective stimulation elements 1314A-1314F are independently controllable for applying a stimulation signal to different target tissue portions (e.g. nerve, muscle, or nerve-muscle combination). One example implementation is shown later in FIG. 9B. At least the later-described FIGS. 13B-13C, 14D, 15C, etc. provide at least some example implementations of such arrangements in which electrical conductors extend within and through a stimulation lead body, stimulation element, and the like.

As further shown in FIG. 9A, in some examples, the distal lead portion 1313 is implanted such that at least some of the respective stimulation elements 1314A-1314F are chronically positioned in stimulating relation to target tissues, with several stimulation elements (e.g. 1314A, 1314B, 1314C) are fixed in stimulating relation to target tissues on one side (e.g. right side 312R) of the patient's body while several stimulation elements (e.g. 1314D, 1314E, 1314F) are fixed in stimulating relation to target tissues on another side (e.g. left side 312L) of the patient's body. The number of stimulation elements on one side (e.g. right side 312R) of the patient's body may be the same as, or different from, the number of stimulation elements on the opposite, other side (e.g. left side) of the patient's body.

Moreover, in some examples, the stimulation elements implanted on one side (e.g. right side) of the patient's body may be positioned at generally the same target tissue locations as the position of the respective implanted stimulation elements located on the other side (e.g. left side) of the patient's body. However, in some examples, the stimulation elements implanted on one side (e.g. right side) of the patient's body may be positioned in at least some target tissue locations which are different from at least some of the target tissue locations at which implanted stimulation elements on the other side (e.g. left side) of the patient's body are positioned.

In some examples, each stimulation element 1314A-1314F may comprise a carrier supporting a single contact electrode. The carrier may comprise an insulative material and is configured to permit electrical connection between the contact electrode and the electrical conductor(s) extending through the distal lead portion 1313 on which the stimulation element (e.g. 1314A, 1314B, etc.) is mounted. However, in some examples, each stimulation element may comprise multiple contact electrodes spaced apart from each other on the carrier.

In some examples, each contact electrode may comprises a ring electrode, a split-ring electrode, or the like.

As shown in FIG. 9A, in some examples at least some of the stimulation elements 1314A, 1314B, etc. are positioned to directly overlap with a nerve portion, such as stimulation element 1314A directly overlapping with nerve (portion) 360R, stimulation element 1314B directly overlapping with nerve portion 372R, stimulation element 1314C directly overlapping with nerve portion 378R, stimulation element 1314D overlapping with nerve portion 377L, stimulation element 1314E overlapping with nerve portion 371L, stimulation element 1314F overlapping with nerve (portion) 360L.

However, in some examples, some stimulation elements may not necessarily directly overlap with a nearby nerve portion, such as one of the stimulation elements being in close proximity to (but not directly overlapping) a nerve portion. This arrangement may occur because of the particular patient anatomy, size and/or shape of the stimulation elements, etc. Nevertheless, to the extent that the particular non-overlapping stimulation element may be close enough to the target nerve or target neuromuscular junction, the stimulation element may still be in stimulating relation to at least the target nerve portion, target muscle portion, and/or target neuromuscular junction such as but not limited to the manner depicted in the example arrangement of FIG. 8C. Moreover, even if the stimulation element is not in stimulating relation to a nearby target nerve and/or neuromuscular junction, the stimulation element may be close enough to a target muscle portion to be in stimulating relation to the target muscle portion (e.g. portion of the genioglossus muscle) suitable to positively affect upper airway patency.

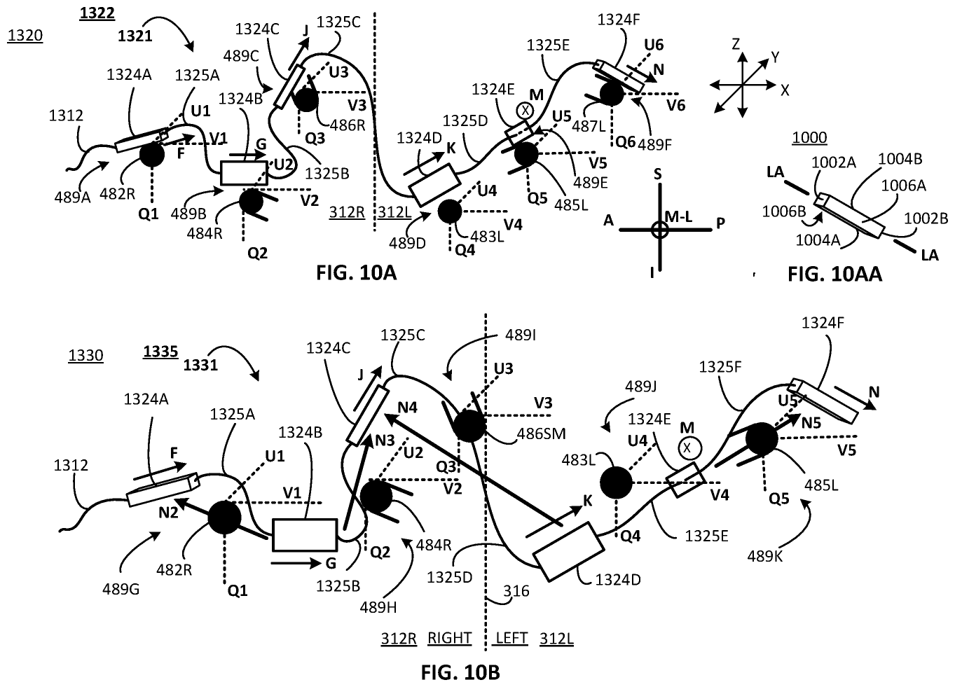

Moreover, in some examples, at least some target tissue (e.g. nerve portion, muscle portion, combinations of nerve portions and muscle portions, neuromuscular junctions, and/or combinations thereof) may be stimulated via stimulation vectors applied across, applied through, etc. target tissue which is located between or near spaced apart stimulation elements (e.g. 1314A-1314F) of the stimulation lead 1310. For instance, at least some such stimulation vectors may be formed and/or operate in a manner similar to that described in association with at least FIGS. 8A-8L and/or as further described below in association with at least FIGS. 10A and/or 10B.

In general terms, an example method of using a device (e.g. 1305) comprises implanting the distal lead portion 1313 via the midline implant-access incision 609A using direct visualization techniques (within minimal or no tunneling) enabled by a size and/or shape of the midline implant-access incision.

However, it will be understood that in some instances, tools may be used to form implant paths, partial tunnels or pockets to enable positioning at least some stimulation elements (e.g. 1314A, etc.) relative to pertinent target tissues.

In some examples, the lead segments (e.g. 1315A, 1315, etc.) between adjacent stimulation elements (e.g. 1314A, 1314B, etc.) may comprise a flexibility greater than a flexibility of lead body (e.g. 1312) proximal to the entire group of stimulation elements. In some such examples, this arrangement may facilitate maneuvering, positioning, and/or orienting the respective stimulation elements within and among target tissues during implantation.

In some examples, the lead segments (e.g. 1315A, 1315B, etc.) comprise a greatest cross-sectional dimension which is less than a greatest cross-sectional dimension of the stimulation elements 1314A-1314F. The lead segments 1315A, 1315B, etc. may have a cross-sectional shape which is the same as, or different from, a cross-sectional shape of the stimulation elements 1314A-1314F. In some examples, the lead segments (e.g. 1315A, 1315B, etc.) comprise a greatest cross-sectional dimension which is substantially less than a greatest cross-sectional dimension of the stimulation elements 1314A-1314F. In some such examples, the substantially less difference comprises a difference of at least 25 percent or at least 50 percent. In some examples, this arrangement which may enhance the flexibility and maneuverability of the lead segments (e.g. 1315A, 1315B, etc.) relative to the stimulation elements 1314A-1314F, which in turn may enhance the maneuverability of stimulation elements 1314A-1314F relative to the target tissues and/or of the stimulation elements 1314A-1314F relative to each other.

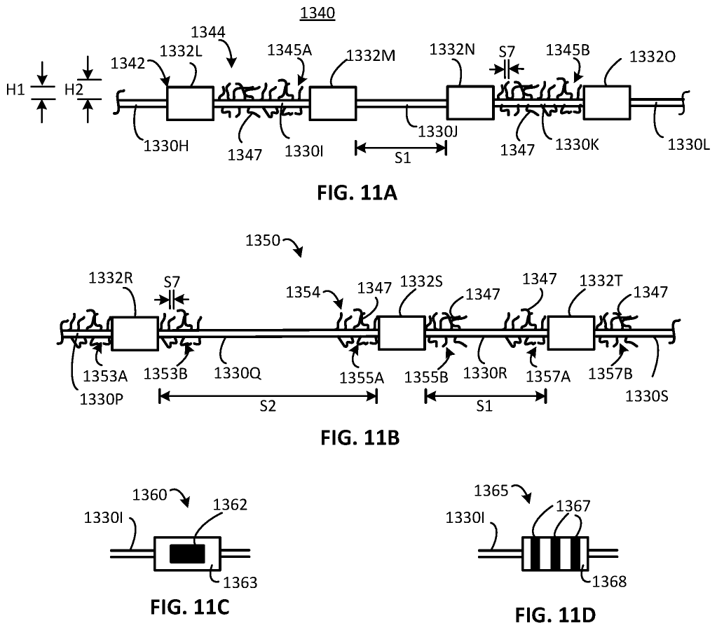

In some examples, the respective lead segments may comprise lengths (e.g. spacing) between adjacent stimulation elements (e.g. 1314A, 1314B, etc.) and/or anchor structures, according to at least some of the features and attributes of the examples described in association with at least FIGS. 11A-11D.

FIG. 9B is a sectional view schematically representing an example lead segment 1316, which may comprise an example implementation of any one or all of the lead segments 1315A-1315F. As shown in FIG. 9B, in some examples, the example, lead segment 1316 may comprise a flexible electrically insulative conduit (e.g. hollow tubular structure) including a side wall 1319A defining an outer surface 1319B. In some examples, a plurality of electrical conductors 1317 extend through and within an interior 1318 of the conduit to establish electrical connection between a pulse generator (or a more proximal lead portion) and a respective contact electrode of one of the respective stimulation elements (e.g. 1314A, 1314B, etc.). It will be understood that, in some examples, a single conductor 1317 (or single conductor group) supports and is electrically connected to a single stimulation element (e.g. 1314A) to enable independently addressable control of individual electrodes of the respective stimulation elements in applying stimulation to the respective target tissues. The interior 1318 may comprise insulative material to electrically insulate the respective conductors 1317 from each other.

In some examples, upon electrical connection of a respective one of the conductors 1317 to a respective one of the more proximal stimulation elements, such as stimulation element 1314A, the respective conductor 1317 may terminate at the location of such electrical connection. In this arrangement, the particular conductor 1317 does not extend distally from the respective stimulation element 1314A while the other respective conductors 1317 extend distally further within and through the conduit (as defined by side wall 1319A) to reach their own respective stimulation element (e.g. 1314B, 1314C, etc.). In a manner similar to the stimulation element 1314A, a conductor which is electrically connected to a respective one of the more distally-located stimulation elements, such as stimulation element 1314B, terminates at that location. This arrangement is repeated throughout the length of the stimulation portion 1311 (in a distal orientation) such that by the time the most distal stimulation element (e.g. 1314F) is reached, just one electrical signal conductor 1317 extends within and through the conduit. Accordingly, as the stimulation portion extends distally from the most proximal stimulation element 1314A, each respective lead segment 1315B, 1315C (and so on) may, in some examples, become progressively more flexible than the preceding lead segment (e.g. 1315A, 1315B, and so on) because fewer conductors 1317 are present within the conduit. This progressively increasing flexibility distally along the length of the stimulation portion 1311 may enhance the ability to introduce, advance, and/or maneuver the respective more distal stimulation elements and corresponding lead segments in and among target tissues. Whether alone or in combination with other described features and attributes of the example of FIGS. 9A-9B, this progressively increasing flexibility may promote independent positioning of the stimulation elements 1314A, 1314B, etc. relative to each other and/or relative to the different target nerve portions, muscle portions, etc.

Via such arrangements, in some examples, a flexibility of the stimulation portion 1311 may increase from about 30 to 50 percent from the most proximal stimulation element 1314A to the most distal stimulation element 1314F.

In contrast, at least some other non-example lead designs may maintain substantially uniform relative flexibility (e.g. stiffness) distally toward a distal tip of a lead in order to enable pushability of such leads through a tunnel or vasculature to place a stimulation electrode (or sensing electrode) at a desired location within patient anatomy. Such stiffness would significantly inhibit the type, manner, and extent of maneuverability contemplated for the example stimulation portion of the examples of the present disclosure, as further described in association with.

With this in mind, one example method of implantation may comprise forming a tunnel, via the implant-access incision 609A, to advance and position more proximal portions of the lead body for connection to a pulse generator (e.g. 333 in FIG. 2A, 1133 in FIG. 17A) while more distal portions of the lead, such as the distal lead portion 1313 including the stimulation portion 1311 comprising the connected array of stimulation elements 1314A-1314F may be placed in and among target tissues without little or no tunneling to achieve an implanted configuration as shown in FIGS. 9A, 10A, 10B. In this arrangement, portions of the lead 1310 which are proximal to distal lead portion 1313 may comprise a stiffness to enable advancement, pushability, etc. within and through a tunnel.

Conversely, because in some examples the distal lead portion 1313 is not being advanced through a tunnel (in some examples), the distally increasing flexibility of the stimulation portion 1311 may enhance maneuverability, positioning, etc. of the stimulation elements 1314A, 1314B, etc. independently of each other relative to the target tissues.

With further reference to at least FIG. 9A, at least one of the respective lead segments 1315A-1315F may comprise a shape, size, and/or length, which enables the particular lead segment 1315A-1315F to exhibit a variable length to provide strain relief for a particular stimulation element, the stimulation portion 1311 as a whole, the distal lead portion 1313, and/or the lead 1310 as a whole.

With this in mind, as shown in FIG. 9A, in one non-limiting example, lead segments 1315C, 1315D, and 1315F comprise a length between adjacent pairs of stimulation elements such that a loop is formed in each respective lead segment 1315C, 1315D, 1315F to provide the above-noted strain relief. It will be understood that just one, just some, or all of the lead segments 1315A-1315F may comprise such a loop to provide a variable length property (e.g. loop) or other shape to provide a variable length, such as later shown in FIG. 9C.

In some examples, the lead segment 1315D may comprise an overall length which is substantially greater than a length of the other respective lead segments (e.g. 1315B, 1315C, 1315E, 1315F) in order to provide sufficient length to straddle the sagittal midline 316 to enable positioning the respective stimulation elements in stimulating relation to target tissues on opposite sides (e.g. 312R, 312L) of the patient's body, while also enabling maneuverability of the stimulation elements 1314A, 1314B, etc. independently of each other in relation to specific target tissues, as further described later in association with at least FIGS. 10A-10B. In some examples, the length of lead segment 1315D is substantially greater than the length of the other respective lead segments (e.g. 1315B, 1315C, etc.). In some such examples, this substantially greater difference may comprise a difference of at least 25 percent, at least 50 percent, at least 75 percent, at least 100 percent, at least 150 percent, or at least 200 percent. At least some additional aspects regarding a length of the lead segments (which extend between adjacent stimulation elements) are further described later.

FIG. 9C is a top plan view schematically representing an example portion 1307 which may comprise one example implementation of at least one of the respective lead segments (e.g. 1315A, 1315B, etc.). As shown in FIG. 9C, the example portion 1307 may comprise one or more undulating curved segments 1308 and/or other shapes which enable the example portion 1307 to have a variable length, which may provide strain relief for a particular lead segment (e.g. between an adjacent pair of stimulation elements), the stimulation portion 1311 as a whole, and/or at least the distal lead portion 1313 of the lead 1310. The undulating shape may comprise a shape (e.g. sinusoidal, sigmoid, other) which repeats or non-repeating arcuate segments. In some examples, the variable length portion 1307 of FIG. 9C may comprise one alternative example implementation of the example lead segments 1315C, 1315D, and/or 1315F in FIG. 9A.

FIG. 10A is a diagram 1320 schematically representing an example device (and/or example method) 1322 including a stimulation portion 1321 in which at least some stimulation elements (e.g. 1324A, 1324B, etc.) extend in different planes within or relative to the target tissue (e.g. target nerve portion, target muscle portion, target neuromuscular junction). In some examples, the example device including stimulation portion 1321 may comprise at least some of substantially the same features and attributes as, and/or an example implementation of, the example device 1305 (including stimulation lead 1310) of FIG. 9A.

As shown in FIG. 10A, the stimulation portion 1321 comprises an array of spaced apart stimulation elements 1324A-1324F with lead segments 1325A, 1325B, 13525C, 1325D, 1325E. interposed between adjacent stimulation elements 1324A-1324F. FIG. 10A also provides one non-limited example of various target nerve portions, as seen in cross-section and in relatively simplistic view for illustrative purposes, relative to which various stimulation elements of stimulation portion 1321 may be implanted.

With this in mind, FIG. 10AA is a diagram 1000 schematically representing a further example implementation of each stimulation element (e.g. 1324-1324 in FIG. 10A) in which each stimulation element may comprise an elongate rectangular cuboid shape, which may represent a paddle-style carrier body supporting a single contact electrode or an array of contact electrode on one or more surfaces of the carrier body. As shown in FIG. 10AA, each stimulation element comprises a major axis (e.g. longitudinal axis LA) extending along (or between/through) opposite ends 1002A, 1002B, as well as comprising opposite sides 1006A, 1006B (behind the plane of FIG. 10AA), and opposite faces 1004A, 1004B. Depending on the particular anatomy at or surrounding a particular target tissue (e.g. nerve portion) and/or depending on which of the faces, sides, and/or ends of the stimulation element carries contact electrodes to be in stimulating relation to the target tissue (e.g. target nerve portion, target muscle portion, etc.), a specific stimulation element may be implanted with one of its faces, sides and/or ends being in primary stimulating relation to the target tissue, In some examples the stimulation element may be configured, oriented, etc. and/or the particular patient target tissue may be configured, oriented, etc. such that more than one of the surfaces (e.g. face, side, ends) of the stimulation element may in stimulating relation to the target tissue such that at least one contact electrode on those respective surfaces may be in stimulating relation to the target tissue.

However, in some examples, the stimulation elements (e.g. 1324A-1324F) may carry (e.g. support) contact electrodes on just one surface (e.g. one of the faces 1004A, 1004B, in some examples) of the body of the stimulation element such that the maneuverability of the respective stimulation elements (e.g. 1324A-1324F), via the length and/or flexibility of the lead segments (e.g. 1325A, 1325B, etc.), relative to each other enables independent positioning in a manner by which the single surface carrying contact electrodes may be placed into robust stimulating relation to (e.g. robust electrical capture of) the target tissue.

With this in mind, it will be further understood that the elongate rectangular cuboid shape shown in FIGS. 10A, 10AA, 10B comprises just one example shape for the stimulation elements (e.g. 1324A-1324F) and that the stimulation elements in the examples of FIGS. 10A-10B (and other examples of the present disclosure) may embody other shapes while still providing the features and attributes of this example. Moreover, in some examples, at least one of the stimulation elements (e.g. 1324A-1324F) may comprise a shape and/or a size different from the shape and/or size of the other respective stimulation elements.

With further reference to FIG. 10A, in some examples stimulation element 1324A is implanted with its major axis (e.g. longitudinal axis LA in FIG. 10AA) extending in a first orientation (as represented by directional arrow F), stimulation element 1324B is implanted with its major axis extending in a different, second orientation (as represented by directional arrow G), and stimulation element 1324C is implanted with its major axis extending in a further different, third orientation (as represented by directional arrow J).

In some examples, the various example nerve portions (e.g. 482R, 484R, 486R and 483L, 485L, 487L) in FIGS. 10A-1B may directly correspond respectively to the various example nerve portions (e.g. 372R, 376R, 378R and 371L, 375L, 377L) in FIG. 2B, while in some examples, one or more of the various example nerve portions (e.g. 482R, 484R, 486R and 483L, 485L, 487L) in FIGS. 10A-1B do not directly correspond to the various example nerve portions (e.g. 372R, 376R, 378R and 371L, 375L, 377L) in FIG. 2B.

On the right side 312R, in some examples, an implant location 489A at which stimulation element 1324A is in stimulating relation to nerve portion 482R, according to first orientation F (by which stimulation element 1324A extends within the patient's body), may be characterized (in some examples) according to a three-axis orthogonally-related reference, including a first plane Q1, second plane V1, and third plane U1. In some examples, the implant location 489B at which stimulation element 1324B is in stimulating relation to nerve portion 484R, according to different, second orientation G (by which stimulation element 1324B extends within the patient's body), may be characterized (in some examples) according to a three-axis orthogonally-related reference, including a first plane Q2, second plane V2, and third plane U2. In some examples, the implant location 489C at which stimulation element 1324C is in stimulating relation to nerve portion 486R, according to further different, third orientation J (by which stimulation element 1324C extends within the patient's body), may be characterized (in some examples) according to a three-axis orthogonally-related reference, including a first plane Q3, second plane V3, and third plane U3.

On the right side 312R of the patient's body, in some examples planes Q1, Q2, Q3 are spaced apart from each other in a generally parallel relationship, planes U1, U2, U3 are spaced apart from each other in a generally parallel relationship, and planes V1, V2, V3 being in spaced apart from each other in a generally parallel relationship. Accordingly, at each respective implant location (e.g. 489A, 489B, 489C), the respective stimulation elements (e.g. 1324A, 1324B, 1324C) extend in at least one different plane (e.g. Q1, U1, V1) than in the other respective implant locations (489A, 489B and/or 489C).

As further shown in FIG. 10A and regarding left side 312L, in some examples the implant location 489D at which stimulation element 1324D is in stimulating relation to nerve portion 483L, according to fourth orientation K (by which stimulation element 1324D extends within the patient's body), may be characterized (in some examples) according to a three-axis orthogonally-related reference, including a first plane Q4, second plane V4, and third plane U4. In some examples, the implant location 489E at which stimulation element 1324E is in stimulating relation to nerve portion 485L, according to fifth orientation M (by which stimulation element 1324E extends within the patient's body), may be characterized (in some examples) according to a three-axis orthogonally-related reference, including a first plane Q5, second plane V5, and third plane U5. In some examples, the implant location 489F at which stimulation element 1324F is in stimulating relation to nerve portion 487L, according to sixth orientation N (by which stimulation element 1324C extends within the patient's body), may be characterized (in some examples) according to a three-axis orthogonally-related reference, including a first plane Q6, second plane V6, and third plane U6.

On the left side 312L of the patient's body, in some examples the different planes Q4, Q5, Q6 are spaced apart from each other in a generally parallel relationship, the different planes U4, U5, U6 are spaced apart from each other in a generally parallel relationship, and the different planes V4, V5, V6 being in spaced apart from each other in a generally parallel relationship. Accordingly, at each respective implant location (e.g. 489D, 489E, 489F), the respective stimulation elements (e.g. 1324D, 1324E, 1324F) extend in at least one different plane (e.g. Q4, U4, V4) than in the other respective implant locations (489D, 489E, and/or 489F).

In view of these relationships, it will be understood that each of the respective stimulation elements (e.g. 1324A-1324F) has a selectable implant location and orientation which varies according to multiple degrees of freedom to accommodate the various target nerve portions extending in different planes relative to each other).

Moreover, as apparent from the example of FIG. 10A, the target nerves may extend in different orientations relative to each other within the patient's body. In other words, in some examples, for any given cross-section of target tissue, at least some of the various target nerve portions may extend at an angle relative to each other.

In some examples, one or more of the planes (e.g. Q1, V1, U1) for a given implant location may correspond to a respective global reference plane such as Z, X, Y as shown in FIG. 10A, or they may be independent from such global reference planes. In some examples, one or more of the planes (e.g. Q1, V1, U1) for a given implant location may correspond to a respective patient anatomy reference plane such as Superior (S)—Inferior (1), Anterior (A)—Posterior (P), and Medial (M)—Lateral (L) as shown in FIG. 10A, or they may be independent from such patient anatomy reference planes.

While the example device and/or example method of FIG. 10A is described in relation to target nerve portions (e.g. 482R-486R, 483L-487L), the example device and/or example method in FIG. 10A is equally applicable to target tissues such as muscle portions, neuromuscular junctions, and/or combinations of nerve portions, muscle portions, and/or neuromuscular junctions.

While FIG. 10A depicts stimulation elements as having an elongate rectangular cuboid shape (e.g. having a paddle-shaped carrier body with rounded corners) at least for purposes of illustrating orientation in and along different planes within and among patient anatomy (including various target tissues, such as but not limited to target nerves), it will be understood that the stimulation elements may have a cylindrical shape or other shapes.

FIG. 10B is a diagram 1330 schematically representing an example device and/or example method 1335 which may comprise at least some of substantially the same features and attributes as the example device (and/or example method) 1322 in FIG. 10A, except with the example of FIG. 10B comprising implant locations (e.g. 489G, 489H, 489I, 489J, 489K) by which at least some nerve portion(s) (e.g. 482R, 484R) are located among or between adjacent pairs of stimulation elements (e.g. 1324A, 1324B, etc.) such that stimulation vectors may be delivered across or through target tissues located between such stimulation elements. This arrangement stands in contrast to the example of FIG. 10A in which a stimulation element (e.g. 1324A in FIG. 10A) may comprise an implant location 489A in FIG. 10A) in direct contact with or highly close proximity to a target nerve portion (e.g. 482R in FIG. 10A) (or other target tissue).

Accordingly, as further shown in FIG. 10B, with at least stimulation elements 1324A and 1324B being implantably positioned on opposite sides (or at different angles, orientations) relative to nerve portion 482R, a stimulation vector N2 may be applied between the respective stimulation elements 1324A, 1324B. Similarly, in some examples, with at least stimulation elements 1324B and 1324C being implantably positioned on opposite sides (or at different angles, orientations) relative to nerve portion 484R, a stimulation vector N3 may be applied between the respective stimulation elements 1324B, 1324C.

In another example, with at least stimulation elements 1324C and 1324D being implantably positioned on at different angles, orientations, etc. relative to nerve portions 484R, 486SM, and/or 483L, a stimulation vector N4 may be applied between the respective stimulation elements 1324C, 1324D. Unlike some of the other example stimulation vectors, the example stimulation vector N4 extends across the sagittal midline 316 such that the stimulation vector N4 may apply stimulation to target tissues (e.g. nerve portions, muscle portions, combinations of nerve portions and muscle portions, neuromuscular junctions, and/or combinations thereof) which straddle the sagittal midline 316 and/or on opposite sides of the sagittal midline. In some such examples, such stimulation may sometimes be referred to as cross-lateral stimulation, cross midline stimulation, and/or cross bilateral stimulation. For example, as shown in FIG. 10B, a nerve portion 486SM may have a location near the sagittal midline 316 such that a stimulation vector N4 would capture and apply a stimulation signal to the nerve portion 486SM (and/or associated muscle portions, neuromuscular junctions, etc.) because of its position between the stimulation elements 1324C on patient right side 312R and the stimulation element 1324D on patient left side 312L. While nerve portion 484R does not directly fall along a line or region through which the stimulation vector N4 may extend, it may be sufficiently captured to also be stimulated via the stimulation vector N4 based on its location somewhat between stimulation elements 1324C and 1324D.

It will be understood that the example of applying stimulation at, along, and/or across a sagittal midline 316 such as for nerve portion 486SM (or a muscle portion, etc.) may be utilized in at least some of the other example implementations throughout the present disclosure.

In some examples, the particular orientation of at least some of the respective stimulation elements (e.g. arrow F for 1324A; arrow G for 1324B; arrow J for 1324C; etc.) may be selectively arranged to cause the respective contact electrodes of the respective spaced apart stimulation elements to be positioned relative to each other to enhance a potential number, orientation, location, etc. of stimulation vectors (e.g. N2, N3, N4, N5, etc.) relative to the target tissues. In some examples, via applying test stimulation vectors, it can be determined which (and how) stimulation elements may be utilized relative to each other to apply the desired stimulation vectors.

In some examples, in situations in which some stimulation elements cannot be placed in ideal locations due to challenges in subcutaneous delivery arising from patient anatomy, stimulation vectors may be identified which use such stimulation elements and other more ideally placed stimulation elements in order to still effectively capture and stimulate target tissues.

With this in mind, in some examples each stimulation element may comprise a printed stimulation element, which has at least one contact electrode on each side of a generally flat body. Having contact electrodes on both (opposite) sides of the body may expand the number, type, orientation, location, etc. of potential stimulation vectors (among the multiple spaced apart stimulation elements) which may be identified and used to apply stimulation.

In some examples, a printed stimulation element may comprise a relatively thin, low profile insulative body formed in the manner of a printed circuit board construction including conductive traces to act as conductors to and between respective contact electrodes. In some examples, the printed stimulation element may be flexible which may sometimes be referred to as flexible printed circuit-type elements. In some such examples, the printed stimulation elements omit other more complex forms of circuitry, such as pulse generating circuitry (e.g. stimulation signal forming circuitry), wireless communication circuitry, and the like. Among other aspects, the low profile (e.g. low thickness or diameter) of the printed stimulation element may facilitate introduction and advancement of the respective stimulation elements in and among target tissues. In some such examples, the low profile arrangement may comprise at least some of substantially the same features as the later described example implementation of a low profile arrangement according to the cross-sectional configuration of stimulation element 1632B in FIG. 16E.

In some examples, at least some of the lead segments also may be formed as printed circuit-type elements with each lead segment comprising an insulative substrate on which is printed conductive traces to carry a signal from or to a stimulation element relative to a pulse generator or other stimulation circuitry. The printed conductive traces are covered with an insulative jacket or coating, which may cover just the conductive traces or the entire assembly of the substrate and conductive traces. In some examples, the insulative jacket also may comprise part of the “printing” of the lead segments.

In some such examples, printed lead segments and printed stimulation elements may be formed as a single, unitary (e.g. monolithic) construction.

In some such examples, the printed lead segments may have a cross-sectional profile, such as a shape (e.g. rectangular, circular, elliptical, etc.) and/or dimensions (e.g. width, height, diameter, greatest cross-sectional dimension, etc.) which are generally the same as a cross-sectional profile (e.g. shape and/or dimensions) of the printed stimulation elements. In some examples, this general matching of the cross-sectional profiles of the lead segments and the stimulation elements may be implemented even for example implementations in which the lead segments and/or the stimulation elements are not printed circuit-type elements. In some examples, whether printed or not, the general matching of the cross-sectional profile of the respective lead segments with the stimulation elements may facilitate certain types of anchor structures, such as but not limited to at least some example anchor structures of the present disclosure.

However, in some examples, the cross-sectional profile (e.g. shape and/or dimensions) of the lead segments may be different from the cross-sectional profile of the stimulation elements. This relationship may be implemented in examples in which one or both of the lead segments and the stimulation elements are printed circuit-type elements or may be implemented in examples in which none of the lead segments or stimulation elements are printed circuit-type elements.

In some examples, whether implemented as printed circuit-type elements or not, the arrangement of providing the respective lead segments with a cross-sectional profile different from the cross-sectional profile of the stimulation elements may facilitate certain types of anchor structures, such as but not limited to at least some example anchor structures of the present disclosure. In some such examples, a cross-sectional profile of the lead segments may be greater than (i.e. larger) which may increase a surface area available to interact with surrounding tissues, thereby effectively increasing the size of an anchor structure.

For example, in example implementations in which the anchor structure may comprise forming all or part of an outer surface of the lead segment with a grouped arrangement (e.g. carpet) of anchor elements, a relatively larger cross-sectional profile of the lead segment will effectively create a relative larger anchor structure. In some such examples, the grouped arrangement of anchor elements may comprise homogenous anchor elements or may comprise heterogeneous elements.

With reference to the at least the examples of FIGS. 9A-10B, in some examples, at least some of lead segments between the respective stimulation elements may comprise a length which is a multiple of a length of a stimulation element, which may facilitate placement of the stimulation element(s) and which may enhance maximizing the number of degrees of freedom by which the stimulation elements may be maneuvered independently from each other (while still being connected via the lead segments between respective stimulation elements) into a desired position in, around, among, etc. the target tissue(s). In some examples, the “multiple” may comprise at least 3 times, 4 times, 5 times, etc. a length of a stimulation element. In some examples, the multiple may comprise on the order of one order of magnitude difference in length, such as the lead segment having a length of at least about 9, 9.5, 10, 10.5, 11 times a length of a stimulation element. Via such example arrangements, in some examples the maneuvering, position, orientation, etc. of one respective stimulation element is generally not limited by the maneuvering, position, orientation, etc. of another (e.g. adjacent) respective stimulation element. In some examples, each of the stimulation elements may be maneuverable according to six degrees of freedom which includes three orthogonally-related rotational axes and three orthogonally-related translational axes, as further described in association with at least FIGS. 18A-21B.

In some examples, the array of stimulation elements may be arranged on two separate distal lead segments in a manner similar to that shown in the examples in association with at least FIGS. 8A, 8K, 16A, 16C, 16F, 22A, 22B, etc. in which a lead body comprises a bifurcation portion to enable a first distal lead segment to be implanted on a first side of the patient's body and a second distal lead segment to be implanted on a second side of the patient's body such the respective first and second distal lead segments (supporting the longitudinally spaced apart stimulation elements) are independently positionable relative to each other on opposite sides of a sagittal midline 316. For instance, a first distal lead segment may support one group of stimulation elements (e.g. 1314A, 1314B, 1314C in FIG. 9A) to be implanted on a first side of the patient's body while a separate second distal lead segment may support a second group of stimulation elements (e.g. 1314D, 1314E, 1314F in FIG. 9A) to be implanted on a second side of the patient's body.

In some such examples, the respective first and second distal lead segments may sometimes be referred to as first and second strands of stimulation elements. In a manner consistent with the examples of FIGS. 9-10B, the stimulation elements on the first strand are spaced apart far enough from each other along the strand to be relatively independently positionable from each other, and the stimulation elements on the second strand are spaced apart far enough from each other to be relatively independently positionable from each other.

FIG. 11A is a side plan view of a portion of an example stimulation lead including a stimulation portion 1340 comprising an anchor structure 1344 including anchor portions (e.g. 1345A, 1345B). In some examples, the stimulation portion 1340 may comprise at least some of substantially the same features and attributes as the stimulation portion of the stimulation lead 1310 of FIG. 9A and/or the stimulation portion 1340 of FIG. 11A may comprise one example implementation of the stimulation portion of the stimulation lead 1310 of FIG. 9A and/or the stimulation portion 1321, 1331 of FIGS. 10A-10B.

As shown in FIG. 11A, the stimulation portion 1340 may comprise an array 1342 of spaced apart stimulation elements 1332L, 1332M, 1332N, 1332O within lead segment 1330I extending between elements 1332L and 1332M, lead segment 1330J extending between elements 1332M and 1332N, lead segment 1330K extending between elements 1332N and 1332O, and so on. Lead segments 1330H and 1330L are present on opposite ends of the array.

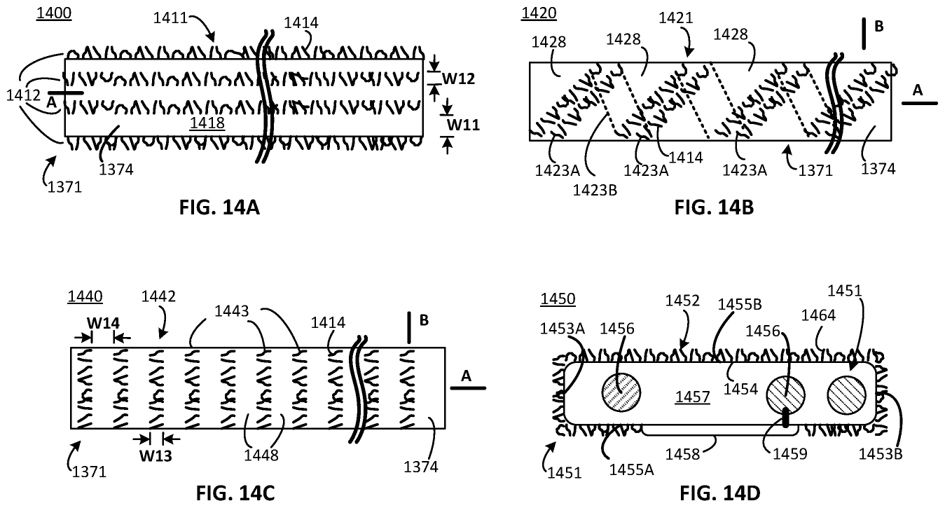

In some examples, each stimulation element 1332L, 1332M, etc., and each lead segment 1330I, 1330J, etc., may comprise a generally cylindrical shape. However, in some examples, each stimulation element 1332L, 1332M, etc. and/or each lead segment 1330I, 1330J, etc., may comprise a shape other than a generally cylindrical shape. Regardless of the particular shape, in some examples, the anchor elements 1347 may extend completely about a circumference of the respective lead segments (e.g. 1330I, 1330J) or in some examples, may extend partially about a circumference of the respective lead segments (e.g. 1330I, 1330J) per at least some of the example implementations later described in association with at least FIGS. 14A-15B, 16J-16K, and FIGS. 24A-24E.

As shown in FIG. 11A, in some examples the stimulation portion 1340 may comprise a first amount of spacing S1 between adjacent pairs of stimulation elements (e.g. 1332M, 1332N) which may be uniform in some examples. However, in some examples, as later shown in FIG. 11B, non-uniform spacing (e.g. S2, S1, and so on) may be implemented.

As further shown in FIG. 11A, each anchor portion 1345A, 1345B comprises an array of anchor elements 1347 arranged on a surface of a lead segment (e.g. 1330I, 1330K) in a pattern, density, thickness, and/or orientation, such that anchor elements 1347 work individually and/or collectively to securely engage surrounding tissue to thereby secure at least a portion of the stimulation portion 1340 relative to the surrounding tissue. In addition to their collective arrangement, each anchor element 1347 may include barbs, protrusions, shapes, sizes, orientations which enhance secure engagement of the surrounding tissue.

In some examples, the anchor portions of FIGS. 11A-11B, such as, but not limited to, the anchor elements 1347 may comprise at least some of substantially the same features and attributes as, and/or an example implementation of, the anchor elements 6924 and related components as described in association with at least FIGS. 8E-8H.

In some examples, at least because such anchor portions 1345A, 1345B (FIGS. 11A-11B) are situated immediately adjacent at least some stimulation elements (e.g. 1332L and 1332M or 1332N and 1332O, respectively), the anchor portions may secure the stimulation elements to be maintained in a stable position in stimulating relation to nearby target tissues (e.g. target nerve portions, target muscle portions, and/or target neuromuscular junctions).

In some examples, the anchor structure 1344 may comprise a spacing S7 between the respective anchor elements 1347 which is substantially less than a spacing S1 between adjacent stimulation elements (e.g. 1332M, 1332N, etc.) on the stimulation portion 1340. In such some examples, the “substantially less” spacing may be implemented via the anchor structure 1344 comprising a spacing S7 between the respective anchor elements 1347 which is at least one order of magnitude less than a spacing S1 between adjacent stimulation elements (e.g. 1332M, 1332N, etc.) on the stimulation portion 1340. In some examples, the substantially less spacing may be implemented via the spacing S7 being at least two orders of magnitude less than the spacing S1 between adjacent stimulation elements (e.g. 1332M, 1332N, etc.). In some examples, the substantially less spacing may be implemented via the spacing S7 being 50 percent less than the spacing S1 between adjacent stimulation elements (e.g. 1332M, 1332N, etc.).

In some examples, these same relationships (of the spacing S7 between anchor elements 1347 being substantially less than the spacing S1) also apply to larger spacing(s) between adjacent stimulation elements, such as when there is a greater spacing between adjacent stimulation elements, such as spacing S2 in FIG. 11B between adjacent stimulation elements 1332R, 1332S which is greater than spacing S1 between adjacent stimulation elements 1332S and 1332T.

In some examples, such compressed spacing between adjacent anchor elements 1347 may be expressed as a density of the anchor elements 1347 in which the density may comprise a selected number of such anchor elements 1347 per area (e.g. square centimeters, square inches, and the like) of the stimulation portion 1340, such as on lead segments 1330I, 1330K, 1330Q, etc. In some such examples, the anchor elements 1347 may exhibit a density which is at least one order of magnitude greater than a density of the stimulation elements (e.g. 1332M, 1332N) among the lead segments, e.g. relative to the stimulation portion 1340 as a whole.

In some examples, the density may be expressed as a first ratio of a total number of anchor elements 1347 per total non-conductive surface area (e.g. surface area of the lead segments (e.g. 1330I, 1330J) of the stimulation portion 1340 being substantially greater than a second ratio of a total number of stimulation elements (e.g. 1332L, 1332M, etc.) per total non-conductive surface area of the stimulation portion 1340. In some examples, the density may be expressed as a first ratio of a number of anchor elements 1347 per total non-conductive surface area (of the stimulation portion 1340) being substantially greater than a second ratio of a total number of stimulation elements (e.g. 1332L, 1332M, etc.) per total conductive surface area (e.g. a sum of the surface area of the exposed electrically conductive stimulation elements) of the stimulation portion 1340. In these examples, the term “substantially greater” may comprise at least one order of magnitude, at least two orders of magnitude, at least 200 percent greater, at least 100 percent greater, or at least 50 percent greater.

In some examples, as noted later in association with at least FIGS. 15A-15B (which may comprise features and attributes by which the anchor elements 1347 may be implemented), various anchor elements 1347 may extend from a support surface (e.g. lead segments 1330I, 1330K, 1330Q, etc.) in different orientations (e.g. angular projection from the support surface) relative to each other and/or in different orientations relative to the stimulation elements 1332L, 1332M, etc. with such different orientations facilitating secure engagement of the collective group (or at least some of) the anchor elements 1347 relative to surrounding target tissue (e.g. non-nerve target tissue in some examples).

In some examples, the anchor elements 1347 may be arranged in patterns to facilitate securely engaging surrounding tissue and/or to facilitate maneuvering a stimulation portion (e.g. 1340) within and through surrounding tissue. In some such examples, one or more patterns may comprise at least some of substantially the same features and attributes as the patterns of anchor elements described in association with at least FIGS. 14A-15B, 16J-16K, and FIGS. 24A-24E and various examples throughout the present disclosure.

It will be understood that the stimulation portion 1340 represents an example segment of a stimulation portion which may comprise a greater or less number of stimulation elements (e.g. 1332L, 1332M, 1332N, 1332O) than shown in FIG. 11A and that the anchor structure 1344 may comprise a greater number or less number of anchor portions 1345A, 1345B, etc. In some examples, as shown in FIG. 11A, anchor portions (e.g. 1345A, 1345B, etc.) are present between just some pairs of adjacent stimulation elements as shown in FIG. 11A such that no anchor portions are present between some stimulation elements, such as stimulation elements 1332M and 1332N. Among other aspects, this arrangement may enhance a degree to which an implanted stimulation portion can flex in a manner complementary with flexion of the head-and-neck region.

However, in some examples, a respective one of the anchor portions 1345A, 1345B, etc. may be present between each and every pair of adjacent stimulation elements (e.g. between 1332L and 1332M, between 1332M and 1332N, between 1332N and 1332O, and so on). Among other aspects, this arrangement may increase a degree to which the stimulation portion 1340 (or certain portions thereof) is secured relative to non-target tissues and/or target tissues.

In some examples, the number and location (along the stimulation portion 1340) of anchor portions (e.g. 1345A, 1345B) may be based on the number and location of stimulation elements 1332L, 1332M, 1332N, 1332L, 1332O, etc. and the relative spacing between such stimulation elements. Moreover, in some examples, the stimulation portion 1340 may comprise the same amount of uniform spacing (S1) between adjacent stimulation elements, as shown in FIGS. 9-11A.

However, in some examples such as the example stimulation portion 1350 in FIG. 11B, a first amount of spacing S1 between one adjacent pair of stimulation elements (e.g. 1332S, 1332T) may be different from a second amount of spacing (S2) between another adjacent pair of stimulation elements (e.g. 1332R, 1332S). This different amount of spacing may be used to cause a stimulation element (e.g. 1332R) to likely become positioned at a particular anatomical location (e.g. second nerve portion, second muscle portion) in view of another stimulation element (e.g. 1332S or 1332T) likely becoming positionable at another particular anatomical location (e.g. first nerve portion, first muscle portion, etc.). In some examples, having different amounts of spacing between at least some of the stimulation elements may enhance the adaptability of the stimulation portion 1350 to accommodate anatomical variations among different patients in whom the stimulation portion 1350 may be implanted.

Moreover, in some examples, at least because of the size (e.g. length, diameter, etc.), shape, flexibility, etc. of the lead segments (e.g. 1325A-1325E in FIGS. 10A and 1325A-1325F in FIG. 10B; 1330I-1330L in FIG. 11A) extending between the adjacent stimulation elements (e.g. 1324A and 1324B, 1324B and 1324C, 1324C and 1324D, 1324D and 1324E, and 1324E and 1324F in FIG. 10A; 1332L and 1332M, 1332M and 1332N, 1332N and 1332O in FIG. 11A), these interposed lead segments (e.g. 1325A-1325E in FIG. 10A; 1330I-1330L in FIG. 11A) may be positioned in desired curved shapes, angles, planes, orientations, etc. to enable placing the respective stimulation elements (e.g. 1324A-1324F, 1332L-1332O) at target stimulation locations of target nerve portions, target muscle portions, neuromuscular junctions, and/or combinations thereof. In some such examples, at least an outerjacket of the flexible lead segments (e.g. 1325A-1325E) may comprise a resilient material such that the lead segments may be flexibly curved, bent, etc. into their desired shape, but will return to an original shape in the absence of some force or restraint holding the flexible lead segment into the shape, configuration, etc. into which it was manipulated.

In some examples, at least some of the respective lead segments 1330H-1330L and/or at least some of the stimulation elements 1332L-1332O may be positionable adjacent significant non-nerve tissues at which anchoring may be beneficial. For instance, some example non-nerve tissues may comprise tendons of external lingual muscles (or muscles of airway patency), more specifically, tendons of the geniohyoid and hyoglossus muscles.

As shown in FIG. 11A, in some examples the stimulation elements (e.g. 1332L) may comprise a height (H1) (e.g. thickness) relative to a surface of the lead segments (e.g. 1330H, 1330I) and the anchor portion 1345A may comprise a height (H2) (e.g. thickness).

In some examples, the height H2 of the anchor portions 1345A, 1345B may comprise a height equal to or less than the height H1 of the stimulation element(s) (e.g. 1332L). In one aspect, this arrangement may facilitate engagement of the stimulation elements (e.g. 1332L) with the target tissues to which the stimulation elements (e.g. 1332L) are to be placed into stimulation relation. Moreover, a relatively low height H2 of the anchor portions 1345A, 1345B may enhance maneuvering of the respective lead segments (e.g. 1330I, 1330J, etc.) and/or stimulation elements (e.g. 1332L, 1332M) for implantation relative to target tissues. Nevertheless, despite their limited height H2, an orientation, shape, diameter, position, spacing, density, etc. of the anchor elements 1347 (in combination with their height) provides for sufficiently robust engagement and fixation relation to the surrounding tissues once a final implant location has been established.

On the other hand, in some examples, the height H2 of anchor portions 1345A, 1345B may comprise a height greater than the height H1 of the stimulation element(s) (e.g. 1332L) as shown in FIG. 11A, which may provide for more aggressive engagement of surrounding tissues. However, in some examples, even if the anchor portions 1345A, 1345B have a height H2 greater than the height H1 of the stimulation elements (e.g. 1332L), the height H2 still may be generally less than a height of conventional tines, etc. which typically have a height significantly exceeding the height H1 of the stimulation elements (e.g. 1332L). Accordingly, the relatively low height H2 of the anchor portions 1345A, 1345B may permit sufficient maneuvering of the stimulation portion for implantation while the height H2, in concert with the orientation, shape, diameter, position, spacing, density, etc. of the anchor elements 1347, provides for sufficiently robust engagement and fixation relation to the surrounding tissues once a final implant location has been established.

In some examples, the anchor portions of FIGS. 11A-11B may be implemented via at least some of the features and attributes of the example anchor structures described in association with at least FIGS. 15A and/or 15B. In one aspect, additional aspects regarding a height (e.g. H2 in FIG. 11A) of an anchor portion (e.g. 1345A, 1345B) are described in association with at least FIGS. 15A-15B.

FIG. 11B is a side plan view of a portion of an example stimulation portion 1350 comprising an anchor structure 1354. In some examples, the stimulation portion 1350 may comprise at least some of substantially the same features and attributes as the stimulation portion of FIGS. 9-11A, and the stimulation portion 1350 of FIG. 11B may comprise one example implementation of the stimulation portions of at least FIG. 9A and/or FIGS. 15A-15B.

As shown in FIG. 11B, the stimulation portion 1350 may comprise an array of spaced apart stimulation elements 1332R, 1332S, 1332T within lead segments 1330Q, 1330R interposed therebetween and lead segments 1330P, 1330S on opposite ends of the array of stimulation elements 1332R, 1332S, 1332T. It will be understood that the stimulation portion 1350 represents an example segment of a stimulation portion which may comprise a greater or less number of stimulation elements (e.g. 1332R, 1332S, 1332T) than shown in FIG. 11B and may comprise a greater or lesser number of anchor portions 1353A, 1353B, 1355A, 1355B, 1357A, 1357B. In some examples, the anchor portions (e.g. 1353A, 1353B; 1355A, 1355B; etc.) may be present for each stimulation elements (e.g. 1332R; 1332S; etc.) or for just some stimulation elements, in some examples.

In some examples, the anchor portions 1353A, 1353B, etc. comprise at least some of substantially the same features and attributes as the anchor portions 1345A, 1345B in FIG. 11A, except not entirely filling the space along a lead segment (e.g. 1330Q) between adjacent stimulation elements (e.g. 1332R, 1332S), a lead segment (e.g. 1330R) between adjacent stimulation elements (e.g. 1332S, 1332T), and so on.

In some examples, such as shown in FIG. 11B, the anchor portions (e.g. 1353A, 1353B) are provided in pairs such that at least some (or all) of the stimulation elements (e.g. 1332R) are sandwiched between the respective pair of anchor portions 1353A, 1353B while still leaving a significant proportion of an adjacent lead segment 1330Q to be free of anchor portions (or at least having significantly fewer anchor elements 1347). In one aspect, this arrangement of omitting anchor elements (e.g. 1347) along portions of the lead segments (e.g. 1330Q) may enhance advancement and maneuverability of the lead segments (e.g. 1330Q, 1330R) while the anchor portions 1353A, 1353B sandwiching the stimulation elements (e.g. 1332R) provides for robust fixation of the stimulation elements (e.g. 1332R) at a desired implant location. In some such examples, the anchor portions 1353A, 1353B may sometimes be referred to be at a respective stimulation element and not along a lead segment extending from the respective stimulation element.

In some examples, just one portion (e.g. 1353A or 1353B) of a pair of anchor portions (e.g. 1353A, 1353B) may be present at some of the stimulation elements. This arrangement may enhance positioning of at least some of the stimulation elements relative to certain types of anatomical variations of patient anatomy. In some such examples, just anchor portions 1353A, 1355A, 1357A (and so on) may be present or just anchor portions 1353B, 1355B, 1357B (and so on) may be present. In one aspect, such example arrangement may provide an anchor portion (e.g. 1353A, 1355A) for each stimulation element, yet may enhance slidability of advancing and positioning the various stimulation elements relative to various anatomical structures.

Moreover, depending on the direction and/or orientation of positioning the various stimulation elements and anchor portions within the patient, starting from the midline implant-access incision 609A (in some examples), it may be desirable to include solely or mostly anchor portions which trail a direction or orientation of advancement of the stimulation portion within the patient anatomy.

Accordingly, in some examples, one may assume that strictly for example purposes, the anchor portions 1353B, 1355B, 1357B may be on the trailing end of an advancement direction (as represented by arrow AD1) of the stimulation portion 1350, which may enhance slidability for initial positioning but still provide sufficient anchoring traction once the initial positioning is complete. In some such examples, only anchor portions 1353B, 1355B, 1357B are included (with anchor portions 1353A, 1355A, 1357A being omitted).

Conversely, in some examples, one may assume that strictly for example purposes, the anchor portions 1353A, 1355A, 1357A may be on the leading end of an advancement direction (as represented by arrow DA) of the stimulation portion, which may enhance anchoring traction as the initial positioning is being implemented. In some such examples, only anchor portions 1353A, 1355A, 1357A are included (with anchor portions 1353B, 1355B, 1357B being omitted).

However, in some examples, advancement of the stimulation elements (e.g. 1332R, 1332S, etc.) and/or the lead segments (e.g. 1330Q, 1330R, etc.) may occur laterally such that the respective anchor portions 1353A, 1353B may neither enhance or hinder advancement and positioning of the stimulation elements (e.g. 1332R, 1332S) during movement of the stimulation portion.

Among other aspects, the low profile (e.g. relatively low height) of the example anchor portions (e.g. FIGS. 11A, 11B) may enhance maneuverability of a stimulation portion among and within target tissues while still maintaining a desirable level of secure fixation. In one aspect, such low profile arrangements stand in contrast to more conventional high profile anchor(s) which may extend outwardly far beyond a surface of a lead or an outer surface of a stimulation element.

FIG. 11C is a side view of an example stimulation element 1360 which may comprise one example implementation of one of the stimulation elements in the previously described examples in association with at least FIGS. 9A-11B. As shown in FIG. 11C, the stimulation element 1360 may comprise a single contact electrode 1362 supported on a body 1363. Upon deployment in one of the example arrangements of FIGS. 9A-9C or FIGS. 10A-11B, the various stimulation elements 1360 may be distributed among various target tissues and may be activated in various, selectable combinations to implement desired stimulation vectors throughout portions of the target tissue.

FIG. 11D is a side view of an example stimulation element 1365 which may comprise one example implementation of one of the stimulation elements in the previously described examples in association with at least FIGS. 9-11B. As shown in FIG. 11D, the stimulation element 1365 may comprise an array of contact electrodes 1367 spaced apart along and supported on a body 1368. Whether or not multiple stimulation elements 1365 are activated in combination, when deployed in one of the example arrangements of FIGS. 9A-9C or FIGS. 10A-11B, each stimulation element 1365 may provide for at least bipolar stimulation at each target tissue at which a respective stimulation element is located.

It will be understood that in some examples, body 1363 (FIG. 11C) and/or body 1368 (FIG. 11D) is not strictly limited to the shape and/or size shown in FIGS. 11C-11D.

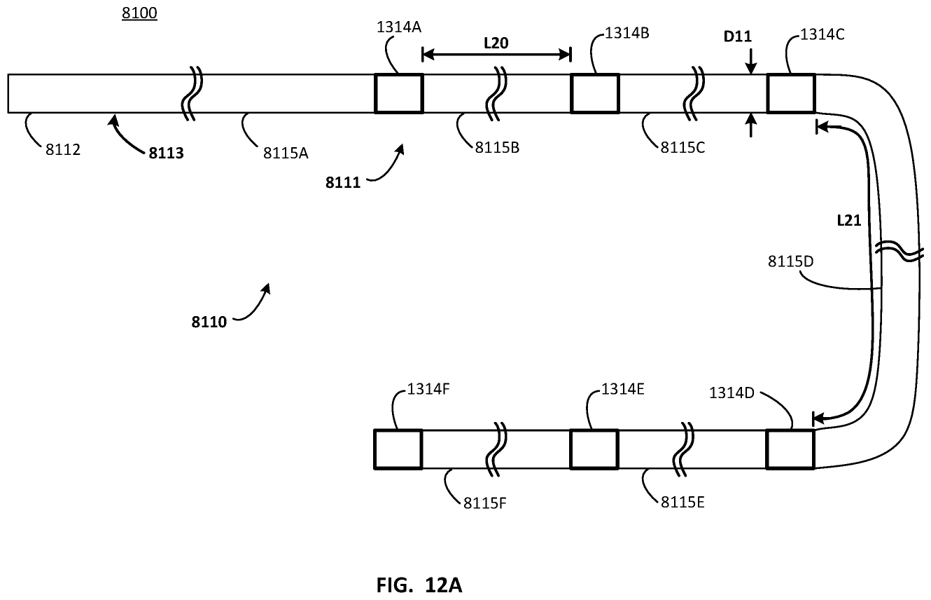

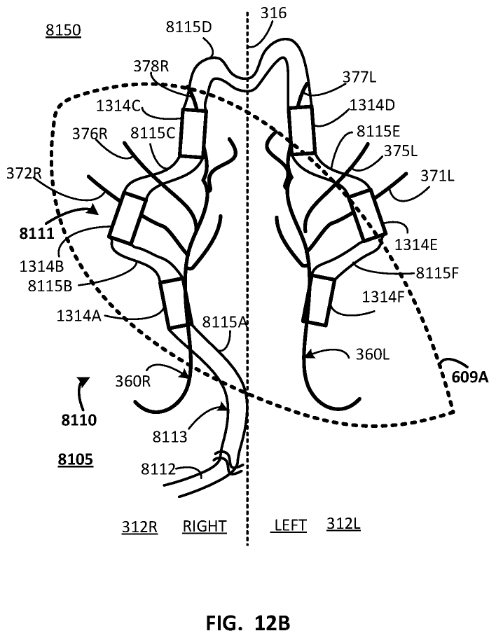

FIG. 12A is a diagram 8100 including a top plan view schematically representing an example stimulation portion 8111 of a distal lead portion 8113 of a lead body 8112 of a stimulation lead 8110. In some examples, the stimulation portion 8111 may comprise at least some of substantially the same features and attributes as the example stimulation portions (e.g. 1311 in FIG. 9A, 1321 in FIG. 10A, 1331 in FIG. 10B) described in association with at least FIGS. 9A-11D. Accordingly, in some examples the stimulation portion 8111 in FIG. 12A comprises similar stimulation elements 1314A-1314F and similar lead segments 8115A-8115F, except with lead segments 8115A-8115F comprising a cross-sectional diameter D11 which is greater than a cross-sectional diameter of the lead segments 1315A-1315F in FIG. 9A. In some examples, the diameter D11 of the lead segments 8115A-8115B of stimulation portion 8111 in FIG. 12A may be substantially the same as a diameter (or greatest cross-sectional dimension) of as the respective stimulation elements 1314A-1314F of stimulation portion 8111 in FIG. 12A such that the entire (or substantially the entire) length of the stimulation portion 8111 comprises a generally uniform cross-sectional shape such as (but not limited to) a generally cylindrical cross-sectional shape. In some such examples, the stimulation elements 1314A-1314F and the lead segments 8115A-8115F of stimulation portion 8111 may be constructed, arranged, etc. relative to each other to provide a generally uniform, generally continuous outer surface without abrupt changes in outer diameter. In some of these examples, each of the stimulation elements 1314A-1314F and each of the respective lead segments 8115A-8115F (e.g. intermediate lead segments) comprise a generally cylindrical shape such that distal lead portion 8113 comprises a generally cylindrical shape.

In some examples, all of the lead segments 8115A-8115F may comprise generally the same length. However, in some examples, some of the respective lead segments 8115A-8115F may comprise a length which differs from a length of the other respective lead segments. In some such examples, the lead segment 8115D may comprise a length which is different from a length L20 of the other respective lead segments 8115A-8115C, 8115E-8115F. In some examples, the length L21 of lead segment 8115D is substantially greater than the length L20 such as, but not limited to, being substantially greater by at least 25 percent, at least 50 percent, at least 75 percent, at least 100 percent, at least 200 percent, and so on. In some examples, the additional length may be provided to better enable the lead segment 8115D to extend across, and straddle, the sagittal midline 316 to enhance positioning stimulation elements 1314A-1314C among target tissues on the first side (e.g. 312R) of patient's body and positioning of stimulation elements 1314D-1314F among target tissues on opposite second side (e.g. 312L) of patient's body. In some examples, the substantially greater length of the lead segment 8115D may enable inclusion of strain relief elements (e.g. 1308 in FIG. 9C) along the length of lead segment 8115D, such as but not limited to undulating shapes (e.g. loop, coil, sigmoid shapes, and the like). In some examples, the principles of the relative lengths of the respective lead segments of stimulation portion 8111 of FIG. 12A is applicable to the example stimulation portions as described in association with at least FIGS. 9A-11D and/or vice versa.

With further reference to FIG. 12A, it will be further understood that the lead segment 8115D generally does not have a pre-formed curved shape such as the shape shown in FIG. 12A, which is merely provided for illustrative simplicity to depict the entire stimulation portion 8111 in a single Figure. However, at least to the extent that it may enhance maneuvering and/or more secure chronic implantation of the stimulation portion 8111, in some examples the lead segment 8115D (or another lead segment 8115A-8115C, 8115E-8115F) may comprise a pre-formed curved shape (e.g. biased shape) like that shown in FIG. 12A or any other shape suitable to enhance such implantation.

FIG. 12B is a diagram 8150 schematically representing an example method 8105 of implantation of the stimulation portion 8111 of FIG. 12A. In some examples, the example method 8105 may comprise at least some of substantially the same features and attributes as the example method (and/or example device 1305) as described in association with at least FIGS. 9A-11D, except with the lead segments 8115A-8115F comprising a greater diameter (or greatest cross-sectional dimension) than a diameter of the lead segments 1315A-1315F in FIG. 9A (and similar elements in FIGS. 10A, 10B). Moreover, it will be understood that the particular manner in which FIG. 12B schematically represents the placement of the various stimulation elements and/or various lead segments relative to the nerve portions (e.g. 372R, 376R, 378R, 371L, 375L, 377L) is merely just one example arrangement.

FIG. 13A is a diagram schematically representing an example stimulation portion 1370. In some examples, the stimulation portion 1370 may comprise at least some of substantially the same features and attributes as, and/or an example implementation of, the example stimulation portions described in association with at least FIGS. 9-12B and/or an example implementation of such previously described stimulation portions.

As further shown in FIG. 13A, in some examples the stimulation portion 1370 comprises an anchor structure 1380 which extends along and around the entire or substantially the entire outer surface 1374 of the stimulation portion 1370 with at least some stimulation elements 1376 interposed between segments of the anchor structure 1380 of anchor elements 1382. Each stimulation element 1376 may comprise one or more contact electrodes 1377. Among other aspects, the anchor structure 1380 stands in contrast to some leads which merely include a limited number of discrete anchor elements. Instead, the anchor structure 1380 provides a continuous or substantially continuous coverage of anchor elements on outer surface 1374 of the stimulation portion 1370. In some examples, the substantially continuous coverage may comprise covering at least about 50 percent of the total surface area of the outer surface 1374 of the stimulation portion 1370. In some examples, the substantially continuous coverage may comprise at least about 60 percent, at least about 65 percent, at least about 70 percent, at least about 75 percent, at least about 80 percent, at least about 85 percent, at least about 90 percent.

In some examples, the continuous or substantially coverage of outer surface 1374 with anchor elements 1382 may sometimes be referred to as a region of indefinite number of anchor elements 1382.

Among other aspects, the anchor structure 1380 of stimulation portion 1370 may facilitate robust fixation of the lead segments 1372A, 1372B, 1372C, 1372D, etc. and/or stimulation elements 1376 relative to surrounding tissues. At the same time, the relatively low profile of the anchor structure 1380 permits at least lateral advancement and maneuvering of the lead segments and/or the stimulation elements of stimulation portion 1370 into the implant positions (and orientations) as shown in FIGS. 9 and 10.

FIG. 13B is a diagram 1390 including a sectional view schematically representing one example implementation of the stimulation portion 1370 of FIG. 13A. As shown in FIG. 13B, the example stimulation portion 1370 may comprise at least some of substantially the same features and attributes as previously described in association with at least FIG. 9B, with similar reference numerals to denote similar elements.

As further shown in FIG. 13B, the example stimulation portion 1370 comprises an anchor structure 1380, which includes a plurality of anchor elements 1382 which are formed on, or defined as part of, the outer surface 1374 of an outer wall 1319 one of the lead segments (e.g. 1372A, 1372B, etc.), which define at least part of the stimulation portion 1370 (FIG. 13A). In some examples, the anchor structure 1380 defines a generally uniform pattern covering the entire or substantially the entire outer surface 1374 of the lead segment(s) (e.g. 1372A, 1372B, etc.) of the stimulation portion 1370.

FIG. 13C is a diagram 1392 including a sectional view schematically representing one example implementation of the stimulation portion 1370 of FIG. 13A (and sectional view of FIG. 13B), while including a contact electrode 1394 in electrical connection with one of the electrical conductors 1317 (e.g. FIG. 9A) extending within an interior 1379 (e.g. 1318 in FIG. 9A) of one of the lead segments (e.g. 1372A, 1372B, etc.) of stimulation portion 1370. As shown in FIG. 13C, in some examples, the anchor elements 1382 may at least partially surround the contact electrode 1394.

Each of FIGS. 14A-14C is a diagram including a side view schematically representing an example stimulation portion (or portion of a stimulation lead body) including an anchor structure formed on, or defined at least partially by, an outer surface of the stimulation portion (or of the stimulation lead body). In some examples, each example anchor structure (1411 in FIG. 14A; 1421 in FIG. 14B; 1442 in FIG. 14C) may comprise at least some of substantially the same features and attributes of an anchor structure (and its associated stimulation portion or portion of a stimulation lead body) of the examples described in association with at least FIGS. 8E-13C, or may comprise an example implementation of the anchor structure (and its associated stimulation portion or portions of a stimulation lead body) described in association with at least FIGS. 8E-13C. It will be further understood that such example anchor structures also may be incorporated into other example devices of the present disclosure, such as on an outer surface of at least a portion of a stimulation lead body, stimulation portion, other type of anchor element, etc.

As shown in the diagram 1400 of FIG. 14A, in some examples anchor structure 1411 may comprise a plurality of rows 1412 of anchor elements 1414 formed on (or defined as at least part of) an outer surface 1374 of a stimulation portion (or portion of a lead body) with spacing 1418 (e.g. absence of anchor elements 1414) interposed between adjacent rows 1412 of the anchor structure 1411. In this arrangement, the rows 1412 are circumferentially spaced apart. In one aspect, each row 1412 is aligned with (e.g. generally parallel to) a longitudinal axis (represented by line A) of the stimulation portion 1371 (or lead body). In some such examples, the size (e.g. width W11) of spacing 1418 and size (e.g. width W12) of the rows 1412 may be selected to implement a desired percentage of coverage of the surface area on the outer surface 1374 of the stimulation portion 1371. However, even with the spacing 1418, in some examples the anchor structure 1411 may sometimes be referred to as extending or covering the entire (or substantially the entire) length of the stimulation portion (or portion of lead body). It will be further understood that even with the inclusion of some minor interruptions (e.g. spaces) along a length of a row 1412 of the anchor structure 1411, the row 1412 (and anchor structure) may still be considered to extend the entire length (or substantially entire length) of the stimulation portion (or portion of stimulation lead body). For instance, one such non-limiting example of an interruption may comprise the presence of a stimulation element which is located along the length of the rows(s) 1412 of the anchor structure 1411.

With regard to the examples of at least FIGS. 14A-14D, in some examples a plurality of anchor elements provide substantially continuous coverage (e.g. occupy a surface area) on an outer surface of at least one of a lead body or a stimulation element. In some such examples, the substantially continuous coverage comprises at least about 25 percent coverage, at least about 30 percent coverage, at least about 35 percent coverage, at least about 40 percent coverage, at least about 45 percent coverage, at least about 50 percent coverage, at least about 60 percent coverage, at least about 65 percent coverage, at least about 70 percent coverage, at least about 75 percent coverage, at least about 80 percent coverage, at least about 85 percent coverage, at least about 90 percent coverage, or at least about 95 percent coverage of the outer surface of at least one of a lead body, a stimulation portion (including distal lead segments and/or a stimulation element), or a stimulation element. It will be further understood that these examples of substantially continuous coverage may be applied to examples of the present disclosure regarding a plurality of anchor elements other than FIGS. 14A-14D.

With regard the example of at least FIG. 14A in which rows 1412 extend longitudinally along length of a lead body, stimulation portion, and/or a stimulation element, the rows 1412 are spaced apart from each other circumferentially, wherein spacing between adjacent rows 1412 comprises an arc length about 5 to about 10 degrees, of about 10 to about 20 degrees, of about 20 to about 30 degrees, of about 30 to about 40 degrees, of about 40 to 50 degrees, of about 50 to about 60 degrees, of about 60 to 70 degrees, of about 70 to about 80 degrees, of about 80 to about 90 degrees, or of about 90 to about 120 degrees.

As shown in the diagram 1420 of FIG. 14B, example anchor structure 1421 may comprise at least some of substantially the same features and attributes of the anchor structure 1411 of FIG. 14A, except with the anchor elements 1414 arranged in a helical pattern of strips 1423A extending about the outer surface 1374 with spacing 1428 (e.g. absence of anchor elements 1414) interposed between adjacent strips 1423A. The dashed lines 1423B represent anchor strips on a backside of the stimulation portion not visible in the view of FIG. 14B, with strips 1423B being in general continuity with strips 1423A, in some examples. Among other aspects, the helically-patterned anchor structure 1421 may provide a desirable combination of sufficient anchorability in both the lateral and longitudinal orientations, while also permitting enough slidability in both the lateral and longitudinal orientations to facilitate implementing desired positioning of the stimulation elements of a stimulation portion at implant locations of target tissues. The helically-patterned anchor structure 1421 may sometimes be referred to as a spiral pattern. In some examples, spacing between adjacent turns about the outer surface 1374 may comprise at least some of substantially the same features regarding coverage and/or spacing as described in association with at least FIGS. 14A and 14C.

As shown in the diagram 1440 of FIG. 14C, example anchor structure 1442 may comprise at least some of substantially the same features and attributes of the anchor structure 1411 of FIG. 14A, except with the anchor elements 1414 on outer surface 1374 arranged in rows 1443 aligned perpendicular to the longitudinal axis (A) of the stimulation portion (or portion of lead body) with spacing 1448 (e.g. absence of anchor elements 1414) interposed between adjacent rows 1443 of anchor elements 1414. In some examples, the particular anchor structure may enhance longitudinal slidability while resisting lateral slidability, particularly after implantation.

In some such examples associated with FIG. 14C, the rows 1443 extend circumferentially with each row 1443 extending transverse to a longitudinal axis of lead (and/or stimulation element), at least in the region in which the rows 1443 are located, with the rows 1443 being spaced apart from each other longitudinally. In some such examples, the spacing (W14) between adjacent rows 1443 comprises at least one multiple, at least two multiples, or at least three multiples of a width (W13) of each row 1443.

FIG. 14D is a diagram 1450 including a sectional view schematically representing an example anchor structure 1452 for a stimulation element 1451A. As shown in FIG. 14D, example anchor structure 1452 may comprise at least some of substantially the same features and attributes of (and/or an example implementation of) the anchor structures as described in association with at least FIGS. 8E-8H, 11A-11B, 13A-14D, with anchor structure 1452 deployed on an outer surface 1454 of a housing of the stimulation element 1451 having at least one contact electrode 1458. As shown in FIG. 14D, in some examples the anchor structure 1452 comprises a plurality of anchor elements 1464 (like anchor elements 1414) extending over the surface area of the entire (or substantially the entire) outer surface 1454 of the stimulation element 1451, including upper and lower surfaces 1455A, 1455B, and side surfaces 1453A, 1453B, (and end surfaces not seen in the sectional view). As further seen in FIG. 14D, electrical conductors 1456 extend within and through the interior 1457 of the stimulation element 1451 with a respective one of the conductors 1456 being electrically connected (via link 1459) to the contact electrode 1458 on lower surface 1455A of the stimulation element 1451. Like the anchor structures present on lead segments (which extend between adjacent stimulation elements), the anchor structure 1452 on an outer surface 1454 of a stimulation element as in FIG. 14D may enhance securely fixing the stimulation element in a position of stimulating relation to target tissues accessible via the midline implant-access incision (e.g. 609A in FIG. 9A).

In some examples, the anchor structures described in association with at least FIGS. 8E-8H, 11A-11B, 13A-14D may be implemented according to at least some of substantially the same features and attributes as the anchor structures 7000, 7100 described in association with FIGS. 15A-15B.

FIG. 15A is a diagram including an enlarged top view schematically representing an example anchor structure 7000 formed on, and including as part of the anchor structure, a base 7002. In some examples, the anchor structure 7000 may comprise an analogous example implementation of the anchor structure 6920 in FIGS. 8E-8H and may comprise at least substantially the same features and attributes as the anchor structure 6920, particularly with respect to providing a matrix of heterogeneous elements. However, in some examples, the anchor structure 7000 of FIG. 15A may have wide applicability to act as an anchor or position-influencing element. In some examples, the anchor structure 7000 in FIG. 15A may comprise one example implementation of the anchor structures, anchor portions, anchor elements in the examples in association with at least FIGS. 8E-8H, 11A-11B, 13A-14D and may comprise at least substantially the same features and attributes as the anchor structures, anchor portions, anchor elements, etc. in the examples in association with at least FIGS. 8E-8H, 11A-11B, 13A-14D.

As shown in FIG. 15A, the anchor structure 7000 may comprise an array 7010 of example heterogeneous elements 7012, 7013, 7016 formed on (and/or extending upward from) a surface 7005 of base 7002. In some examples, the surface 7005 may comprise a planar surface and in some examples, the surface 7005 may comprise a non-planar surface. Together, the heterogeneous elements 7012, 7013, 7016 may form a matrix, network, or the like which may overlap or otherwise be juxtaposed relative to each other to create a generally traction-favoring surface profile. It will be understood that in some examples, the various heterogeneous elements of array 7010 may be positioned much closer to each other than shown in FIG. 10A in order to touch, overlap, partially interlock or interfere with each other, etc. so as to increase the frictional properties (e.g. slide-resistance) of the anchor structure or to reduce the frictional properties (e.g. slidability) of the anchor structure, depending on the type, size, orientation, coating, etc. of the particular arrangement of elements of the array 7010.

In general terms, the various elements of the array 7010 may comprise a flexible, resilient material. However, depending on the goals re slidability or slide-resistance, some elements may be firmer or softer.

In some examples, the particular types, spacing between, orientation, position, relative flexibility, etc. of the heterogeneous elements of the array 7010 may be selected and formed to correspond to a selectable coefficient of kinetic friction to enable a desired bias for controlled slidable movement relative to tissues within a patient's body and/or relative to lumen within a patient's body and/or to correspond to a selectable coefficient of static friction to enable a desired bias to remain statically positioned at a chose location relative to tissues or within a lumen.

In some examples, whether or not expressed formally as a coefficient of kinetic or static friction, the various heterogeneous elements of the array 7010 are selected and formed according to their height, size, shape, position, spacing, orientation relative to each other, relative flexibility, etc. to create a desired anchoring effect while still permitting some degree of slidable advancement.

As shown in FIG. 15A, at least some example shapes (as seen in cross-section from a top view) may comprise elements with shapes which are triangular 7012, circular 7013, rectangular 7016, and the like. The elements also may have different sizes (e.g. diameter, greatest cross-sectional dimension, width, and the like such as represented by S4), and spacing (e.g. S3) between each other or relative to an edge 7031 (e.g. S8) of the base 7002. In some examples, at least some of the elements of array 7010 may comprise hook-shapes, J-shapes, U-shapes, etc. In some examples, at least some of the elements or the juxtaposed pattern of such elements, may promote tissue in-growth and long term fixation, such as but not limited to, apertures formed in such elements or by the juxtaposition of some of the respective elements.

The various elements also may be organized in directional patterns, such as being in rows aligned in a first orientation (R1) or second orientation (R2) which are orthogonal to each other, or in other non-orthogonal orientations. Such orientations may be used to effect selectable bias to permit or prevent slidable movement in various directions, which may enhance positioning and/or anchoring of the medical element on which the anchor structure 7000 is located.

In some examples, at least some elements of the array 7010 may be arranged along a periphery 7030 of the base 7002 in a row or other organizational pattern. The elements 7034 in one example row 7032 may have the same height, size, shape, positions, etc. or may have heights, sizes, shapes, positions different from each other. By providing this configuration along one or more edges 7031 of the base 7002, the anchor structure 7000 may influence slidability or slide-resistance in particular directions. In a related aspect, the presence or absence of elements of array 7010 in an interior portion 7040 also may provide analogous influences, with or without the edge-type rows, etc. of such elements.

In some examples, the interior portion 7040 of the base 7002 and/or the elements of array 7010 also may comprise a coating with desired lubricous and/or frictional qualities, which may be selected to work synergistically with the various shapes, sizes, positions, spacing, orientation, etc. of the elements of array 7010.

FIG. 15B is a diagram including an enlarged side view schematically representing an example anchor structure 7100 formed on, and including as part of the anchor structure, a base 7002. In some examples, the anchor structure 7100 may comprise an analogous example implementation of the anchor structure 6920 in association with at least FIGS. 8E-8H and may comprise at least substantially the same features and attributes as the anchor structure 6920, particularly with respect to providing a matrix or network of heterogeneous elements. However, in some examples, the anchor structure 7100 may have wide applicability to act as an anchor or position-influencing element.

In some examples, the anchor structure 7100 in FIG. 15B may comprise at least some of substantially the same features and attributes as anchor structure 7000 in FIG. 15A.

As shown in FIG. 15B, the anchor structure 7100 comprises an array 7110 of elements comprise different shapes, sizes (e.g. heights, diameters, etc.), positions, spacing, orientations, etc. For example, rectangular elements 7130A, 7130B, 7130C, 7130D exhibit differing angular orientations (e.g. relative to a horizontal plane through which base 7002 extends), which may sometimes be referred to as being bi-directional or multi-directional. Other elements may comprise spherical shaped elements 7120A, 7120B, pyramid-shaped elements 7122, etc. The respective elements of array 7110 may be formed according to a selectable height (per height arrow H3), which may vary from each other as part of a desired effect to promote slidability or slide-resistance, depending on the intended use of the anchor structure and medical element to which is formed/attached.

It will be further understood that some shapes, such as the spherical elements 7120A, 7120B may be more likely to enhance slidability because of their smooth convex surface while some shapes, such as the pyramid element 7122 or rectangular elements (7130A-7130D), may enhance slide-resistance, depending on their orientation. In some examples, directional arrow S10 may represent relative horizontal spacing between elements of array 7110.

In some examples, the base 7002 may formed in a two-dimensional plate shape such that the anchor structure 7000 (FIG. 15A) or 7100 (FIG. 15B) may be readily formed or attached to a back side of a carrier opposite to an electrode side of a stimulation portion, such as a paddle-shaped carrier which carries contact electrodes. However, in some examples, the base may comprise a cylindrical shape such that the elements of array 7010 (FIG. 15A) and/or array 7110 (FIG. 15B) may extend circumferentially outward from a cylindrically shaped lead on which the array 7010 (FIG. 15A) or 7110 (FIG. 15B) is formed or attached. Examples are not so limited and the base may comprise other shapes, as well.

In some examples, and with general reference to anchoring examples in association with at least FIGS. 8E-8H, 11A-11B, 13A-14D, 15A-15B, an anchor structure comprising a plurality of anchor elements may comprise homogeneous elements and/or heterogeneous elements. In some such examples, at least a majority of the homogeneous anchor elements may comprise substantially the same size, shape, position, and/or orientation relative to each other. In some examples, the percentage of anchor elements which are homogeneous relative to each other may comprise at least about 55%, at least about 60%, at least about 65%, at least about 70%, at least about 75%, at least about 80%, at least about 85%, at least about 90%, or at least about 95%.

In some such examples, at least a majority of the heterogeneous anchor elements (e.g. FIGS. 15A-15B) may comprise a different size, different shape, different position, and/or different orientation relative to each other. In some examples, the percentage of anchor elements which are heterogeneous relative to each may comprise at least about 55%, at least about 60%, at least about 65%, at least about 70%, at least about 75%, at least about 80%, at least about 85%, at least about 90%, or at least about 95%.

With regard to at least some of the example homogeneous anchor elements and/or example heterogeneous anchor elements of the present disclosure, each respective anchor element is separate from other respective anchor elements, and a quantity of the plurality of anchor elements is substantially different from, being greater than, at least one of: (A) a quantity of electrodes on at least one of: (1) a single stimulation element of multiple stimulation elements; and (2) all of the stimulation elements for a lead, and (B) a quantity of all the stimulation elements.

With regard to at least some of the example homogeneous anchor elements and/or example heterogeneous anchor elements of the present disclosure, at least some of the respective anchor elements extend outwardly from an external surface of a lead segment (or lead body) by a first distance which is substantially different from, being less than, at least one of a diameter of, a greatest cross-sectional dimension of, or a thickness of the lead segment (or lead body).

With regard to at least some of the example homogeneous anchor elements and/or example heterogeneous anchor elements of the present disclosure, at least some of the respective anchor elements extend outwardly from an external surface of a carrier body of the a stimulation element (e.g. a carrier body supporting contact electrodes) by a first distance which is substantially different from, being less than, at least one of a diameter of, a greatest cross-sectional dimension of, or a thickness of the stimulation element (e.g. the carrier body of the stimulation element).

With regard to at least some of the example homogeneous anchor elements and/or example heterogeneous anchor elements of the present disclosure, at least some of the respective anchor elements comprise a diameter or a greatest cross-sectional dimension which is substantially different from, being less than, a surface area of a respective one of the electrodes of the stimulation element. In some such examples, in this context the diameter (or greatest cross-sectional dimension) of the anchor elements is substantially less than a total surface area of all electrodes of a respective one of the first and second stimulation elements.

With regard to at least some of the example homogeneous anchor elements and/or example heterogeneous anchor elements of the present disclosure, in some examples the plurality of anchor elements may be located on a distal lead segment distal to a bifurcation portion of the lead body. In some such examples, the plurality of anchor elements may be located on a distal lead segment solely distal to a bifurcation portion of the lead body.

FIG. 15C is a diagram 1470 including a sectional view schematically representing an example stimulation portion 1471 (or portion of a stimulation lead body) of an example device and/or example method comprising at least some of substantially the same features and attributes as (but not limited to) the examples described in association with at least FIGS. 9-15B, except comprising an anchor structure 1474 comprising a plurality of tines 1475 extending about the outer surface 1472 of the stimulation portion 1471. As shown in FIGS. 15C-15D, the tines 1475 extend generally perpendicular to a longitudinal axis (reference line A) of the stimulation portion 1471 (or portion of stimulation lead body) and parallel to a minor axis (reference line B) of the stimulation portion 1471.

Moreover, as further shown in FIGS. 15C-15D, in some examples, the stimulation portion (or portion of stimulation lead body) 1471 may be advanced within the patient's body in an orientation (represented by directional arrow LT) which is lateral (e.g. transverse) to a longitudinal axis (line A) of the stimulation portion 1471, which stands in contrast to the typical advancement of a stimulation lead portion in alignment with a longitudinal axis (A) of the stimulation portion 1471. Accordingly, the tines 1475 are aligned to enhance lateral stability of the stimulation portion 1471 more substantially than longitudinal stability of the stimulation portion 1471 while also making the stimulation portion 1471 more maneuverable in a lateral orientation (as represented by directional arrow LT) in order to advance and place the stimulation lead portion(s) in the arrangement in the example method of FIGS. 9A-9C and FIGS. 10A-10B, in which the stimulation elements and lead segments of the stimulation portion are implanted via the midline implant-access incision (e.g. 609A in FIG. 9A) with minor tunneling or no longitudinal tunneling, such as via a direct visualization of the target tissues at which the stimulation elements (and supporting lead segments) may be maneuvered more directly to their implant locations at which stimulating relation (relative to target tissues) is established.

As in other examples, electrical conductors 1456 may extend through and within an interior of the stimulation portion (or portion of stimulation lead body) 1471.

In some examples, the anchor structure may sometimes be referred to as generally providing sideways tines (e.g. being oriented laterally) in at least some lead segments of a stimulation portion versus longitudinal-oriented tines.

FIG. 15D is a diagram 1480 including a top plan view schematically representing the stimulation portion 1471 of FIG. 15C. As shown in FIG. 15D, tines 1475 are spaced apart from each other along a length (e.g. longitudinal axis A) of the stimulation portion 1471 with a longitudinal axis of tines 1475 aligned with an expected generally lateral orientation (LT) (versus a more traditional longitudinal orientation) of advancement of the stimulation portion 1471 within the patient's body during at least some of the implantation of the stimulation portion 1471 in some examples. FIG. 15D also further illustrates the stimulation portion 1471 having opposite ends 1481A, 1481B and opposite sides 1473A, 1473B.

FIG. 15E is a diagram 1483 including a top plan view like that of FIG. 15D schematically representing an example stimulation portion 1482 comprising at least some of substantially the same features and attributes as the stimulation portion 1471 of FIG. 15C-15D, except with tines 1485 (like tines 1475) arranged at a slant (e.g. an angle A) such that a length (e.g. longitudinal axis LA) of the tines 1485 are not perpendicular to the longitudinal axis (line A) of the stimulation portion 1471 or not parallel to the minor axis B of the stimulation portion 1471.

The angle A is selected such that the tines 1485 help to resist “backing out” of the stimulation portion 1491 from an implanted location along the lateral orientation LT (along or parallel to line B) while simultaneously preventing any significant shifting of the stimulation portion 1491 in the longitudinal orientation (along line A).

Via this arrangement, the angled tines 1485 may facilitate slidable advancement of the stimulation portion 1482 in the lateral orientation LT by which a length of the stimulation portion 1482 (or portion of a stimulation lead body) may be inserted and advanced, via an implant-access incision (e.g. midline implant-access incision 609A in FIG. 9A, in some examples), within a patient's body to become positioned at an implant location in stimulating relation to a target tissue (e.g. target nerve portion, target muscle portion, and/or neuromuscular junction) to increase or maintain upper airway patency. In addition, the angled tines 1485 help to maintain longitudinal stability of the stimulation portion at the implant location relative to target tissue.

FIG. 15F is a diagram 1490 including a top plan view like that of FIGS. 15D-15E schematically representing an example stimulation portion 1491 comprising at least some of substantially the same features and attributes as the stimulation portion 1471 of FIG. 15C-15E, except with the addition of at least some tines 1492A, 1492B (like tines 1475) arranged in at angle like tines 1485 in FIG. 15E with some tines (e.g. 1492A) oriented divergently from some tines (e.g. 1492B). Among other aspects, this example arrangement of providing some of the tines 1492A, 1492B at angle (like angle A in FIG. 15E) but in different orientations may provide a more robust fixation in some implementations by providing some back-out resistance in divergent orientations.

FIG. 15G is a diagram 1493A including a top plan view like that of FIGS. 15D-15E schematically representing an example stimulation portion 1493B comprising at least some of substantially the same features and attributes as the stimulation portion 1471 of FIG. 15C-15D, except with tines 1494 (like tines 1475) arranged in a staggered relationship in a lateral insertion orientation (LT) such that a length of some of the tines 1494 are offset from each other in the circumferential orientation. Among other aspects, this example arrangement of tines may provide a more robust fixation in some implementations by providing more variability in anchor points in both a circumferential orientation (along or parallel to line B) and longitudinal orientation (along or parallel to line A). In some such examples, the example anchor arrangement may inhibit or prevent longitudinal migration of the stimulation portion 1493B, which sometimes may be referred to as “lead ratcheting” or “inch worming.” Similarly, the example anchor arrangement may inhibit or prevent lateral migration of the stimulation portion 1493B.

FIG. 15H is a diagram 1495 including a top plan view like that of FIGS. 15D-15E schematically representing an example stimulation portion 1496 comprising at least some of substantially the same features and attributes as the stimulation portions of FIGS. 15C-15D, except with tines 1497A, 1497B (like tines 1492A, 1492B in FIG. 15F) arranged in at angle (like angle A in FIG. 15E) but in a divergent orientation relative to each other. Among other aspects, this example arrangement of tines may provide a more robust fixation in some implementations by providing by providing some back-out resistance in divergent orientations.

In some examples, an anchor structure for a stimulation portion (e.g. lead body, stimulation element) of various examples of the present disclosure and/or of a pulse generator (333 in FIG. 3, 1133 in FIG. 17A) may comprise varying combinations of the features and attributes of the example implementations of FIGS. 15C-15H.

Moreover, in some examples, a stimulation portion may be implemented comprising an anchor structure comprising at least some of substantially the same features of the anchor structures of FIGS. 15C-15H combined with at least some of substantially the same features and attributes of the anchor structure(s) of at least FIGS. 15A-15B and/or FIGS. 8E-8H, 11A-11B, 13A-13C, 14A-14D, 16J-16K, 23A-24E, and/or 25H.

FIG. 16A is a diagram 1500 schematically representing an example method and/or example device 1505 for treating sleep disordered breathing and including a pair of independently positionable, elongate stimulation elements 1532A, 1532B. Each stimulation element 1532A, 1532B comprises an axial array of contact electrodes 1572A, 1572B, 1572C, 1572D, 1572E.

As shown in FIG. 16A, one example device and/or example device 1505 comprises a stimulation lead 1510 comprising at least some of substantially the same features and attributes as in FIGS. 1A-15G and being implanted via a midline implant-access incision 609A (in some examples) via at least some of substantially the same features and attributes as in FIGS. 1A-15G, except with a stimulation portion comprising stimulation elements 1532A, 1532B each arranged as an axial array of contact electrodes 1572A-1572E, among other differences.

Like some of the previously-described example devices and/or example methods, the stimulation lead 1510 comprises lead body 1522 and a bifurcation portion 1528 from which two separate flexible distal lead segments 1530A, 1530B extend to support the respective stimulation elements 1532A, 1532B.

Each stimulation element 1532A, 1532B comprises an elongate body 1537 extending between a distal end 1536 and a proximal end 1534, which extends from, and which is electrically and mechanically connected to, the distal end 1533 of transition portion (e.g. body) 1531 of the respective distal lead segments 1530A, 1530B. As shown in FIG. 16A, in some examples the distal ends 1533 of the respective distal lead segments 1530A, 1530B are separate from and independent of each other such that lead 1510 is arranged without a structure extending directly (e.g. in a generally linear manner) between proximal ends 1534 of the respective stimulation elements 1532A, 1532B. Instead, at least because of the lengths of the distal lead segments 1530A, 1530B and/or their bifurcated relationship, the distal lead segment 1530A (including the transition portion 1531, distal end 1533, and stimulation element 1532A) is positionable separately from, and independent of, distal lead segment 1530B (including the transition portion 1531, distal end 1533, and stimulation element 1532B).

Accordingly, one stimulation element 1532A can be positioned (e.g. translated) along a first orientation (as represented by arrow V13) on a right side 312R (of the patient's body) toward or away from the midline 316 independently of the positioning of the other stimulation element 1532B along a second orientation (as represented by arrow V14) on a left side 312L (of the patient's body) toward or away from the sagittal midline 316 of the patient's body. In some examples, one or both of the stimulation elements 1532A, 1532B may be implanted in a manner which protrudes more deeply into at least some target tissues such as, but not limited to, the later described example arrangements of FIGS. 16C-16G.

In one aspect, as schematically represented in the diagram 1590 of FIG. 16B, while independently positioning each stimulation element 1532A, 1532B within the patient's body, the example method may comprise orienting one stimulation element 1532A (e.g. on right side 312R, as represented by line V15a) at an angle Ω relative to the orientation of the other stimulation element 1532B (e.g. on left side 312L, as represented by line V15b) and/or relative to the sagittal midline 316. In some examples, a first orientation of one or both of the stimulation elements 1532, 1532B may be aligned generally parallel to the sagittal midline 316 of the patient's body. However, in some examples, the first orientation of one or both of the stimulation elements 1532A, 1532B extends at an acute first angle relative to the sagittal midline between about 1 degree and 60 degrees, between about 1 degree and 45 degrees, and/or between about 1 degree and about 30 degrees.

In one aspect, as schematically represented in the diagram 1590 of FIG. 16B, while independently positioning each stimulation element 1532A, 1532B within the patient's body, the example method may comprise orienting one stimulation element 1532A (e.g. on right side 312R) at an angle Ω relative to the orientation of the other stimulation element 1532B (e.g. on left side 312L) according to at least one degree of freedom (e.g. one rotational plane/axis, such as yaw). In some such examples, this arrangement may comprise positioning a proximal end 1534 of each respective stimulation element 1532A (e.g. a first and second carrier body supporting electrodes) closer to a chin and a distal end 1536 of each respective first and second stimulation element further from the chin, wherein the distal ends 1536 of the respective first and second stimulation elements 1532A, 1532B are oriented to diverge from each other. In some examples, the angle Ω may comprise between about 0 degrees (e.g. parallel) and 110 degrees. In some examples, the angle Ω may comprise between about 5 and 100 degrees, between about 10 and 90 degrees, between about 15 and 80 degrees, between about 20 and 70 degrees, between about 25 and 60 degrees, and between about 30 and 50 degrees.

It will be further understood that the respective stimulation elements 1532A, 1532B may be oriented rotationally at a selectable angle according to other rotational planes/axes (e.g. pitch and/or roll). Moreover, within the space constraints of the patient's anatomy, the respective stimulation elements 1532A, 1532B also may be selectably positioned according to three translational degrees of freedom.

However, in view of the generally elongate, cylindrically shape of the respective stimulation elements and/or the general position and orientation of the target tissue, at least some example methods comprise generally attempting to align a longitudinal axis of each stimulation element 1532A, 1532B to be generally parallel to a longitudinal axis of each mandible (e.g. right mandible and left mandible) of the patient's body. In such examples, the extent to which the respective stimulation elements may be manipulated according all six degrees of freedom may be relatively limited, at least as compared to some later examples such as (but not limited to) the examples of FIGS. 18A-25 or FIGS. 9-15E in which the respective stimulation elements, stimulation elements, etc. may be manipulated within a much fuller range of motion in each of three orthogonally-related rotational degrees of freedom (e.g. yaw, pitch, and roll) and/or three orthogonally-related translational degrees of freedom, as described in association with at least FIGS. 18A-21B and/or FIGS. 9A-9C, 10A-10B.

As further shown in FIG. 16A, contact electrodes 1572A-1572E are at least partially exposed on an outer surface of body 1537 of each stimulation element 1532A, 1532B with insulative portions 1571 interposed between the spaced apart contact electrodes 1572A, 1572B, 1572C, 1572D, 1572E or positioned at distal end 1536 and/or proximal end 1534. In some examples, the contact electrodes (e.g. 1572A, 1572B, etc.) may comprise ring electrodes or split-ring electrodes. Once each stimulation element 1532A, 1532B is secured into a fixed position (such as via the later described anchors) relative to various nerve portions of each respective nerve (e.g. 360R, 360L), then an example method may comprise determining, upon test stimulation, which target nerve portions, target muscle portions, combination of target nerve portions and muscle nerve portions, neuromuscular junctions, and/or combinations thereof likely will produce a desired response of contraction of protrusor muscles to cause tongue protrusion to increase or maintain upper airway patency. In doing so, the example method also may comprise determining which contact electrodes (e.g. 1572A, 1572B, etc.) of the stimulation element 1532A or 1532B will be in stimulating relation to the target nerve portions, target muscle portions, combination of such target nerve portions and target muscle portions, neuromuscular junctions, and/or combinations thereof to produce the desired response of protrusor muscles. As shown in FIG. 16A, at least some example target nerve portions comprise 372R, 376R, 378R, 371L, 375L, 377L, 366R and/or 366L. In some examples, the nerve portion 366R may comprise a more proximal portion of a nerve branch 360R (e.g. FIGS. 2A-2B) from which nerve portions 372R, 376R, 378R distally extend and the nerve portion 366L may comprise a more proximal portion of a nerve branch 360L (e.g. FIGS. 2A-2B) from which nerve portions 371L, 375L, 377L extend.

In some examples, the nerve portions 372R, 376R, 378R, 371L, 375L, 377L comprise substantially the same features and attributes as described previously in association with at least FIGS. 2A-2B and/or other examples throughout the present disclosure.

However, in some examples, nerve portion 366R may comprise a target nerve portion unrelated to the nerve portions 372R, 376R, 378R and/or the nerve portion 366L may comprise a target nerve portion unrelated to the nerve portions 371L, 375L, 377L.

In some examples, each stimulation element 1532A, 1532B may comprise an anchor structure 1580A, such as a plurality of tines 1581. The tines 1581 may comprise resilient flexible elements which are biased to extend in an outward orientation relative to body 1537 as shown in FIG. 16A, but which may be collapsible against body 1537 during delivery of the stimulation element 1532A, 1532B into a desired position at or after which the tines 1581 may be released into their biased outward orientation for engaging surrounding non-nerve tissue to anchor at least the distal end 1536 of the stimulation element 1532A, 1532B into a stable position to thereby secure the contact electrodes (e.g. 1572A, 1572B, etc.) into a robust position of stimulating relation to a target tissue, such as but not limited to, target nerve portion(s), target muscle portion(s), a combination of target nerve portion(s) and target muscle portion(s), neuromuscular junctions, and/or combinations thereof.

It will be understood that tines (e.g. 1581) also may be located at more proximal locations along the body 1537 of the stimulation elements 1532A, 1532B, including being positioned at portions 1571 between adjacent contact electrodes (e.g. 1572B and 1572C, 1572C and 1572D, etc.). Moreover, in some examples, some tines 1581 in the more proximal locations may be oriented in an opposite direction than the orientation of tines 1581 at distal end 1536, as shown in FIG. 16A.

In some examples, the transition portions 1531 of the distal lead segments 1530A, 1530B may omit anchor elements (e.g. tines, other), which may enhance or facilitate positioning of the stimulation elements 1532A, 1532B according to a fuller range of motion according to rotational degrees of freedom and/or translational degrees of freedom.

However, in some examples, anchors may be added later after positioning during implantation is completed and/or the transition portions 1531 may comprise anchor portions, anchor structures, etc. which are configured in a manner to enable positioning during implantation while still achieving robust fixation once the implantation is completed. For instance, in some examples, an anchor structure may comprise at least some of substantially the same features and attributes as described in association with at least FIGS. 9-15E, 16C-16D, and 23A-25.

As shown in FIG. 16A, via the position and orientation of the stimulation element 1532A relative to nerve portions of nerve 360R and of the stimulation element 1532B relative to nerve portions of nerve 360L, activation of some electrodes such as 1572A will result in stimulation of more proximal target nerve portions while activation of more proximal electrodes (e.g. 1572E) may result in stimulation of more distal branches (including but not limited to terminal branches) of nerve 360R and nerve 360L, respectively. Accordingly, via selection of a single electrode or combination of electrodes from the array of available electrodes (e.g. 1572A, 1572B, etc.) along the length (L9) of each stimulation element 1532A, 1532B, the example method may determine a best fit of which electrodes to activate (and when) to cause protrusor muscle contraction to increase or maintain upper airway patency via tongue protrusion.

FIG. 16C is a diagram 1600 including a bottom view of at least a patient's submandibular region of a target tissue environment 7209 (e.g. see also at least FIGS. 81-8L) and schematically representing an example method and/or example device 1605 for treating sleep disordered breathing including a pair of implanted stimulation elements 1632A, 1632B in stimulating relation to an upper airway patency-related tissues within the target tissue environment 7209.

In some examples, example device 1605 may comprise at least some of substantially the same features and attributes as at least example device (and/or example method) including stimulation lead 1510 of FIGS. 16A-16B, except with stimulation elements 1632A, 1632B of an example stimulation lead 1610 which are configured to at least partially protrude, in their implanted position, upward in a superior orientation away from a mandibular plane.

With this in mind, FIGS. 16C, 16D, 16G provide a bottom view, a side view, and a front view, respectively, which schematically represent various aspects of the example stimulation lead 1610 and associated example device 1605 (and/or example method) in the context of target tissue environment 7209 of patient anatomy 7509 such as a patient's head-and-neck region. In some examples, the target tissue environment 7209 in FIGS. 16C-16E may comprise at least some of substantially the same features and attributes as the example target tissue environment 7209 described in association with at least FIGS. 81-8L.

As shown in FIGS. 16C-16G, the stimulation lead 1610 comprises a pair of stimulation elements 1632A, 1623A. As shown in FIG. 16C, the stimulation lead 1610 comprises a proximal portion 1622 which extends to and is connectable with a pulse generator (e.g. 333 in FIG. 2A or 1133 in FIG. 17A) and a distal portion 1624 which extends from the proximal portion 1622 to be located near a sagittal midline 316 in a patient's head-and-neck region such as, but not limited to, a region located inferior and posterior to the chin 315. This region may sometimes be referred to as a submental region. The distal portion 1624 of lead 1610 may comprise bifurcation portion 1628 from which two separate and independent flexible distal lead segments 1630A, 1630B extend further distally to support stimulation elements 1632A, 1632B, respectively. Each flexible distal lead segment 1630A, 1630B may comprise a transition portion 1631 (e.g. body) and a distal end 1633. The distal end 1633 of each lead segment 1630A, 1630B is connected (electrically and mechanically) to a proximal end 1634 of the stimulation elements 1632A, 1632B, respectively so that electrical conductors extending within and through the lead segments 1630A, 1630B are in electrical communication with the individually addressable contact electrodes 1672Y and 1672X (shown in FIG. 16D) of the respective stimulation elements 1632A, 1632B. As further described later and as apparent from at least FIGS. 16D (side view) and 16E (front view), a proximal portion 1638 of the stimulation elements 1632A, 1632B shown in FIG. 16C comprise just a portion of the entire respective stimulation elements as a distal portion (e.g. 1639) of each stimulation element (1632A, 1632B) extends superiorly into, within, and among the target tissues (e.g. nerve portions, muscle portions, combinations of nerve portions and muscle portions, neuromuscular junctions of nerve portions and muscle portions, and combinations thereof) within the target tissue environment 7209, as shown in at least FIG. 16D.

As further shown in FIG. 16C (bottom view) and FIG. 16D (side view), the distal portion 1624 (of stimulation lead 1610) is introduced and placed via midline implant-access incision 609A (FIG. 16C) for chronic implantation in which the proximal portions 1638 of the stimulation elements 1632A, 1632B extend in a generally mandibular plane with at least the proximal portion 1638 (of the respective stimulation elements 1632A, 1632B) juxtaposed with a respective hypoglossal nerve portion (e.g. 361HR on patient right side 312R; 361HL on patient left side 312L) and genioglossus muscle portion (e.g. 1619R on patient right side 312R; 1619L on patient left side 312L).

Further details regarding the stimulation elements 1632A, 1632B as represented in FIG. 16C will be described later in the context of describing the example side view of FIG. 16D and/or front view of FIG. 16G.

As shown in FIG. 16D, each stimulation element 1632A, 1632B comprises a first surface 7653A and opposite second surface 7653B. In some examples, the first surface 7653A of each stimulation element 1632A, 1632B may sometimes be referred to as inner surface, at least to the extent that upon implantation the first surface 7653A may face inward toward deeper portions of the target tissue within the patient's head-and-neck region). Meanwhile, the opposite second surface 7653B of each stimulation element 1632A, 1632B may sometimes be referred to as an outer surface, at least to the extent that upon implantation the second surface 7653B may face outward toward more superficial portions of the target tissue and the patient's head-and-neck region (e.g. toward the chin).

As further shown in FIG. 16D, in some examples the stimulation elements 1632A, 1632B may exhibit a curved shape in their chronically implanted state. In some such examples, the stimulation element 1632A, 1632B may comprise a curved shape prior to implantation with the pre-implantation curved shape being substantially the same as the post-implantation curved shape of the stimulation elements. In some examples, this arrangement may sometimes be referred to as a pre-formed curvilinear-shaped stimulation element or a pre-formed curvilinear-shaped carrier body.

In some examples, the pre-formed curvilinear shape comprises a first radius of curvature which generally corresponds to a second radius of curvature of an arcuately-shaped implant path formed prior to implantation of the stimulation elements 1632A, 1632B. In some examples, the pre-formed curvilinear shape comprises a third radius of curvature which is substantially greater than the second radius of curvature. In some examples, the implant path may sometimes be referred to as a partial tunnel or a tunnel.

In some examples, in examples in which the first carrier body comprises a pre-formed shape, the substantially greater radius of curvature of the first carrier body has a radius of curvature about 25%, 50%, 75%, 2×, the radius of curvature of the implant path so that the first carrier body has a more relaxed curvature prior to insertion and advancement into the implant path. Via this example, the pre-formed shape (e.g. pre-curved shape) of the first carrier body has a gentler curve so it is easier to introduce the first carrier body into the implant path (than if sharper curved shape) and to advance the first carrier body through the implant path. At the same time, providing some pre-curved shape facilitates introduction/advancement to be easier/faster than if the first carrier body had no pre-curved shape.

However, in some examples, prior to implantation each stimulation element 1632A, 1632B may comprise a generally straight or linear shape which may be flexed, bent, manipulated etc. into a curved shaped (e.g. the curvilinear shape) during and/or after implantation. In some examples, the flexible stimulation elements 1632A, 1632B may assume such a curved shape at least because of an implant access pathway (e.g. shown via dashed lines 7675 in FIG. 16F) formed in the target tissue environment 7209 which can be used to deliver the flexible stimulation elements 1632A, 1632B into a desired position and the curved shape. In some such examples, the stimulation element 1632A, 1632B may sometimes be referred to as taking on a shape and orientation generally corresponding the shape and orientation of the arcuately-shaped implant access pathway 7675. In some examples, the implant access pathway 7675 may sometimes be referred to as an arcuately-shaped tunnel.

In some examples, the access pathway 7675 (FIG. 16F) may be formed via various tools such as, but not limited to, an introducer, stylet, guide catheter, guidewire, etc. In some examples, the stimulation element 1632A, 1632B may comprise a steering wire or other structure housed internally within a body 1671 of the stimulation element which is adapted to help cause the stimulation element 1632A, 1632B to assume a flexed, bent, etc. shape during implantation.

With further reference to at least FIGS. 16C-16F, upon implantation the stimulation element 1632A, 1632B is to become chronically positioned in an arcuate shape within and among target tissues, with the stimulation element generally extending in an anterior-posterior orientation and a superior-inferior orientation, with a proximal portion 1638 of the stimulation element (e.g. 1632A, 1632B) extending within or generally parallel to the sub/mandibular plane (Mand), and a distal portion 1639 of the stimulation element extending a first angle relative to the sub-mandibular plane (Mand).

In some examples, first angle may comprise about 80 degrees to about 140 degrees, about 85 to about 130 degrees, about 85 to about 120 degrees, about 85 to about 110 degrees, about 85 to about 100 degrees, or about 85 to about 95 degrees.

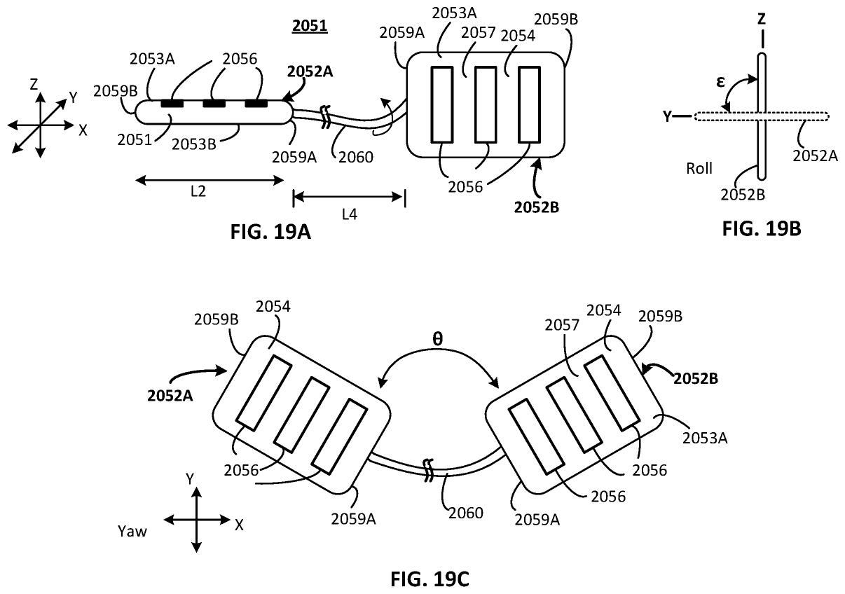

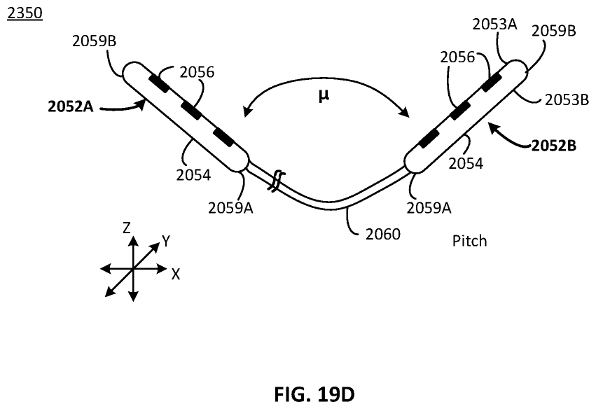

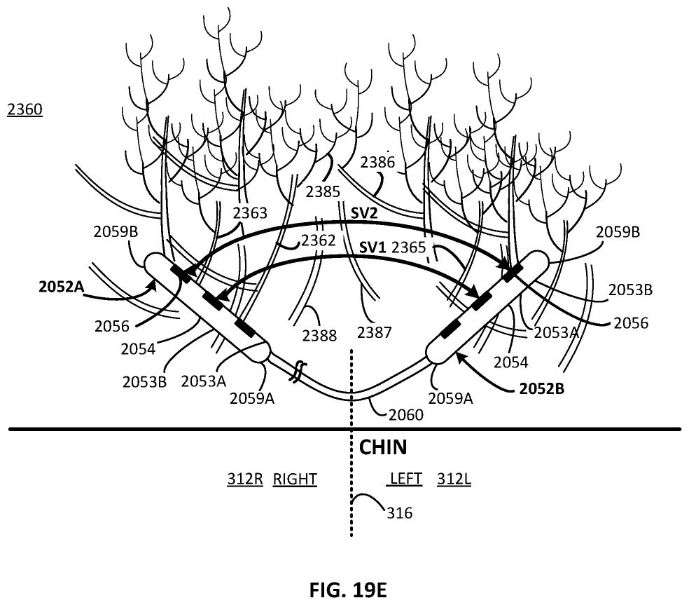

In some examples, other than its width, the stimulation element 1632A, 1632B does not extend in the medial-lateral orientation. However, in some examples, the stimulation element may be positioned per a roll parameter (e.g. FIGS. 19A, 19B) (rotated about an anterior-posterior orientation) to be tilted inward toward a centerline of the tongue or tilted outward away from centerline of tongue.

In some examples, each stimulation element 1632A, 1632B may comprise a printed circuit-type element comprising at least some of substantially the same features and attributes as the stimulation elements (e.g. 1314A, 1314B, etc.) of FIGS. 9A-9B and/or stimulation elements (e.g. 1324A, 1324B, etc.) of FIGS. 10A-10B. In some such examples, the body 1671 of each stimulation element 1632A, 1632B comprises a dielectric or insulative material and the contact electrodes 1672X, 1672Y are printed onto the body 1671. It will be understood that conductive elements extend from each contact electrode, separately from each other, through or on the body 1671 for connection to a pulse generator (e.g. 333 in FIG. 3; 1133 in FIG. 17A) via conductive elements extending within, on, and/or through the body 1623 of lead 1610. Like the example stimulation elements of FIGS. 9A-10B, in some examples the stimulation elements 1632A, 1632B may comprise a low profile (e.g. cross-sectional size and/or shape) to facilitate introduction and delivery of the stimulation elements 1632A, 1632B into the chronically implanted position shown in FIGS. 16C-16G.

With this in mind, FIG. 16E is a sectional view as taken along lines 16E-16E of FIG. 16D which schematically represents one example implementation of a cross-sectional configuration of each stimulation element (e.g. 1632A, 1632B). In particular, as shown in FIG. 16E, a body 1671 of the stimulation element 1632B may comprise a width (L8) extending between opposite side edges 7657A, 7657B and a thickness (T8) (e.g. height) extending between the inner and outer surfaces 7653A, 7653B. Meanwhile, FIG. 16D depicts a length extending between distal end 1636 and proximal end 1634. With further reference to FIG. 16E, in some examples the width (L8) may be substantially greater than the thickness (T8) to provide the low profile configuration. In some examples, the term “substantially greater” may comprise a difference of the width (L8) being at least 3 times, 4 times, or 5 times greater than the thickness (T8).

In some examples, the body 1671 of the stimulation element 1632A, 1632B may comprise a rounded rectangular cross-sectional shape as shown in FIG. 16D. However, in some examples, the body 1671 may comprise a rectangular cross-sectional shape, an elliptical cross-sectional shape, an obround cross-sectional shape, and the like in which a width (L8) may be substantially greater than its thickness (T8). In view of its length being at least one or more orders of magnitude greater than its thickness or width, in some examples the stimulation element 1632A, 1632B may sometimes be referred to as a ribbon or ribbon-like element but with enough rigidity to prevent or minimize collapse or folding during introduction and advancement into a target tissue environment 7209.

In order to enhance the low profile, cross-sectional configuration of the stimulation element 1632B, in some examples, an outer surface of the contact electrode 1672X may be flush with the surface (e.g. inner surface 7653A) of body 1671 of the stimulation element. Similarly, in some examples, the contact electrodes 1672Y on the opposite outer surface 7653B may be flush with the surface of body 1671.

As represented by just one stimulation element 1632B shown in FIG. 16D, each stimulation element 1632A, 1632B may be introduced and advanced into and through implant-access incision 609A (FIG. 16C) in an orientation along the directional arrows V16 (FIGS. 16C-16D) such that the stimulation element 1632B becomes chronically implanted with the distal end 1636 of the stimulation element 1632B extending superiorly away from the general mandibular plane (see plane designators Mand) while extending somewhat anteriorly toward a front of the mouth, while a proximal end 1634 (e.g. base 7654 of FIG. 16E) of the stimulation element 1632B remains at or near the mandibular plane (see indicator Mand). In this regard, FIG. 16D generally schematically represents a bottom of a mandible bony portion via indicator 7241.

While FIG. 16D depicts the stimulation element 1632B as exhibiting a particular curved shape as one example, it will be understood that in its chronically implanted configuration, the stimulation element 1632B may exhibit degrees of curvature other than that shown in FIG. 16D.

In its chronically implanted position as further shown in at least FIG. 16D, in some examples the stimulation element 1632B (also representative of stimulation element 1632A) may extend alongside at least one segment of the nerve portion 7280. In some examples, nerve portion 7280 may comprise a more proximal portion of a medial branch of the hypoglossal nerve 7260. Via this example arrangement, several of the contact electrodes 1672X (or several adjacent pairs of contacts electrodes 1672X) on inner surface 7653A of stimulation element 1632B are positioned to be in stimulating relation to at least some segments of the nerve portion 7280 as the nerve portion 7280 extends distally, which may at least partially include a superior orientation.

Moreover, while FIG. 16D depicts a two-dimensional sectional view, it will be understood that the various nerve portions may extend in three-dimensions such that in some examples, different stimulation vectors may be applied among the various contact electrodes 1672X (on inner surface 7653A) relative to the segments (and/or branches extending therefrom) of nerve portion 7280. As can be further seen in FIG. 16D, other branches (e.g. nerve portions 7270, 7272, 7262, etc.) extend outwardly from the nerve portion 7280 and also can be captured and stimulated via different contact electrodes 1672X (e.g. adjacent pairs of such electrodes) along inner surface 7653A and/or via different stimulation vectors generated among the contact electrodes 1672X spaced apart along the curved inner surface 7653A of stimulation element 1632B.

Moreover, in some examples, by capturing the more proximal nerve portions (e.g. 7280, 7261) of the hypoglossal nerve via various potential stimulation signals applied via the more proximal contact electrodes 1672X, 1672Y, the stimulation element 1632B may be provide therapeutic stimulation therapy signals indirectly to the distal terminal nerve portions 7264, 7285, 7292 and/or less proximal nerve portions (e.g. 7290 which supports distal terminal nerve portion 7292).

As further shown in FIG. 16D, in some examples the distal portion 1639 of the stimulation element 1632B may extend more superiorly into, among, and/or within the target tissues, at least some of the more distally located contact electrodes 1672X on inner surface 7653A and/or contact electrodes 1672Y on outer surface 7653B may be positioned to be in stimulating relation to at least some example distal terminal nerve portions 7285 of a group or region 7282 of such distal terminal nerve portions.

As further shown in the diagram 7600 of FIG. 16D, the more distal terminal nerve portions (e.g. 7264) extending from nerve portion 7262 may innervate muscle portions 7244A, 7244B, 7244C which originate from an interior portion of chin 7240. Moreover, the more distal terminal nerve portions (e.g. 7292) may innervate muscle portions 7247 closer to a top surface portion of the tongue 7246. Other distal terminal nerve portions 7285 (also 7282, 7274, and the like) may innervate more proximal muscle portions of the tongue (genioglossus muscle), at least some of which are involved in causing protrusion of the tongue and hence which may sometimes be referred to as protrusor muscles.

With this general arrangement in mind for the example method and/or example device 1605 including stimulation lead 1610, FIG. 16G provides a diagram 7700 including a front view schematically representing the example stimulation elements 1632A, 1632B of lead 1610 in a chronically implanted position (e.g. arrangement 7350) generally corresponding to the example of FIGS. 16C and/or 16D with some aspects of the stimulation elements 1632A, 1632B shown in a simplified manner for illustrative clarity.

Consistent with FIGS. 16C-16D, as shown in FIG. 16G the stimulation elements 1632A, 1632B are located on opposite sides of a sagittal midline 316 to be positioned for bilateral stimulation, or unilateral stimulation as desired. As shown in FIG. 16G, a distal portion 1639 of each stimulation element 1632A, 1632B extends upward in a general superior orientation with FIG. 16E depicting an outer surface 7653B of the stimulation elements 1632A, 1632B facing forward in an anterior orientation. In this configuration, the plurality of contact electrodes 1672Y (on outer surface 7653A), 1672X (on inner surface 7653A as seen in FIG. 16D) extend, in a vertically spaced apart relationship, in a column-like manner generally vertically upward in the superior orientation to be positioned to be in stimulating relation with target tissues (e.g. nerve portion(s), muscle portion(s), combinations of nerve portion(s) and muscle portion(s), neuromuscular junctions of nerve portion(s) and muscle portion(s), and combinations thereof). In some examples, the target tissues may comprise a first nerve portion innervating a genioglossus oblique (GGo) muscle portion, the genioglossus oblique (GGo) muscle portion, and/or a neuromuscular junction of the first nerve portion and the genioglossus oblique (GGo) muscle portion. In some examples, the target tissues may comprise a second nerve portion innervating a genioglossus horizontal (GGh) muscle portion, the genioglossus horizontal (GGh) muscle portion, and/or a neuromuscular junction of the second nerve portion and the genioglossus horizontal oblique (GGh) muscle portion. In some examples, the target tissues may comprise a third nerve portion innervating a geniohyoid (GH) muscle portion, the geniohyoid (GH) muscle portion, and/or a neuromuscular junction of the third nerve portion and the geniohyoid (GH) muscle portion.

As further shown in FIG. 16G, example contact electrode portion 1672YP represents a visible distal portion of a contact electrode (e.g. 1672Y on outer surface 7653B) with the proximal portion of the contact electrode being unseen in FIG. 16G at least because the more proximal portions of the respective stimulation elements 1632A, 1632B extend posteriorly out of view. Moreover, portion 1635 represents a transition portion at which each stimulation element 1632A, 1632B extends posteriorly to a degree such that the remaining proximal portion of each stimulation element 1632A, 1632B is no longer visible in FIG. 16E.

With at least some of these example arrangements in mind in which stimulation may be applied via electrodes on the distal portion 1639 of the respective stimulation elements 1632A, 1632B, in some examples the proximal portion 1638 (FIGS. 16C, 16D) of the respective stimulation elements 1632A, 1632B may omit contact electrodes (e.g. stimulation electrodes) and/or such electrodes may remain dormant while just the contact electrodes of the distal portion 1639 (FIGS. 16D, 16G) are utilized to stimulate the deeper (e.g. less superficial) target tissues as noted above. In some such examples, the transition portion 1635 also may omit contact electrodes such that just the distal portion of the stimulation elements 1632A, 1632B include contact electrodes for applying stimulation to target tissues. With such arrangements, the proximal portion and/or transition portion of the respective stimulation elements 1632A, 1632B may sometimes be referred to as being electrode-free. However, in some such examples, the transition portion 1635 (FIGS. 16C, 16D, 16G) and/or proximal portion 1638 (FIGS. 16C, 16D) may comprise sensor(s), which may comprise sensing electrodes and/or other forms of sensors. In such examples, the transition portion 1635 and/or proximal portion 1638 may sometimes be referred to as being stimulation-electrode-free. In some examples, the sensors may comprise EMG sensors, impedance sensors, and/or other types of sensors.

As further shown in FIG. 16G, at least some of the nerve portions (e.g. 7671, 7673, 7675, etc.) may extend along a path intersecting with, or being in sufficiently close proximity, to be in stimulating relation to at least some of the contact electrodes (e.g. 1672Y, 1672X), adjacent pairs thereof, and/or a stimulation vector between various combinations of contact electrodes, in a manner similar to that previously described in association with at least FIGS. 8A-8J.

In some examples, each stimulation element 1632A, 1632B, the distal lead segments 1630A, 1630B, and/or other portions of stimulation lead 1610 may comprise at least some of substantially the same features and attributes of the anchor structures, portions, anchor elements, etc. as previously described in association with at least FIGS. 11A-15H as appropriate. Via such anchoring arrangements, each stimulation element 1632A, 1632B may be securely fixed in a position within and among bodily tissues within target tissue environment 7209 to maintain the contact electrodes 1672Y (and/or body 1671 generally) in stimulating relation to the target tissues. As shown in FIGS. 16D-16G, such target tissues may comprise nerve portions, muscle portions, combinations of nerve portions and muscle portions, neuromuscular junctions of nerve portions and muscle portions, and/or combinations thereof.

In some examples, stimulation elements 1632A, 1632B may be understood as extending primarily in an anterior-posterior, and superior-inferior orientation which stands in sharp contrast to some stimulation elements which extend significantly in a medial-lateral orientation, such as extending in a path aligned between or across a span between a left and right mandible.

FIG. 16H is a diagram 7800 including a bottom view of at least a patient's submandibular region of a target tissue environment 7209 (e.g. see also at least FIGS. 16C-16G) and schematically representing an example method and/or example device 7805 for treating sleep disordered breathing including a stimulation lead 7810 (e.g. stimulation portion) including a pair of implanted stimulation elements 7832A, 7832B in stimulating relation to an upper airway patency-related tissues within the target tissue environment 7209.

In some examples, example device 7805 may comprise at least some of substantially the same features and attributes as at least example device 1605 (and/or example method) including stimulation lead 1610 of FIGS. 16C-16G, except with stimulation elements 7832A, 7832B of an example stimulation lead 7810 which are configured to protrude, in their implanted position, in a posterior orientation away from a chin and upward in a superior orientation away from the mandibular plane (Mand).

With this in mind, FIGS. 16H and 161 provide a bottom view and a side view, respectively, which schematically represent various aspects of the example stimulation lead 7810 and associated example device (and/or example method) 7805 in the context of target tissue environment 7209 within a patient's head-and-neck region. In some examples, the target tissue environment 7209 in FIGS. 16F-16G may comprise at least some of substantially the same features and attributes as the example target tissue environment 7209 described in association with at least FIGS. 16C-16E.

As shown in FIG. 16H, the stimulation lead 7810 comprises proximal portion 7822 (like 1622 in FIG. 16C) and distal portion 7824 extending from the proximal portion 7822. The distal portion 7824 may comprise a bifurcation portion 7828, from which extends distally a pair of distal lead segments 7830A, 7830B, each of which comprise a body 7831 and distal end 7833. Meanwhile, each stimulation element 7832A, 7832B may comprise a proximal end 1634, transition portion 1635, and distal end 1636 (FIG. 16I) with the proximal end 1634 extending from and being supported by the distal end 7833 of the respective distal lead segments 7830A, 7830B.

Like the example stimulation elements 7232A, 7232B of FIGS. 81-8J, the stimulation elements 7832A, 7832B of FIGS. 16H-161 may have a chin-centric origin in which the stimulation elements 7832A, 7832B, with their proximal ends 1634 near chin, extend posteriorly away from the chin as shown in FIGS. 16F-16G and with a distal portion 1639 of each stimulation element 7832A, 7832B extending in a predominantly superior orientation upward away from the mandibular plane (Mand) into, among, and within portions of the target tissue environment 7209 as shown in the diagram 7900 of FIG. 16I. Via this example arrangement shown in at least FIG. 16G, at least some of contact electrodes 1672X, 1672Y of the distal portion 1639 become positioned into stimulating relation with some distal terminal nerve portions (e.g. 7285) of a region 7282 of such distal terminal portions of the medial branch of the hypoglossal nerve.

Meanwhile, the proximal portion (1638 of FIG. 16H) and/or transition portion (1635 of FIG. 16H) of each stimulation element (e.g. 7832B) may be in stimulating relation to more proximal nerve portions (e.g. 7262, 7280, etc.) of the hypoglossal nerve 7260.

In some examples, the stimulation elements 7832A, 7832B of FIGS. 16H-161 may be implanted in a manner substantially the same as for stimulation elements 1632A, 1632B of FIGS. 16C-16E, such as via an implant-access incision 609A (FIG. 16F) except starting with a more chin-centric location than in the example device) 1605 of FIGS. 16C-16G. Accordingly, as shown in FIG. 16H, the body 7831 of the distal lead segment 7830A, 7830B first extend in an anterior orientation from the bifurcation portion 7828 (e.g. along sagittal midline 316) and then make a 180 reversal in orientation adjacent to or at their distal ends 7833 to support stimulation elements 7832A, 7832B in their predominantly posterior orientation from the proximal ends 1634 of the stimulation elements 7832A, 7832B having a chin-centric origin.

In a manner similar to that shown and described for example implementations in FIG. 16C, as shown in FIG. 16H the proximal portion 1638 of the stimulation elements 7832A, 7832B are juxtaposed alongside nerve portion 361HR and 361HL of the hypoglossal nerve, as well as alongside muscle portion 1619R, 1619L of the genioglossus muscle.

In contrast to the example implementation(s) of FIGS. 16C-16G, the example implementation of FIGS. 16H-161 may ease implantation for some patients and/or may enhance positioning of contact electrodes 1672X, 1672Y (of the distal portion of the stimulation elements 7832A, 7832B) into stimulating relation with distal terminal nerve portions for at least some types of patient anatomy.

FIG. 16J is a diagram 1800 including a side plan view of an example stimulation element 1832 in which an anchor structure 1802 is arranged on outer surface 1827 of the stimulation element 1832 (between ends 1826A, 1826B). In some examples, the stimulation element 1832 may comprise one example implementation of the stimulation elements 1532A, 1532B in FIG. 16A, the stimulation elements 1632A, 1632B in FIGS. 16C-16G, or the stimulation elements 7832A, 7832B in FIGS. 16H-161. In general terms, the anchor structure 1802 may comprise at least some of substantially the same features and attributes as one of the anchor structures described in association with at least FIGS. 8E-15H, such as (but not limited to) the example anchor structure 1421 in FIG. 14B in which strips 1823A (solid lines), 1823B (dashed lines) of anchor structure 1802 in FIG. 16H forms a helical pattern extending about the outer surface 1827. Each of the helically patterned strips 1823A, 1823B may comprise a plurality of anchor elements like anchor elements 1414 in FIG. 14B and/or at least some of the other sized, shaped, oriented etc. anchor elements described in association with at least FIGS. 8B-15H.

Among other aspects, in some examples the helical pattern of anchor elements (in strips 1823A, 1823B) is interposed between and among the spaced apart contact electrodes 1572A-1572E in which some exposed portions 1824 of the outer surface 1827 of the stimulation element 1832 are not covered with or by the anchor structure 1802. Via this combination of non-covered anchor portions and the helically-arranged covered portions, the stimulation element 1832 may facilitate both some lateral and longitudinal maneuverability within a patient's body during implantation while providing sufficient resistance to lateral and/or longitudinal movement once the stimulation element has become chronically positioned in its intended implant location.

FIG. 16K is a diagram 1840 including a side plan view of an example stimulation element 1843 in which an anchor structure 1841 is arranged on outer surface 1827 of the stimulation element 1843. In some examples, the stimulation element 1843 may comprise one example implementation of the stimulation elements 1532A, 1532B in FIG. 16A, the stimulation elements 1632A, 1632B in FIGS. 16C-16G, or the stimulation elements 7832A, 7832B in FIGS. 16H-161. In general terms, the anchor structure 1841 may comprise at least some of substantially the same features and attributes as one of the anchor structures described in association with at least FIGS. 8E-15H, such as (but not limited to) the example anchor structure 1411 in FIG. 14A in which columns 1842 of anchor structure 1841 in FIG. 16K extend generally longitudinally along a length of the stimulation element 1843 arranged on each of the lead segments 1855A, 1855B, 1855C, 1855D, 1855E, 1855F between respective adjacent pairs of contact electrodes 1572A and 1572B, 1572B and 1572C, 1572C and 1572D, 1572D and 1572E, respectively. In some examples, each column 1842 may comprise a plurality of anchor elements like anchor elements 1414 in FIG. 14B and/or at least some of the other sized, shaped, oriented etc. anchor elements described in association with at least FIGS. 8E-15H. Meanwhile, the non-covered portions (e.g. spaces) 1848 between adjacent columns 1842 lack or omit such anchor elements (e.g. like 1414 in FIG. 14B). As previously noted elsewhere, this pattern of columns 1842, which are spaced apart circumferentially, may help provide both lateral and longitudinal stability to maintain the stimulation element (or portion of a stimulation lead portion) at the implant location in stimulating relation to the target tissue.

Among other aspects, in some examples the anchor elements (in columns 1842) extend between adjacent pairs of the spaced apart contact electrodes 1572A-1572E such that the columns extend generally the entire length of the stimulation element (or portion of the stimulation lead body) but for the periodic interruption of the contact electrodes 1572A-1572E. Via this combination of covered anchor portions (e.g. columns 1842) and the non-covered portions 1848, the stimulation element 1843 exhibits both some lateral and longitudinal maneuverability within a patient's body while providing sufficient resistance to lateral and/or longitudinal movement once the stimulation element has become positioned in its intended implant location.

FIG. 17A is a diagram 1900 schematically representing an example method and/or example device 1905 for treating sleep disordered breathing and including a stimulation lead 1910 comprising a pair of independently positionable, elongate stimulation elements 1932A, 1932B, with each including an axial array of electrodes 1572A-1572E and with each stimulation element 1932A, 1932B extending from a common body portion 1929.

In some examples, the example method and/or example device 1905 of FIG. 17A may comprise at least some of substantially the same features and attributes as the example method and/or example device of FIG. 16A, except comprising a common body portion 1929 (instead of the bifurcation portion 1528 and the transition portions 1531 of distal lead segments 1530A, 1530B in FIG. 16A) from which the stimulation elements 1932A, 1932B extend as shown in FIG. 17A.

On the one hand, the common body portion 1929 may sometimes be referred to as a bifurcation portion, at least to the extent that the point of bifurcation occurs at or adjacent the proximal ends 1534 of the respective stimulation elements 1932A, 1932B instead of occurring more proximally, such as for the bifurcation portion 1528 in FIG. 16A. On the other hand, the lead body 1922 of stimulation lead 1910 in FIG. 17A may sometimes be viewed as omitting a bifurcation portion, at least to the extent that just a single lead segment of lead body 1922 extends from its proximal end 1920 connected to the IPG 1133 with no point of bifurcation occurring proximal to common body portion 1929 such that distal portion 1924 of lead body 1922 omits separate/independent distal lead segments (e.g. 1530A, 1530B in FIG. 16A). Accordingly, a distal end 1925 of the lead body 1922 is connected to and/or transitions directly into common body portion 1929.

In some examples, the common body portion 1929 comprises a flexible member which permits flexibly bending of the respective stimulation elements 1932A, 1932B relative to each other with the point(s) or region(s) of bending occurring along some portion(s) of the common body portion 1929. As further shown in FIG. 17B, a longitudinal axis of each elongate stimulation element 1932A, 1932B is represented by the two lines V7, V8 (respectively) which extend at an acute angle (Q) relative to each other, such as between 1 and 89 degrees.

In some examples, the angle may comprise between about 5 and 80 degrees, between about 10 and 70 degrees, between about 15 and 60 degrees, between about 20 and 50 degrees, or between about 25 and 40 degrees. In some such examples, the stimulation elements 1932A, 1932B exhibit one of these example angular relationships while both stimulation elements 1932A, 1932B extend generally in a mandibular plane, i.e. plane through which both of the patient's left and right mandibular planes extend or extend in a plane generally parallel to a mandibular plane.

However, in some examples, the stimulation elements 1932A, 1932B may exhibit the above-noted angular relationship even when one or both stimulation elements 1932A, 1932B extend in a plane other than a generally mandibular plane.

It will be understood that in some examples, the respective stimulation elements 1932A, 1932B may at least temporarily extend (relative to each other) as an obtuse angle (e.g. extending between 90 and 180 degrees) in order to facilitate introduction of the stimulation element into and through the implant-access incision 609A.

With further reference to at least FIG. 17B, in some examples, the common body portion 1929 may be formed of materials(s) and/or structure(s) to allow selective manual positioning of the stimulation elements 1932A, 1932B relative to each other to select the relative angle (Q) with respect to each other and for the selected angle to be retained once manual manipulation of the relative angle (during implant) ceases.

Such selective manual positioning (of common body portion 1929) may be performed to determine and retain at least some of the contact electrodes 1572A-1572E in FIG. 17A (like contact electrodes 1572A-1572E in FIG. 16A) to be in stimulating relation to target tissues, e.g. target nerve portion(s), target muscle portion(s), combinations of target nerve portion(s) and target muscle portion(s), neuromuscular junctions of target nerve portions and muscle nerve portions, and/or combinations thereof.

In some such examples, such manual positioning (via common body portion 1929) also may performed to achieve and/or enhance anchoring of each respective stimulation element 1932A, 1932B relative to the surrounding non-nerve tissues while still achieving and/or maintaining at least some of the electrodes 1572A-1572E of the respective stimulation elements 1932A, 1932B in stimulating relation to target nerve portion(s), target muscle portion(s), combinations of target nerve portion(s) and target muscle portion(s), neuromuscular junction(s) of target nerve portion(s) and muscle portion(s), and/or combinations thereof.

However, in some examples, a body of each stimulation element 1932A, 1932B may be formed of a shape-retaining material or others materials, structures, etc. to enable configuring the shape of the stimulation element 1932A, 1932B such as introducing bends or curves along a length of the respective stimulation element 1932A, 1932B which will be retained after such manipulation.

In some examples, via its structure and/or material(s), the common body portion 1929 may be at least temporarily configurable into an implant position in which the entire common body portion 1929 and stimulation elements 1932A, 1932B may be implantably delivered via the midline implant-access incision (609A) with the stimulation elements 1932A, 1932B remaining electrically and mechanically connected to the common body portion 1929.

However, in some examples, in order to enhance ease of implantable delivery of the common body portion 1929 and/or the stimulation elements 1932A, 1932B, one or both of the stimulation elements 1932A, 1932B may be removably connectable relative to the common body portion 1929 before, during, or after the common body portion 1929 and/or stimulation elements 1932A, 1932B have been introduced into the patient's body via the midline implant-access incision 609A.

For instance, in one non-limiting example the lead 1910 including the common body portion 1929 may be implanted via the midline implant-access incision 609A and after such implantation, each stimulation element 1932A, 1932B may be introduced into the patient's body via the midline implant-access incision 609A and then positioned relative to target tissues. In some examples, the stimulation elements 1932A, 1932B are introduced one-at-a-time with a first stimulation element (e.g. 1932A or 1932B) being introduced, positioned, and connected to the common body portion 1929 before a second stimulation element (e.g. other respective stimulation element 1932A or 1932B) is introduced, positioned, and connected to the common body portion 1929.

In some examples, one or both of the stimulation elements 1932A, 1932B may comprise an adjustably variable length to facilitate introduction and implantation of the stimulation elements 1932A, 1932B.

The stimulation elements 1932A, 1932B may have a length shorter or longer than shown in FIG. 17A, and in some examples, the respective stimulation elements 1932A, 1932B may have the same length as shown in FIG. 17A, or in some examples, one stimulation element (e.g. 1932A or 1932B may have a length different from (e.g. shorter or longer) the other respective stimulation element (1932A or 1932B).

It will be understood that lead body 1922, common body portion 1929, and each stimulation element 1932A, 1932B of stimulation lead 1910 of FIG. 17A may comprise a plurality of electrical conductors (e.g. like conductors 1317 in FIG. 13B, 13C or other electrical conductors) extending through and within an interior of each of those respective components (e.g. 1922, 1929, 1932A, 1932B) to establish independent electrical connection between the pulse generator 1133 (like 333 in FIG. 2A) and each respective contact electrode 1572A-1572E of the respective stimulation elements 1932A, 1932B.

As shown in FIG. 17A, in some examples the pulse generator 1133 (to which the lead 1910 is electrically and mechanically connected) may be sized and shaped to be implanted within a head-and-neck region in manner accessible via the midline implant-access incision 609A and in relation to the stimulation elements 1932A, 1932B. In some such examples, the size and/or shape of the pulse generator 1133 enables the midline implant-access incision 609A to comprise the sole implant-access incision 609A used to implant all the implantable components of the example device 1905, including the pulse generator 1133. In some examples, a tunnel T6 may be used to provide a path to implant the pulse generator 1133 even though the stimulation element 1932A, 1932B may be implanted without tunneling in some examples. However, it will be understood that whether any tunneling is performed to implant the respective stimulation elements 1932A, 1932B may depend on a length of the stimulation elements 1932A, 1932B being implanted. In some examples, no such tunneling would be performed because of the manner in which the midline implant-access incision 609A generally facilitates direct visualization of the target tissues (e.g. target nerve portions, target muscle portions, combinations of the nerve portion(s) and muscle portions, target neuromuscular junctions including nerve portion(s) and muscle portion(s), and/or combinations thereof) to which the stimulation elements 1932A, 1932B would be placed into stimulating relation.

In some examples, the pulse generator 1133 may sometimes be referred to as a microstimulator when pulse generator 1133 comprises a size and/or shape conducive to implantation in locations in which more traditional pectorally-implanted pulse generators would not be implantable due to their size and/or shape.

However, in some examples, the pulse generator 1133 may be implanted via a different location, such as shown in FIG. 3, with such example methods comprising formation of a second implant-access incision (e.g. 609B) via which the pulse generator (e.g. 333) may be implanted.

FIG. 18A is a diagram 2000 schematically representing an example method and/or example device 2005 for treating sleep disordered breathing. In some examples, the example device 2005 (and/or example method) includes a stimulation lead 2010 comprising a stimulation portion 2051 including a pair of independently positionable, paddle-style stimulation elements 2052A, 2052B each including an array 2055 of electrodes 2056.

In some examples, the lead 2010 may comprise at least some of substantially the same features and attributes as lead 1910, as previously described in association with at least FIG. 17A, except omitting a common body portion (e.g. 1929 in FIG. 17A), including differently shaped/sized stimulation elements 2052A, 2052B, among other differences as further described below.

In some examples, the distal portion 2024 of the stimulation lead body 2022 (of lead 2010) may comprise a flexible connector segment 2060 which extends between and at least mechanically connects the stimulation elements 2052A, 2052B of stimulation portion 2051 relative to each other. In some examples, each stimulation element 2052A, 2052B is electrically connected via lead body 2022 to a pulse generator (e.g. 1133 in FIG. 17A; 333 in FIG. 3; and the like).

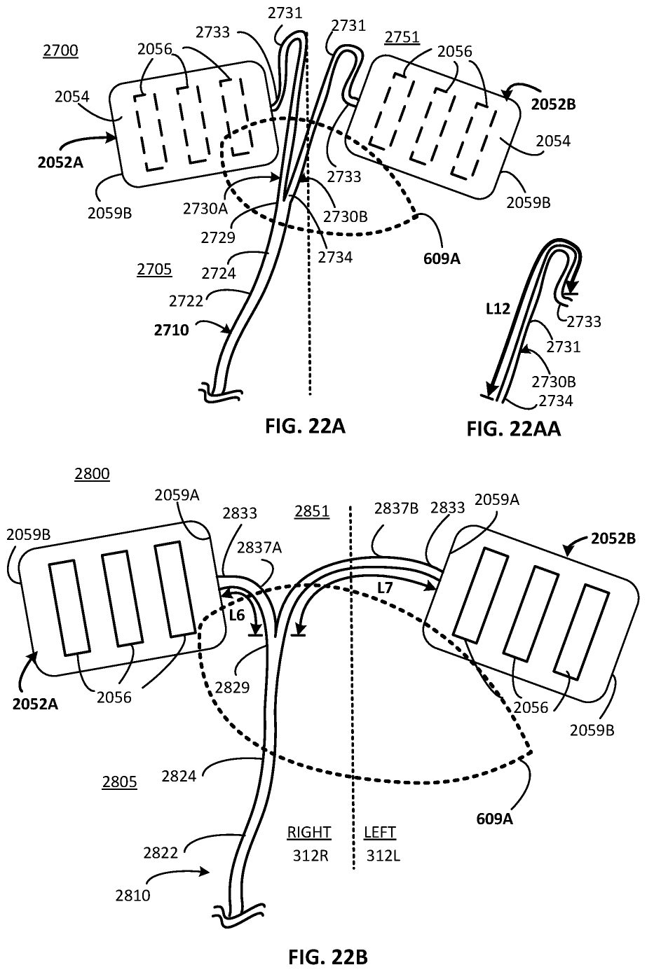

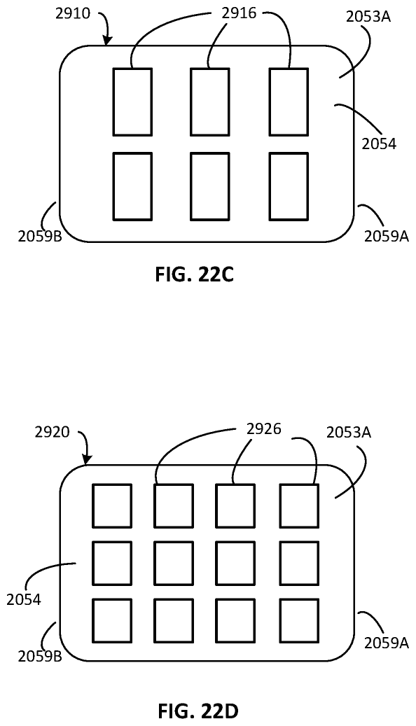

With further reference to FIG. 18A, in some examples, together with an end 2029 of distal portion 2024 of lead body 2022, the connector segment 2060 may form a T-shaped junction 2065. As shown later in FIG. 22A or 22B, instead of comprising a T-shaped junction 2065, in some examples the distal portion 2024 of the lead 2010 may comprise independent connector segments (e.g. 2730A, 2730B in FIG. 22A), each of which extend from (or form) a bifurcated portion in a manner similar to the example shown in at least FIG. 16A.

In some examples, the flexible connector segment 2060 may comprise a single or a plurality of independent electrical conductors (e.g. 1317 in FIG. 13B; 1456 in FIG. 14D) with each such independent electrical conductor establishing electrical connection between a respective one of the contact electrodes 2056 (of a respective one of the stimulation elements 2052A, 2052B) and corresponding independent electrical conductors (e.g. like 1317 in FIG. 13B) within proximal portions of lead body 2022, which in turn are in electrical connection with electrical contact portions of a port and/or of a pulse generator (e.g. 1133, 333) which delivers electrical stimulation signals to the contact electrodes 2056. In this regard, in some such examples, the flexible connector segment 2060 omits stimulation generation circuitry, omits wireless power-receiving circuitry, and/or omits wireless communication circuitry. In some examples, the flexible connector segment 2060 may have a generally cylindrical shape, such that is has a generally circular cross-sectional shape.

With this in mind, in some examples, the flexible connector segment 2060 generally does not perform functions other than transmitting stimulation signals from a pulse generator/microstimulator (e.g. 333 in FIG. 3 or 1133 in FIG. 17A) to the contact electrodes 2056 or transmit sensing signals from the contact electrodes 2056 to the pulse generator/microstimulator (333 or 1133). In some such examples, the electrical conductor(s) extending within and through the flexible connector segment 2060 generally comprise the sole electrically conductive elements within the flexible connector segment 2060.

In general terms, the connector segment 2060 is flexible to permit independent positioning of each respective stimulation element 2052A, 2052B relative to target tissues, such as nerve portions, muscle portions, combinations of nerve portions and muscle portions, neuromuscular junctions of nerve portions and muscle portions, and/or combinations thereof. In some examples, the flexible connector segment 2060 comprises a flexible material which is selectively bendable into a desired shape, orientation, etc. and which may be maintained in the achieved shape, orientation, etc. with the support of an anchor structure used to help maintain the shape, orientation relative to the surrounding tissues so that each respective stimulation element 2052A, 2052B is retained in a fixed position of stimulating relation to target tissues.

In some examples, the flexible connector segment 2060 comprises a material which is selectively manipulable (e.g. bendable, rotatable, etc.) into a desired shape, orientation, etc. and which is made of a material which can retain the selectively manipulated shape, orientation, etc. in order to cause each respective stimulation element 2052A, 2052B to be retained in its chronically implanted position having a desired orientation, position, etc. of stimulating relation to target tissues. In some examples, the stimulation lead 2010 may comprise and/or be deployed with an anchor structure to help maintain the shape, orientation, position of the connector segment 2060 relative to surrounding tissues and of the stimulation elements 2052A, 2052B relative to the target tissues to achieve and maintain the above-noted stimulation relationship(s).

In some examples, the flexible connector segment 2060 may sometimes be referred to as omitting a discrete hinge or hinges to connect the respective stimulation elements 2052A, 2052B relative to each other.

In some examples, the flexible connector segment 2060 of FIG. 18A comprises a material which may be bent, curved, twisted into different configurations and retain its new shape. In some such examples, the material may sometimes be referred to as a shape-retaining material or the flexible connector segment 2060 may sometimes be referred to as a shape-retaining, flexible connector segment 2060. In order to provide the above-noted shape-retaining properties, in at least some of these examples the flexible connector segment 2060 may comprise a material incorporated into, or paired with, the electrical conductor(s) which extend through and/or within an insulative jacket (e.g. generally containing the electrical conductors extending therethrough) of the flexible connector segment 2060.

In some examples, the flexible connector segment 2060 comprises a material which is resilient such that the connector segment 2060 may be flexed, twisted, etc. into different shapes, configurations and return to its original shape when a force causing such flexing, twisting, etc. is removed.

In some examples, the flexible connector segment 2060 (e.g. FIG. 18A) may have a pre-formed shape such that flexible connector segment 2060 may comprise at least some of substantially the features and attributes as the common body portion 1929 in FIGS. 17A-17B.

In some examples, the flexible connector segment 2060 comprises a material which is selectively bendable into a desired shape, orientation, etc. and which includes shape memory properties to achieve a desired shape, orientation, etc. with an original shape, orientation being re-obtained upon the flexible connector segment 2060 being subject to certain temperatures, conditions, etc.

In some examples, one material comprising such shape memory properties may comprise alloys (e.g. Nitonol, polymers, hybrid materials, etc.

In some examples, the connector segment 2060 may comprise at least one variable length portion, such as but not limited to a pre-formed sigmoid-shaped portion. However, in some examples, the connector segment 2060 may have a length greater than an expected area in which the connector segment 2060 is to be deployed such that in the course of placing the connector segment 2060 within and among the target tissues, the flexible connector segment 2060 will have a sufficient number and locations of curves to achieve suitable strain relief properties and ability to achieve the desired shapes, orientations, positions, etc.

In some examples, the connector segment 2060 in FIG. 18A may comprise a cross-sectional dimension (e.g. diameter, area, etc.) which is substantially less than a cross-sectional dimension of the common body portion 1929 of the stimulation lead 1910 in FIG. 17A. In some such examples, a greatest cross-sectional dimension (e.g. diameter, major axis, and/or the like) of the flexible connector segment 2060 may be substantially less than a width (W2 in FIG. 18B) of the paddle-style body 2054 of the stimulation element 2052A, 2052B and/or substantially less than a length (L2 in FIG. 18B) of the paddle-style body of the stimulation element 2052A, 2052B. In some examples, the greatest cross-sectional dimension of the flexible connector segment 2060 may comprise at least about 5 times less than the respective width (W2) or respective length (L2) of the paddle-style body 2054 of the respective stimulation elements 2052A, 2052B. In some examples, the greatest cross-sectional dimension of the flexible connector segment 2060 comprises at least about 6 times less, at least about 7 times less, at least about 8 times less, at least about 9 times less, etc. than the respective width (W2) or respective length (L2) of the paddle-style body 2054 of the respective stimulation elements 2052A, 2052B. In some examples, the greatest cross-sectional dimension of the flexible connector segment 2060 may comprise at least one order of magnitude (e.g. at least 10 times) less than the respective width or respective length of the paddle-style body 2054 of the respective stimulation elements 2052A, 2052B.

In some examples this arrangement may stand in contrast to at least some examples associated with the stimulation lead 1910 of FIG. 17A in which a greatest cross-sectional dimension of the common body portion 1929 (at or near the point of connection to the stimulation elements 1932A, 1932B) is substantially the same as or similar to the greatest cross-sectional dimension (e.g. diameter, area) of a respective one of the stimulation elements 1932A, 1932B in the example of FIG. 17A.

In some examples, the T-shaped junction 2065 of lead 2010 in FIG. 18A may be omitted and each stimulation element 2052A, 2052B may extend from and be supported by separate and independent distal lead segments (e.g. 2730A, 2730B) such as shown in the later described example arrangement in FIG. 22A, in which the separate and independent distal segments are bifurcated from each other at a location (e.g. 2729 in FIG. 22A) along lead body 2022 (2722 in FIG. 22A) more proximal than shown in FIG. 18A. In some such examples, the longer distal lead segments 2730A, 2730B in the example of FIG. 22A may facilitate manipulation and positioning of each respective stimulation element 2052A, 2052B according to a fuller range of movement in each of six degrees of freedom (e.g. 3 orthogonal rotational axes and 3 orthogonal translational axes).

In some examples, in a manner similar to the later-described example of FIG. 22A, the separate, independent distal lead segments (e.g. 2730A, 2730B) are bifurcated from each other at location more proximal than shown in FIG. 13A except with the distal lead segments 2837A, 2837B (extending to each respective stimulation element 2052A, 2052B) having different lengths as shown in the example arrangement of FIG. 22B. Via this arrangement, the distal lead segments 2837A, 2837B may be referred to as being positioned asymmetric relative to the patient's sagittal midline 316 and/or relative to the point of bifurcation 2829 along lead body 2822. In one aspect, this asymmetric configuration of the distal lead segments may enhance implantation of the lead 2810 and stimulation elements 2052A, 2052B when patient anatomical anomalies may hinder a delivery approach which is along or very close to the sagittal midline 316. In some such examples, the bifurcation point 2829 may be located within distance of about 1 to about 5 centimeters of the patient's sagittal midline 316 and/or at least the distal portion 2824 of the lead 2810 may be implanted via the midline implant-access incision 609A.

With further reference to at least the example arrangement of FIG. 18A, via the midline implant-access incision 609A, the flexible connector segment 2060 may become positioned underneath, or superficial to, the genioglossus, or even underneath, or superficial to, the geniohyoid muscle, while remaining deep to the anterior belly of the digastric and mylohyoid muscles near the midline region of the genioglossus group of muscles with the stimulation elements extending alongside, within, and/or among the various muscles and nerves of the genioglossus group/complex.

With further reference to the example arrangement of FIGS. 18A-25 (e.g. FIGS. 18A-18C in particular), in some examples the stimulation portion 2051 (which comprises the stimulation elements 2052A, 2052B and the flexible connector segment 2060) does not define (i.e. omits) a central substantially planar body and/or does not define (i.e. omits) discrete hinges. For instance, in some such examples, the entire (or substantially entire) connector segment 2060 does not comprise a pre-formed shape, such as a substantially planar central body and/or such as discrete hinges on opposites sides of such a central body.

Instead, in some examples the flexible connector segment 2060 of FIG. 18A may comprise an element comprising a substantially uniform shape (e.g. circular cross-section) and/or substantially uniform cross-sectional dimension (e.g. diameter) throughout the entire length or substantially the entire length of the flexible connector segment 2060.

In some examples, the body 2054 of each stimulation element 2052A, 2052B comprises a substantially planar member, i.e. a structure which generally extends through and within one plane. In some examples, the body 2054 may comprise sufficient rigidity to maintain its generally planar configuration.

However, in some examples the body 2054 may comprise at least some resilient flexibility to permit at least some flexibility of the body 2054 to permit minor flexing and bending to position the stimulation element 2052A, 2052B within and/or among target tissues without substantially losing its overall planar shape.

Accordingly, in some such examples, the body 2054 may be considered to be formed of a semi-rigid material.

In some examples, the non-electrically conductive portions of the body 2054 of each stimulation element 2052A, 2052B of FIG. 18A may be generally uniform in size and/or shape throughout a length (L2 in FIG. 18B) of body 2054.

In this regard, in some examples, the body 2054 of each stimulation element 2052A, 2052B may sometimes be referred to as omitting discrete hinges, pivot-type structures, articulation-type structures, etc. within, or along, the body 2054, such as but not limited to, single axis-type hinges or fold lines. Similarly, in some examples, the body 2054 omits pre-fold lines in or along the body 2054 at which portions of the body 2054 would otherwise be foldable or pivotable relative to each other in a hinge-like manner. Stated differently, the body 2054 of the stimulation elements 2052A, 2052B may sometimes be referred to as being hinge-free or hinge-less.

However, in some examples, the entire body 2054 (or substantially the entire body 2054) of each stimulation element 2052A, 2052B may comprise a conformable, compliant member to enable at least partially wrapping or conforming the body 2054 relative to the target tissues (e.g. nerve portions, muscle portions, combinations of nerve portions and muscle portions, neuromuscular junctions of nerve portions and muscle portions, and/or combinations thereof), which may enhance positioning of the contact electrodes 2056 to be in stimulating relation to the target tissues. In some such examples, the compliant member at least partially defining body 2054 may retain a shape into which it has been manipulated in order to conform to the target tissue on which it engages. In such examples, the body 2054 may sometimes be referred to as comprising shape-retaining properties. Accordingly, in some examples, the paddle-style body 2054 of stimulation elements 2052A, 2052B may comprise material which is sufficiently flexible to be conformable to the target tissues and other tissues within which the stimulation element 2052A, 2052B is to implanted such that wrappability facilitates engagement of the contact electrodes (by contact or by achieving close proximity) and/or associated anchor structures relative to the target tissues.

In some examples, body 2054 of stimulation elements 2052A, 2052B may comprise flexible circuitry extending within or through the body 2054 in order to provide electrical connection between the conductive connector segment 2060 and the contact electrodes 2056 of each stimulation element 2052A, 2052B.

In some examples, each stimulation element 2052A, 2052B may comprise a printed circuit-type element comprising at least some of substantially the same features and attributes as the printed circuit-type elements, as previously described in association with at least FIGS. 9A-10B.

The paddle-style body 2054 of each stimulation element 2052A, 2052B may sometimes be referred to as a paddle-style carrier body 2054.

As shown in at least FIGS. 18B-C, in some examples the paddle-style body 2054 of each stimulation element 2052A, 2052B comprises a width (W2) and a length (L2), and a thickness (B2). In some examples, the paddle-style of the body 2054 may be at least partially defined by the width (W2) being substantially greater than the thickness (B2).

In some examples, in one context this “substantially greater” dimensional relationship comprises the width (W2) being at least 5 times (or at least 3 times or at least 4 times) greater than the thickness (B2). In some such examples, the “substantially greater” dimensional relationship comprises the width (W2) being at least one order of magnitude greater than the thickness (B2).

In some examples, in one context the paddle-style of the body 2054 may be at least partially defined by the length (L2) being substantially greater than the thickness (B2). In some such examples, the “substantially greater” dimensional relationship comprises the length (L2) being at least 5 times (or at least 3 times or at least 4 times) greater than the thickness (B2). In some such examples, the “substantially greater” dimensional relationship comprises the length (L2) being at least one order of magnitude greater than the thickness (B2).

In some examples, in one context the paddle-style of the body 2054 of each stimulation element 2052A, 2052B comprises a width (W2) on the order of 10 times (order of magnitude) greater than a diameter of a target nerve at which the carrier body is deployed.

With further reference to FIGS. 18A-18C, in some examples, the body 2054 supports an array of contact electrodes 2056 which extend in a generally parallel, spaced apart relationship with electrically non-conductive portions 2057 of the body 2054 on first surface 2053A interposed between adjacent pairs of contact electrodes 2056. The body 2054 includes an opposite second surface 2053B (FIG. 18C and with portions 2056, 2057 being on first surface 2053A in FIG. 18C). In some examples, the second surface 2053B is electrically non-conductive.

In accordance with some examples, the illustration of FIG. 18A shows the second surface 2053B of the body 2054 of each stimulation element 2052A, 2052B, with the first surface 2053A of the body 2054 of each stimulation element 2052A, 2052B facing toward the target tissue (e.g., nerves 360L, 360R), as illustrated by the contact electrodes 2056 being in dashed lines in FIG. 18A. In some such examples, the contact electrodes 2056 may face the target tissue, with at least portions of the target tissue (e.g., nerves 360L, 360R) being behind (e.g., deeper) the body 2054 of each stimulation element 2052A, 2052B. As may be appreciated and in some examples, portions of nerves 360L, 360R are shown in solid lines at locations at which the portions of the nerves 360L, 360R are behind the body 2054 of each stimulation element 2052A, 2052B (e.g., behind the paddles) for simplicity purposes. Similar representations are made throughout the disclosure.

In some examples, an exposed surface of the contact electrodes 2056 may be flush with the exposed surfaces (e.g. 2057, border portion 2058) of the body 2054.

However, in some examples, the contact electrodes 2056 may form protrusions relative to the exposed surfaces (e.g. 2057, 2058) of body 2054 of the stimulation elements (e.g. 2052A, 2052B). In some such examples, the raised protrusions may enhance engagement of the contact electrodes 2056 relative to the target tissue (e.g. nerve portions, muscle portions, etc.). In some examples, each stimulation portion 2052A, 2052B may comprise an anchor structure(s) on the exposed surfaces (e.g. 2057, 2058) of the carrier body 2054. In some examples, such anchor structure(s) may comprise at least some of substantially the same features and attributes as the anchor structure(s) as described in association with at least FIGS. 8E-15H, 16J-16K, 23A-24E, and/or 25H.

In some examples, each contact electrode 2056 may comprise a length L3 and a width W3. In some examples, the space provided by non-conductive portions 2057 extending between respective electrodes 2056 may be similar to the width W3 with such space being sufficient for adjacent electrodes 2056 to be independent controlled and to independently apply stimulation signals, in some examples. In some examples, the length L3 of each respective electrode 2056 may be large enough to intersect with more than one nerve portion (e.g. 371L, 375L in FIG. 18A, in just one example).

FIGS. 19A-19D are a group of diagrams schematically representing an example device (and/or example method) of implanting the respective paddle-style stimulation elements 2052A, 2052B of FIGS. 18A-18C in an example arrangement in which the respective stimulation elements 2052A, 2052B are rotated relative to each other according to any one or more of three orthogonal rotational degrees of freedom, including a roll configuration (FIG. 19A-19B), a yaw configuration (FIG. 19C), and a pitch configuration (FIG. 19D). Any single one or a combination of these configurations may occur during implantation (e.g. a transitory position) and/or as a final position of the respective stimulation elements 2052A, 2052B upon completion of the chronic implantation.

With further reference to FIGS. 19A-19D, it will be understood that the orthogonal arrangement of the Z reference plane, Y reference plane, and X reference plane is maintained at all times, and that the representation of the stimulation elements 2052A, 2052B being in a rotated position (relative to each other) according to a roll parameter, a yaw parameter, and/or a pitch parameter does not strictly depend on the three reference planes (Z, Y, X) defining or corresponding to an absolute orientation in a patient's body. Nevertheless, in some examples, the Z reference plane may correspond to a superior-inferior orientation (e.g. axis) within the patient's body, the Y reference plane may correspond with an anterior-posterior orientation (e.g. axis) within the patient's body, and the X reference plane may correspond with a medial-lateral orientation (e.g. axis) within the patient's body.

As shown in FIG. 19A-19B, in some examples a first stimulation element 2052A and a second stimulation element 2052B are in a position rotated relative to each other according to a roll parameter (FIG. 19A), as represented by the angle (E) between the Z reference plane and the Y reference plane (FIG. 19B).

In one aspect, the rotational movement of the stimulation elements 2052A, 2052B relative to each other according to a roll parameter may sometimes be referred to as, or be implemented via, a twist (e.g. twisting) of first and second stimulation elements 2052A, 2052B relative to each other which will include some rolling or twisting the flexible connector segment 2060.

It will be understood that the acute angle (E) shown in FIG. 19A-19B is merely an example and the angle (E) may be any angle from 0 to 360 degrees.

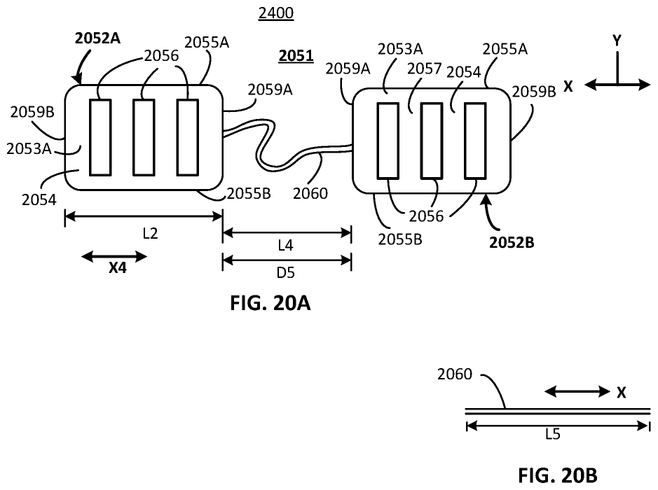

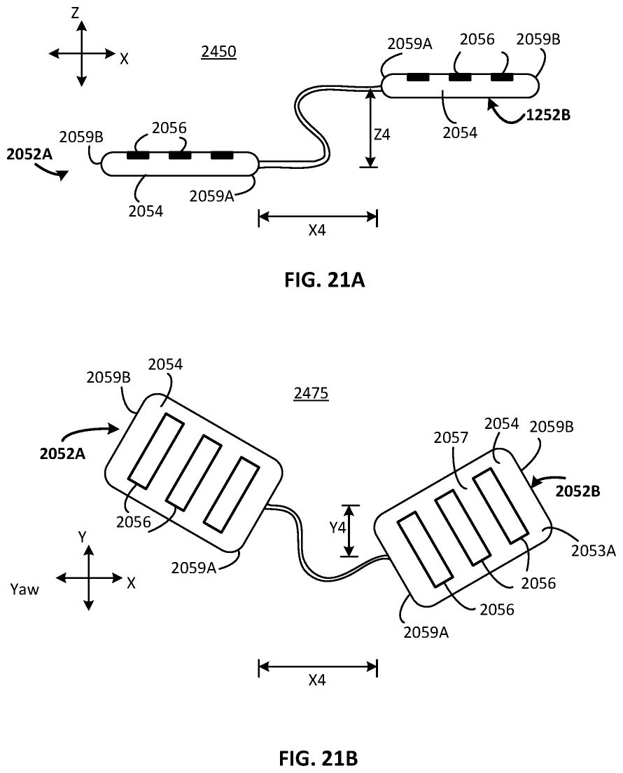

Moreover, in some examples, for any given angle (E), the respective stimulation elements 2052A, 2052B also may be rotated relative to each other by a particular angle according to a yaw parameter (FIG. 19C) and/or by a particular angle according to a pitch parameter (FIG. 19D). Furthermore, in some examples, the respective stimulation elements 2052A, 2052B may be translated relative to each other along one or more of the reference planes/orientations (Z, Y, X), as shown later in FIGS. 20A-21B.

In some examples, the flexible connector segment 2060 is configured in a manner (according to a length, shape, and/or type of material, etc.) to permit maintaining a particular rotational Z, Y, X orientation while simultaneously translating the stimulation elements 2052A, 2052B relative to each other according to one or more Z, Y, X orientations within the patient's body. In some examples, the flexible connector segment 2060 is configured in a manner (according to a length, shape, etc.) to permit changing one or more rotational Z, Y, X orientations while simultaneously translating the stimulation elements 2052A, 2052B relative to each other according to one or more Z, Y, X orientations within the patient's body. In this way, the flexible connector segment 2060 permits comprehensive movement according to six degrees of freedom (including rotation and translation) of the stimulation element 2052A, 2052B relative to each other during implantation or in a chronically implanted position within the patient's body.

As described above, in some instances, the location of the target nerve of the patient may not be symmetrical on the left side and right side of the patient.

For example, the target nerve may be more inferior to the mandible, at a different angle and/or distance to the sagittal middle on the left side as compared to the right side of the patient and/or vice versa or combinations thereof. The flexible rotational and/or translational degrees of freedom of the stimulation elements 2052A, 2052B and/or the variability of the length of the flexible connector segment 2060 may enhance bilateral stimulation of the target nerve on the left and right sides of the patient by allowing for greater flexibility in placement of the stimulation elements 2052A, 2052B with respect to one another and to better capture the target nerves on both sides. Without such flexibility and degrees of freedom exhibited by examples of the present disclosure, some other non-example stimulation arrangements might be able to electrically capture a nerve on one side of the body but not adequately electrically capture the target nerve on the other side of the body, such that bilateral stimulation cannot be implemented in a manner to achieve efficacious stimulation therapy.

While FIG. 19A illustrates paddle-style carrier bodies, in some examples, the stimulation elements may include other types of orientations and/or carrier bodies having other shapes/sizes, which are connected by a flexible connector segment and/or which otherwise have various rotational and/or translational degrees of freedom.

With further reference to FIG. 19A, it will be understood that together the stimulation elements 2052A, 2052B generally extend at least partially in a medial-lateral orientation of the patient's body with one stimulation element 2052A on a right side of the patient's body and the other stimulation element 2052B on a left side (312L) of the patient's body such that the stimulation elements 2052A, 2052B are arranged (relative to target tissue) for bilateral stimulation of the left and right hypoglossal nerves and/or unilateral stimulation of just one of the left or right hypoglossal nerves. In the example of bilateral stimulation, it will be understood that such stimulation may be applied simultaneously, alternately, sequentially, etc. to the left and right hypoglossal nerves.

Moreover, as further described later in association with at least FIGS. 19E-19F, in some examples stimulation may be applied via stimulation vectors extending between a stimulation element 2052A on a patient's right side 312R and a stimulation element 2052B on a patient's left side 312L. In some examples, such stimulation may sometimes be referred to as cross-lateral stimulation or cross midline stimulation.

With further reference to at least FIGS. 19A-19B, in some examples, during implantation and/or in a chronically implanted position, the flexible connector segment 2060 permits a rolling rotation of the stimulation elements 2052A, 2052B in orientation transverse (e.g. across) the submental region (e.g. extending between left mandible and right mandible) so that a minor axis of the body 2054 of either or both of the first and second paddle-style stimulation elements 2052A, 2052B are rotatable in an anterior-posterior orientation about a medial-lateral orientation (e.g. axis). In some examples of such rolling rotation, the first and second paddle-style stimulation elements 2052A, 2052B may be manipulated to maintain a major axis of the body 2054 of each respective stimulation element 2052A, 2052B in their existing general orientation (prior to initiation of the rolling rotation) according to a medial-lateral orientation and a superior-inferior orientation in order to achieve a desired position relative to target tissues. However, in some examples of such rolling rotation, the first and second paddle-style stimulation elements 2052A, 2052B are maneuvered to alter the orientation of a major axis of the body 2054 of each respective stimulation element 2052A, 2052B according to a medial-lateral orientation and a superior-inferior orientation in order to achieve a desired position relative to target tissues.

It will be further understood that a desired positioning of the stimulation elements 2052A, 2052B within the patient's body can be implemented without a rotational movement according to a particular one (e.g. rolling) of the three orthogonal reference orientations.

Moreover, in some examples, one of the stimulation elements (e.g. 2052A) may be maintained in a stationary position while the other respective stimulation element (e.g. 2052B) is rollingly rotated, or vice versa. In some examples, both stimulation elements 2052A, 2052B may be moved (e.g. rolled) to achieve a desired roll configuration (angle E) relative to each other and relative to the target tissue within the patient's body.

In this regard, in some examples, the flexible connector segment 2060 comprises a size (e.g. length, diameter, etc.), shape, and relative flexibility to enable the stimulation elements 2052A, 2052B to be maneuvered independently from each other with their degrees of freedom being independent of (e.g. not limited by) any central carrier body. Among other examples within the present disclosure, at least the example arrangements in FIGS. 18A-22B may facilitate such independent maneuvering and implantation of the stimulation elements 2052A, 2052B in which the degrees of freedom of one stimulation element (e.g. 2052A) is not limited by the degrees of freedom of another stimulation element (e.g. 2052B) and/or any carrier body between such stimulation elements 2052A, 2052B.

In some examples, the above-noted positioning of the stimulation elements 2052A, 2052B to extend in a diverging orientation (relative to each other and/or a midline implant-access incision 609A) may be regarded as occurring according to yaw parameter as shown in FIG. 19C by which rotational movement of the stimulation elements 2052A, 2052B relative to each other occurs and rotational movement predominantly about a superior-inferior orientation (e.g. axis) occurs, as the distal ends 2059B of the stimulation elements 2052A, 2052B are advanced primarily in a posterior direction according to an anterior-posterior orientation. As the distal ends 2059B of the stimulation elements 2052A, 2052B are advanced primarily in this anterior-posterior orientation, it will be understood that at least some of this movement occurs in the medial-orientation as the stimulation elements 2052A, 2052B are advanced divergently in the posterior direction within the patient's body from a starting point adjacent the chin.

In some examples, in addition to the respective stimulation elements straddling the sagittal midline 316 in a general medial-lateral orientation as shown in FIG. 19A, the respective stimulation elements 2052A, 2052B may be regarded as extending in divergent orientations relative to each other (and relative to an implant-access incision 609A, such as at or in close proximity to a sagittal midline 316), which may (among other factors) enhance introduction and/or navigation of each respective stimulation element 2052A, 2052B relative to tissues in or along an access pathway to the target tissues, as well as among and/or within the target tissues.

In some such examples, each respective stimulation element 2052A, 2052B may be implanted to extend in an orientation generally parallel to a mandible, such as one stimulation element 2052A extending generally parallel to a right mandible (e.g. a longitudinal axis of the right mandible) and one stimulation element 2052B extending generally parallel to a left mandible (e.g. a longitudinal axis of the left mandible).

In addition to, or instead of, movement of the stimulation elements 2052A, 2052B according a roll parameter (e.g. FIGS. 14A-14B) and/or a yaw parameter (e.g. FIG. 14C), in some examples rotational movement of the stimulation elements 2052A, 2052B (relative to each other) may be performed during implantation or in an implanted position according to a pitch parameter as shown in FIG. 19D by which rotational movement of the stimulation elements 2052A, 2052B relative to each other occurs and with such rotational movement predominantly about an anterior-posterior orientation (e.g. axis), as the distal ends 2059B of the stimulation elements 2052A, 2052B are advanced primarily in a superior direction according to a superior-inferior orientation in the patient's body. As the distal ends 2059B of the stimulation elements 2052A, 2052B are advanced primarily in this superior-inferior orientation, it will be understood that at least some of this movement may include translation of the stimulation elements 2052A, 2052B in the superior-inferior orientation, as well as in the medial-lateral orientation and/or anterior-posterior orientation. In some examples, the distal ends 2059B may sometimes be referred to as outer ends of the body 2054 of the respective stimulation elements 2052A, 2052B.

With regard to at least some of the examples of FIGS. 18A-19D, the flexible connector segment 2060 may be regarded as providing an indefinite number of rotatable regions (e.g. portions) along its length between the respective stimulation elements 2052A, 2052B which may be manipulated to achieve a desired degree and orientation of rotation of the stimulation elements 2052A, 2052B relative to each other according to one or more three independent rotational degrees of freedom (e.g. roll, yaw, pitch). Similarly, in some examples, one may regard the flexible connector segment as omitting a discrete hinge or as being hinge-less or hinge-free. Stated differently, the rotational movement of one stimulation element (e.g. one of 2052A, 2052B) relative to other stimulation element (e.g. one of 2052A, 2052B) is spread over a length (e.g. whole length, a majority, a super majority, etc.) of flexible connector segment 2060 versus a discrete hinge structure (s) or spaced apart discrete hinge structure(s).

FIG. 19E is a diagram 2360 like diagram 2350 of FIG. 19D, except further schematically representing at least some target tissues with which the stimulation elements 2052A, 2052B may be in stimulating relation. As further shown in FIG. 19E, in some examples stimulation may be applied via example stimulation vectors SV1, SV2 extending between a stimulation element 2052A on a patient's right side 312R and a stimulation element 2052B on a patient's left side 312L. In some examples, such stimulation may sometimes be referred to as cross-lateral stimulation.

As shown in FIG. 19E, the stimulation vectors SV1 and/or SV2 may capture target tissues such as any one or more of the nerve portion(s) 2385, 2386, 2387, and/or 2388 which are located near the sagittal midline 316, which in some examples may comprise more distal segments of the medial branch of the hypoglossal nerve. For illustrative simplicity, it will be understood that the identified example nerve portions in FIG. 19E may be representative of other target tissues captured via such example stimulation vectors such as muscle portions innervated by the nerve portions, combinations of nerve portions and muscle portions, neuromuscular junctions of nerve portions and muscle portions, and combinations thereof.

Moreover, the stimulation vectors SV1 and/or SV2 (extending between stimulation elements 2052A, 2052B on opposite sides of the sagittal midline 316) also may capture nerve portion(s) such as but not limited to nerve portions 2362, 2363, 2388, 2365, and the like which are relatively closer to a respective one of the stimulation elements 2052A, 2052B.

In one aspect, the application of stimulation, at least part of treatment period (e.g. a sleeping period), via such cross-lateral stimulation vectors (e.g. SV1, SV2) do not necessarily exclude or prohibit the stimulation vectors applied via the contact electrodes 2056 of just one of the respective stimulation elements 2052A, 2052B. With this in mind, stimulation vectors other than stimulation vectors SV1, SV2 may capture (and stimulate) nerve portions such as but not limited to nerve portions 2362, 2363, 2388, 2365, and the like. As previously mentioned, such example nerve portions 2362, 2363, 2388, 2365 also may be representative of other target tissues captured via such example stimulation vectors such as muscle portions innervated by the nerve portions, combinations of nerve portions and muscle portions, neuromuscular junctions of nerve portions and muscle portions, and combinations thereof.

In viewing FIG. 19E, it will be understood that the particular depicted stimulation vectors SV1, SV2 are merely examples that stimulation vectors between other combinations of contact electrodes 2056 of one stimulation element 2052A and the contact electrodes 2056 of the other respective stimulation elements 2052B are contemplated.

In this regard, it will be understood that in some examples, the respective stimulation elements 2052A, 2052B may comprise an array of stimulation elements such as shown in FIGS. 22C-22D in which an array of contact electrodes may form a grid of contact electrodes in rows and columns such as, but not limited to, a 2×2 array, 2×3 array, 3×3 array, and the like.

It will be understood that such examples cross-lateral stimulation described in association with at least FIG. 19E also may be applied in at least some of the various example implementations of FIGURES other than those shown in association with FIGS. 19D-19E throughout the present disclosure, such as but not limited to the examples of at least FIGS. 8A-8D, 8I-10B, 16A-16G, 17A-22B, in which contact electrodes of stimulation elements on opposite sides of the sagittal midline 316 may be positioned in proximity sufficient to each other to form stimulation vectors between the contact electrodes on respectively opposite sides of the sagittal midline 316 with such stimulation vectors capturing at least some protrusor-related target tissues (e.g. nerve portion(s), muscle portion(s), combinations of nerve portion(s) and muscle portion(s), neuromuscular junctions of nerve portion(s) and muscle portion(s), and/or combinations thereof.

At least some of the above-described principles of operation regarding the rotational degrees of freedom (e.g. roll, yaw, pitch) in association with at least FIGS. 19A-19E may be extended to the movement of the stimulation elements 2052A, 2052B according to the translational degrees of freedom as further described below in association with at least FIGS. 20A-21B. Accordingly, the six degrees of freedom of movement of one stimulation element (e.g. one of 2052A, 2052B) relative to the other stimulation element (e.g. one of 2052A, 2052B) occur via the flexible connector segment 2060 and not via a discrete hinge(s). In some examples, the flexible connector segment 2060 may have a construction in which the elements forming the flexible connector segment 2060 are generally uniform throughout a length (or at least a majority or supermajority of a length) of the flexible connector segment 2060 such that the flexible connector segment 2060 may be regarded, in some examples, as being generally homogenous throughout its length, thereby omitting different discrete elements (e.g. hinge) occurring at some locations along a length of the flexible connector segment 2060.

In some examples, in its pre-implant state, by virtue of at least the flexible connector segment 2060, the stimulation portion 2051 comprises stimulation elements 2052A, 2052B which have a high selectability in their rotational configuration (e.g. roll, yaw, pitch) and translational configuration relative to each other which may enhance initial introduction and advancement of the stimulation portion 2051 into and through the midline implant-access incision 609A. Among other structural attributes, the flexible connector segment 2060 may have a length which permits the stimulation element 2052A, 2052B to be introduced into and through the implant-access incision 609A in a one-at-a-time manner, which may allow a smaller implant-access incision 609A and/or greater ability to rotate and/or translate each stimulation element 2052A, 2052B independently of the other respective stimulation element 2052A, 2052B.

In some examples, a plane through which the stimulation surface (e.g. including the exposed surfaces of the contact electrodes 2056) extends may sometimes be referred to as an electrode contact plane.

Moreover, in at least some pre-implant configurations of the stimulation portion 2051, the stimulation surface (e.g. contact electrode surface(s)) of the stimulation elements 2052A, 2052B need not be facing each other as in some commercially available electrode devices. Rather, in some examples, in a pre-implant configuration, the stimulation surface (of the respective stimulation elements 2052A, 2052B) are oriented (e.g. face) in a parallel direction such that they do not face each other. Furthermore, in at least some examples, the flexible connector segment 2060 is not biased to orient the stimulation surface (of the respective stimulation elements 2052A, 2052B) to face in a particular configuration relative to each other or in any absolute orientation. Conversely, in some examples, the flexible connector segment 2060 may be biased to cause the stimulation surface of the respective stimulation elements 2052A, 2052B to face in a particular orientation.

Moreover, in at least some examples, each stimulation element 2052A, 2052B may extend in an orientation relative to the flexible connector segment 2060 which is not predetermined, such as being at a right angle relative to each other.

In some examples, one pre-implant configuration comprises the edges of the stimulation surface/portion of one stimulation element 2052A, 2052B to face the edges of the stimulation surface/portion of one stimulation element 2052A, 2052B. In this regard, the stimulation surfaces may be regarded as extending in generally the same plane or orientation without facing each other, i.e. a non-facing orientation relative to each other.

Moreover, as will be further illustrated in association with at least FIGS. 20A-21B, the stimulation portion 2051 may implement a translational degree of freedom for either one (or both) stimulation elements 2052A, 2052B without limiting or causing a rotational degree of freedom or other translational degree of freedom of the same stimulation element or other stimulation element.

FIGS. 20A-21B are a series of diagrams schematically representing movement of stimulation elements 2052A, 2052B of stimulation portion 2051 in translational orientation relative to each other. Each of FIGS. 20A, 21A, and 21B schematically represent an example device (and/or example method) including a stimulation portion 2051 which comprises at least some of substantially the same features and attributes as described in association with at least FIGS. 18A-19E.

As shown in the diagram 2400 of FIG. 20A, in some examples, when in a relaxed configuration the flexible connector segment 2060 of stimulation portion 2051 exhibits a nominal effective length L4 extending between the respective stimulation elements 2052A, 2052B. It will be understood that the nominal effective length L4 may vary depending on the number of curves, bends, etc. which may occur in a random manner along the flexible connector segment 2060, which corresponds depends on a distance D5 between the stimulation element 2052A (e.g. end 2059A (and/or side edge 2055A) of element 2052A)) and stimulation element 2052B (e.g. end 2059A (and/or side edge 2055B) of element 2052B)). Conversely, as shown in FIG. 20B, when in a fully extended configuration, the flexible connector segment 2060 exhibits a maximum length L5 between the respective stimulation elements 2052A, 2052B, which is greater than the relaxed length L4 in FIG. 20A. In some examples, the maximum length L5 may correspond generally to a length L2 of the body 2054 of each respective stimulation element 2052A, 2052B.

As further represented in the diagram 2400 of FIG. 20A and in FIG. 20B, in some examples a change in distance between the respective elements 2052A, 2052B corresponds to translational movement along an x orientation (e.g. axis) as represented by directional arrow X4 and which corresponds to one translational degree of freedom. Such translation according to one reference orientation (X) may be implemented with or without rotational movement of the respective stimulation elements 2052A, 2052B relative to each other according to one or a combination of the roll parameter (FIGS. 19A-19B), yaw parameter (FIG. 19C), and a pitch parameter (FIG. 19D). Such translation according to one reference orientation (X) may be implemented with or without translational movement of the respective stimulation elements 2052A, 2052B relative to each other according to the other translational orientations (e.g. Z in FIG. 21A, Y in FIG. 21B).

As further represented in the diagram 2450 of FIG. 21A, in some examples the stimulation elements 2052A, 2052B also may be translated according to a Z reference orientation (as represented by directional arrow Z4), and which corresponds to one translational degree of freedom. Such translation may be implemented with or without rotational movement of the respective stimulation elements 2052A, 2052B relative to each other according to one or a combination of the roll parameter (FIGS. 19A-19B), yaw parameter (FIG. 19C), and a pitch parameter (FIG. 14D). Such translation according to one reference orientation (Z) may be implemented with or without translational movement of the respective stimulation elements 2052A, 2052B relative to each other according to the other translational orientations (e.g. Y in FIG. 21B, X in FIGS. 20A-20B).

As further represented in the diagram 2475 of FIG. 21B, in some examples the stimulation elements 2052A, 2052B also may be translated according to a Y reference orientation (as represented by directional arrow Y4), and which corresponds to one translational degree of freedom. Such translation may be implemented with or without rotational movement of the respective stimulation elements 2052A, 2052B relative to each other according to one or a combination of the roll parameter (FIGS. 19A-19B), yaw parameter (FIG. 19C), and a pitch parameter (FIG. 19D). Such translation according to one reference orientation (Y) also may be implemented with or without translational movement of the respective stimulation elements 2052A, 2052B relative to each other according to the other translational orientations (e.g. Z in FIG. 21A, X in FIG. 21B).

FIG. 22A is a diagram schematically representing example device 2705 (and/or example method 2700) including a stimulation lead 2710 comprising a stimulation portion 2751, which comprises at least some of substantially the same features and attributes as the example devices/methods described in association with at least FIGS. 18A-21B, except with a distal portion 2724 of the lead 2710 including two independent flexible connector segments 2730A, 2730B, which extend from a bifurcation point 2729 along distal portion 2724 of the lead 2710.

As shown in FIG. 22A, each flexible connector segment 2730A, 2730B has a length L12 (FIG. 22AA) which may enhance positioning of each stimulation element 2052A, 2052B of the stimulation portion 2751 according to six degrees of freedom independent of the other respective stimulation element 2052A, 2052B. Similar to FIG. 18A, FIG. 22A is an illustration showing the second surface (e.g., 2053A of FIG. 18A) of the body 2054 of each stimulation element 2052A, 2052B, such that, in some examples, the contact electrodes 2056 may face toward target tissue as illustrated by the contact electrodes 2056 being in dashed lines. As shown in FIG. 22AA, length L12 of each flexible connector segment 2730A, 2730B extends between distal end 2733 and proximal end 2734, which joins to or extends from bifurcation portion 2729.

Accordingly, via this length (L12), portions 2731 of the flexible connector segments 2730A, 2730B may be manipulated into various orientations, angles, curves, and/or straight segments in order to manipulate a respective one of the stimulation elements 2052A, 2052B into various orientations, angles, positions, etc., independent of the manipulation of the other respective one of the stimulation elements 2052A, 2052B. Via this arrangement, one stimulation element (e.g. 2052A may be positioned into stimulating relation to different target nerves (e.g. on different sides of the patients' body) without being limited by the positioning of the other stimulation element (e.g. 2052B) such that each respective stimulation element 2052A, 2052B may establish robust electrical capture of the respective nerve portions being targeted for stimulation (and/or sensing in some examples). In some examples, the length L12 (FIG. 22AA) of each flexible connector segment 2730A, 2730B (e.g. extending from proximal end 2734 at bifurcation point 2729 to a distal end 2733) may be the same as or greater than a length (L2 in FIG. 18B) of the body 2054 of the respective stimulation elements 2052A, 2052B. In some examples, a length (L12) of each flexible connector segment 2730A, 2730B may be a multiple (e.g. 2x, 3x, etc.) of the length of the body 2054 or otherwise substantially greater than (e.g. 25% more, 50% more, 75% more) the length (L2 in FIG. 18B) of the body 2054. In contrast to the single flexible connector segment 2060 in the example arrangement in FIG. 18A, the pair of independent flexible connector segments 2060 in the example of FIG. 22A may originate from a bifurcation point 2729 which is located more proximally than the T-shaped junction 2065 in the example of FIG. 18A.

As previously mentioned, the length of the lead segments 2730A, 2730B of the stimulation lead 2710 may enhance introduction and advancement of the stimulation elements 2052A, 2052B (and ensuing maneuvering and chronic implantation) via the midline implant-access incision 609A.

Among other aspects, in some examples the flexible connector segments 2730A, 2730B comprise materials and/or a construction which is unbiased in any particular shape such as a curved right angle, hairpin (e.g. 180 degree curve), which may increase overall maneuverability and flexibility of the connector segments 2730A, 2730B to be positioned relative to target tissues and independently relative to each other.

FIG. 22B is a diagram schematically representing example device 2805 (and/or example method 2800) including a stimulation lead 2810 comprising a stimulation portion 2851, which comprises at least some of substantially the same features and attributes as the example devices/methods described in association with at least FIGS. 18A-21B and/or FIG. 22A, except (among other differences) comprising two independent flexible connector segments 2837A, 2837B which have different lengths (L6, L7) extending from a bifurcation point 2829 along distal portion 2824 of the body 2822 of stimulation lead 2810. As further shown in FIG. 22B, an utmost distal end 2833 of the respective connector segments 2837A, 2837B are connected to the respective stimulation elements 2052A, 2052B of the stimulation portion 2851 (such as at end 2059A in some examples). In some such examples, via the different lengths (L6, L7) of the respective flexible connector segments 2837A, 2837B, the bifurcation point 2829 may be implanted in a position significantly lateral to a sagittal midline 316. This configuration may enhance implantation of the stimulation portion 2851 in some situations, such as but not limited to, anatomical challenges along or near the sagittal midline 316. In one aspect, this configuration may sometimes be referred to as distal connector segments exhibiting asymmetric lengths and/or an asymmetric deployment of distal lead portions for the respective stimulation elements 2052A, 2052B. Among other features, this arrangement may facilitate implementing a greater degree of rotation or translation of a stimulation element 2052A, 2052B on one side of the body than on another side of the patient's body to accommodate variances in patient anatomy. In this regard, the relative degree of rotation and/or translation of the flexible connector segment 2060 may be regarded as being asymmetric.

In some such examples, the length L7 of flexible connector segment 2837B may be greater than the nominal length L4 or extended length L5 of flexible connector segment 2060 in FIGS. 20A, 20B, respectively.

FIGS. 22C and 22D are diagrams schematically representing stimulation elements 2910 and 2920, respectively, which comprise at least some of substantially the same features and attributes as the stimulation elements 2052A, 2052B described in FIGS. 18A-22B, except having a number, shape, and/or size of contact electrodes 2916 and 2926, respectively, different from the number, shape, and/or size of contact electrodes 2056 for stimulation elements 2052A, 2052B. In particular, as shown in FIG. 22C, in some examples stimulation element 2910 comprises an array of contact electrodes 2916 arranged as split contact electrodes, thereby doubling the number of contact electrodes as compared to the contact electrodes 2056 for stimulation elements 2052A, 2052B. Among other features, the greater number of contact electrodes and increased number of different positions on the stimulation surface (e.g. 2053A) may enhance selectivity of target tissues, such as nerve portions, muscle portions, combinations of nerve portions and muscle portions, neuromuscular junctions of nerve portions and muscle portions, and/or combinations thereof. In some examples, the greater number of contact electrodes 2916 of stimulation element 2910 may enhance a number, type, orientation, etc. of potential stimulation vectors among the contact electrodes 2916 of one stimulation element of a pair of stimulation elements (e.g. 2052A, 2052B) and/or between contact electrodes 2916 of one stimulation element (e.g. 2052A or 2052B) and the contact electrodes 2916 of the other respective stimulation element (e.g. 2052A or 2052B).

As shown in FIG. 22D, in some examples stimulation element 2920 comprises an array of contact electrodes 2926 arranged as an array or grid of contact electrodes 2926 arranged in rows and columns (e.g. 3×4, 2×4, 3×3, 4 x 4, etc.) thereby significantly increasing the number of contact electrodes 2926 as compared to the contact electrodes 2056 for stimulation elements 2052A, 2052B. Among other features, the greater number of contact electrodes 2926 and increased number of different positions on the stimulation surface (e.g. 2053A) may enhance selective stimulation of target tissues such as nerve portions, muscle portions, combinations of nerve portions and muscle portions, neuromuscular junctions of nerve portions and muscle portions, and/or combinations thereof. In some examples, the greater number of contact electrodes 2926 of stimulation element 2920 may enhance a number, type, orientation, etc. of potential stimulation vectors among the contact electrodes 2926 of one stimulation element of a pair of stimulation elements (e.g. 2052A, 2052B) and/or between contact electrodes 2926 of one stimulation element (e.g. 2052A or 2052B) and the contact electrodes 2926 of the other respective stimulation element (e.g. 2052A or 2052B).

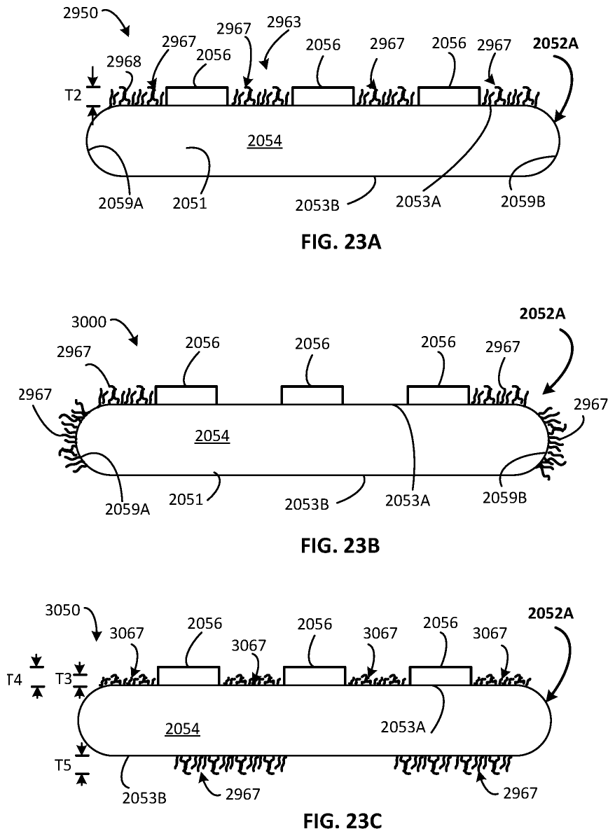

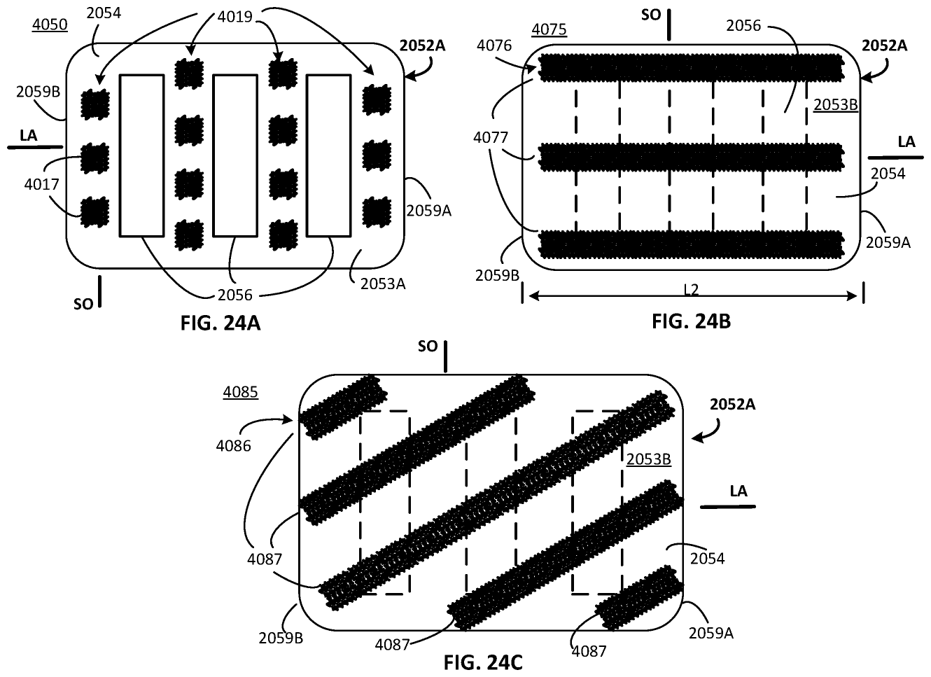

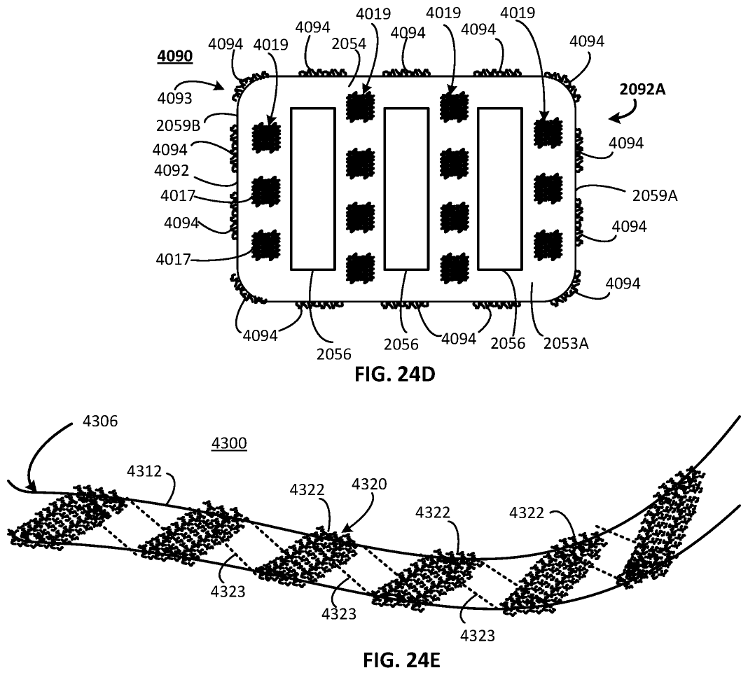

FIGS. 23A-24C are a series of diagrams schematically representing various example anchor arrangements by which the stimulation elements (e.g. 2052A, 2052B) of FIGS. 18A-22D may be secured relative to target tissues and/or surrounding non-target tissues. The various example anchor arrangements in FIGS. 23A-24C also may comprise example implementations by which other stimulation portions, stimulation elements, etc. throughout various examples of the present disclosure may be secured, fixed, etc. relative to target tissues and/or surrounding non-target tissues.

As previously noted in association with at least FIGS. 18A-22D and/or other previously described examples of the present disclosure, various type of anchors such as selectively deployable tines, barbs, etc. may be provided as part of the stimulation elements.

As shown in FIG. 23A, in some examples, one anchor structure 2963 may comprise a plurality of anchor portions 2967, each of which comprise a plurality of anchor elements 2968. In some examples, each anchor portion 2967 may comprise at least some of substantially the same features and attributes as the example anchor portions described in association with at least FIGS. 8E-8H, 11A-11B, 13A-13C, 14A-14D, 15A-15H, 16H-1I, and/or 23A-25 in which a plurality of anchor elements are configured to engage surrounding tissues (e.g. target tissues and/or non-target tissues) to secure the stimulation element generally and to secure the contact electrodes 2056 into stimulating relation to the target tissues such as nerve portions, muscle portions, combinations of nerve portions and muscle portions, neuromuscular junctions of nerve portions and muscle portions, and/or combinations thereof.

As shown in the diagram 2950 of FIG. 23A, the various anchor portions 2967 are located on the stimulation surface 2053A of the stimulation element 2052A and interposed between adjacent contact electrodes 2056 and in some examples, also may be located on the outer ends of the plurality of contact electrodes 2056, such as shown in FIG. 23B. In this configuration, the anchor portions 2967 act to engage target tissues and/or non-target tissues immediately adjacent to the contact electrodes 2056 to facilitate engagement of the contact electrodes 2056 in stimulating relation to the target tissues.

As shown in the diagram 3000 of FIG. 23B, in some example arrangements, anchor portions 2967 are located on the ends 2059A, 2059B (and/or side edges) of the body 2054 of the stimulation elements 2052A, 2052B but are omitted from the locations between adjacent contact electrodes 2056. In some such examples, this configuration may enhance engagement of the contact electrodes 2056 with the surrounding target tissues and non-target tissues while still providing anchor portions in close proximity to the contact electrodes 2056.

In some examples, this configuration may be desirable in example stimulation elements in which contact electrodes 2056 are flush (or have a low profile) relative to surface 2053A because the absence of anchor portions 2967 between contact electrodes 2056 may facilitate more direct engagement of the contact electrodes 2056 with the target tissues.

As shown in the diagram 3050 of FIG. 23C, in some examples anchor portions 2967 may be located on a non-stimulation surface 2053B (e.g. a back side) of the stimulation element 2052A while some anchor portions 3067 may be located on the stimulation surface 2053A (as shown in FIG. 18C) or omitted from the stimulation surface 2053A. The anchor portions 3067 on the non-stimulation surface 2053B may enhance securing the stimulation element 2052A relative to surrounding non-target tissues. For example, upon closing an implant-access incision (e.g. 609A), anchor portions 2967 on the non-stimulation surface 2053B may engage more superficially-located tissues above the stimulation element 2052A, thereby providing additional fixation.

While FIG. 18C shows non-stimulation surface 2053B partially covered by anchor portions 2967, it will be understood that in some examples, the entire (or substantially the entire) non-stimulation surface 2053B may be covered by anchor portions 2967.

As further shown in FIG. 23C, in some examples the anchor portions 3067 may comprise a thickness T3 (e.g. height) which is less than a distance T4 (e.g. height) by which contact electrodes 2056 may protrude from first surface 2053A such that the anchor portions 3067 may enhance securing the stimulation element 2052A but have a low profile to also help facilitate robust engagement of the contact electrodes 2056 with the target tissues. In some examples, the anchor portions 2967 in the example of FIG. 23C may have a thickness T5 (e.g. height) which is substantially thicker than (e.g. greater than) the low profile thickness T3 (e.g. height) of the anchor portions 3067 so that anchor portions 2967 (on the non-stimulation surface 2053B) may provide for more aggressive engagement of surrounding tissues.

FIG. 24A is a diagram 4050 including a top plan view schematically representing an example device (and/or example method) including a stimulation element 2052A (or 2052B) comprising an array of anchor portions 4017 distributed in a pattern spaced apart from each other on a first surface 2053A (e.g. stimulation surface) of the stimulation element 2052A, with at least some of the various anchor portions 4017 interposed between adjacent contact electrodes 2056 such that the anchor portions 4017 are spaced apart from each other in a first orientation parallel to a length (e.g. a longitudinal axis LA) of the body 2054 of the stimulation element 2052A. The anchor portions 4017 also are spaced apart from each other in a second orientation (SO) perpendicular to the first orientation, with such rows 4019 of anchor portions extending generally parallel to a length of the contact electrodes 2056. In some examples, each anchor portion 4017 comprises a plurality of anchor elements, which comprise at least some of substantially the same features and attributes as the anchor portions, anchor elements, etc. as described in association with at least FIGS. 8E-8H, 11A-11B, 13A-13C, 14A-14D, 15A-15H, 16J-16K, 23A-24E, and/or 25H in which a plurality of anchor elements are configured to engage surrounding tissues (e.g. target tissues and/or non-target tissues) to secure the stimulation element generally and to secure the contact electrodes 2056 into stimulating relation to the target tissues such as nerve portions, muscle portions, combinations of nerve portions and muscle portions, neuromuscular junctions of nerve portions and muscle portions, and/or combinations thereof.

FIG. 24B is a diagram 4075 including a top plan view schematically representing an example device (and/or example method) including a stimulation element 2052A (or 2052B) comprising an array 4076 of anchor portions 4077 distributed in a pattern of columns spaced apart from each other on an opposite second surface 2053B (e.g. non-stimulation surface) of the stimulation element 2052A, with at least some of the various anchor portions 4077 spaced apart from each other in a second orientation (SO) perpendicular to a length (e.g. a longitudinal axis LA) of the body 2054 of the stimulation element 2052A. Each anchor portion 4077 extends generally perpendicular to a length of the contact electrodes 2056 and extend generally parallel to a length (L2) of the body 2054 of the stimulation element 2052A. In some examples, the anchor portions 4077 may comprise at least some of substantially the same features and attributes as anchor portions 4017 of the example arrangement in FIG. 24A, except for comprising a different shape, size, and/or orientation.

FIG. 24C is a diagram 4085 including a top plan view schematically representing an example device (and/or example method) including a stimulation element 2052A (or 2052B) comprising an array 4086 of anchor portions 4087 distributed in a pattern spaced apart from each other in a generally parallel relationship on an opposite second surface 2053B (e.g. non-stimulation surface) of the stimulation element 2052A. In one aspect, the anchor portions 4087 may sometimes be referred to as extending diagonally across the body 2054 of the stimulation element 2052A. In one aspect, in this diagonal configuration, the various anchor portions 4087 extend in long strips which may enhance securing the stimulation element in (or generally parallel to) both a major axis orientation (e.g. lengthwise orientation, along longitudinal axis LA) and a minor axis orientation (e.g. transverse orientation SO) of the body 2054 of the stimulation element 2052A (or 2052B). In some examples, the anchor portions 4087 may comprise at least some of substantially the same features and attributes as anchor portions 4077 of the example arrangement in FIG. 24B, except for comprising a different shape, size, and/or orientation.

FIG. 24D is a diagram 4090 including a top plan view schematically representing an example stimulation element 2092A comprising at least some of substantially the same features and attributes as the stimulation element 2052A of FIGS. 24A (and/or 24B, 24C), except further comprising an array 4093 of anchor portions 4094 located on a periphery or outer side edge 4092 of the body 2054 of the stimulation element 2092A. In some examples, the anchor portions 4094 may comprise at least some of substantially the same features and attributes as anchor portions (e.g. 4017, 4077, etc.) of the example arrangement in FIGS. 24A, 24B, etc., respectively, except for comprising a different shape, size, and/or orientation as represented by FIG. 24D.

In some examples, the respective anchor portions 4094 are spaced apart from each other about the periphery 4092 of body 2054, which may provide a desired combination of slidability for initial positioning and of fixation once the stimulation element 2092A has been maneuvered into a location of chronic implantation. However, in some examples, the respective anchor portions 4094 are provided with little or no spacing between respective anchor portions 4094 such that the periphery 4092 may be considered to comprise a continuous or substantially continuous anchor portion.

In one aspect, in some examples the example arrangement periphery-located anchor portions 4094 of FIG. 24D may enhance anchoring within or among certain types of tissues while potentially lessening an amount of the surface area of other portions (e.g. 2053A, 2053B) of a body 2054 to be partially covered with some anchor portions. In another aspect, in some examples such arrangements may enhance anchoring for certain orientations (e.g. anterior-posterior, superior-inferior, medial-lateral) in view of a direction, orientation, etc. in which muscle portions of the target tissues (or surrounding non-target tissues) may move.

FIG. 24E is a diagram 4300 including a top plan view of an example flexible connector segment 4306 which may comprise at least some of substantially the same features and attributes as (or comprise an example implementation of) one of the previously described, flexible connector segments or distal lead segments (FIGS. 18A-22B) extending between the respective stimulation elements 2052A, 2052B, while also comprising an anchor structure 4320 extending along a length of the flexible connector segment 4306.

As shown in FIG. 24E, the anchor structure 4320 forms a helical pattern on an exterior surface 4312 of the flexible connector segment 4306, with the anchor structure 4320 comprising anchor portions 4322 and anchor portions 4323 (shown in dashed lines to represent an opposite side of the flexible connector segment 4306).

In some examples, the anchor portions 4322 and the anchor portions 4323 may be spaced apart from each other by some distance, while in some examples, the anchor portions 4322 and anchor portions 4323 form part of a single, continuous anchor structure.

In some examples, each anchor portion comprises a plurality of anchor elements, which comprise at least some of substantially the same features and attributes as the anchor portions, anchor elements, etc. as described in association with at least 16C-16D and more generally in association with at least FIGS. 8E-8H, 11A-11B, 13A-13C, 14A-14D, 15A-15H, 16J-16K, 23A-24D, and/or 25H in which a plurality of anchor elements are configured to engage surrounding tissues (e.g. target tissues and/or non-target tissues) to secure the flexible connector segment (or distal lead segments) relative to surrounding tissues. This arrangement also acts to secure associated stimulation elements relative to the target tissues such as nerve portions, muscle portions, combinations of nerve portions and muscle portions, neuromuscular junctions of nerve portions and muscle portions, and/or combinations thereof.

In considering the various anchor portions described throughout the examples of at least FIGS. 8E-24E, it will be understood that anchor portions may be located on just the stimulation elements, on just the flexible connector segments (or distal lead segments), or on both the stimulation elements and the flexible connector segments (or distal lead segments).

FIGS. 25A-25K schematically representing an example device 4405 (and/or example method) for treating obstructive sleep apnea, with FIG. 25A including a diagram 4400 schematically representing the example device (and/or example method) relative to patient anatomy. In some examples, the example device 4405 may comprise at least some of substantially the same features as the examples described in association with at least FIGS. 18A-24E (and other examples further associated with FIGS. 18A-24E), except with stimulation portion 4451 comprising at least some shapes, features, etc. different from stimulation portion 2051 in FIGS. 18A-24E.

As shown in FIG. 25A, the example device 4405 comprises a lead 4410 including a main lead body 2022 and distal portion 2024 having an end 2029 which is connected to, and electrically communicates with, a stimulation portion 4451. The stimulation portion 4451 comprises a single carrier body 4454, which comprises an elongate flexible member. The single carrier body comprises a common portion 4452C interposed between two opposite arms 4452A, 4452B, with first arm 4452A comprising a first group 4455A of spaced apart electrodes 4456 (e.g. like 2056 in FIGS. 18A-18C) exposed on first surface 4453A of body 4454 and second arm 4452B comprising a second group 4455B of spaced apart electrodes 4456 exposed on first surface 4453A of body 4454. As shown in FIGS. 25C-25D, the stimulation portion 4451 comprises a second surface 4453B opposite the first surface 4453A, with the second surface 4453B omitting any such electrodes, in some examples. The arms 4452A, 4452B extend in opposite orientations outwardly from the common portion 4452C and each comprising an end 4459A, 4459B, respectively. In some examples, the arms 4452A, 4452B of stimulation portion 4451 may sometimes be referred to as stimulation elements 4452A, 4452B, respectively. In some examples, the first group 4455A of electrodes is spaced apart from the second group 4455B of electrodes such that common portion 4452C omits any electrodes, i.e. is electrode-free as shown in FIG. 25A. However, in some examples, the common portion 4452C may comprise at least one electrode like or dissimilar from electrodes 4456.

As further shown in FIG. 25A, upon chronically implanting the stimulation portion 4451 (as in FIG. 18A) the first group 4455A of electrodes 4456 of first arm 4452A are in stimulating relation to at least some target tissues on the first side (e.g. 312R) of the patient's body and the second group 4455B of electrodes 4456 of second arm 4452B are in stimulating relation to at least some target tissues on the second side (e.g. 312L) of the patient's body with common portion 4452C extending generally transversely across (e.g. straddling) the sagittal midline 316.

In a manner similar to that shown in at least FIG. 18B, FIGS. 25B-25C schematically represent that each respective arm 4452A, 4452B (i.e. stimulation elements) of the paddle-style carrier body 4454 comprises a width (W2) and a length (L2), and a thickness (B2). In some examples and as shown in both FIGS. 25B. 25C, and 25D, the paddle-style of the body 4454 may comprise a generally rounded rectangular cross-sectional shape, which is at least partially defined by the width (W2) being substantially greater than the thickness (B2). In some examples, in one context this “substantially greater” dimensional relationship comprises the width (W2) being at least 3 times (or at least 4 times or at least 5 times) greater than the thickness (B2). In some such examples, the “substantially greater” dimensional relationship comprises the width (W2) being at least one order of magnitude greater than the thickness (B2).

In some examples, in one context each respective arm 4452A, 4452B of the paddle-style, carrier body 4454 (of stimulation portion 4451) may be at least partially defined by the length (L2) being substantially greater than the thickness (B2). In some such examples, the “substantially greater” dimensional relationship comprises the length (L2) being at least 5 times greater (or at least 3 times or at least 4 times) than the thickness (B2). In some such examples, the “substantially greater” dimensional relationship comprises the length (L2) being at least one order of magnitude greater than the thickness (B2).

In some examples, in one context each respective arm 4452A, 4452B (i.e. stimulation element) of the paddle-style, carrier body 4454 (of stimulation portion 4451) comprises a width (W2) on the order of 3 times, 4 times, 5 times, 6 times, 7 times, 8 times, 9 times, or at least 10 times (order of magnitude) greater than a diameter of a target nerve at which the carrier body 4454 is deployed.

In some examples, the elongate flexible member forming the carrier body 4454 may comprise a substantially uniform cross-sectional shape and/or substantially uniform cross-sectional size, which extends an entire length (or a substantially entire length) of stimulation portion 4451. In some examples, the substantially uniform cross-sectional size and/or substantially uniform cross-sectional size may extend throughout an entire length (or substantially the entire length) of the common portion 4452C of the elongate flexible member but not necessarily throughout the respective arms 4452A, 4452A (i.e. stimulation elements).

In some examples, the contact electrodes 4456 of FIGS. 25A-25C may comprise at least some of substantially the same features and attributes as the contact electrode 2056 and associated portions of body 2054 of FIGS. 18A-18C.

In a manner similar to the stimulation portion 2051 in at least FIGS. 18A-18C, in some examples each arm (e.g. stimulation element) 4452A, 4452B of the stimulation portion 4451 may comprise an anchor structure(s) on the exposed non-conductive surfaces (e.g. 4457, 4458, etc.) of the carrier body 4454. In some examples, the common portion 4452C may comprise an anchor structure(s) on the exposed surfaces of the carrier body 4454. In some such examples, later described transition portions 4472A, 4472B also may comprise an anchor structure(s) on the exposed surfaces of the carrier body 4454. In some examples, such anchor structure(s) may comprise at least some of substantially the same features and attributes as the anchor structure(s) as described in association with at least FIGS. 8E-15H, 16J-16K, and/or 23A-24E.

As shown in FIG. 25DD, in some examples the stimulation portion 4451 comprises a carrier body 4460, which may include a generally circular, cross-sectional shape instead of a rounded rectangular shape in FIGS. 25A-25D. In this example implementation, a central portion 4462 may comprise several electrical conductors extending within and throughout the carrier body 4460 with each respective conductor being electrically connected to a respective contact electrode like a contact electrode 4456 in FIGS. 25A-25D. As further shown in FIG. 25DD, one example implementation of a contact electrode suitable for use with the cross-sectional circular shape may comprise a ring electrode 4466 (or split ring electrode). The central portion 4462 also comprises insulative material to electrically isolate each of the respective electrical conductors from each other and from the outer ring electrode 4466. In other respects, the stimulation portion 4451 may comprise at least some of substantially the same features and attributes as for FIGS. 25A-25D. In some examples, the cross-sectional shape may comprise an elliptical shape instead of a circular shape.

With this in mind, in some examples, and as further shown in the diagram 4500 of FIG. 25E, opposite end portions of the common portion 4452C may comprise a first transition portion 4472A and an opposite second transition portion 4472A. In some examples, the first transition portion 4472A comprises a region (indicated by a dotted box) extending between an inner end (represented by dotted line 4470A) of the first arm 4452A and a central portion 4473 of the common portion 4452C. In some examples, the second transition portion 4472B comprises a region (indicated by a dotted box) extending between an inner end (represented by dotted line 4470B) of the first arm 4452B and a central portion 4473 of the common portion 4452C.

In some such examples, each of the respective first and second transition portions 4472A, 4472B comprise a flexibility greater than a flexibility of the central portion 4473 of the common portion 4452C and/or of arms 4452A, 4452B. In some of these examples, the relatively greater flexibility may be implemented within the carrier body 4454 providing wires, an insulating jacket, other members, etc. which are more flexible than in the central portion 4473 and/or the arms 4452A, 4452B. In some of these examples, the relatively greater flexibility is implemented while the transition portion 4472A, 4472B comprises a cross-sectional shape and/or size which remains substantially the same as the arms 4452A, 4452B and/or the central portion 4473. In some such examples, the central portion 4473 of the common portion 4452C and the transition portions 4472A, 4472B together comprise a single, unitary member (e.g. monolithic) and not separate, discrete structures.

However, in some examples, the relatively greater flexibility of the transition portions 4472A, 4472B may be implemented via constructing the transition portions as comprising a reduced thickness and/or a reduced width as compared to a thickness and/or width (or diameter, or greatest cross-sectional dimension) of the central portion 4473 and/or each respective arm 4452A, 4452B.

In some examples, regardless of the particular construction, the greater flexibility comprises a substantially greater flexibility in which the flexibility of the transition portions 4472A, 4472B may comprise a greater flexibility of at least 25 percent, at least 50 percent, at least 75 percent, or at least 100 percent.

As further shown in FIG. 25E, implanting the stimulation portion 4451 such as via the implant-access incision 609A (FIG. 25A), comprises selectively bending the first and second transition portions 4472A, 4472B to cause the first arm 4452A to extend at an angle μ of between about 45 and 180 degrees relative to the second arm 4452B to position each of the respective first and second group 4455A, 4455B of electrodes 4456 into stimulating relation to target upper airway patency-related tissues on the first side (e.g. 312R) of the patient's body and the second side (e.g. 312L) of the patient's body, respectively. In some examples, the angle μ may comprise between about 80 and 160 degrees, between about 90 and 150 degrees, or between about 100 and 140 degrees.

In some examples, in addition to their greater flexibility, at least one of the common portion 4452C (including the transition portions 4472A, 4472B) and the arms 4452A, 4452B are biased to cause the respective first and second arms 4452A, 4452B (i.e. stimulation elements) to extend at the above-mentioned angles μ relative to each other prior to implantation. Accordingly, during implantation the different portions of the flexible, resilient carrier body 4454 (including the common portion 4452C, the transition portions 4472A, 4472B, and/or arms 4452A, 4452B) are manipulated to cause at least the arms 4452A, 4452B to be placed in a desired orientation to be in stimulating relation relative to target tissue with the biasing property helping to maintain this orientation during chronic implantation. Conversely, in some examples, the transition portions 4472A, 4472B (and/or central portion of common portion 4452C) are not biased at the above-mentioned angles (p) such that the entire carrier body 4454 may be generally planar (i.e. may lay flat) prior to implantation, and then is manipulated into a desired angle during implantation via bending at the transition portions 4472A, 4472B and maintained at a desired angle via anchoring and/or how the stimulation portion 4451 is positioned relative to the surrounding tissues in the patient anatomy.

In some example implementations of the above-mentioned biasing property, the common portion 4452C (including the transition portions 4472A, 4472B) and/or the respective first and second arms 4452A, 4452B may comprise a pre-formed arcuate shape (e.g. a concave surface) to face and engage the upper airway patency-related tissues to be stimulated. In some of these examples, each transition portion 4472A, 4472B may comprise a pre-formed first arcuate surface (e.g. 4480) to face the upper airway patency-related tissues and which is non-recessed along its arc length.

However, in some examples, the common portion 4452C, the transition portions 4472A, 4472B, and/or the arms 4452A, 4452B form generally planar elements, which are not pre-formed arcuate shape and instead are manipulated into a desired arcuate shape during implantation and which may be maintained during chronic implantation. In some examples, anchor structures may be used to maintain a desired bent or curved shapes during chronic implantation, whether or not the respective common portion 4452C, transition portions 4472A, 4472B, and/or arms 4452A, 4452B have pre-formed arcuate shapes.

In some examples, the first surface 4480 (e.g. inner surface) of each transition portion 4472A, 4472B is non-recessed relative to a first surface 4482 of each respective arm 4452A, 4452B and relative to a first surface 4484 of the central portion (or segment) 4473 of the common portion 4452C.

In some examples, the first surface 4480 of each transition portion 4472A, 4472B comprises less than about 10 percent to about 30 percent change in curvature along its arc length. In some examples, a curvature along the first surface 4480 of each transition portion 4472A, 4472B is less than 10 percent different from a curvature along an arc length of the first surface 4484 of the central portion 4473 of the common portion 4452C and/or different from a curvature (which may be zero in some examples) along a length of the first surface 4482 of each respective arm 4452A, 4452B.

In some examples, at least the transition portions 4472A, 4472B may comprise a shape-retaining material, and wherein the shape-retaining material enables maintaining the selectively bent configuration of the arms 4452A, 4452B relative to each other in the chronically implanted configuration. As previously noted, one or more of the different types of example anchor structures described within the present disclosure may be employed to help maintain the desired configuration during chronic implantation.

In some examples, the transition portions 4472A, 4472B may comprise a resilient material without (or minimal) shape-retaining properties such that example anchor structures in combination with various anatomical structures may be used to maintain a desired bent configuration of the respective arms 4452A, 4452B relative to each other during initial implantation and chronic implantation.

At least some non-limiting anchor structures which may form part of stimulation portion 4451 are described later in association with at least FIG. 25H.

In some examples, the common portion 4452C of the stimulation portion 4451 omits at least one of a power element, a wireless communication element, and control circuitry. In some such examples, the omission of circuitry comprises the common portion 4452C including solely signal conductive elements extending, within an insulator and, to provide electrical connection to separate electrodes within each respective arm 4452A, 4452B.

However, in some examples, the common portion 4452C of the stimulation portion 4451 may comprise a power element, a wireless communication element, and/or control circuitry.

In some examples, at least one electrode of the first group 4455A of electrodes of first arm 4452A and/or of the second group 4455B of electrodes of the second arm 4452B may be activated to apply stimulation vectors among the first group 4455A of electrodes, among the second group 4455B of electrodes, and/or between at least one electrode of the first group 4455A of electrodes and at least one electrode of the second group 4455B of electrodes. In some such examples, the various stimulation vectors may comprise at least some of substantially the same features and attributes as described in association with at least FIGS. 19D-19E.

As noted elsewhere among other examples of the present disclosure, the upper airway patency-related tissues to be stimulated via stimulation portion 4451 may comprise nerve portion(s), muscle portion(s), a combination of nerve portion(s) and muscle portion(s), a neuromuscular junction of nerve portion(s) and muscle portion(s), and/or combinations thereof. In some such examples, the nerve portion comprise portions of the hypoglossal nerve and the muscle portions comprise portions of muscles innervated by portions of the hypoglossal nerve.

With this in mind, FIG. 25F is a diagram 4600 schematically representing the stimulation portion 4451 juxtaposed with at least some upper airway patency-related target tissues such as, but not limited to, a muscle portion 4490L, 4490R (e.g. cross-section of a muscle) belly and a nerve portion 4492R, 4492L.

Stimulation may be applied to at least these example target tissues via stimulation vectors among the first group 4455A of electrodes (e.g. unilateral stimulation), among the second group 4455B of electrodes (e.g. unilateral stimulation), among both the first group 4455A and the second group 4455B (e.g. bilateral stimulation, which may be alternating, simultaneous, etc.) and/or between at least one electrode of the first group 4455A of electrodes and at least one electrode of the second group 4455B of electrodes (e.g. cross-lateral stimulation).

FIG. 25G is a diagram 4700 schematically representing an example stimulation portion 4751 comprising at least substantially the features and attributes as stimulation portion 4451 of FIGS. 25A-25F, except with the common portion 4452C comprising two regions 4775A, 4775B of varying flexibility in which a flexibility of each region 4775A, 4775B gradually increases (as represented by directional arrows F1, F2) from a central reference 4710 distally toward the inner ends 4470A, 4470B of the respective arms 4452A, 4452B. These regions 4775A, 4775B are provided instead of the distinct transition portions 4472A, 4472B provided in the examples of at least FIGS. 25E-25F). Other than the different manner in which the varying distal flexibility is provided in regions 4775A, 4775B, the stimulation portion 4751 may comprise at least some of substantially the same features and attributes as the example stimulation portion 4451 as described in association with at least FIGS. 25A-25E and 25H.

FIG. 25H is a diagram 4800 schematically representing an example stimulation portion 4851, which comprises at least some of substantially the same features and attributes as the stimulation portion 4451 (also 4751) of FIGS. 25A-25G, except with diagram 4800 explicitly illustrating just one example implementation of many different example implementations of example anchor arrangements of the present disclosure. With this in mind, as shown in FIG. 25H, the example stimulation portion 4851 comprises a first anchor structure 4810 and/or a second anchor structure 4820. The second anchor structure 4820 comprises a plurality of anchor portions 4822 located on an exterior surface of the carrier body 4454 for securely engaging surrounding tissues to fixate the arms 4452A, 4452B and/or common portion 4452C to maintain the electrodes (of first group 4455A and second group 4455B) in stimulating relation to desired target upper airway patency-related tissues. It will be understood that the particular size, shape, percentage of coverage area, location, etc. of the anchor portions 4822 are merely examples, and that the anchor structure 4820 may comprise any one of, or a combination of, the various example anchor structures of the present disclosure such as, but not limited to, the examples of FIGS. 8B-8L, 11A-11B, 13A-16A, 16J-16K, and/or 23A-24E. Accordingly, in some examples, at least some of the respective anchor portions 4822 may comprise a plurality of homogeneous anchor elements and/or a plurality of heterogeneous anchor elements, which may further comprise micro anchor elements in some examples, as further previously described such as (but not limited to) in association with at least some of FIGS. FIGS. 8B-8L, 11A-11B, 13A-16A, 16J-16K, and/or 23A-24E.

In some examples, in addition to or instead of second anchor structure 4820, the stimulation portion 4851 may comprise the first anchor structure 4810 which comprises a plurality of holes 4812 formed in, and spaced apart from each other, at or about an outer surface of the carrier body 4454. While in some examples, the holes 4812 provide a mechanism for using sutures (or hooks, barbs, etc.) to secure the carrier body 4454 relative to surrounding tissues, in some examples, the holes 4812 may be used for other types of securing. For instance, in some examples, at least some of the holes 4812 may be used to encourage tissue growth within, around, and through the holes 4812 to secure (via the passage of time) the carrier body 4454 relative to the surrounding tissue during a chronic implantation. It will be understood that the type, size, shape, location, quantity of holes 4812 may vary depending on whether the holes 4812 are used for suturing-type anchoring or for sutureless anchoring via tissue growth.

In some examples in which the holes 4812 of first anchor structure 4810 are used to promote tissue growth to secure the carrier body 4454 (via the passage of time), relative to surrounding tissue, and in which the second anchor structure 4820 of anchor portions 4822 is also deployed, at least some of the anchor portions 4822 may be resorbable in some examples. In such examples, the anchor portions 4822 may provide immediate fixation of the carrier body 4454 (relative to surrounding tissues) and then slowly become resorbed overtime while the carrier body 4454 becomes increasingly secured by accumulating tissue growth via the holes 4812 of the first anchor structure 4810. The type, volume, and/or shape of the material forming the anchor portions 4822 (as well as their location, quantity, etc.) may be selected to ensure sufficient fixation until adequate tissue growth has occurred within, through, and/or around the holes 4812 to securely maintain the stimulation portion 4851 in the desired location during chronic implantation. Of course, in such arrangements, a combination of resorbable and non-resorbable anchor elements of anchor portions 4822 may be used such that at least some of the non-resorbable anchor elements remain in place for chronic implantation such that both holes 4812 and anchor portions 4822 provide a portion of the overall fixation arrangement.

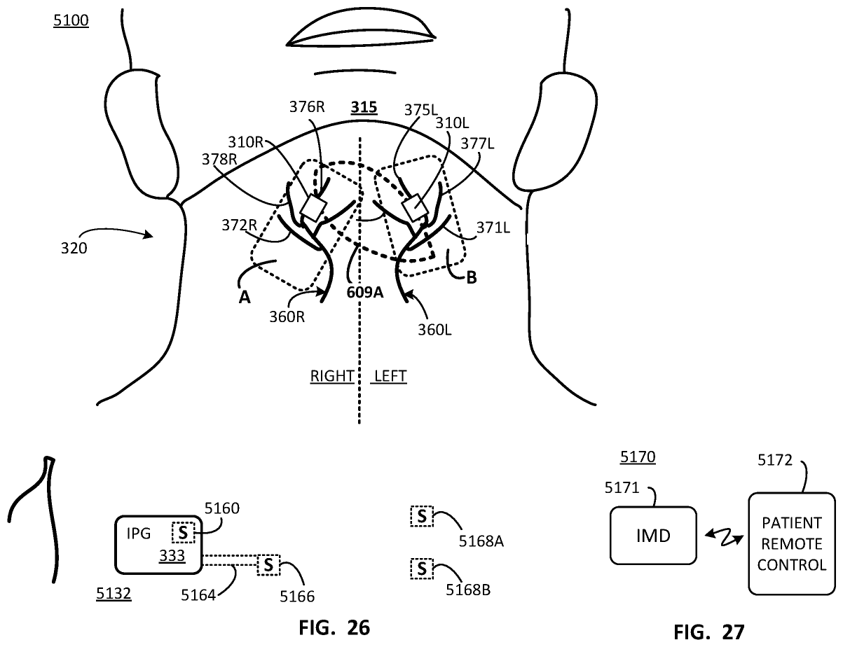

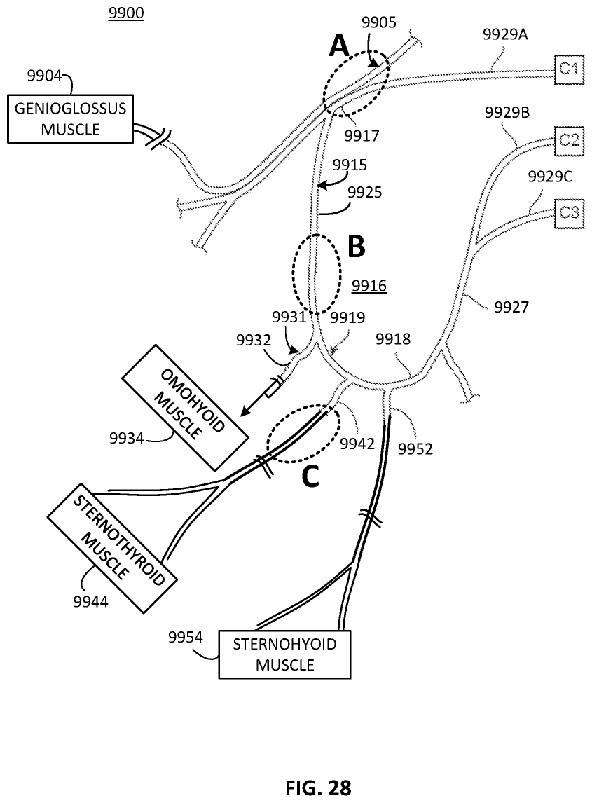

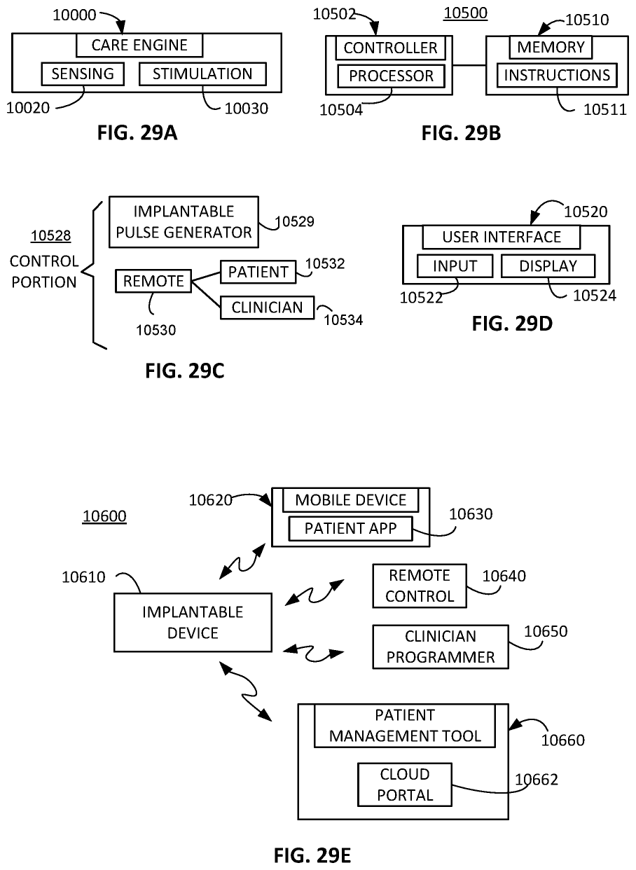

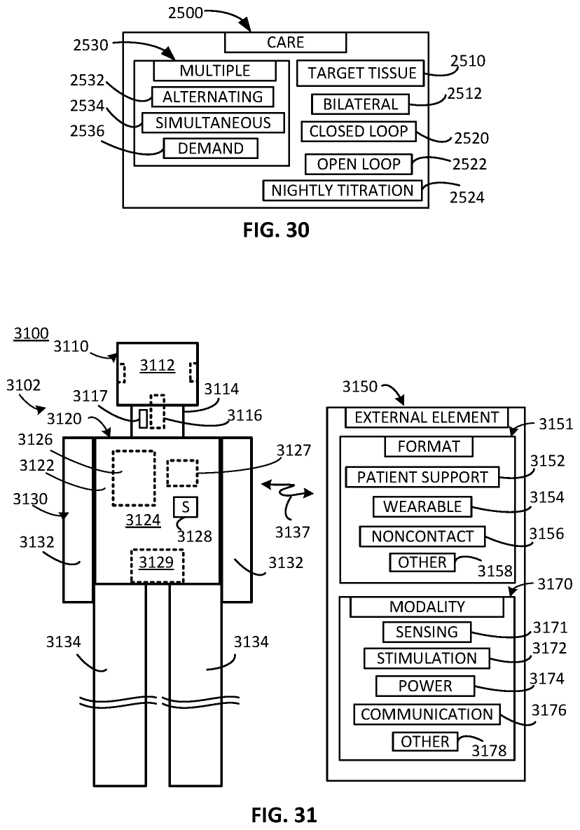

FIGS. 251, 25J, 25K are each a top plan view schematically representing an example device including an example paddle-style stimulation portion 4951, 5051, 5071, respectively, each comprising a particular pattern of spaced apart electrodes. In general terms, each of the respective stimulation portions 4951 (FIG. 25I), 5051 (FIG. 25J), 5071 (FIG. 25K) may comprise at least some of substantially the same features and attributes of the example stimulation portion 4451 as described in association with at least FIGS. 25A-25H, except with the electrodes (on the respective stimulation elements) having orientations, shapes, sizes, patterns, etc. different from those shown for electrodes 4456 in FIGS. 25A-251.

As shown in FIG. 25I, in some examples a stimulation portion 4951 comprises first and second arms 4952A, 4952B, each of which comprise a respective group 4955A, 4955B of individually controllable electrodes 4956. Each electrode 4956 comprises a length L13 and width W5, which may comprise at least some of substantially the same features and attributes (e.g. width, length, spacing, etc.) as electrodes 4956 in FIGS. 25A-25D. However, in stimulation portion 4951 of FIG. 25I, a length L13 of the electrodes 4956 are aligned to be generally parallel to a longitudinal axis (LA) of, and generally perpendicular to a short axis (M1) of, the elongate carrier body 4454 of the stimulation portion 4951.

As shown in FIG. 25J, in some examples a stimulation portion 5051 comprises first and second arms 5052A, 5052B, each of which comprise a respective group 5055A, 5055B of individually controllable electrodes 5056. Each electrode 5056 may comprise at least some of substantially the same features and attributes (e.g. width, length, spacing, etc.) as electrodes 4956 in FIGS. 25A-25D, except with a length L14 of the electrodes 5056 being aligned to extend in an orientation (M2) which forms an acute angle θ1 relative to a longitudinal axis (LA) of the elongate carrier body 4454 of the stimulation portion 5051. In some examples, the angle θ1 is between about 30 and 60 degrees.

With reference to FIGS. 251 and 25J, among other aspects, orienting the electrodes (4956 in FIG. 25I; 5056 in FIG. 25J) at such angles may enhance signal capture (e.g. establishing stimulating relation) of target tissues (e.g. nerve portion) for certain anatomical situations or depending on whether the target nerve portion(s) are more distal or proximal along the length of a nerve, etc. For instance, in some examples, providing an angled orientation for electrodes (4956 in FIG. 25I, 5056 in FIG. 25J) may enhance capturing target tissues promoting protrusion of the tongue and/or enable avoiding capture of target tissues promoting retraction of the tongue.

Moreover, as further shown in FIG. 25J, in some examples the electrodes 5056 may comprise a length L14 which is shorter than a length L13 of the electrodes 4956 in the stimulation portion 4951 of FIG. 25I such that electrodes 5056 in FIG. 25J may be arranged in a grid pattern (e.g. 2×2, 2×3, 3×3, etc.) for each arm 5052A, 5052B. In some such examples, the ends of the electrodes 5056 spaced apart by a distance S5 and the sides of the electrodes 5056 spaced apart by a distance S6. In one aspect, arranging the electrodes 5056 in such patterns may enhance the ability, via independent control of the respective electrodes, to capture target tissues promoting protrusion of the tongue and/or enable avoid capture of target tissues promoting retraction of the tongue.

As shown in FIG. 25K, in some examples a stimulation portion 5071 comprises first and second arms 5072A, 5072B, each of which comprise a respective group 5075A, 5075B of individually controllable electrodes. In particular, each group 5075A, 5075B comprises first electrodes 5076A and second electrodes 5076B which are arranged in a non-parallel relationship relative to each other. In some examples, as further shown in FIG. 25K, the first electrodes 5076A and second electrodes 5076B are oriented transversely relative to each other.

In addition, as further shown in FIG. 25K, the respective second electrodes 5076B extend in a first orientation M3 and are placed end to end, with the pair of second electrodes 5076B interposed between first electrodes 5076A.

Each first electrode 5076A extends in a second orientation M4, which extends generally perpendicular to the first orientation M3. The first electrodes 5076A are located on opposite ends of the pair of second electrodes 5076B. As shown in FIG. 25K, in each group 5075A, 5075B, the first and second electrodes 5076A, 5076B are arranged together in a zig-zag pattern which extends along a length of each respective arm 5072A, 5072B.

It will be understood that the first electrodes 5076A and second electrodes 5076B can extend in orientations which are not generally perpendicular to each other while still being placed end-to-end or in other patterns in which the electrodes are spaced apart from each other in a non-uniform manner.

In a manner analogous to the previously described examples of FIGS. 251-25J, the zig-zag pattern of electrodes 5076A, 5076B (or other pattern) may enhance the ability, via independent control of the respective electrodes, to capture target tissues promoting protrusion of the tongue and/or enable avoid capture of target tissues promoting retraction of the tongue.

FIG. 25L is a diagram schematically representing an array 5080 of different shapes 5082, 5084, 5085, 5086, 5088 in which an electrode can be embodied for deployment on a stimulation element. As shown in FIG. 25L, the array 5080 comprises a circular shape 5082, a triangular shape 5084, a cross shape 5085, a V shape (e.g. chevron) 5086, and a diamond shape electrode 5088.

With this in mind, in some examples all of, or just some of the, electrodes 2056 on the stimulation elements of the stimulation portion 2051 of FIGS. 18A-24E may comprise one of the different shapes 5082, 5084, 5085, 5086, 5088, or another shape. However, in some examples, all of, or just some of the, electrodes 2056 on the stimulation elements of the stimulation portion 2051 of FIGS. 18A-24E may comprise of several of the respective different shapes 5082, 5084, 5085, 5086, 5088, or another shape.

In some examples all of, or just some of the, electrodes 4456 on the stimulation elements of the stimulation portion 4451 of FIGS. 25A-25H may comprise one of the different shapes 5082, 5084, 5085, 5086, 5088, or another shape. However, in some examples, all of, or just some of the, electrodes 4456 on the stimulation elements of the stimulation portion 4451 of FIGS. 25A-25H may comprise several of the respective different shapes 5082, 5084, 5085, 5086, 5088, or another shape.

In some examples all of, or just some of the, electrodes on the stimulation elements of the stimulation portion 4951 of FIG. 25I, 5051 of FIG. 25J, or 5071 of FIG. 25k may comprise one of the different shapes 5082, 5084, 5085, 5086, 5088, or another shape. However, in some examples, all of, or just some of the, electrodes on the stimulation elements of the stimulation portion 4951 of FIG. 25I, 5051 of FIG. 25J, or 5071 of FIG. 25k may comprise several of the respective different shapes 5082, 5084, 5085, 5086, 5088, or another shape.

In some examples, when embodied as an electrode, the different shapes of array 5080 of FIG. 25L may enhance establishing stimulating relation relative to a target tissue (e.g. nerve portion, muscle portion, etc.) depending on the particular anatomical situation in which the stimulation elements are placed.

With reference to each of the various examples of different shapes, patterns, and/or orientations of electrodes on the stimulation portions of FIGS. 18A-25L, it will be understood that each of the respective separate electrodes are individually addressable (i.e. programmable, controllable) by a control portion, stimulation controller, and the like in order to determine a desired combination or pattern of electrodes to optimize stimulation of target tissues in order to promote protrusion of the tongue to increase or maintain upper airway patency.

FIG. 26 is a diagram on an example arrangement 5100 comprising at least some of substantially the same features and attributes as example stimulation arrangement in FIG. 2A, except including various examples of sensors which may form part of the IPG 333 (or 1133 in FIG. 17A) and/or may be independent of the IPG 333 (or 1133 in FIG. 17A). In some examples, the example arrangement 5100 may comprise one example implementation of the care engine 10000 (FIG. 29A), control portions 10500, 10528, 10600 (FIG. 29C, 29E), and/or user interface 10520 (FIG. 29D), as described later.

In some examples IPG 333 (or 1133 in FIG. 17A) may comprise an on-board sensor 5160 which is incorporated within a housing of the IPG (333, 1133) and/or be exposed on an external surface of the housing of the IPG (333, 1133), which may be implanted in the pectoral region 5132 of the patient in some examples or in other locations such as a head-and-neck region (e.g. FIG. 17A).

In some examples, the sensor 5160 may comprise an accelerometer, which may comprise a single axis accelerometer or a multiple axis (e.g. 3 axis) accelerometer. In some examples, the accelerometer may be used to sense various physiologic information, such as but not limited to body position, respiration, sleep, disease burden, and/or other physiologic phenomenon. In some examples, the sensed respiration may be used for timing application of stimulation to treat sleep disordered breathing, to evaluate the severity of the sleep disordered breathing or other disease burdens, the effectiveness of the stimulation therapy, and/or other physiologic information.

In a manner similar to sensing body position, in some examples the accelerometer may be used to sense posture and/or activity based on gross body movements. The accelerometer also may be used to sense at least ballistocardiography, seismocardiography, heart rate (HR), sleep, and/or disease burden. In some such examples, via at least such accelerometer sensing, the disease burden may comprise a cardiovascular burden and/or be determined via a cardiac output and/or cardiac waveform morphology.

In some examples, the on-board sensor 5160 may comprise an electrode formed on the external surface of a housing of the IPG 333, and may be used for sensing impedance in combination with other implanted sensors, such as but not limited to sensors 5168A, 5168B, which may be located on the torso of the patient.

As further described later, sensor 5160 also may be used in combination with sensing elements such as electrodes implanted in the head-and-neck region 320.

In some examples, a stimulation element (e.g. 310R, 310L, 314R, 314R in FIG. 2A) may comprise electrodes which may serve in combination with sensor 5160 (as an electrode) to sense impedance. In some such examples, the sensed impedance may be used to determine respiration, which may be used for at least some of the above-identified purposes and/or other purposes.

In some examples, sensed impedance may indicate a degree of upper airway patency. For example, a smaller cross-sectional upper airway, which reflects less upper airway patency, may be sensed as a lower impedance.

Conversely, a larger cross-sectional upper airway, which reflects more upper airway patency, may be sensed as a higher impedance. Accordingly, maximal patency (measured as a higher impedance) may general correspond to periods of stimulation (for a hypoglossal nerve and/or IHM-innervating nerve) or correspond to peak expiration of a respiratory cycle. Meanwhile, minimal patency (measured as a lower impedance) generally corresponds to inspiration, just prior to inspiration, or the onset of stimulation for hypoglossal nerve and/or IHM-innervating nerve.

In some examples, the on-board sensor 5160 may comprise an ECG sensor or may comprise an electrode, which when used in combination with other electrodes (e.g. 5168A, 5168B), may be used to sense electrocardiogram (ECG) information.

As further shown in FIG. 26, in some example implementations the example arrangement 5100 may comprise a sensor lead 5164 which supports a sensor 5166, which in turn in some examples may comprise a pressure sensor to measure differential pressure or other forms of pressure. Among other physiologic parameters, the pressure sensor may be used to sense respiration, which may be used for at least some of the above-identified purposes and/or other purposes. In some examples, the sensor 5166 may sense physiologic parameters other than pressure.

It will be understood that the on-board sensor 5160 may comprise multiple types of sensors, at least some of which are described above, such as but not limited to impedance sensors, accelerometer(s), etc. In some examples in which the on-board sensor 5160 is implemented, the lead 5164 may be omitted such that the IPG 333 may comprise a leadless sensing arrangement.

In some examples, the example arrangement 5100 may be implemented via at least some external sensors relating to at least some of the sensing types, modalities, physiologic parameters, etc. which were described above as being implemented via implantable sensors.

FIG. 27 is a diagram schematically representing an example arrangement 5170 comprising an example device for, and/or example method of, communication between an implantable medical device (IMD) 5171 and a patient remote control 5172. In some examples, the implantable medical device 5171 may comprise an implantable pulse generator (IPG) (e.g. 333 in FIG. 2A, etc.), microstimulator (e.g. 1133 in FIG. 17A), or the like and is therefore applicable to example implementations throughout the present disclosure.

The patient remote control 5172 comprises inputs to change stimulation strength settings, activate or deactivate therapy, etc. The patient remote controls 5172 also may receive control data, sensed data, therapy data, and/or other data from the IMD 5171. The patient remote control 5172 may communicate wirelessly with the IMD 5171 via telemetry or other wireless communication protocols. At least some aspects of initiating, terminating, adjusting stimulation settings and/or other settings of the IMD 5171 will be further described later in association with various examples throughout the present disclosure.

In some examples, the example arrangement 5170 may comprise one example implementation of the care engine 10000 (FIG. 29A), control portions 10500, 10528, 10600 (FIG. 29B, 29C, 29E), and/or user interface 10520 (FIG. 29D), as described later. With this in mind, the patient remote control 5172 may comprise one example implementation of the remote control 10530 in FIG. 29C and/or of the patient remote control 10640 in FIG. 29E.

In some examples, at least some functions of the patient remote control 5172 may be implemented via the mobile device 10620 (and patient app 1063) of FIG. 29E and vice versa.

As previously noted in connection with at least FIG. 2A, in some examples an upper airway patency-related nerve may comprise an IHM-innervating in addition to, or instead of, a hypoglossal nerve.

In some examples, an IHM-innervating nerve may comprise a nerve or nerve branch which innervates (directly or indirectly) at least one infrahyoid muscle, which may sometimes be referred to as an infrahyoid strap muscle. In some examples, IHM-innervating nerves/nerve branches extend from (e.g. originates) from a nerve loop called the ansa cervicalis (AC) or the “AC loop nerve”, which stems from the cervical plexus, e.g. extending from cranial nerves C1-C3. Accordingly, in some examples, at least some IHM-innervating nerves may correspond to an ansa cervicalis (AC)-related nerve in the sense that such nerves/nerve branches (e.g. IHM-innervating nerves) do not form the AC loop nerve but extend from the AC loop nerve. At least because the AC loop nerve is the origin for some nerves which innervate muscles other than the infrahyoid muscles, some AC-related nerves do not comprise IHM-innervating nerves.

Moreover, it will be understood that in some examples, stimulation applied to a portion (e.g., superior root) of the AC loop nerve (and/or to nerves from which the AC loop nerve originates) may activate IHM-innervating nerves/nerve branches, which extend from the AC loop nerve. However, implementing stimulation (e.g. to influence upper airway patency) occurring at more proximal locations, such as along the superior root of the AC loop nerve may be more complex because of the number/type of different nerves and number/type of different muscles innervated via a superior root of the AC loop nerve such that selective activation of a particular infrahyoid muscle (via stimulation along the superior root) may be quite challenging in some circumstances.

With this background in mind, FIG. 28 is a diagram 9900 schematically representing patient anatomy and providing further details regarding example devices and/or example methods for stimulating an IHM-innervating nerve and/or hypoglossal nerve. As shown in FIG. 28, diagram 9900 includes a side view schematically representing an AC-main nerve 9915, in context with a hypoglossal nerve 9905 and with cranial nerves C1, C2, C3. As shown in FIG. 28, portion 9929A of the AC-main nerve 9915 (e.g. a portion or trunk connecting to the AC loop nerve 9919) extends anteriorly from a first cranial nerve C1 with a segment 9917 running alongside (e.g. coextensive with) the hypoglossal nerve 9905 for a length until the AC-main nerve 9915 diverges from the hypoglossal nerve 9905 to form a superior root 9925 of the AC-main nerve 9915, which forms part of the AC loop nerve 9919. A portion of the hypoglossal nerve 9905 extends distally to innervate the genioglossus muscle 9904. As further shown in FIG. 28, the superior root 9925 of the AC-main nerve 9915 extends inferiorly (i.e. downward) until reaching near bottom portion 9918 of the AC loop nerve 9919, from which the AC loop nerve 9919 extends superiorly (i.e. upward) to form an lesser root 9927 (i.e. inferior root) which joins to the second and third cranial nerves, C2 and C3, respectively and via portions 9929B, 9929C.

As further shown in FIG. 28, several branches 9931 extend off the AC loop nerve 9919, including branch 9932 which innervates the omohyoid muscle group 9934, branch 9942 which innervates the sternothyroid muscle group 9944 and at least a portion (e.g. inferior portion) of the sternohyoid muscle group 9954.

Another branch 9952, near bottom portion 9918 of the AC loop nerve 9919, innervates at least a portion (e.g. superior portion) of the sternohyoid muscle group 9954. In some examples, the collective arrangement of the AC-main nerve 9915 (including at least superior root 9925 of the AC loop nerve 9919) and its related branches (e.g. at least 9932, 9942, 9952) when considered together, or any of those elements individually, may sometimes be referred to as an IHM-innervating nerve 9916. It will be further understood that at least one such IHM-innervating nerve 9916 is present on both sides (e.g. right and left) of the patient's body.

In some examples, stimulation of the superior root 9925 of AC loop nerve 9919 and/or at least some of the branches 9931 extending from the AC loop nerve 9919, may influence upper airway patency. However, in some examples, upper airway patency also may be increased and/or maintained by directly stimulating the above-identified muscle groups, such as the omohyoid, sternothyroid, and/or sternohyoid muscle groups. Accordingly, in some examples, such stimulation also may comprise stimulation of just a nerve portion(s), just muscle portion(s), a combination of nerve portion(s) and muscle portion(s), a neuromuscular junction of nerve portion(s) and muscle portion(s), and combinations thereof. Among other effects, in some examples stimulation of such nerves and/or muscles (and/or neuromuscular junctions, combinations, etc.) may act to bring the larynx inferiorly, which may increase upper airway patency.

Stimulation may be delivered to many different locations of an IHM-innervating nerve 9916/nerve branches. Of these various potential stimulation locations, FIG. 28 generally illustrates three example stimulation locations A, B, and C. A stimulation element may be placed at all three of these locations or just some (e.g. one or two) of these example stimulation locations. At each location, a wide variety of types of stimulation elements (e.g. cuff electrode, axial array, paddle electrode, etc.) may be implanted depending on the particular delivery path, method, etc. For example, any one or a combination of the various example stimulation elements (and associated manner of access, delivery, etc.) described in association with at least FIGS. 1A-25 may be used to deliver such stimulation.

In some such examples, a scale of the various stimulation elements, anchors, access tools, and/or other elements in some of the examples in FIGS. 1A-25L may be reduced to accommodate a generally smaller diameter of the IHM-innervating nerve/nerve branches 9916 as compared to some other nerve portions, such as at least some portions of the hypoglossal nerve.

With further reference to FIG. 28, at each example stimulation A, B, C, a stimulation element may be delivered subcutaneously, intravascularly, etc. At each stimulation location, in some examples the stimulation element may comprise a microstimulator.

In some example implementations, a stimulation element may be percutaneously delivered to a position to be in stimulating relation to the upper airway patency-related muscle. In some such examples, a percutaneous access point may be formed and located intermediate between a hyoid bone and a sternum and lateral to a midline. As with other implantation methods described herein, in some examples the implantation may comprise monitoring nerves during the percutaneous delivery and doing so via a nerve integrity monitor (NIM) in some examples. However, as with some other implantation methods described herein (e.g. at least FIGS. 2A-2D), in some examples, the implantation may omit monitoring nerves during the implantation, at least with regard to use of a NIM to distinguish between protractor and retractor muscles of the genioglossus muscle when identifying a target nerve location at or near which a stimulation element is to be implanted.

It will be understood that these example stimulation locations A, B, C are not limiting and that other portions along the IHM-innervating nerve 9916/nerve branches may comprise suitable stimulation locations, depending on the particular objectives of the stimulation therapy, on the available access/delivery issues, etc.

Among the different physiologic effects resulting from stimulation of the various portions of the IHM-innervating nerve 9916/nerve branches (and/or innervated muscle portions, neuromuscular junctions, etc.), in some examples stimulation of nerve branches which cause contraction of the sternothyroid muscle and/or the sternohyoid muscle may cause the larynx to be pulled inferiorly, which in turn may increase and/or maintain upper airway patency in at least some patients. Such stimulation may be applied without stimulation of the hypoglossal nerve or may be applied in coordination with stimulation of the hypoglossal nerve 9905.

FIG. 29A is a block diagram schematically representing an example care engine 10000. In some examples, the sleep disordered breathing (SDB) care engine 10000 may form part of a control portion 10500, as later described in association with at least FIG. 29B, such as but not limited to comprising at least part of the instructions 10511. In some examples, the SDB care engine 10000 may be used to implement at least some of the various example devices and/or example methods of the present disclosure as previously described in association with FIGS. 1A-28 and/or in later described examples devices and/or methods, such as FIGS. 29B-31. In some such examples, the SDB care engine 10000 may comprise a sensing engine 10020 and/or a stimulation engine 10030. In some examples, the sensing engine 10020 may be implemented via at least some of the sensing elements, sensor types/modalities, etc. as described in association with at least FIGS. 1B, 26, 31 and throughout the various examples of the present disclosure. In some examples, the stimulation engine 10030 may be implemented via at least some of the stimulation elements (e.g. stimulation portions, electrode arrays, cuff electrodes, paddle electrodes, axial leads/electrodes, IPG 333 or 1133, etc.), example methods, described throughout the various examples of the present disclosure. In some examples, the SDB care engine 10000 (FIG. 29A) and/or control portion 10500 (FIG. 29B) may form part of, and/or be in communication with, the example arrangements, stimulation elements, sensing elements, microstimulators, pulse generators, etc. such as a portion of the devices and methods described in association with at least FIGS. 1A-28 and/or the later described examples, such as FIGS. 29B-31.

It will be understood that various functions and parameters of SDB care engine 10000 may be operated interdependently and/or in coordination with each other, in at least some examples.

FIG. 29B is a block diagram schematically representing an example control portion 10500. In some examples, control portion 10500 provides one example implementation of a control portion forming a part of, implementing, and/or generally managing the example arrangements, the stimulation elements, sensing elements, microstimulators, pulse generators, control portion, instructions, engines, functions, parameters, and/or methods, as described throughout examples of the present disclosure in association with FIGS. 1A-29A and/or 29C-31. In some examples, control portion 10500 includes a controller 10502 and a memory 10510. In general terms, controller 10502 of control portion 10500 comprises at least one processor 10504 and associated memories. The controller 10502 is electrically couplable to, and in communication with, memory 10510 to generate control signals to direct operation of at least some of the example arrangements, stimulation elements, sensing elements, microstimulators, pulse generators, control portion, instructions, engines, functions, parameters, and/or methods, as described throughout examples of the present disclosure. In some examples, these generated control signals include, but are not limited to, employing instructions 10511 stored in memory 10510 to at least direct and manage sleep disordered breathing (SDB) care (e.g. sensing, stimulation, etc.) in the manner described in at least some examples of the present disclosure. In some instances, the controller 10502 or control portion 10500 may sometimes be referred to as being programmed to perform the above-identified actions, functions, etc.

In response to or based upon commands received via a user interface (e.g. user interface 10520 in FIG. 29C) and/or via machine readable instructions, controller 10502 generates control signals as described above in accordance with at least some of the examples of the present disclosure. In some examples, controller 10502 is embodied in a general purpose computing device while in some examples, controller 10502 is incorporated into or associated with at least some of the example arrangements, stimulation elements, sensing elements, microstimulators, pulse generators, control portion, instructions, engines, functions, parameters, and/or methods, etc. as described throughout examples of the present disclosure.

For purposes of this application, in reference to the controller 10502, the term “processor” shall mean a presently developed or future developed processor (or processing resources) that executes machine readable instructions contained in a memory or that includes circuitry to perform computations. In some examples, execution of the machine readable instructions, such as those provided via memory 10510 of control portion 10500 cause the processor to perform the above-identified actions, such as operating controller 10502 to implement sleep disordered breathing (SDB) care (e.g. stimulation, sensing, etc.) via the various example implementations as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g. non-transitory tangible medium or non-volatile tangible medium), as represented by memory 10510. The machine readable instructions may include a sequence of instructions, a processor-executable machine learning model, or the like. In some examples, memory 10510 comprises a computer readable tangible medium providing non-volatile storage of the machine readable instructions executable by a process of controller 10502. In some examples, the computer readable tangible medium may sometimes be referred to as, and/or comprise at least a portion of, a computer program product. In other examples, hard wired circuitry may be used in place of or in combination with machine readable instructions to implement the functions described. For example, controller 10502 may be embodied as part of at least one application-specific integrated circuit (ASIC), at least one field-programmable gate array (FPGA), and/or the like. In at least some examples, the controller 10502 is not limited to any specific combination of hardware circuitry and machine readable instructions, nor limited to any particular source for the machine readable instructions executed by the controller 10502.

In some examples, control portion 10500 may be entirely implemented within or by a stand-alone device.

In some examples, the control portion 10500 may be partially implemented in one of the example arrangements, stimulation elements, sensing elements, microstimulators, pulse generators, etc. and partially implemented in a computing resource separate from, and independent of, the example arrangements, stimulation elements, sensing elements, microstimulators, pulse generators, etc. but in communication with such example arrangements, etc. For instance, in some examples control portion 10500 may be implemented via a server accessible via the cloud and/or other network pathways. In some examples, the control portion 10500 may be distributed or apportioned among multiple devices or resources such as among a server, an example arrangement, and/or a user interface.

FIG. 29C is a diagram schematically illustrating at least some example arrangements of a control portion 10528 by which the control portion 10500 (FIG. 29B) can be implemented, according to one example of the present disclosure. In some examples, control portion 10528 is entirely implemented within or by an IPG assembly 10529, which has at least some of substantially the same features and attributes as a pulse generator (e.g. IPG 333 in FIG. 2A, 1133 in FIG. 17A, etc.) as previously described throughout the present disclosure. In some examples, control portion 10528 is entirely implemented within or by a remote control 10530 (e.g. a programmer) external to the patient's body, such as a patient control 10532 and/or a physician control 10534. Patient remote control 5172 in FIG. 27 may comprise one example implementation of the patient control 10532. In some examples, the control portion 10500 is partially implemented in the IPG assembly 10529 and partially implemented in the remote control 5172 (at least one of patient control 10532 and physician control 10534).

In some examples, control portion 10500 includes, and/or is in communication with, a user interface 10520 as shown in FIG. 29D. In some examples, user interface 10520 forms part or and/or is accessible via a device external to the patient and by which the therapy system may be at least partially controlled and/or monitored. The external device which hosts user interface 10520 may be a patient remote (e.g. 10532 in FIG. 29C), a physician remote (e.g. 10534 in FIG. 29C) and/or a clinician portal. In some examples, user interface 10520 comprises a user interface or other display that provides for the simultaneous display, activation, and/or operation of at least some of the example arrangements, stimulation elements, sensing elements, microstimulators, pulse generators, control portion, instructions, engines, functions, parameters, and/or methods, etc., as described in association with FIGS. 1A-29C and 30-31. In some examples, at least some portions or aspects of the user interface 10520 are provided via a graphical user interface (GUI), and may comprise a display 10524 and input 10522.

In support of, and/or as an example implementation of, the examples of the present disclosure described in association with at least FIGS. 1A-29D and 30-31, FIG. 29E is a block diagram 10600 which schematically represents some example implementations by which an implantable device (IMD) 10610 (e.g. IPG 333, 1133, sensors, microstimulators, etc.) may communicate wirelessly with external devices outside the patient. As shown in FIG. 29E, in some examples, the IMD 10610 may communicate with at least one of patient app 10630 on a mobile device 10620, a patient remote control 10640, a clinician programmer 10650, and a patient management tool 10660. Patient remote control 5172 in FIG. 27 may comprise one example implementation of the patient remote control 10640. The patient management tool 10660 may be implemented via a cloud-based portal 10662, the patient app 10630, and/or the patient remote control 10640. Among other types of data, these communication arrangements enable the IMD 10610 to communicate, display, manage, etc. patient therapy information as well as to allow for adjustment to the various elements, portions, etc. of the example devices and methods if and where needed.

FIG. 30 is a block diagram schematically representing a care engine 2500 of a control portion. In some examples, the care engine 2500 may comprise an example implementation of, and/or at least some of substantially the same features and attributes as, any of the stimulation elements, stimulation portion, stimulation leads, pulse generators (implantable and/or external), the control portions, and/or other components, aspects, etc. of the present disclosure as described in association with at least FIGS. 1A-29E, 31. Accordingly, the various functions and parameters of the care engine 2500 may be implemented in a manner supportive of, and/or complementary with, the various functions, parameters, portions, etc., of any of the devices and control portions and/or various functions, parameters, portions, etc., relating to stimulation throughout examples of the present disclosure.

As shown by FIG. 30, in some examples, via target tissue parameter 2510, stimulation may be delivered to and/or mechanically maneuvering may be applied to select target tissue such as, but not limited to, upper airway patency-related tissue.

In some examples, the upper airway patency-related tissue may comprise a hypoglossal nerve and/or muscle (e.g., genioglossus muscle) innervated by the hypoglossal nerve to cause contraction of at least the protrusion muscles and to thereby cause protrusion of the tongue to increase and/or maintain upper airway patency. In some examples, the upper airway patency-related tissue may comprise IHM-innervating nerves, as previously described in association with at least FIG. 28.

In some examples, in addition to or instead of selecting different tissue for stimulation and/or for mechanical maneuvering, the target tissue parameter 2510 may comprise adjusting care parameters (e.g., stimulation parameters) via selecting between (or using a combination of) various locations along a nerve such as stimulating multiple different sites along a particular nerve.

In some examples, in addition to or instead of selecting different nerves for stimulation and/or for mechanical maneuvering, the target tissue parameter 2510 may comprise adjusting care parameters via selecting between (or using a combination of) different fascicles within a particular nerve in order to selectively stimulate target efferent fibers while omitting (or minimally impacting) stimulation of other, non-target fibers and/or to selectively stimulate target efferent fibers while omitting (or minimally impacting) stimulation of other, non-target fibers.

In some examples, the care engine 2500 may implement stimulation according to a bilateral parameter 2512 in which stimulation is applied to target tissue on both sides (e.g., left and right) of the patient's body. In some such examples, the bilateral stimulation may be delivered to the same target tissue (e.g., hypoglossal nerve and/or IHM-innervating nerve) on both sides of the body.

However, in some examples, the bilateral stimulation may be delivered to different target tissue (e.g., hypoglossal nerve, IHM-innervating nerve) such as stimulating one nerve (e.g., hypoglossal nerve) or tissue on a left side of the body while stimulating another nerve (e.g., IHM-innervating nerve) or tissue on a right side of the body, or vice versa. In some examples, in which CSA may be treated, such as part of treating multi-type sleep apnea (e.g., both OSA and CSA), stimulation of a phrenic nerve (or diaphragm muscle) may be included in a bilateral stimulation method to implement the stimulation aspects directed to treating the central sleep apnea.

In some examples, the bilateral parameter 2512 may be implemented in a manner complementary with the alternating parameter 2532, simultaneous parameter 2534, or demand parameter 2536 of multiple function 2530, as further described below.

In some examples, the care engine 2500 may comprise a multiple function 2530 by which various care parameters may be implemented in dynamic arrangements. In some such examples, the care engine 2500 may comprise an alternating parameter 2532 by which care provided to one target tissue (e.g., hypoglossal nerve) may be alternated with care provided to at least one other target tissue (e.g., IHM-innervating nerves). However, the alternating parameter 2532 also may be applied in combination with the bilateral parameter 2512 to apply care to the target tissue (or different target tissue) on opposite sides of the body in which care may be applied on a left side of the body and then applied on the right side of the body in an alternating manner. As used herein, applying or providing care or SDB care to target tissue may include, but is not limited to, applying stimulation.

In some examples, the care engine 2500 may comprise a simultaneous parameter 2534 by which care may be applied simultaneously to at least two different target tissues. In some examples, the at least two different target tissues comprise two different tissues, such as (but not limited to) the hypoglossal nerve and the IHM-innervating nerves. In some examples, the at least two different target tissues may comprise two different locations along the same tissue or two different fascicles of the same nerve. In some examples, the simultaneous parameter 2534 may apply stimulation per bilateral parameter 2512 simultaneously on opposite sides of the body to the same tissue or different tissue.

In some examples, the care engine 2500 may comprise a demand parameter 2536 by which care may be applied to at least one target tissue on a demand basis. For example, stimulation may be applied to one nerve (e.g., hypoglossal nerve) which may be sufficient to achieve a patient metric (e.g., therapy outcome and/or usage) for most nights, for most sleeping positions (e.g., left and right lateral decubitis, prone), etc., but may become insufficient for some nights (e.g., after consuming alcohol or certain drugs which relax upper airway muscles), some sleeping positions (e.g., supine). In the latter situation, to achieve the target patient metric, via the demand parameter 2536, stimulation of a different nerve (e.g., IHM-innervating nerve) may be implemented in addition to, or instead of, stimulation of the first nerve (e.g., hypoglossal nerve) which was previously being stimulated. In some examples, the first or primary nerve being stimulated may be a nerve (e.g. IHM-innervating nerve) other than the hypoglossal nerve and then the hypoglossal nerve may be stimulated on an on-demand basis.

In some examples, the demand parameter 2536 may be implemented such that one of the left or right hypoglossal nerves is regularly stimulated and then the other respective one of the left and right hypoglossal nerves may be stimulated on an on-demand basis as described above. In some examples, the demand parameter 2536 may be implemented such that one of the left or right IHM-innervating nerve/nerve branches is regularly stimulated and then the other respective one of the left and right IHM-innervating nerve/nerve branches may be stimulated on an on-demand basis as described above.

In some examples, the care engine 2500 also may further implement at least some aspects of the control portion of FIGS. 29A-29E and/or other aspects of the examples of the present disclosure, according to at least one of a closed loop parameter 2520, open loop parameter 2522, and nightly titration parameter 2524.

In some examples, the care engine 2500 comprises a closed loop parameter 2520 to deliver care based on sensed patient physiologic information and/or other information (e.g., environmental, temporal, etc.). In some such examples, via the closed loop parameter 2520 the sensed information may be used to control the particular timing of the care according to respiratory information, in which delivery of stimulation to target tissue is timed relative to, triggered by, or synchronized with specific portions (e.g., inspiratory phase) of the patient's respiratory cycle(s). In some such examples and as previously described, the respiratory information and/or other information used with the closed loop parameter 2520 may be determined via the sensors, devices, sensing portions, as described in association with at least FIG. 1B, FIG. 26, FIG. 31, and/or throughout various examples of the present disclosure.

In some examples, with or without timing care relative to sensed respiratory information, the closed loop mode (2520) may comprise delivering SDB care therapy in response to sensed disease burden, such as the average number of apnea events per a time period (e.g., AHI of average number of apnea events per hour) and/or other therapy outcome metrics (e.g., arousals, patient feedback, Epworth Sleepiness Scale (ESS) and/or other metrics). For example, for some periods of time within a nightly treatment period or over the course of several days/weeks, a patient may experience few SDB events (e.g., apnea events), such that therapy may be not delivered. However, upon the patient beginning to experience SDB at a level high enough to warrant therapy, then via the closed loop parameter 2520, stimulation therapy may be delivered to achieve a therapy outcome.

In some examples, the care engine 2500 comprises an open loop parameter (e.g., 2522 in FIG. 14) by which SDB care (e.g., “use”) is applied without a feedback loop of sensed physiologic information. In some such examples, in an open loop mode the SDB care is applied during a treatment period without (e.g., independent of) information sensed regarding the patient's sleep quality, sleep state, respiratory phase, AHI, etc. In some examples, in an open loop mode the SDB care is applied during a treatment period without (e.g., independent of) particular knowledge of respiratory information.

In some examples, the care engine 2500 comprises a nightly titration parameter 2524 by which an intensity of the SDB therapy may be titrated (e.g., adjusted) to be more intense (e.g. higher stimulation amplitude, greater frequency, and/or greater pulse width) or to be less intense within a nightly treatment period. However, it will be understood that the previously described examples in association with at least FIGS. 1A-29E, 31 may be performed without (e.g., independent of) a nightly titration parameter 2524 and instead be based on titration according to a time period parameter of more than a day, such as supra-day time period. Accordingly, in some examples, guiding therapy per a patient metric may be implemented solely according to time period of more than a nightly treatment period.

In some such examples, such titration may be implemented at least partially based on sleep quality, which may be obtained via sensed physiologic information, in some examples. It will be understood that such examples may be employed with timing stimulation relative to (and/or in response to) sensed respiratory information (e.g., closed loop stimulation) or may be employed without timing stimulation relative to (and/or in response to) sensed respiratory information (e.g., open loop stimulation).

In some examples, at least some aspects of the titration parameter 2524 of the care engine 2500 and/or at least some aspects of titration as generally disclosed throughout FIGS. 1A-29E, 31 in examples of the present disclosure may comprise (and/or may be implemented) in a manner complementary with and/or via at least some of substantially the same features and attributes as described in U.S. Pat. No. 8,938,299, issued on Jan. 20, 2015, and entitled “SYSTEM FOR TREATING SLEEP DISORDERED BREATHING.”

FIG. 31 is a block diagram schematically representing an example arrangement 3100 including patient's body 3102, including example target portions 3110-3134 at which at least some example sensing element(s) and/or stimulation elements may be employed to implement at least some examples of the present disclosure. In some examples, the arrangement 3100 may comprise at least some of substantially the same features and attributes of, may be implemented in a complementary manner with, and/or as an example implementation of, the various examples throughout the present disclosure.

As shown in FIG. 31, patient's body 3102 comprises a head-and-neck portion 3110, including head 3112 and neck 3114. Head 3112 comprises cranial tissue, nerves, etc., and upper airway 3116 (e.g., nerves, muscles, tissues), etc. As further shown in FIG. 31, the patient's body 3102 comprises a torso 3120, which comprises various organs, muscles, nerves, other tissues, such as but not limited to those in pectoral region 3122 (e.g., lungs 3126, cardiac 3127), abdomen 3124, and/or pelvic region 3129 (e.g., urinary/bladder, anal, reproductive, etc.). As further shown in FIG. 15, the patient's body 3102 comprises limbs 3130, such as arms 3132 and legs 3134.

It will be understood that various sensing elements (and/or stimulation elements, stimulation portions, stimulation leads, etc.) as described throughout the various examples of the present disclosure may be deployed within the various regions of the patient's body 3102 to sense and/or otherwise diagnose, monitor, treat various physiologic conditions such as, but not limited to the above-described examples in association with FIGS. 1A-30. In some such examples, a stimulation element 3117 may be located in or near the upper airway 3116 for treating SDB (and/or near other nerves/muscles at the same or different location to treat SDB and/or other conditions) and/or a sensing element 3128 may be located anywhere within the neck 3114 and/or torso 3120 (or other body regions) to sense physiologic information for providing patient care (e.g., SDB, other).

In some examples, at least a portion of the stimulation element 3117 may comprise part of an implantable component/device, such as an IPG whether full sized or sized as a microstimulator. The implantable components (e.g., IPG, other) may comprise a stimulation/control circuit, a power supply (e.g., non-rechargeable, rechargeable), communication elements, and/or other components. In some examples, the stimulation element 3117 also may comprise a stimulation electrode and/or stimulation lead connected to the implantable pulse generator.

Further details regarding a location, structure, operation and/or use of the sensing element 3128, external element(s) 3150, and/or stimulation element 3117 are described above in association with at least FIGS. 1A-30.

In some examples, at least a portion of the stimulation element 3117 may comprise part of an external component/device such as, but not limited to, the external component comprising a pulse generator (e.g., stimulation/control circuitry), power supply (e.g., rechargeable, non-rechargeable), and/other components. In some examples, a portion of the stimulation element 3117 may be implantable and a portion of the stimulation element 3117 may be external to the patient.

Accordingly, as further shown in FIG. 31, the various sensing element(s) 3128 and/or stimulation element(s) 3117 implanted in the patient's body may be in wireless communication (e.g., connection 3137) with at least one external element 3150.

As further shown in FIG. 31, in some examples, the external element(s) may be implemented via a wide variety of formats such as, but not limited to, at least one of the formats 3151 including a patient support 3152 (e.g., bed, chair, sleep mat, other), wearable elements 3154 (e.g., finger, wrist, head, neck, shirt), noncontact elements 3156 (e.g., watch, camera, mobile device, other), and/or other elements 3158.

As further shown in FIG. 31, in some examples, the external element(s) 3150 may comprise one or more different modalities 3170 such as (but not limited to) a sensing portion 3171, stimulation portion 3172, power portion 3174, communication portion 3176, and/or other portion 3178. The different portions 3171, 3172, 3174, 3176, 3178 may be combined into a single physical structure (e.g., package, arrangement, assembly), may be implemented in multiple different physical structures, and/or with just some of the different portions 3171, 3172, 3174, 3176, 3178 combined together in a single physical structure.

Among other such details, in some examples the external sensing portion 3171 and/or implanted sensing element 3128 may comprise an example implementation of, and/or at least some of substantially the same features and attributes as, the examples further described above in association with FIGS. 1A-30.

In some examples, the external stimulation portion 3172 and/or implanted stimulation element 3117 may comprise at least some of substantially the same features and attributes of (and/or an example implementation of) at least the stimulation arrangements, as further described above in association with at least FIGS. 1A-30, and/or other examples throughout the present disclosure.

In some examples, the external power portion 3174 and/or power components associated with implanted stimulation element 3117 may comprise at least some of substantially the same features and attributes of at least the stimulation arrangements (and/or an example implementation of), as further described below in association with at least FIGS. 1A-30 (and specifically examples employing a microstimulator such as, but not limited to, example of FIG. 17A) and/or other examples throughout the present disclosure. In some such examples, the respective power portion, components, etc. may comprise a rechargeable power element (e.g., supply, battery, circuitry elements) and/or non-rechargeable power elements (e.g., battery). In some examples, the external power portion 3174 may comprise a power source by which a power component of the implanted stimulation element 3117 may be recharged.

In some examples, stimulation element 3128 may comprise an implantable stimulation electrode array and related components to receive signals (e.g. for power, control, delivering stimulation, and/or sensing data) from one or more external components to supply power, control, deliverable stimulation, and/or sensing data to the implantable stimulation electrode array and related components. In some such examples, the signals may be communicated via the communication portion 3176.

In some examples, the (wireless) communication portion 3176 (e.g., connection/link at 3137) may be implemented via various forms of radiofrequency communication and/or other forms of wireless communication, such as (but not limited to) magnetic induction telemetry, BT, Bluetooth Low Energy (BLE), near infrared (NIF), near-field protocols, Wi-Fi, Ultra-Wideband (UWB), and/or other short range or long range wireless communication protocols suitable for use in communicating between implanted components and external components in a medical device environment.

Examples are not so limited as expressed by other portion 3178 via which other aspects of implementing medical care may be embodied in external element(s) 3150 to relate to the various implanted and/or external components described above.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

The following examples may comprise at least some of substantially the same features and attributes as, and/or example implementations of, the previously described examples of the present disclosure. The following examples may be implemented alone or together, which may comprise any various complementary combinations.

Example A1. A device, comprising: at least one implantable stimulation element disposed in stimulating relation to at least one hypoglossal nerve portion, and optionally comprising a control portion configured to stimulate, via the at least one stimulation element, the at least one hypoglossal nerve portion.

Example A2. The device of example A1, wherein the at least one stimulation element is configured to be implantable via a first implant-access incision in a patient's body for chronic implantation in a position at least partially overlapping with, or in close proximity to, a sagittal midline in a submental region.

Example A2B. The device of example A1, wherein the device comprises an implantable medical device comprising the control portion and the at least one stimulation element disposed in stimulating relation to the at least one hypoglossal nerve portion.

Example A3. The device of example A1, wherein the at least one stimulation element comprises a first stimulation element and a second stimulation element, and the first stimulation element is positionable on the first side of the body in stimulating relation to at least the first hypoglossal nerve portion and the second stimulation element is positionable on the opposite second side of the body in stimulating relation to at least the second hypoglossal nerve portion.

Example A4. The device of example A3, wherein the first stimulation element comprises a first carrier body on which the at least one electrode is mounted as a plurality of spaced apart electrodes and wherein the second stimulation element comprises a second carrier body on which a plurality of electrodes is mounted, and wherein the implanting comprises the first stimulation element and the second stimulation element which are independently positionable relative to each other within a patient's body relative to a sagittal midline in a submental region of the patient's body.

Example A5. The device of example A4, wherein the electrodes of the first stimulation element are aligned axially along a length of the respective first carrier body and wherein the electrodes of the first stimulation element are aligned axially along a length of the respective first carrier body.

Example A6. The device of example A5, wherein the first and second stimulation components are configured to form an arcuately-shaped implant path within target tissues on each of the first and second sides of the patient's body, with each respective first and second carrier body comprising a flexible, resilient member comprising a pre-formed curvilinear shape prior to, during and after introduction and advancement into, within, and through the implant path.

Example A7. The device of example A5, wherein the first and second stimulation components are configured to form an arcuately-shaped implant path within target tissues on each of the first and second sides of the patient's body, with each respective first and second carrier body comprising a flexible, resilient member comprising a linear shape prior to introduction and advancement into, within, and through the implant path, with the first carrier body taking on a shape and orientation generally corresponding the shape and orientation of the arcuately-shaped implant path.

Example A8. The device of example A4, wherein the proximal end of the first stimulation element is connected to and extends from a first distal lead segment and the proximal end of the second stimulation element is connected to and extends from a second distal lead segment, the first distal lead segment and the second distal lead segment being independently positionable relative to each other and relative to the sagittal midline.

Example A9. The device of example A4, wherein being independent positionable comprises at least a proximal end of the respective first and second stimulation elements having at least two degrees of freedom of movement relative to each other.

Example A10. The device of example A4, wherein the first and second stimulation elements are implantable on their respectively opposite sides of the patient's body and into stimulating relation to the at least one hypoglossal nerve portion comprising each of the first and second stimulation elements being in stimulating relation to target tissues including at least one of: the at least one hypoglossal nerve portion including a protrusor-related nerve portion; at least one genioglossus muscle portion including a protrusor-related muscle portion; and at least one neuromuscular junction of the at least one genioglossus muscle portion and the at least one hypoglossal nerve portion.

Example A11. The device of example A4, wherein each of the first and second stimulation elements are independently positionable in a selectable orientation, according to at least one of: up to three rotational degrees of freedom including a roll parameter, a yaw parameter, and a pitch parameter; and up to three translational degrees of freedom.

Example A12. The device of example A4, wherein each of the respective first and second carrier body comprise a paddle-style carrier body.

Example A13. The device of example A12, further comprises a flexible connector segment electrically and mechanically connected to, and extending between, the first and second stimulation elements relative to each other, and with the flexible connector segment configured to straddle the sagittal midline.

Example A14. The device of example A13, wherein the flexible connector segment comprises an elongate, non-planar member comprising a greatest cross-sectional dimension substantially smaller than a greatest cross-sectional dimension of each respective paddle-style carrier body comprising each of the respective first and second stimulation elements, and wherein the first and second stimulation elements are implantable by varying a distance between a proximal end of the first stimulation element and a proximal end of the second stimulation elements via manipulation of a shape of the flexible connector segment via one or more bends along a length of the flexible connector segment.

Example A15. The device of example A13, wherein each of the first and second stimulation elements are independently positionable in a selectable orientation according to at least one of: up to three rotational degrees of freedom including a roll parameter, a yaw parameter, and a pitch parameter; and up to three translational degrees of freedom.

Example A16. The device of example A3, wherein the at least one stimulation element comprises a single, elongate carrier body including: a first paddle-style arm including a plurality of spaced apart electrodes to define a first stimulation element; a second paddle-style arm opposite the first paddle-style arm including a plurality of spaced apart electrodes to define a second stimulation element; and a common portion interposed between the respective first and second arms and being electrode-free.

Example A17. The device of example A16, wherein the elongate, single carrier body comprises a substantially uniform cross-sectional size, a substantially uniform cross-sectional shape, and a length to position the common portion to straddle the sagittal midline.

Example A18. The device of example A3, wherein the first and second stimulations elements are configured to deliver stimulation via at least one of: across the sagittal midline as at least one first vector between at least one electrode of the first stimulation element on a first side of the patient's body and at least one electrode of the second stimulation element on a second side of the patient's body; and as at least one second vector between at least one first electrode of the first stimulation element on the first side of the patient's body and a different at least one second electrode of the first stimulation element on the first side of the patient's body.

Example A19. The device of example A13, wherein each of the respective first and second stimulation elements are anchorable within patient anatomy to maintain the stimulating relation relative to target tissues, including the at least one hypoglossal nerve portion, via a plurality of anchor elements on at least one of: an outer surface of the respective first and second stimulation elements, and an outer surface of lead portions supporting the respective first and second stimulation elements. Wherein the plurality anchor elements comprise: homogeneous anchor elements, wherein at least a majority of the anchor elements comprise at least one of substantially the same size, shape, position, and orientation relative to each other; or heterogeneous anchor elements, wherein at least a majority of the anchor elements comprise at least one of a different size, different shape, different position, and different orientation relative to each other.

Example A20. The device of example A19, wherein the plurality of anchor elements provide substantially continuous coverage on an outer surface of at least one of: the first and second stimulation elements, and lead portions supporting the first and second stimulation elements.

Example AA1. A method comprising: implanting at least one stimulation element, including at least one electrode, at a submental region in stimulating relation to at least one hypoglossal nerve portion of a patient; and stimulating, via the at least one stimulation element, the at least one hypoglossal nerve portion.

Example AA2. The method of example AA1, wherein the implanting comprises: forming a first implant-access incision which is characterized by at least one of partially overlapping with and being in close proximity to a sagittal midline of the patient's body; and performing the implantation via the first implant-access incision.

Example AA2A. The method of example AA2, wherein the first implant-access incision is characterized by at least one of: extending along the sagittal midline of the patient's body; and extending along a transverse plane of the patient's body and being in close proximity to the sagittal midline of the patient's body.

Example AA3. The method of example claim AA2, wherein the first implant-access incision comprises the sole implant-access incision for implanting the at least one stimulation element.

Example AA4. The method of example AA2, wherein the at least one stimulation element comprises a first stimulation element and a second stimulation element and the at least one hypoglossal nerve portion comprises a first hypoglossal nerve portion and a second hypoglossal nerve portion, and wherein the implanting comprises: implanting the first stimulation element on a first side of the body in stimulating relation to at least the first hypoglossal nerve portion, and implanting the second stimulation element on an opposite second side of the body in stimulating relation to at least the second hypoglossal nerve portion.

Example AA5. The method of example AA 4, wherein the first stimulation element comprises a first carrier body on which the at least one electrode is mounted as a plurality of spaced apart electrodes and wherein the second stimulation element comprises a second carrier body on which a plurality of electrodes is mounted, and wherein the implanting comprises: independently positioning the first stimulation element and the second stimulation element relative to the sagittal midline and relative to each other.

Example AA6. The method of example AA5, wherein the electrodes of the first stimulation element are aligned axially along a length of the respective first carrier body and wherein the electrodes of the first stimulation element are aligned axially along a length of the respective first carrier body.

Example AA7. The method of example AA5, comprising: performing the implanting with a proximal end of the first stimulation element connected to and extending from a first distal lead segment and with the proximal end of the second stimulation element connected to and extending from a second distal lead segment, wherein the first distal lead segment and the second distal lead segment are independently positionable relative to each other and relative to the sagittal midline.

Example AA8. The method of example AA5, wherein the independent positioning comprises performing the independent positioning with at least a proximal end of the respective first and second stimulation elements having at least two degrees of freedom of movement relative to each other.

Example AA9. The method of example AA6, wherein the implanting comprises: forming an arcuately-shaped implant path within target tissues on each of the first and second sides of the patient's body, with each respective first and second carrier body comprising a flexible, resilient member comprising a pre-formed curvilinear shape prior to, during and after introduction and advancement into, within, and through the implant path.

Example AA10. The method of example AA6, wherein the implanting comprises: forming an arcuately-shaped implant path within target tissues on each of the first and second sides of the patient's body, with each respective first and second carrier body comprising a flexible, resilient member comprising a linear shape prior to introduction and advancement into, within, and through the implant path, with the first carrier body taking on a shape and orientation generally corresponding the shape and orientation of the arcuately-shaped implant path.

Example AA11. The method of example AA5, wherein the implanting the first and second stimulation elements, on their respectively opposite sides of the patient's body, into stimulating relation to the at least one hypoglossal nerve portion comprises: implanting each of the first and second stimulation elements to be in stimulating relation to target tissues including at least one of: the at least one hypoglossal nerve portion including a protrusor-related nerve portion; at least one genioglossus muscle portion including a protrusor-related muscle portion; and at least one neuromuscular junction of the at least one genioglossus muscle portion and the at least one hypoglossal nerve portion.

Example AA12. The method of example AA5, wherein each of the respective first and second carrier body comprise a paddle-style carrier body.

Example AA13. The method of example AA12, wherein the implanting comprises performing the implanting with a flexible connector segment electrically and mechanically connecting, and extending between, the first and second stimulation elements relative to each other, and with the flexible connector segment straddling the sagittal midline.

Example AA14. The method of example AA13, wherein the flexible connector segment comprises an elongate, non-planar member comprising a greatest cross-sectional dimension substantially smaller than a greatest cross-sectional dimension of each respective paddle-style carrier body comprising each of the respective first and second stimulation elements, and wherein the implanting comprises: implanting the first and second stimulation elements by varying a distance between a proximal end of the first stimulation element and a proximal end of the second stimulation elements via manipulating a shape of the flexible connector segment via one or more bends along a length of the flexible connector segment.

Example AA15. The method of example AA13, wherein the implanting comprises: independently positioning each of the first and second stimulation elements in a selectable orientation, according to at least one of: up to three rotational degrees of freedom including a roll parameter, a yaw parameter, and a pitch parameter; and up to three translational degrees of freedom.

Example AA16. The method of example AA4, comprising implanting the at least one stimulation element as: a single, elongate carrier body including: a first paddle-style arm including a plurality of spaced apart electrodes to define a first stimulation element; a second paddle-style arm opposite the first paddle-style arm including a plurality of spaced apart electrodes to define a second stimulation element; and a common portion interposed between the respective first and second arms and being electrode-free.

Example AA17. The method of example AA16, wherein the single, elongate carrier body comprises a substantially uniform cross-sectional size, a substantially uniform cross-sectional shape, and a length to position the common portion to straddle the sagittal midline.

Example AA18. The method of example AA4, wherein the stimulating comprises at least one of: delivering stimulation across the sagittal midline as at least one first vector between at least one electrode of the first stimulation element on a first side of the patient's body and at least one electrode of the second stimulation element on a second side of the patient's body; and delivering stimulation as at least one second vector between at least one first electrode of the first stimulation element on the first side of the patient's body and a different at least one second electrode of the first stimulation element on the first side of the patient's body.

Example AA19. The method of example AA13, wherein the implanting comprises: anchoring each of the respective first and second stimulation elements within patient anatomy to maintain the stimulating relation relative to target tissues, including the at least one hypoglossal nerve portion, via a plurality of anchor elements on at least one of: an outer surface of the respective first and second stimulation elements; and an outer surface of lead portions supporting the respective first and second stimulation elements, wherein the plurality of anchor elements comprise: homogeneous anchor elements, wherein at least a majority of the anchor elements comprise at least one of substantially the same size, shape, position, and orientation relative to each other; or heterogeneous anchor elements, wherein at least a majority of the anchor elements comprise at least one of a different size, different shape, different position, and different orientation relative to each other.

Example AA20. The method of example AA19, wherein the anchoring comprises implementing the anchoring with the plurality of anchor elements providing substantially continuous coverage on an outer surface of at least one of: the first and second stimulation elements; and lead portions supporting the first and second stimulation elements.

Example B21. A method comprising: implanting at least one stimulation element, including at least one electrode, at a submental region in stimulating relation to at least one hypoglossal nerve portion; and stimulating, via the at least one stimulation element, the at least one hypoglossal nerve portion.

Example B22. The method of example B21, wherein the implanting comprises forming a first implant-access incision which is characterized by at least one of partially overlapping with, and being in close proximity to, a sagittal midline of the patient's body, and performing the implantation via the first implant-access incision.

Example B23. The method of example B22, wherein the first implant-access incision comprises the sole implant-access incision for implanting the at least one stimulation element.

Example B24. The method of example B22, wherein the implanting comprises visualizing, during the implantation, via the first implant-access incision: the at least one hypoglossal nerve portion; or muscles that are at least one of innervated by, and contain, the at least one hypoglossal nerve portion.

Example B25. The method of example B24, wherein the visualization of the muscles during implantation comprises visualizing portions of the muscles including distal terminal portions of the at least one hypoglossal nerve portion.

Example B26. The method of claim B22, wherein the at least one hypoglossal nerve portion comprises a first hypoglossal nerve portion and a second hypoglossal nerve portion.

Example B27. The method of example B22, wherein both the first and second distal hypoglossal nerve portions are on a same side of the patient's body.

Example B28. The method of example B26, wherein the first hypoglossal nerve portion is on a first side of the patient's body and the second hypoglossal nerve portion is on a second side of the patient's body.

Example B29. The method of example B28, wherein the at least one stimulation element comprises a first stimulation element and a second stimulation element.

Example B30. The method of example B29, wherein the implanting comprises implanting the first stimulation element on the first side of the body in stimulating relation to the first hypoglossal nerve portion, and implanting the second stimulation element on the opposite second side of the body in stimulating relation to the second hypoglossal nerve portion.

Example B31. The method of example B29, wherein the first stimulation element comprises a first array of electrodes and the second stimulation element comprises a second array of electrodes.

Example B32. The method of example B31, wherein the first stimulation element is spaced apart from the second stimulation element along a length of a lead on which the first and second stimulation elements are located.

Example B33. The method of example B22, wherein at least one stimulation element comprises a first stimulation element comprising a first carrier body on which the at least one electrode is mounted as a plurality of spaced apart electrodes.

Example B34. The method of example B33, wherein the at least one stimulation element comprises the first stimulation element and a second stimulation element including a second carrier body on which a plurality of electrodes is mounted.

Example B35. The method of example B33, wherein the implanting comprises implanting the at least one stimulation element to at least partially extend generally in or parallel to a submandibular plane

Example B36. The method of example B33, wherein the electrodes are aligned axially on the first carrier body.

Example B37. The method of example B33, wherein the implanting comprises positioning a first end of the at least one stimulation element closer to a chin and a second end of the at least one stimulation element further from the chin than the first end, wherein the at least one stimulation element extends in a first orientation.

Example B38. The method of example B37, wherein the second end of the at least one stimulation element comprises a distal end, and wherein the first end of the at least one stimulation element comprises a proximal end, which is supported by and extends from a lead body connectable to IPG.

Example B39. The method of example B37, wherein the first end of the at least one stimulation element comprises the distal end, and wherein the second end of the at least one stimulation element comprises the proximal end, which is supported by and extends from the lead body connectable to IPG.

Example B40. The method of example B35, wherein the implanting comprises implanting the at least one stimulation element to align the plurality of electrodes to extend in a first orientation.

Example B41. The method of example B40, wherein the first orientation is generally parallel to the sagittal midline of the patient's body.

Example B42. The method of example B41, wherein the first orientation extends at an acute first angle relative to the sagittal midline between at least one of about 1 degree and 60 degrees, about 1 degree and 45 degrees, and about 1 degree and about 30 degrees.

Example B43. The method of example B40, wherein the at least one stimulation element comprises the first stimulation element and a second stimulation element including a second carrier body on which a plurality of electrodes is mounted, and wherein the implanting comprises implanting the second stimulation element to align the plurality of electrodes to extend in a second orientation, wherein at least one of at least one of: the first and second orientations is generally parallel to the sagittal midline; and the respective first and second orientations diverge from each other by a second angle relative to another between about 5 and 110 degrees.

Example B44. The method of example B35, wherein the implanting the at least one stimulation element comprises forming the first implant-access incision adjacent a chin in the submental region in close proximity to the sagittal midline of the patient's body, forming a first tunnel on a first side of the body to extend in along the first orientation inferior and posterior relative to the first implant access-incision, and advancing, via the first implant-access incision, the at least one stimulation element into and within the first tunnel.

Example B45. The method of example B44, wherein the at least one stimulation element comprises a first stimulation element and a second stimulation element, and wherein the implanting comprises implanting the first stimulation element via the first tunnel, and wherein implanting the at least one stimulation element further comprises: forming a second tunnel on the opposite second side of the body to extend along a second orientation inferior and posterior relative to the first implant-access incision; and advancing, via the first implant access incision, the second stimulation element into and within the second tunnel.

Example B46. The method of example B33, wherein the implanting comprises performing the implanting with the first carrier body of the at least one stimulation element comprising an elongate, generally cylindrical body and the electrodes comprising at least one of ring electrodes and split-ring electrodes extending circumferentially about an external surface of the generally cylindrical body.

Example B47. The method of example B33, wherein the implanting comprises performing the implanting with the first carrier body of the at least one stimulation element including an elongate, generally rectangular sheet comprising a width substantially greater than a thickness of the generally rectangular sheet and comprising a length substantially greater than the width.

Example B48. The method of example B47, wherein the implanting comprises implanting the at least one stimulation element comprising a printed circuit-type construction including the generally rectangular sheet and the electrodes.

Example B49. The method of example B22, wherein the implanting comprising performing the implanting with the at least one stimulation element comprising: a first stimulation element comprising a first carrier body on which the at least one electrode is mounted as a plurality of spaced apart electrodes aligned axially along a length of the first carrier body; and a second stimulation element comprising a second carrier body on which the at least one electrode is mounted as a plurality of spaced apart electrodes aligned axially along a length of the second carrier body.

Example B50. The method of example B49, wherein the implanting comprises independently positioning the first stimulation element and the second stimulation element relative to the sagittal midline and relative to each other, with a proximal end of each respective first and second stimulation elements being connected to, and extending from, separate distal lead segments of a lead body.

Example B51. The method of example B50, wherein the independently positioning comprises performing the independent positioning with at least a proximal end of the respective first and second stimulation elements having at least two degrees of freedom of movement relative to each other.

Example B52. The method of example B51, wherein the at least two degrees of freedom comprises six degrees of freedom of movement.

Example B53. The method of example B51, wherein the implanting comprises performing the implanting with the proximal end of the first stimulation element connected to and extending from a first distal lead segment and with the proximal end of the second stimulation element connected to and extending from a second distal lead segment, wherein the first distal lead segment and the second distal lead segment are independently positionable relative to each other and relative to the sagittal midline.

Example B54. The method of example B49, wherein the implanting comprises performing the implanting with the first and second stimulation elements arranged with a common body portion extending between, and connecting, a proximal end of the first carrier body and a proximal end of the second carrier body, and with the common body portion connected to, and extending from, a lead body connected to an implantable pulse generator.

Example B55. The method of example B54, wherein the implanting comprises implanting the common body portion at least one of in close proximity to the sagittal midline and along the sagittal midline, with the common body portion comprising a shape to orient the first and second stimulation elements at an acute angle relative to each other.

Example B56. The method of example B55, wherein the common body portion comprises a flexible, shape-retaining material to permit selective adjustment of the acute angle and to retain the orientation of the selected angle between the respective first and second stimulation elements.

Example B57. The method of example B54, wherein the implanting comprises performing the implanting with the common body portion configured as a flexible joint enabling multiple rotational degrees of freedom and multiple translational degrees of freedom.

Example B58. The method of example B33, wherein the implanting comprises implanting the first stimulation element to become chronically positioned in an arcuate shape within and among target tissue, with the first stimulation element generally extending in an anterior-posterior orientation and a superior-inferior orientation, the target tissue comprising at least the at least one hypoglossal nerve portion.

Example B59. The method of example B58, wherein a proximal portion of the first stimulation element extends within or generally parallel to the mandibular plane, and a distal portion of the first stimulation element extends a first angle relative to the sub-mandibular plane.

Example B60. The method of example B58, wherein the first angle may comprise about 80 degrees to about 140 degrees, about 85 to about 130 degrees, about 85 to about 120 degrees, about 85 to about 110 degrees, about 85 to about 100 degrees, or about 85 to about 95 degrees.

Example B61. The method of example B60, wherein the distal portion of the first stimulation element extends distally from the proximal portion of the first stimulation element in a superior orientation.

Example B62. The method of example B61, comprising forming an arcuately-shaped implant path within target tissues, wherein the first stimulation element comprises a flexible, resilient member.

Example B63. The method of example B62, wherein the flexible, resilient member comprises a pre-formed curvilinear shape prior to, during and after introduction and advancement into, within, and through the implant path.

Example B64. The method of example B62, wherein the flexible, resilient member comprises a linear shape prior to introduction and advancement into, within, and through the implant path, with the first carrier body taking on a shape and orientation generally corresponding the shape and orientation of the arcuately-shaped implant path.

Example B65. The method of example B62, wherein the implanting comprises advancing and positioning the first stimulation element through and within the arcuately-shaped implant path to cause a concave portion of the first stimulation element to face superiorly and anteriorly, with the concave portion at least partially induced by the arcuate shape of the implant path.

Example B66. The method of example B65, wherein the forming the implant path comprises forming an entry point of the implant path, via the first implant-access incision, and forming a remainder of the implant path to extend in at least one of an anterior orientation and a superior orientation relative to the entry point.

Example B67. The method of example B62, wherein the implanting comprises advancing and positioning the first stimulation element through and within the arcuately-shaped implant path to cause a concave portion of the first stimulation element to face superiorly and posteriorly, with the concave portion at least partially induced by the arcuate shape of the implant path.

Example B68. The method of example B67, wherein the forming the implant path comprises forming an entry point of the implant path, via the first implant-access incision, and forming a remainder of the implant path to extend in at least one of a posterior orientation and a superior orientation relative to the entry point.

Example B69. The method of example B58, wherein the implanting comprises implanting the first stimulation element to position the distal portion to extend superiorly, relative to the sub-mandibular plane, to position electrodes on the distal portion into stimulating relation to at least one of: neuromuscular junctions of hypoglossal nerve portions and genioglossus muscle portions which contribute to protrusion of the genioglossus muscle; hypoglossal nerve portions, including protrusor-related nerve portions; genioglossus muscle portions, including protrusor muscle portions; and a combination of the hypoglossal nerve portions, the genioglossus muscle portions, and the neuromuscular junctions.

Example B70. The method of claim B58, wherein the implanting comprises implanting the first stimulation element to align the electrodes within and among the target tissues to position the electrodes of the distal portion of the first stimulation element in stimulating relation with at least one of: a first nerve portion innervating at least one of a genioglossus oblique (GGo) muscle portion, the genioglossus oblique (GGo) muscle portion, and a neuromuscular junction of the first nerve portion and the genioglossus oblique (GGo) muscle portion; a second nerve portion innervating at least one of a genioglossus horizontal (GGh) muscle portion, the genioglossus horizontal (GGh) muscle portion, and a neuromuscular junction of the second nerve portion and the genioglossus horizontal oblique (GGh) muscle portion; and a third nerve portion innervating at least one of a geniohyoid (GH) muscle portion, the geniohyoid (GH) muscle portion, and a neuromuscular junction of the third nerve portion and the geniohyoid (GH) muscle portion.

Example B71. The method of examples B69 or B70, wherein the proximal portion of the first stimulation element is electrode-free.

Example B72. The method of examples B69 or B70, wherein the first stimulation element comprises a transition portion between the distal portion and the proximal portion, and the transition portion is electrode-free.

Example B73. The method of example B69, wherein the first stimulation element comprises a transition portion between the distal portion and the proximal portion, and where the implanting comprises positioning the transition portion and the proximal portion of the first stimulation element to cause electrodes on at least one of the transition portion and the proximal portion into stimulating relation to at least one of: hypoglossal nerve portions, including protrusor-related nerve portions; genioglossus muscle portions, including protrusor muscle portions; and a combination of the hypoglossal nerve portions and the genioglossus muscle portions.

Example B74. The method of example B58, wherein the implanting comprises exposing some of the electrodes on a first side of the first stimulation element to face anteriorly and some of the electrodes on an opposite second side of the first stimulation element to face posteriorly.

Example B75. The method of example B33, wherein the implanting comprises implanting the first stimulation element with the first carrier body comprising a U-shaped body.

Example B76. The method of example B75, wherein the implanting comprises implanting the U-shaped body of the first stimulation element as comprising: a proximal, closed base portion; and two arms extending, in a spaced apart relationship, from the proximal closed base portion to define an opposite distal open portion.

Example B77. The method of example B75, wherein the implanting comprises implanting the first stimulation element with the electrodes mounted relative to, and exposed at, at least one of an inner surface and an outer surface of the arms of the U-shaped body.

Example B78. The method of example B76, wherein a length of each respective arm is substantially greater than a width of each respective arm.

Example B79. The method of example B76, wherein the implanting comprises positioning and advancing the arms, while maintaining their spaced apart relationship, to encompass a first target tissue portion within and between the respective arms of the first stimulation element.

Example B80. The method of example B79, wherein the target tissue portion comprises at least one genioglossus muscle portion and at least one hypoglossal nerve portion, the at least one genioglossus muscle portion including a protrusor muscle portion and the at least one hypoglossal nerve portion including a protrusor-related nerve portion.

Example B81. The method of example B80, wherein the first target tissue portion comprises at least one neuromuscular junction of the at least one genioglossus muscle portion and the at least one hypoglossal nerve portion, and the at least one neuromuscular junction comprises a protrusor-related neuromuscular junction.

Example B82. The method of example B79, wherein the implanting comprises positioning and advancing the arms, while maintaining their spaced apart relationship, to encompass a first cross-sectional area of the target tissue portion within and between the respective arms of a respective one of the stimulation portion, with the target tissue portion that is substantially greater than a second cross-sectional area of at least one hypoglossal nerve portion extending within and between the respective arms of a respective one of the stimulation portion.

Example B83. The method of example B79, wherein the implanting comprises positioning and advancing the arms, while maintaining their spaced apart relationship, to encompass a first volume of the target tissue portion within and between the respective arms of a respective one of the stimulation portion, with the target tissue portion that is substantially greater than a second volume of at least one hypoglossal nerve portion extending within and between the respective arms of the respective one of the stimulation portion.

Example B84. The method of example B76, wherein the at least some electrodes on inner surface of the first arm are offset, staggered relative to the at least some electrodes on the inner surface of the second arm.

Example B85. The method of example B76, wherein the arms of the U-shaped body extend generally parallel to each other.

Example B86. The method of example B76, wherein at least the closed base portion comprises a flexible, resilient material, and the implanting comprises manipulating a position of one respective arm relative to other respective arm to form a non-parallel angle between the respective arms to facilitate positioning and advancement within the target tissue to encompass target tissue between the respective arms.

Example B87. The method of example B76, wherein the implanting comprises forming at least one implant path within and among target tissue to provide a path and implantation location for the first stimulation element.

Example B88. The method of example B87, wherein the implanting comprises advancing and positioning the first stimulation element through and within the at least one implant path to cause the distal open portion of the first stimulation element to face superiorly and anteriorly with a longitudinal axis of at least one arm of the first stimulation element forming an acute angle relative to a submandibular plane.

Example B89. The method of example B88, wherein the forming the implant path comprises forming an entry point of the implant path, via the first implant-access incision, and forming a remainder of the implant path to extend in at least one of an anterior orientation and a superior orientation relative to the entry point.

Example B90. The method of example B76, wherein the implanting comprises advancing and positioning the first stimulation element through and within the at least one implant path to cause the distal open portion of the first stimulation element to face superiorly and posteriorly, with a longitudinal axis of at least one arm of the first stimulation element forming an acute angle relative to a submandibular plane.

Example B91. The method of example B76, wherein the forming the at least one implant path comprises forming an entry point of the at least one implant path, via the first implant-access incision, and forming a remainder of the at least one implant path to extend in at least one of a posterior orientation and a superior orientation relative to the entry point.

Example B92. The method of example B76, wherein the implanting comprises implanting the first stimulation element to align the electrodes within and among the target tissues to position the electrodes in stimulating relation with at least one of: a first nerve portion innervating at least one of a genioglossus oblique (GGo) muscle portion, the genioglossus oblique (GGo) muscle portion, and a neuromuscular junction of the first nerve portion and the genioglossus oblique (GGo) muscle portion; a second nerve portion innervating at least one of a genioglossus horizontal (GGh) muscle portion, the genioglossus horizontal (GGh) muscle portion, and a neuromuscular junction of the second nerve portion and the genioglossus horizontal oblique (GGh) muscle portion; and a third nerve portion innervating at least one of a geniohyoid (GH) muscle portion, the geniohyoid (GH) muscle portion, and a neuromuscular junction of the third nerve portion and the geniohyoid (GH) muscle portion.

Example B93. The method of example B76, wherein the implanting comprises implanting one arm of the first stimulation element to extend within a first plane more superficial than the target tissues and the other arm of the first stimulation element to extend within a second plane less superficial than the target tissues, wherein the target tissues comprise at least one of: a first nerve portion innervating at least one of a genioglossus oblique (GGo) muscle portion, the genioglossus oblique (GGo) muscle portion, and a neuromuscular junction of the first nerve portion and the genioglossus oblique (GGo) muscle portion; a second nerve portion innervating at least one of a genioglossus horizontal (GGh) muscle portion, the genioglossus horizontal (GGh) muscle portion, and a neuromuscular junction of the second nerve portion and the genioglossus horizontal oblique (GGh) muscle portion; and a third nerve portion innervating at least one of a geniohyoid (GH) muscle portion, the geniohyoid (GH) muscle portion, and a neuromuscular junction of the third nerve portion and the geniohyoid (GH) muscle portion.

Example B94. The method of example B93, comprising prior to the implanting, dissecting tissue layers until at least some of the target tissues are directly visualizable via the first implant-access incision, and forming an implant path within the second plane underneath the respective target tissues.

Example B95. The method of example B94, comprising the implanting comprising slidably inserting the first stimulation element into the implant path.

Example B96. The method of example B33, wherein the implanting comprises performing the implanting on a first side of the patient's body with the first carrier body of the first stimulation element comprising a paddle-style carrier.

Example B97. The method of example B96, wherein the at least one stimulation element comprises a second stimulation element in addition to the first stimulation element, and wherein the second stimulation element comprises a second carrier body as a paddle-style carrier, and further comprising implanting the second stimulation element on an opposite second side of the patient's body.

Example B98. The method of example B97, wherein the implanting comprises performing the implanting with a flexible connector segment electrically and mechanically connecting, and extending between, the first and second stimulation elements relative to each other, and with the flexible connector segment straddling the sagittal midline.

Example B99. The method of example B98, wherein the flexible connector segment comprises an elongate, non-planar member and wherein the implanting comprising implanting the first and second stimulation elements includes varying a distance between a proximal end the first stimulation element and a proximal end of the second stimulation elements via manipulating the shape of the flexible connector segment via one or more bends along a length of the flexible connector segment.

Example B100. The method of example B99, wherein the flexible connector segment comprises a plurality of elongate electrical conductors within an insulative jacket, while omitting stimulation pulse generating circuitry.

Example B101. The method of example B97, wherein the implanting comprises performing the implanting including at least one of positioning and orienting the first stimulation element and the second stimulation element relative to at least one of each other and a sagittal midline within the patient's body for chronic implantation.

Example B102. The method of example B101, wherein the implanting comprises performing the positioning and orienting, independently for each of the first and second stimulation elements, according to at least one of: up to three rotational degrees of freedom; and up to three translational degrees of freedom.

Example B103. The method of example B102, wherein the performing the implanting, according to the three rotational degrees of freedom, via at least one of: a roll parameter; a yaw parameter; and a pitch parameter.

Example B104. The method of example B103, wherein the implanting comprises performing the implanting with the flexible connector segment being made of shape-retaining material to the position and orientation of the first and second stimulation elements relative to each other to be selectably, retained for chronic implantation.

Example B105. The method of example B101, wherein the implanting comprises performing the implanting with the flexible connector segment being made of a flexible, resilient material and maintaining the position and orientation of the first and second stimulation elements relative to each other for chronic implantation via anchoring at least one of the flexible connector segment, the first stimulation element, and the second stimulation element.

Example B106. The method of example B101, comprising performing the implanting via at least one of positioning and orienting with the flexible connector segment comprising a hinge-less flexible connector segment.

Example B107. The method of example B101, performing the implanting via at least one of positioning and orienting with each first and second stimulation element comprising a hinge-less carrier body.

Example B108. The method of example B101, wherein the positioning and orienting comprises implanting each first and second stimulation element to be rotated about an anterior-posterior orientation to cause the electrodes of each respective stimulation element to face at least partially medially toward each other along a medial-lateral orientation with the target tissues interposed between the respective first and second stimulation elements.

Example B109. The method of example B108, wherein the stimulating comprises at least one of: delivering stimulation across the sagittal midline as at least one first vector between at least one electrode of the first stimulation element on a first side of the patient's body and at least one electrode of the second stimulation element on a second side of the patient's body; and delivering stimulation as at least one second vector between at least one first electrode of the first stimulation element on the first side of the patient's body and a different at least one second electrode of the first stimulation element on the first side of the patient's body.

Example B110. The method of example B108, wherein the at least one of positioning and orienting comprises causing the respective first and second stimulation elements to at least partially extend in superior orientation in which respective faces of the respective first and stimulation elements form an acute angle relative to each other.

Example B111. The method of example B22, wherein at least one stimulation element comprises a single carrier body on which the at least one electrode is mounted as a plurality of spaced apart electrodes, wherein the single carrier body comprises an elongate flexible member.

Example B112. The method of example B111, wherein the elongate flexible member comprises at least one of a substantially uniform cross-sectional shape and a substantially uniform size, which extend substantially an entire length of the elongate flexible member.

Example B113. The method of example B112, wherein the cross-sectional shape comprises a rounded rectangular shape having a width substantially greater than a thickness.

Example B114. The method of example B111, wherein the elongate flexible member comprises: a first arm comprising a first group of the electrodes to define the first stimulation element; an opposite second arm comprising a second group of the electrodes to define the second stimulation element; a common portion interposed between the respective first and second arms, wherein the implanting comprises: implanting the first arm on a first side of the patient's body and the second arm on an opposite second side of the patient's body with the common portion of the carrier body extending generally transversely across the sagittal midline.

Example B115. The method of example B114, wherein at least the common portion comprises a substantially uniform cross-sectional shape and a substantially uniform cross-sectional size.

Example B116. The method of example B114, wherein the common portion includes: a first end portion including a first transition portion connected to the first arm; and a second end portion including a second transition portion connected to the second arm.

Example B117. The method of example B116, wherein each respective first and second transition portion comprises a greater flexibility than the common portion.

Example B118. The method of claim B116, the common portion comprises a central portion and wherein at least one of the central portion and the respective transition portions are flexibly biased to cause the first and second arms to extend at an angle of about 45 and 180 degrees relative to each other to position the respective first and second stimulation elements into stimulating relation to target upper airway patency-related tissues on the first side of the patient's body and the second side of the patient's body, respectively.

Example B1119. The method of example B1116, wherein the implanting comprises selectively bending the first and second transition portions to cause the first arm to extend at an angle between 90 and 180 degrees relative to the second arm to position each of the respective first and second group of electrodes into stimulating relation to target upper airway patency-related tissues on the first side of the patient's body and the second side of the patient's body, respectively.

Example B120. The method of example B1116, wherein each transition portion comprises a first arcuate surface to face the upper airway patency-related tissues and which is non-recessed along its arc length.

Example B121. The method of example B120, wherein the first surface of each transition portion is non-recessed relative to a first surface of each respective arm and relative to a first surface of the common portion.

Example B122. The method of example B120, wherein each transition portion comprises less than about 10 percent to about 30 percent change in curvature along its arc length.

Example B123. The method of example B122, wherein the curvature along the first surface of each transition portion is less than 10 percent different from a curvature along at least one of an arc length of the common portion and along an length of the respective arm.

Example B124. The method of example B118, wherein at least the transition portions comprise a shape-retaining material, and wherein the implanting comprises maintaining, via the shape-retaining material, the selectively bent configuration of the elongate flexible member of the first carrier body.

Example B125. The method of example B1114, wherein the first carrier body comprises a resilient material.

Example B126. The method of example B114, wherein the common portion omits at least one of: a power element; a wireless communication element; and circuitry.

Example B127. The method of example B114, wherein the common portion is electrode-free.

Example B128. The method of example B22, wherein the implanting comprises performing the implanting with the at least one stimulation element as a plurality of first stimulation elements spaced apart along at least one distal lead segment of a stimulation lead body.

Example B129. The method of example B128, wherein the implanting comprises implanting the plurality of first stimulation elements on a first side of the patient's body with the at least one distal segment comprising a first distal lead segment on which the respective first stimulation elements are located.

Example B130. The method of example B129 wherein the first distal lead segment comprises intermediate lead segments connecting, and extending between, adjacent first stimulation elements.

Example B131. The method of example B130, wherein a size and shape of the first stimulation elements and of the intermediate lead segments together form a generally cylindrical shape.

Example B132. The method of example B130, wherein the intermediate lead segments comprise a greatest cross-sectional dimension substantially less than a greatest cross-sectional dimension of the first stimulation elements.

Example B133. The method of example B128, wherein the at least one stimulation element comprises a plurality of second stimulation elements spaced apart along a second distal lead segment of the at least one distal lead segment, and wherein the implanting comprises: implanting the second distal lead segment, including the plurality of second stimulation elements, on an opposite second side of the patient's body.

Example B134. The method of example B133, wherein the first distal lead segment is separate from, and independent of, the second distal lead segment with both of the first and second distal lead segments extending from a bifurcation portion of a lead body.

Example B135. The method of example B133, wherein the second distal lead segment is connected to, and extends distally from, the first distal lead segment with a transition lead segment interposed between the first distal lead segment and the second distal lead segment, and wherein the implanting comprises straddling the transition portion across the sagittal midline of the patient's body.

Example B136. The method of example B133 wherein the second distal lead segment comprises intermediate lead segments connecting, and extending between, adjacent second stimulation elements.

Example B137. The method of example B128, wherein the implanting comprises positioning and orienting each first stimulation element within and among the target tissues on the first side of the patient's body to cause at least some of the first stimulation elements to be in stimulating relation with the target tissues as at least one of: a first nerve portion innervating at least one of a genioglossus oblique (GGo) muscle portion, the genioglossus oblique (GGo) muscle portion, and a neuromuscular junction of the first nerve portion and the genioglossus oblique (GGo) muscle portion; a second nerve portion innervating at least one of a genioglossus horizontal (GGh) muscle portion, the genioglossus horizontal (GGh) muscle portion, and a neuromuscular junction of the second nerve portion and the genioglossus horizontal oblique (GGh) muscle portion; and a third nerve portion innervating at least one of a geniohyoid (GH) muscle portion, the geniohyoid (GH) muscle portion, and a neuromuscular junction of the third nerve portion and the geniohyoid (GH) muscle portion.

Example B138. The method of example B137, wherein the stimulating comprises delivering stimulation as at least one first vector between at least one first stimulation element on the first side of the patient's body and a different at least one first stimulation element on the first side of the patient's body.

Example B139. The method of example B137, wherein the positioning and orienting comprises performing the positioning and orienting according to at least two rotational degrees of freedom and at least two translational degrees of freedom for each first stimulation element.

Example B140. The method of example B139, wherein the least two rotational degrees of freedom comprises three rotational degrees of freedom and the at least two translational degrees of freedom comprises three translational degrees of freedom.

Example B141. The method of example B138, wherein the at least one stimulation element comprises a plurality of second stimulation elements spaced apart along a second distal lead segment of the at least one distal lead segment, and wherein the implanting comprises implanting the second distal lead segment, including the plurality of second stimulation elements, on an opposite second side of the patient's body.

Example B142. The method of example B141, wherein the implanting comprises positioning and orienting each second stimulation element within and among the target tissues on the second side of the patient's body to cause at least some of the second stimulation elements to be in stimulating relation with the target tissues as at least one of: a first nerve portion innervating at least one of a genioglossus oblique (GGo) muscle portion, the genioglossus oblique (GGo) muscle portion, and a neuromuscular junction of the first nerve portion and the genioglossus oblique (GGo) muscle portion; a second nerve portion innervating at least one of a genioglossus horizontal (GGh) muscle portion, the genioglossus horizontal (GGh) muscle portion, and a neuromuscular junction of the second nerve portion and the genioglossus horizontal oblique (GGh) muscle portion; and a third nerve portion innervating at least one of a geniohyoid (GH) muscle portion, the geniohyoid (GH) muscle portion, and a neuromuscular junction of the third nerve portion and the geniohyoid (GH) muscle portion.

Example B143. The method of example B141, wherein the stimulating comprises: delivering stimulation as at least one second vector between at least one second stimulation element on the second side of the patient's body and a different at least one second stimulation element on the second side of the patient's body.

Example B144. The method of example B141, wherein the stimulating comprises delivering stimulation within target tissue across the sagittal midline as at least one third vector between at least one first stimulation elements on the first side of the patient's body and at least one second stimulation element on the second side of the patient's body.

Example B145. The method of example B141, wherein the implanting comprises performing the implanting with a quantity of first stimulation elements on the first side of the patient's body is different from a quantity of second stimulation elements one the second side of the patients' body.

Example B146. The method of example B141, wherein the stimulating comprises at least one of: delivering stimulation across the sagittal midline as at least one first vector between at least one of the first stimulation elements on a first side of the patient's body and at least one of the second stimulation elements on a second side of the patient's body; and delivering stimulation as at least one second vector between at least one first stimulation element on the first side of the patient's body and different at least one first stimulation elements on the first side of the patient's body.

Example B147. The method of example B129, wherein the implanting comprises implanting at least one first stimulation element to extend in a first plane different from a second plane in which at least one first stimulation elements extend upon implantation, wherein a respective one of the first and second planes is more superficial within patient's body and the other respective one of the first and second planes is less superficial within the patient's body.

Example B148. The method of example B22, wherein the at least one stimulation element comprises a first stimulation element, which comprises a first cuff electrode, and the implanting comprises implanting the first cuff electrode to be in stimulating relation to at least one hypoglossal nerve portion on a first side of the patient's body.

Example B149. The method of example B148, wherein the implanting comprises prior to implanting the cuff electrode, inserting a test stimulation tool to identify the at least one hypoglossal nerve portion on which to secure the cuff electrode.

Example B150. The method of example B148, wherein the at least one stimulation element comprises a second stimulation element.

Example B151. The method of example B150, wherein the second stimulation element comprises a second cuff electrode, and wherein the implanting comprises implanting the second cuff electrode to be in stimulating relation to at least one hypoglossal nerve portion on an opposite second side of the patient's body.

Example B152. The method of example B150, wherein the implanting comprises implanting the second stimulation element to be in stimulating relation to at least one hypoglossal nerve portion on an opposite second side of the patient's body.

Example B153. The method of example B152, wherein the second stimulation element comprises a plurality of spaced apart electrodes, which are supported via at least one paddle-style carrier body.

Example B154. The method of example B152, wherein the second stimulation element comprises a plurality of spaced apart electrodes, which are supported via an elongate carrier body on which the electrodes are aligned axially.

Example B155. The method of example B152, wherein the second stimulation element comprises a plurality of second stimulation elements on a distal lead portion in which the respective second stimulation elements are spaced apart from each other along a length of the distal lead portion with distal lead segments interposed between the respective adjacent second stimulation elements.

Example B156. The method of example B152, wherein the second stimulation element comprises a plurality of spaced apart electrodes supported on a U-shaped carrier body.

Example B157. The method of example B152, wherein the implanting of the second stimulation element comprises anchoring the second stimulation element to non-nerve surrounding tissues and the implanting of the first stimulation element as the first cuff electrode comprises self-anchoring relative to the at least one hypoglossal nerve portion.

Example B158. The method of one or more of examples B21-B32, of one or more of examples B33-B57, of one or more of examples B58-B74, of one or more of examples B75-B95, of one or more examples B96-B110, of one or more of examples B111-B127, of one or more of examples B128-B147, and of one or more of examples B148-B157, wherein the implanting comprises implanting the respective at least one first stimulation elements comprises fixating, via at least one first anchor structure, the at least one stimulation element relative to non-nerve tissue on the first side of the body.

Example B159. The method of example B158, wherein the at least one electrode comprises a plurality of spaced apart electrodes, and wherein the fixating comprises implementing the fixating via passive engagement of the at least one first anchor structure relative to the non-nerve tissue on the first side of the body, including locating the first anchor structure at least one of: distally of the electrodes of the at least one stimulation element; proximally of the electrode of the at least one stimulation element; and between at least some of the adjacent respective electrodes of the at least one stimulation element.

Example B160. The method of example B158, wherein the at least one stimulation element comprises a first stimulation element implanted on the first side of the patient's body and a second stimulation element, wherein the implanting comprises implanting the second stimulation element on the second side of the patient's body, and comprising fixating, via at least one second anchor structure, the second stimulation element relative to non-nerve tissue on the opposite second side of the body.

Example B161. The method of example B160, comprising: wherein the at least one electrode of the second stimulation element comprises a plurality of spaced apart electrodes, comprising: implementing the fixating via passive engagement of the at least one second anchor structure relative to the non-nerve tissue on the opposite second side of the body, including locating the second anchor structure at least one of: distally of the electrodes of the second stimulation element; proximally of the electrode of the second stimulation element; and between at least some of the adjacent respective electrodes of the second stimulation element.

Example B162. The method of example B158, wherein the at least one stimulation element comprises a first stimulation element and a second stimulation element, including locating the least one first anchor structure and the at least one second anchor structure at least one of: distally of the array of electrodes of each respective first and second stimulation elements; proximally of the array of electrodes of each respective first and second stimulation elements; and between at least some of the adjacent respective electrodes of each respective first and second stimulation elements.

Example B163. The method of example B158, wherein the at least one stimulation element comprises a first stimulation element on a first side of the patient's body and a second stimulation element on a second side of the patient's body, including locating a third anchor structure on: a flexible connector segment extending between the first and second stimulation elements, which each comprise a paddle-style carrier body on which the electrodes are mounted; or a common body portion extending between the first and second stimulation elements, which each comprise an axial-style carrier body on which the electrodes are mounted.

Example B164. The method of example B158, wherein the at least one stimulation element comprises a plurality of first stimulation elements, and the at least one first anchor structure comprises a plurality of anchor element, and comprising implementing the fixating via passive engagement of the at least one first anchor structure relative to the non-nerve tissue on the first side of the body, including locating the at least one first anchor structure at least one of: distally of all the first stimulation elements; proximally of all first stimulation elements; distally of each first stimulation element; proximally of each first stimulation element; on each first stimulation element; and on at least some of the lead segments extending between adjacent pairs of the respective first stimulation elements.

Example B165. The method of any of examples B158, B159, B160, B161, B162, B163, and B164, wherein the at least one first anchor structure comprises a plurality of anchor elements, wherein the fixating comprises implementing the fixating via each anchor element comprising a flexible, resilient tine which is biased to protrude at least one of at an acute angle outwardly from an external surface of a first stimulation element and from an external surface of a second stimulation element and which is collapsible against the external surface of the respective first and second carrier bodies upon contact with an external structure.

Example B166. The method of example B165, wherein the tines protrude in at least one of a distal orientation and a proximal orientation.

Example B167. The method of example B165, wherein at least some of the tines are mounted in rows, with both a length of the tines and the rows oriented transverse to longitudinal axis of at least one of the at least one stimulation element and a lead segment supporting the at least one stimulation element, at least in a region of the at least one stimulation element or lead segment in which the tines are located.

Example B168. The method of example B165, wherein at least some of the tines are mounted in rows, with both a length of the tines and the rows extending in an orientation which is at an non-perpendicular angle relative to at least one of a longitudinal axis of the lead segment and at least one stimulation element, at least in a region of the at least one stimulation element or lead segment in which the tines are located.

Example B169. The method of example B165, wherein at least some of the tines are mounted in a first orientation in which a first plurality of the tines extend at a first angle and a second orientation in which a second plurality of the tines extend at a different second angle relative to at least one of a longitudinal axis of the lead segment and at least one stimulation element, at least in a region of the at least one stimulation element or lead segment in which the tines are located.

Example B170. The method of any examples B165, B167, B168, and B169, wherein each tine comprises: a fixed end, which is secured relative to at least one of the outer surface of the lead segment and the at least one stimulation element, which corresponds to a leading end of the insertion of tines during lateral slidable insertion of at least one of the lead segment and at least one stimulation element; and an opposite free end which corresponds to a trailing end during lateral slidable insertion of at least one of the lead segment and at least one stimulation element.

Example B171. The method of example B169, wherein a first orientation of the leading ends of the first plurality of tines for lateral slidable insertion and a second orientation of the leading ends of the second plurality of tines for lateral slidable insertion are aligned to converge relative to each other.

Example B172. The method of any of examples B158, B159, B160, B161, B162, B163, and B164, wherein the plurality of anchor elements comprise: homogeneous anchor elements, wherein at least a majority of the anchor elements comprise at least one of substantially the same size, shape, position, and orientation relative to each other; or heterogeneous anchor elements, wherein at least a majority of the anchor elements comprise at least one of a different size, different shape, different position, and different orientation relative to each other.

Example B173. The method of example B172, wherein at least one of: a percentage of anchor elements which are heterogeneous relative to each other comprises at least 55%, 60%, 65%, 70%, 75%, 80%, 85%, 90%, or 95%; and a percentage of anchor elements which are homogeneous relative to each other comprises at least 55%, 60%, 65%, 70%, 75%, 80%, 85%, 90%, or 95%.

Example B174. The method of example B172, wherein each respective anchor element is separate from other respective anchor elements, and wherein a quantity of the plurality of anchor elements is substantially different from, being greater than, at least one of: a quantity of electrodes on at least one of: a single stimulation element of multiple stimulation elements; and all of the stimulation elements for a lead; and a quantity of all the stimulation elements.

Example B175. The method of example B172, wherein at least some of the anchor elements extend outwardly from an external surface of a lead segment by a first distance which is substantially different from, being less than, at least one of a diameter of, a greatest cross-sectional dimension of, or a thickness of the lead body.

Example B176. The method of example B172, wherein at least some of the anchor elements extend outwardly from an external surface of a carrier body of the at least one stimulation element by a first distance which is substantially different from, being less than, at least one of a diameter of, a greatest cross-sectional dimension of, or a thickness of the carrier body.

Example B177. The method of example B172, wherein at least some of the anchor elements comprise a diameter or a greatest cross-sectional dimension which is substantially different from, being less than, a surface area of a respective one of the electrodes of the stimulation element.

Example B178. The method of any one of examples B174, B175, B176, and/or B177, wherein the substantially different quantity or distance comprises a difference of at least one of: at least 3 times, 4 times, 5 times, or 6 times; at least one order of magnitude; at least two orders of magnitude; and at least three orders of magnitude.

Example B179. The method of example B172, wherein the fixating comprises implementing the fixating with at least some of the anchor elements being located on at least one of: lead body segments which extend between adjacent, spaced apart stimulation elements; portions of a carrier body of the stimulation element exposed between adjacent spaced apart electrodes of a stimulation element; a portion of a carrier body of the stimulation element which omits electrodes; a flexible connector segment extending between paddle-type carrier bodies on which the electrodes are mounted; and a common body portion extending between axial-type carrier bodies on which the electrodes are mounted.

Example B180. The method of example B172, wherein the fixating comprises the anchor elements providing substantially continuous coverage on an outer surface of at least one of a lead body or a stimulation element.

Example B181. The method of example B180, wherein the substantially continuous coverage comprises at least about 50 percent coverage, at least about 60 percent coverage, at least about 65 percent coverage, at least about 70 percent coverage, at least about 75 percent coverage, at least about 80 percent coverage, at least about 85 percent coverage, at least about 90 percent coverage, or at least about 95 percent coverage.

Example B182. The method of any one of examples B172, B173, B174, B175, B176, B177, B178, B179, and B180, wherein the coverage is implemented via: rows extending longitudinally along length of at least one of the lead and stimulation element, the rows spaced apart from each other circumferentially, wherein spacing between adjacent rows comprises an arc length of about 5 to about 10 degrees, of about 10 to about 20 degrees, of about 20 to about 30 degrees, of about 30 to about 40 degrees, of about 40 to 50 degrees, of about 50 to about 60 degrees, of about 60 to 70 degrees, of about 70 to about 80 degrees, of about 80 to about 90 degrees, or of about 90 to about 120 degrees.

Example B183. The method of any one of examples B172, B173, B174, B175, B176, B177, B178, B179, and B180, the coverage implemented via: rows extending circumferentially transverse to a longitudinal axis of at least one of the lead and stimulation element, at least in the region in which the rows are located, the rows spaced apart from each other longitudinally, wherein spacing between adjacent rows comprises at least one multiple, at least two multiples, or at least three multiples of a width of each row.

Example B184. The method of any one of examples B172, B173, B174, B175, B176, B177, B178, B179, and B180, the coverage implemented via at least one rows arranged in helical pattern wrapped about at least one of an outer surface of the distal lead segment and the at least one stimulation element.

Example B185. The method of example B172, wherein the fixating comprises implementing the fixating via the anchor elements being located on a distal lead segment distal to a bifurcation portion of the lead body.

Example B186. The method of example B172, wherein the anchor elements occupy the entire space or substantially the entire space between adjacent stimulation elements or adjacent electrodes.

Example B187. The method of any of examples B163, B164, and B172, wherein the anchor elements are spaced apart from each other along one of the lead segments between adjacent stimulation elements by a uniform distance or a non-uniform distance such that a location of each anchor element acts as point of articulation in at least one of positioning and orienting the respective stimulation elements within and among the target tissue.

Example B188. The method of example B172, wherein the anchor elements exhibit a lower kinetic coefficient of friction during implantation and a higher coefficient of friction after implantation.

Example B189. The method of one or more of examples B21-B32, of one or more of examples B33-B57, of one or more of examples B58-B74, of one or more of examples B75-B95, of one or more examples B96-B110, of one or more of examples B111-B127, of one or more of examples B128-B147, and of one or more of examples B148-B157, wherein the implanting comprises: implanting an implantable pulse generator (IPG) in at least one of a torso region and a head-and-neck region, and electrically and mechanically connecting the implantable pulse generator to the at least one stimulation element.

Example B190. The method of example B189, wherein the implantable pulse generator comprises a microstimulator.

Example B191. The method of example B189, wherein the electrically and mechanically connecting comprises implanting a lead body comprising a proximal portion connectable to the IPG and an opposite distal portion connected to, or connectable to, a first distal lead segment from which extends the at least one stimulation element.

Example B192. The method of example B189, wherein the implanting of the IPG comprises implanting the IPG, via the first implant-access incision.

Example B193. The method of example B189, wherein the first implant-access incision comprises the sole implant-access incision for implanting both the at least one stimulation element and the IPG.

Example B194. The method of any of examples B191 or B192, wherein the implanting comprises forming a first tunnel from the first implant-access incision within and through the head-and-neck region, or within and through both of the head-and-neck and the torso region, to a subcutaneous location at which the IPG will be chronically implanted.

Example B195. The method of example B194, comprising introducing and advancing the lead body within and through the first tunnel to extend between the first implant-access incision and the location of chronic implantation of the IPG, and electrically connecting the proximal portion of the lead body to the IPG.

Example B196. The method of example B194, wherein implanting the IPG comprises forming a second implant-access incision in proximity to the location of chronic implantation of the IPG, wherein the first tunnel generally extends between the respective first and second implant-access incisions and the implanting of the lead body comprises using the second implant-access incision during the introducing and advancing of the lead body within and through the first tunnel.

Example B197. The method of example B191, wherein in the configuration in which the distal portion of the lead body is connectable to the first distal lead segment, the distal portion of the lead body is removably connectable relative to the first distal lead segment from which the at least one stimulation extends.

Example B198. The method of example B191, wherein the at least one stimulation element comprises a first stimulation element and a second stimulation element, and wherein in the configuration in which the distal portion of the lead body is already connected to the first distal lead segment from which the first stimulation element extends, the distal portion comprises a second port to which a second distal lead segment is connectable, with the second stimulation element extending from the second distal lead segment.

Example B199. The method of example B198, comprising performing the connecting of the second distal lead segment to the second port distal portion of the lead body at at least one of: generally during the same first surgical procedure in which the first distal lead segment and the first stimulation element are chronically implanted, and in a separate second surgical procedure after completion of the first surgical procedure.

Example B200. The method of example B191, wherein the at least one stimulation element comprises a first stimulation element and a second stimulation element, and wherein in the configuration in which the distal portion of the lead body is connectable to the distal lead segment from which the first stimulation element extends, the distal portion comprises a first port to which the first distal lead segment is connectable and a second port to which a second distal lead segment is connectable, with the second stimulation element extending from the second distal lead segment.

Example B201. The method of example B200, wherein the first and second ports of the distal portion of the lead body extend generally parallel to each other, and positioning a proximal portion of the respective first and second distal lead segments to extend in divergent orientations relative to each other to opposite sides of the body.

Example B202. The method of example B200, wherein the distal portion of the lead body comprises a bifurcation portion from which the respective first and second ports extend in a spaced apart relationship.

Example B203. The method of example B202, wherein each of the first and second distal lead segments have the same first length, with a first length greater than a length of a respective one of the first and second stimulation elements.

Example B204. The method of example B203, wherein the implanting comprises implanting the bifurcation portion in close proximity to the sagittal midline.

Example B205. The method of example B202, wherein the first distal lead segment has a second length which is substantially less than a third length of the second distal lead segment, and wherein the implanting comprises implanting the bifurcation portion laterally from the sagittal midline.

Example B206. The method of one or more of examples B21-B32, of one or more of examples B33-B57, of one or more of examples B58-B74, of one or more of examples B75-B95, of one or more examples B96-B110, of one or more of examples B111-B127, of one or more of examples B128-B147, and of one or more of examples B148-B157, wherein the implanting comprises: performing the implanting with the at least one stimulation element, or a lead segment supporting the at least one stimulation element, comprising a printed circuit-type construction.

Example B207. The method of example B206, wherein the at least one stimulation element comprises a thickness substantially less than a width of first stimulation element.

Example B208. The method of one or more of examples B21-B32, of one or more of examples B33-B57, of one or more of examples B58-B74, of one or more of examples B75-B95, of one or more examples B96-B110, of one or more of examples B111-B127, of one or more of examples B128-B147, and of one or more of examples B148-B157, wherein the stimulating comprises stimulating target tissues including at least one of: a first nerve portion innervating at least one of a genioglossus oblique (GGo) muscle portion, the genioglossus oblique (GGo) muscle portion, and a neuromuscular junction of the first nerve portion and the genioglossus oblique (GGo) muscle portion; a second nerve portion innervating at least one of a genioglossus horizontal (GGh) muscle portion, the genioglossus horizontal (GGh) muscle portion, and a neuromuscular junction of the second nerve portion and the genioglossus horizontal oblique (GGh) muscle portion; and a third nerve portion innervating at least one of a geniohyoid (GH) muscle portion, the geniohyoid (GH) muscle portion, and a neuromuscular junction of the third nerve portion and the geniohyoid (GH) muscle portion.

Example B209. The method of example B208, wherein the stimulating comprises at least one of: applying the stimulation to the target tissues as at least one first stimulation vector among a plurality of electrodes of at least one first stimulation element on a first side of the patient's body; applying the stimulation to the target tissues as at least one second stimulation vector among a plurality of electrodes of the at least one second stimulation element on a second side of the patient's body; and applying the stimulation to the target tissues as at least one third stimulation vector between the first side and the second side of the patient's body among the plurality of electrodes of the at least one first stimulation element on the first side of the patient's body and among the plurality of electrodes of the at least one second stimulation element on the second side of the patient's body.

Example B210. The method of any one of example B208 and B209, wherein the stimulating comprises: applying the stimulation to the target tissues as at least one first stimulation vector among a plurality first stimulation elements on a first side of the patient's body; applying the stimulation to the target tissues as at least one second stimulation vector among a plurality second stimulation elements on a second side of the patient's body; and applying the stimulation to the target tissues as at least one third stimulation vector between the first side and the second side of the patient's body via the respective first stimulation elements on the first side of the patient's body and the respective second stimulation elements on the second side of the patient's body.

Example B211. The method of any one of example B208, B209 and B210, wherein the stimulating comprises at least one of: applying different stimulation thresholds for target tissues on the first side of the patients' body and on the second side of the patient's body; applying different stimulation thresholds for at least one of: the respective different first, second, and third nerve portions; the respective different muscle portions innervated by the respective first, second, and third nerve portions; and the respective neuromuscular junctions associated with the respective first, second, and third nerve portions.

1. A device comprising:
at least one implantable stimulation element to be in stimulating relation to at least one hypoglossal nerve portion, wherein the at least one stimulation element is configured to be implantable via a first implant-access incision in a patient's body for chronic implantation in a position at least partially overlapping with, or in close proximity to, sagittal midline in a submental region.

2. (canceled)

3. The device of claim 1, wherein the at least one stimulation element comprises a first stimulation element and a second stimulation element and the at least one hypoglossal nerve portion comprises at least a first hypoglossal nerve portion and a second hypoglossal nerve portion, and the first stimulation element is positionable on a first side of a body of a patient in stimulating relation to at least the first hypoglossal nerve portion and the second stimulation element is positionable on an opposite second side of the body in stimulating relation to at least the second hypoglossal nerve portion.

4. The device of claim 3, wherein the first stimulation element comprises a first carrier body on which at least one electrode is mounted as a plurality of spaced apart electrodes, and
wherein the second stimulation element comprises a second carrier body on which a plurality of electrodes is mounted, and
wherein the first stimulation element and the second stimulation element are configured to be independently positionable relative to each other within a patient's body relative to a sagittal midline in a submental region of the patient's body.

5. (canceled)

6. (canceled)

7. The device of claim 4, wherein the first and second stimulation elements are configured to form an arcuately-shaped implant path within target tissues on each of the first and second sides of the patient's body, and
wherein each respective first and second carrier body comprises a flexible, resilient member comprising a linear shape prior to introduction and advancement into, within, and through an implant path, with the first carrier body taking on a shape and orientation generally corresponding the shape and orientation of the arcuately-shaped implant path.

8. The device of claim 4, wherein a proximal end of the first stimulation element is connected to and extends from a first distal lead segment and a proximal end of the second stimulation element is connected to and extends from a second distal lead segment, and
wherein the first distal lead segment and the second distal lead segment are configured to be independently positionable relative to each other and relative to the sagittal midline.

9. The device of claim 4, wherein being independent positionable comprises at least a proximal end of the respective first and second stimulation elements having at least two degrees of freedom of movement relative to each other.

10. The device of claim 4, wherein the first and second stimulation elements are configured to be implantable on their respectively opposite sides of the patient's body and into stimulating relation to the at least one hypoglossal nerve portion comprising each of the first and second stimulation elements being in stimulating relation to target tissues including at least one of:
the at least one hypoglossal nerve portion including a protrusor-related nerve portion;
at least one genioglossus muscle portion including a protrusor-related muscle portion; and
at least one neuromuscular junction of the at least one genioglossus muscle portion and the at least one hypoglossal nerve portion.

11. The device of claim 4, wherein each of the first and second stimulation elements are independently positionable in a selectable orientation, according to at least one of:
up to three rotational degrees of freedom including a roll parameter, a yaw parameter, and a pitch parameter; and
up to three translational degrees of freedom.

12. The device of claim 4, wherein each of the respective first carrier body and second carrier body comprise a paddle-style carrier body.

13. The device of claim 12, further comprising a flexible connector segment electrically and mechanically connected to, and extending between, the first and second stimulation elements relative to each other, and with the flexible connector segment configured to straddle the sagittal midline.

14. The device of example 13, wherein the flexible connector segment comprises an elongate, non-planar member comprising a greatest cross-sectional dimension substantially smaller than a greatest cross-sectional dimension of each respective paddle-style carrier body comprising each of the respective first and second stimulation elements, and
wherein the first and second stimulation elements are configured to be implantable by varying a distance between a proximal end of the first stimulation element and a proximal end of the second stimulation elements via manipulation of a shape of the flexible connector segment via one or more bends along a length of the flexible connector segment.

15. The device of claim 13, wherein each of the first and second stimulation elements are configured to be independently positionable in a selectable orientation according to at least one of:
up to three rotational degrees of freedom including a roll parameter, a yaw parameter, and a pitch parameter; and
up to three translational degrees of freedom.

16. The device of claim 3, wherein the at least one stimulation element comprises a single, elongate carrier body including:
a first paddle-style arm including a plurality of spaced apart electrodes to define a first stimulation element;
a second paddle-style arm opposite the first paddle-style arm including a plurality of spaced apart electrodes to define a second stimulation element; and
a common portion interposed between the respective first and second arms and being electrode-free.

17. The device of claim 16, wherein the single, elongate carrier body comprises a substantially uniform cross-sectional size, a substantially uniform cross-sectional shape, and a length to position the common portion to straddle a sagittal midline of the patient's body.

18. The device of claim 3, wherein the first and second stimulations elements are configured to deliver stimulation via at least one of:
across a sagittal midline of the patient's body as at least one first vector between at least one electrode of the first stimulation element on a first side of the patient's body and at least one electrode of the second stimulation element on a second side of the patient's body; and
as at least one second vector between at least one first electrode of the first stimulation element on the first side of the patient's body and a different at least one second electrode of the first stimulation element on the first side of the patient's body.

19. The device of claim 12, wherein each of the respective first and second stimulation elements are configured to be anchorable within patient anatomy to maintain the stimulating relation relative to target tissues, including the at least one hypoglossal nerve portion, via a plurality of anchor elements on at least one of:
an outer surface of the respective first and second stimulation elements, and
an outer surface of lead portions supporting the respective first and second stimulation elements,
wherein the plurality of anchor elements comprise:
homogeneous anchor elements, wherein at least a majority of the anchor elements comprise at least one of substantially the same size, shape, position, and orientation relative to each other; or
heterogeneous anchor elements, wherein at least a majority of the anchor elements comprise at least one of a different size, different shape, different position, and different orientation relative to each other.

20. The device of claim 19, wherein the plurality of anchor elements provide substantially continuous coverage on an outer surface of at least one of: the first and second stimulation elements, and lead portions supporting the first and second stimulation elements.
